(19) United States
(12) Patent Application Publication
(10) Pub. No.: US 2007/0141009 A1
(43) Pub. Date: Jun. 21, 2007

(54) Sirna mediated post-transriptional gene silencing of genes involved in alopecia

(76) Inventor: Shaharyar Khan, North Garden, VA (US)

Correspondence Address: PATREA L. PABST;PABST PATENT GROUP LLP, 400 COLONY SQUARE, SUITE 1200, 1201 PEACHTREE STREET, ATLANTA, GA, 30361, US

(21) Appl. No.: 10/544,320

(22) PCT Filed: Jan. 5, 2004
(86) PCT No.: PCT/US04/00128

§ 371 Date: Aug. 23, 2006

(30) Foreign Application Priority Data
Jan. 3, 2003 (US) 60437842

(51) Int. Cl. A61K 48/00; A61K 8/73

(52) U.S. Cl. 424/70.13; 514/44

(57) ABSTRACT

Compositions and methods for the use of inhibitory nucleic acids, for example small inhibitory ribonucleic acids (siRNA), to adjust, manipulate, prevent, inhibit, interfere, or block the androgen signal transduction pathway in a host cell, for example in a host's hair cell are provided. Aspects of the disclosure provide compositions and methods for interfering with the androgen signal transduction pathway by down regulating the expression of proteins involved in the androgen signal transduction pathway. Exemplary gene targets encoding proteins involved in the androgen signal transduction pathway include but are not limited to isozymes I and II of 5-α reductase, the androgen receptor, aromatase, 3-α-hydroxysteroiddehydrogenase, 3-β-hydroxysteroiddehydrogenase , 3-β-hydroxysteroiddehydrogenase-4-5-isomerase, 17-β-hydroxysteroidoxidoreductase, and steroid sulfatase. In some aspects, the inhibitory nucleic acids, for example siRNAs, interfere with the expression of targeted genes by preventing, reducing, or inhibiting the translation of mRNA transcribed from the targeted gene.

### BACKGROUND

1. Technical Field

The present disclosure relates generally to the methods and compositions for manipulating hair growth or hair loss. In particular, aspects of the present disclosure are directed to methods and compositions that interfere with genetic expression of genes involved in or related to hair loss or hair growth, for example using inhibitory nucleic acids in cases of androgenic alopecia.

2. Related Art

Hair loss in both men and women has garnered much attention from the medical and pharmaceutical industry because of the high demand for effective and reliable treatments by those suffering from this condition. In human beings, hair growth and its renewal are mainly determined by the activity of the hair follicles. Their activity is cyclical and comprises essentially three phases, namely the anagenic phase, the catagenic phase and the telogenic phase. The active anagenic phase or growth phase, which lasts several years and during which the hair grows longer, is followed by a very short and transitory catagenic or involution phase, which lasts a few weeks, and then by a resting phase, known as the telogenic phase, which lasts a few months. At the end of the resting period, the hair falls out and another cycle recommences. The head of hair is thus constantly renewed and, of the approximately 150,000 hairs, which a head of hair contains, at each instant, approximately 10% of them are at rest and will therefore be replaced in a few months.

In a significant number of cases, early hair loss takes place in subjects who are genetically predisposed to the condition. Hair loss affects men in particular and is more particularly androgenetic in character. Hair loss is also referred to as androgenic alopecia or alternatively androgeno-genetic alopecia (AGA).

Because of the link between androgens and hair loss, most hair loss therapies are designed to interfere with androgenic action or to include some form of hormone therapy. Although therapies for hair loss do exist, these therapies are not effective in many individuals, and in some cases are only available to specific genders of suffers.

For example, U.S. Pat. Nos. 5,571,817; 5,670,643 and 6,124,362 disclose a composition and method for producing and using 17β-N-t butylcarbamoyl4-aza-5α-androst-1-en-3-one known generically as finesteride. The efficacy of compounds such as finesteride relies upon their ability to inhibit the action of various proteins involved in androgen mediated hair loss. Finesteride is commercially disturbed under the tradename Propecia® and is a testosterone-5α-reductase inhibitor. 5α-reductase is an enzyme that coverts testosterone into dihydroxy testosterone (DHT). Thus, finesteride decreases the amount of DHT. Finesteride is reported to be less effective in women and may potentially cause serious birth defects if used by pregnant women.

Another well known treatment for hair loss uses a compound known as minoxidil. U.S. Pat. No. 4,596,812 discloses methods of using minoxidil, 6-amino-1,2-dihydro-1-hydroxy-2-imino-4-piperodinopyrimidine, for treating male pattern baldness. Minoxidil is a vasodilator developed for the treatment of high blood pressure. Minoxidil is believed to increase hair growth by increasing the delivery of blood to a desired area. It is generally recognized that not all individuals will respond to minoxidil, and those who do respond require treatment application for life to maintain efficacy. Additionally, minoxidil may cause itchiness and redness of the scalp.

Thus, there is a need for new and effective treatments for androgen related disorders, including hair loss, for both men and women.

There is also a need for new and effective compositions for the treatment of hair loss including the prevention of hair loss or the induction of hair growth.

### SUMMARY

Aspects of the present disclosure are directed to compositions and methods for the use of inhibitory nucleic acids, for example small inhibitory ribonucleic acids (siRNA), to adjust, manipulate, prevent, inhibit, interfere, or block the androgen signal transduction pathway in a host cell, for example in a host's hair cell. Other aspects of the disclosure provide compositions and methods for interfering with the androgen signal transduction pathway by down regulating the expression of proteins involved in the androgen signal transduction pathway. Exemplary gene targets encoding proteins involved in the androgen signal transduction pathway include but are not limited to isozymes I and II of 5-α reductase, the androgen receptor, aromatase, 3-α-hydroxysteroiddehydrogenase, 3-β-hydroxysteroiddehydrogenase , 3-β-hydroxysteroiddehydrogenase-4-5-isomerase, 17-β-hydroxysteroidoxidoreductase, and steroid sulfatase. In some aspects, the inhibitory nucleic acids, for example siRNAs, interfere with the expression of targeted genes by preventing, reducing, or inhibiting the translation of mRNA transcribed from the targeted gene.

Another aspect of the disclosure provides compositions and methods for the treatment of androgen related diseases by decreasing the sensitivity of androgens in a host or host cell. The sensitivity of a cell to androgens can be reduced or regulated using siRNAs that interfere with or interrupt the androgen signal transduction pathway, for example by interfering or blocking the expression or function of any protein in the androgen signal transduction cascade. An exemplary aspect provides methods and compositions for treating hair loss using siRNAs, for example, to regulate the cell cycle of hair cells by manipulating the androgen signal transduction pathway.

Still another aspect or the disclosure provides compositions and methods for treating hair loss in a mammal, for example a human, by interfering with or inhibiting, the androgen signal transduction pathway of a hair cell. For example, siRNAs can be used to inhibit or reduce the production of DHT in a host or host cell using siRNAs targeted to enzymes involved in the production of DHT, for example 5-α reductase. Contacting mRNAs encoding 5α-reductase with a corresponding siRNA interferes with the translation of the mRNA and thereby interferes with the expression of the 5α-reductase. Some aspects provide siRNAs that induce the degradation, typically enzymatic degradation, of the target mRNA. The less 5α-reductase, the less DHT or ability to produce DHT. By reducing or inhibiting the production of DHT, the cycle of hair growth and renewal can be manipulated resulting in reduced hair loss or promoting new hair growth. Accordingly, other aspects of the present disclosure are directed to compositions and methods that inhibit the production of proteins that mediate the effects of androgens in androgenetic alopecia. Exemplary proteins included those proteins encoded by the genes for both isozymes I and II of 5-α reductase, the androgen receptor, aromatase, 3-α-hydroxysteroiddehydrogenase, 3-β-hydroxysteroiddehydrogenase,3-β-hydroxysteroiddehydrogenase-4-5-isomerase, 17-β-hydroxysteroidoxidoreductase, and steroid sulfatase.

Still other aspects of the disclosure provide compositions and methods for manipulating the androgen receptor signal transduction pathway in a host cell to reduce, inhibit, or prevent hair loss or to induce hair growth. In one aspect, inhibitory nucleic acids, typically ribonucleic acids, and more typically double stranded ribonucleic acids are provided that interfere with, inhibit, or interrupt the expression of proteins involved in the androgen signal transduction pathway. In particular, inhibitory ribonucleic acids (iRNA) or small inhibitory ribonucleic acids (siRNA) that comprise at least a partial sequence of nucleic acids encoding proteins involved with or related to the androgen signal transduction pathway are disclosed that inhibit or interfere with the expression of these proteins. The active siRNAs can be from about 10 to about 25 nucleotides in length although it will be appreciated that longer RNAs can be employed that are processed to result in siRNAs of smaller sizes, typically from about 10 to about 25 nucleotides in length.

Accordingly, aspects of the present discisoure describe a method of treating the hyperandrogenic conditions of androgenic alopecia, including male pattern alopecia, acne vulgaris, seborrhea, and female hirsutism by topical administration, and a method of treating all of the above conditions as well as benign prostatic hypertrophy, by systemic administration, of the novel compounds of the present disclosure. In one method, an siRNA is administered to a host, wherein the siRNA interrupts or interferes with the expression of a gene involved in the androgen signal transduction pathway that results in the reduction or inhibition of DHT production or androgen receptor expression.

Still another aspect provides a method of screening siRNAs to identify siRNAs that inhibit, interfere or otherwise disrupt the androgen signal transduction pathway, for example in hair cells. The screening can be accomplished using in vitro cell lines. Alternatively, the screening method can be used to identify compounds that mimic or synergize with siRNAs.

Yet another aspect provides a vector, typically a viral vector, that encodes a siRNA that interferes with or inhibits the androgen signal transduction pathway. The vector can be designed to express double stranded siRNA directly or indirectly wherein the siRNA comprises at least a portion of the target mRNA nucleic acid sequence, typically a mRNA encoding a protein in the androgen signal transduction pathway. Alternatively, the vector can encode a nucleic acid sequence containing specific cleavage sites such that the vector expresses a nucleic acid that can be cleaved to produce inhibitory nucleic acids of less than about 30 nucleotides in length. The expressed inhibitory nucleic acids are typically RNA, and more typically are double-stranded RNA. An exemplary vector includes a promoter for human RNA polymerase III, flanked on the 3′ end by a termination signal composed of five T residues. Between start and stop signals is a 21-nucleotide DNA template and its inverted repeat separated by a short non-homologous nucleotide spacer.

The present disclosure also encompasses a cell comprising a siRNA targeted to a mRNA encoding a protein in the androgen signal transduction pathway, for example isozymes I and II of 5-α reductase, the androgen receptor, aromatase, 3-α-hydroxysteroiddehydrogenase, 3-β-hydroxysteroiddehydrogenase, 3-β-hydroxysteroiddehydrogenase-4-5-isomerase, 17-β-hydroxysteroidoxidoreductase, and steroid sulfatase. Alternatively, the cell comprises a vector that when expressed, produces a siRNA that interferes with or inhibits the expression of a protein in the androgen signal transduction pathway. Suitable vectors containing functional elements such as promoters and other regulatory elements are known in the art. The sequence of representative siRNAs is provided in Table 1.

The inhibitory nucleic acids of the present disclosure can be formulated for administration by conventional means including topical, oral, and intravenous administration. These topical pharmaceutical compositions containing the compounds of the present disclosure ordinarily include about 0.1% to 15%, preferably about 0.1 to 5%, and more preferably about 0.1% to 2%, of the active compound, in admixture with a pharmaceutically acceptable carrier.

The compositions according to the disclosure may be in any form suitable for application to hair and scalp, especially in the form of an aqueous, alcoholic or aqueous-alcoholic preparation, such as a solution, gel, cream, emulsion or dispersion.

### BRIEF DESCRIPTION OF THE DRAWINGS

FIG. 1 is a diagram showing the cycle of hair growth.

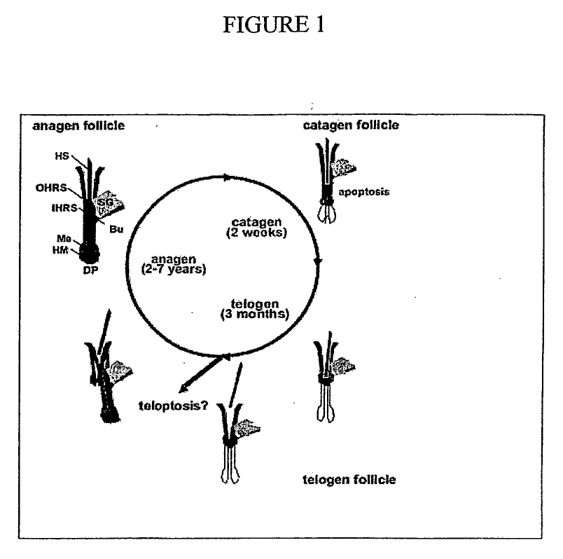

FIG. 2 is a flow diagram illustrating the genes involved in en exemplary androgen signal transduction pathway.

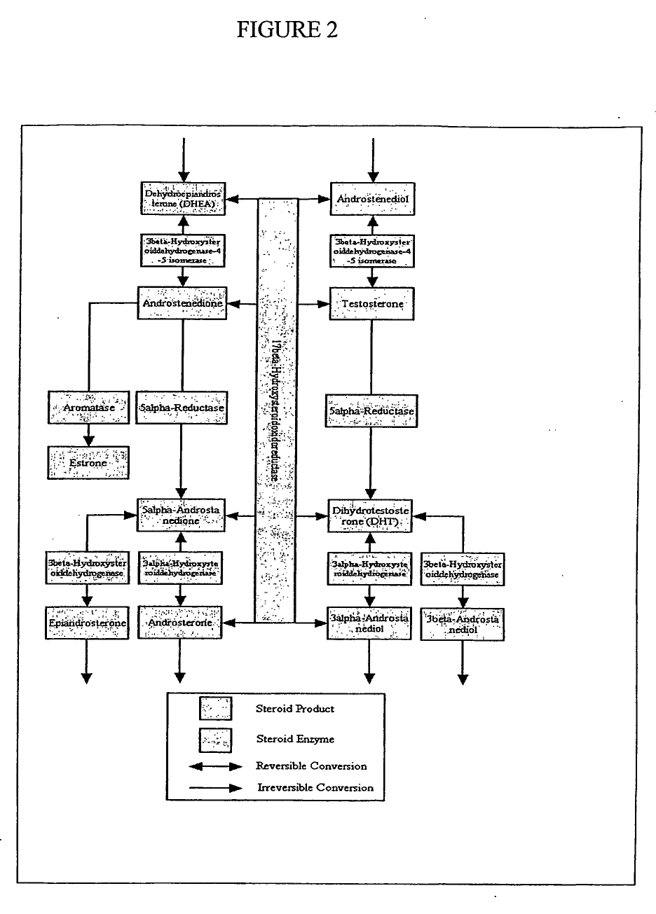

FIG. 3 is an autoradiograph showing the full length clone of steroid 5-alpha-reductase.

FIG. 4 is a diagram of the PCR amplicon that was TA cloned (Invitrogen) to generate pcDNA 3.1/SRD5a-GFP.

FIGS. 5A-C are fluorescence micrographs of Sy5y cells transfected to express with GFP-tagged steroid 5-alpha-reductase and rhodamine labelled dsRNA specific for the reporter protein.

### DETAILED DESCRIPTION

Definitions

Unless otherwise indicated the following terms used in the specification and claims have the meanings discussed below:

The term “organism” refers to any living entity comprised of at least one cell. A living organism can be as simple as, for example, a single eukaryotic cell or as complex as a mammal, including a human being.

The term “therapeutically effective amount” as used herein refers to that amount of the compound being administered which will relieve to some extent one or more of the symptoms of the disorder being treated. In reference to hair loss, a therapeutically effective amount refers to that amount which has the effect of (1) reducing the amount of hair loss, (2) inhibiting (that is, slowing to some extent, preferably stopping) androgen sensitivity, (3) inducing the grow of hair, and/or, (4) relieving to some extent (or, preferably, eliminating) one or more symptoms associated with the androgen related disease including but not limited to hair loss.

“Pharmaceutically acceptable salt” refers to those salts which retain the biological effectiveness and properties of the free bases and which are obtained by reaction with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid, malic acid, maleic acid, succinic acid, tartaric acid, citric acid, and the like.

A “pharmaceutical composition” refers to a mixture of one or more of the compounds described herein, or a pharmaceutically acceptable salts thereof, with other chemical components, such as physiologically acceptable carriers and excipients. The purpose of a pharmaceutical composition is to facilitate administration of a compound to an organism.

As used herein, a “pharmaceutically acceptable carrier” refers to a carrier or diluent that does not cause significant irritation to an organism and does not abrogate the biological activity and properties of the administered compound.

An “excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

“Treating” or “treatment” of a disease includes preventing the disease from occurring in an animal that may be predisposed to the disease but does not yet experience or exhibit symptoms of the disease (prophylactic treatment), inhibiting the disease (slowing or arresting its development), providing relief from the symptoms or side-effects of the disease (including palliative treatment), and relieving the disease (causing regression of the disease). With regard to cancer, these terms simply mean that the life expectancy of an individual affected with a cancer will be increased or that one or more of the symptoms of the disease will be reduced.

The term “prodrug” refers to an agent, including nucleic acids and proteins, which is converted into a biologically active form in vivo. Prodrugs are often useful because, in some situations, they may be easier to administer than the parent compound. They may, for instance, be bioavailable by oral administration whereas the parent compound is not. The prodrug may also have improved solubility in pharmaceutical compositions over the parent drug. A prodrug may be converted into the parent drug by various mechanisms, including enzymatic processes and metabolic hydrolysis. Harper, N.J. (1962). Drug Latentiation in Jucker, ed. Progress in Drug Research, 4:221-294; Morozowich et al. (1977). Application of Physical Organic Principles to Prodrug Design in E. B. Roche ed. Design of Biopharmaceutical Properties through Prodrugs and Analogs, APhA; Acad. Pharm. Sci.; E. B. Roche, ed. (1977). Bioreversible Carriers in Drug in Drug Design, Theory and Application, APhA; H. Bundgaard, ed. (1985) Design of Prodrugs, Elsevier; Wang et al. (1999) Prodrug approaches to the improved delivery of peptide drug, Curr. Pharm. Design. 5(4):265-287; Pauletti et al. (1997). Improvement in peptide bioavailability: Peptidomimetics and Prodrug Strategies, Adv. Drug. Delivery Rev. 27:235-256; Mizen et al. (1998). The Use of Esters as Prodrugs for Oral Delivery of β-Lactam antibiotics, Pharm. Biotech. 11:345-365; Gaignault et al. (1996). Designing Prodrugs and Bioprecursors I. Carrier Prodrugs, Pract. Med. Chem. 671-696; M. Asgharnejad (2000). Improving Oral Drug Transport Via Prodrugs, in G. L. Amidon, P. I. Lee and E. M. Topp, Eds., Transport Processes in Pharmaceutical Systems, Marcell Dekker, p. 185-218; Balant et al. (1990) Prodrugs for the improvement of drug absorption via different routes of administration, Eur. J. Drug Metab. Pharnmacokinet., 15(2): 143-53; Balimane and Sinko (1999). Involvement of multiple transporters in the oral absorption of nucleoside analogues, Adv. Drug Deliveiy Rev., 39(1-3):183-209; Browne (1997). Fosphenytoin (Cerebyx), Clin. Neuropharmacol. 20(1): 1-12; Bundgaard (1979). Bioreversible derivatization of drugs—principle and applicability to improve the therapeutic effects of drugs, Arch. Pharm. Chemi. 86(1): 1-39; H. Bundgaard, ed. (1985) Design of Prodrugs, New York: Elsevier; Fleisher et al. (1996). Improved oral drug delivery: solubility limitations overcome by the use of prodrugs, Adv. Drug Deliveiy Rev. 19(2): 115-130; Fleisher et al. (1985). Design of prodrugs for improved gastrointestinal absorption by intestinal enzyme targeting, Methods Enzymol. 112: 360-81; Farquhar D, et al. (1983). Biologically Reversible Phosphate-Protective Groups, J. Pharm. Sci., 72(3): 324-325; Han, H. K. et al. (2000). Targeted prodrug design to optimize drug delivery, AAPS PharmSci., 2(1): E6; Sadzuka Y. (2000). Effective prodrug liposome and conversion to active metabolite, Cur.r Drug Metab., 1(1):31-48; D. M. Lambert (2000) Rationale and applications of lipids as prodrug carriers, Eur. J. Pharm. Sci., 11 Suppl 2:S15-27; Wang, W. et al. (1999) Prodrug approaches to the improved delivery of peptide drugs. Curr. Pharm. Des., 5(4):265-87.

As used herein, the term “topically active agents” refers to compositions of the present disclosure that elicit pharmacological responses at the site of application (contact) to a host

As used herein, the term “topically” refers to application of the compositions of the present disclosure to the surface of the skin and mucosal cells and tissues.

As used herein, the term “surface” is used in its broadest sense. In one sense, the term refers to the outermost boundaries of an organism or inanimate object (e.g., vehicles, buildings, and food processing equipment, etc.) that are capable of being contacted by the compositions of the present disclosure (e.g., for animals: the skin, hair, and fur, etc., and for plants: the leaves, stems, flowering parts, and fruiting bodies, etc.). In another sense, the term also refers to the inner membranes and surfaces of animals and plants (e.g., for animals: the digestive-tract, vascular tissues, and the like, and for plants: the vascular tissues, etc.) capable of being contacted by compositions by any of a number of transdermal delivery routes (e.g., injection, ingestion, transdermal delivery, inhalation, and the like).

Androgen signal transduction pathway means the physiological and or biological sequence of events and the proteins and genes involved therein, from the production of androgen through the binding to the androgen receptor and the subsequent binding and activation of the androgen receptor and associated proteins and cofactors resulting in the induction of gene transcription.

The term “nucleic acid” is a term of art that refers to a string of at least two base-sugar-phosphate combinations. For naked DNA delivery, a polynucleotide contains more than 120 monomeric units since it must be distinguished from an oligonucleotide. However, for purposes of delivering RNA, RNAi and siRNA, either single or double stranded, a polynucleotide contains 2 or more monomeric units. Nucleotides are the monomeric units of nucleic acid polymers. The term includes deoxyribonucleic acid (DNA) and ribonucleic acid (RNA) in the form of a messenger RNA, anti-sense, plasmid DNA, parts of a plasmid DNA or genetic material derived from a virus. Anti-sense is a polynucleotide that interferes with the function of DNA and/or RNA. The term nucleic acids—refers to a string of at least two base-sugar-phosphate combinations. Natural nucleic acids have a phosphate backbone, artificial nucleic acids may contain other types of backbones, but contain the same bases. Nucleotides are the monomeric units of nucleic acid polymers. The term includes deoxyribonucleic acid (DNA) and ribonucleic acid (RNA). RNA may be in the form of an tRNA (transfer RNA), snRNA (small nuclear RNA), rRNA (ribosomal RNA), mRNA (messenger RNA), anti-sense RNA, RNAi, siRNA, and ribozymes. The term also includes PNAs (peptide nucleic acids), phosphorothioates, and other variants of the phosphate backbone of native nucleic acids.

The term “siRNA” means a small inhibitory ribonucleic acid. The siRNA are typically less than 30 nucleotides in length and can be single or double stranded. The ribonucleotides can be natural or artificial and can be chemically modified. Longer siRNAs can comprise cleavage sites that can be enzymatically or chemically cleaved to produce siRNAs having lengths less than 30 nucleotides, typically 21 to 23 nucleotides. siRNAs share sequence homology with corresponding target mRNAs. The sequence homology can be 100 percent or less but sufficient to result is sequence specific association between the siRNA and the targeted mRNA. Exemplary siRNAs do not activate the interferon signal transduction pathway.

The term “inhibitory nucleic acid” means an RNA, DNA, or combination thereof that interferes or interrupts the translation of mRNA. Inhibitory nucleic acids can be single or double stranded. The nucleotides of the inhibitory nucleic acid can be chemically modified, natural or artificial.

### EMBODIMENTS

Embodiments of present disclosure are directed, in part, to preventing, reducing, or inhibiting hair loss in a host, for example a mammal, using compositions comprising inhibitory nucleic acids such as siRNAs or salts or prodrugs thereof. The siRNAs of the present disclosure are designed to inhibit or interfere with the translation of mRNA encoding proteins involved in the androgen signal transduction pathway. In some aspects, the siRNAs induce the enzymatic cleavage of target mRNAs. Proteins involved in the androgen signal transduction pathway include but are not limited to: the androgen receptor, 5-α reductase, aromatase, 3-α-hydroxysteroiddehydrogenase, 3-β-hydroxysteroiddehydrogenase, 3-β-hydroxysteroiddehydrogenase-4-5-isomerase, 17-β-hydroxysteroidoxidoreductase, and steroid sulfatase. Alternatively, aspects of the disclosure is also directed to inducing or increasing the growth of hair, for example using compositions comprising inhibitory nucleic acids such as siRNAs. The sequence of exemplary siRNAs and their targets is provided in Table 1.

In one embodiment, the expression of androgen receptor in a host cell, typically a hair cell, is reduced or inhibited by contacting the hair cell with an siRNA. siRNAs of the present disclosure comprise at least a partial sequence of the target mRNAs, for example mRNA encoding the androgen receptor. The sequence homology between an siRNA and a target mRNA can be 100 percent or less but greater than about 50 percent, and typically 90 percent or greater. The percentage of sequence homology between siRNA and a target mRNA should be sufficient to result in sequence specific association between the siRNA and the target mRNA under cytoplasmic conditions.

### The Physiology of Androgen Related Hair Loss

One exemplary embodiment of the present disclosure is directed to the treatment or prevention of androgen related hair loss in an organism, for example the treatment or prevention of alopecia using inhibitory nucleic acids to interfere or interrupt the androgen receptor signal transduction pathway. Alopecia is essentially due to a disturbance in hair renewal which results, at first, in an acceleration in the frequency of the cycles at the expense of the quality of the hair and then of its amount. A progressive thinning of the head of hair takes place by regression of the so-called “terminal” hairs to the downy stage. Regions are preferentially affected, in particular the temple or frontal bulbs in men and, in women, a diff-use alopecia of the vertex is observed. The term alopecia covers a whole family of complaints of the hair follicle, whose final consequence is the partial or general permanent loss of the hair.

Accordingly, the present disclosure encompasses the treatment of hair loss in men and women related including alopecia, pattern baldness, or androgen related hair loss. In one embodiment, inhibitory nucleic acids prevent hair loss by down-regulating, inhibiting, or interrupting the expression of the androgen receptor in a host cell, for example a hair cell. By down-regulating the androgen receptor in hair cells, the hair cells are desensitized to androgens and the cell cycle of the hair cell is unaffected by androgens. Thus, another embodiment of the present disclosure provides a hair cell desensitized to androgens by inhibitory nucleic acids, for example siRNAs homologous to the androgen receptor that inhibit or reduce the expression of the androgen receptor in a host hair cell. It will be appreciated by those of skill in the art that the present disclosure encompasses siRNAs designed to interrupt or interfere with the expression or function of any protein in the androgen signal transduction pathway, either directly or indirectly, for the treatment, prevention, or reduction of hair loss in an organism.

### Inhibitory Nucleic Acids

The inhibitory nucleic acids of certain embodiments of the present disclosure are directed to inhibiting or interfering with the expression of proteins involved in the androgen signal transduction pathway. The inhibitory nucleic acids disclosed herein include small inhibitory ribonucleic acids (siRNAs) that are typically less than 30 nucleotides in length, more typically 21 to 23 nucleotides in length, and can be single or double stranded. One strand of a double-stranded siRNA comprises at least a partial sequence complementary to a target mRNA. The ribonucleotides of the siRNA can be natural or artificial and can be chemically modified. Longer siRNAs can comprise cleavage sites that can be enzymatically or chemically cleaved to produce siRNAs having lengths less than 30 nucleotides. siRNAs share sequence homology with corresponding target mRNAs. The phosphate backbones of the siRNAs can be chemically modified to resist enzymatic degradation. The sequence homology can be about 100 percent or less, but sufficient to result is sequence specific association between the siRNA and the targeted mRNA.

Nucleic acids, in particular RNA, are known to participate in a form of post-transcriptional gene silencing termed “RNA interference” or RNAi. First observed in plants, reduction of expression of specific mRNA sequences was found to be inducible in Drosophila melanogaster and Caenorhabditis elegans by introduction of double-stranded RNA (dsRNA) molecules mimicking the sequence of the mRNA. The effect was found to be potent and extremely long-lived in these experimental model organisms, generally extending to the F1 progeny of a treated adult specimen. Additionally, the effect was found to be exquisitely sequence-specific; discrepancy of even a few base pairs between the dsRNA and the target mRNA virtually abolished the silencing. RNAi has been used experimentally in these non-mammalian systems to generate transient silencing of specific genes of interest, especially those which are not amenable to more traditional gene knockout methods (e.g., those that produce embryonic lethality and thus cannot be studied in the adult animal).

The first evidence that dsRNA could lead to gene silencing came from work in the nematode Caenorhabditis elegans. Researchers Guo and Kemphues used antisense RNA to shut down expression of the par-1 gene in order to assess its function. As expected, injection of the antisense RNA disrupted expression of par-1, but quizzically, injection of the sense-strand control did too. This result was a puzzle until three years later. It was then that Fire and Mello first injected dsRNA—a mixture of both sense and antisense strands—into C. elegans. This injection resulted in much more efficient silencing than injection of either the sense or the antisense strands alone. Injection of just a few molecules of dsRNA per cell was sufficient to completely silence the homologous gene's expression. Furthermore, injection of dsRNA into the gut of the worm caused gene silencing not only throughout the worm, but also in its first generation offspring. The potency of RNAi inspired Fire and Timmons to try feeding nematodes bacteria that had been engineered to express dsRNA homologous to the C. elegans unc-22 gene. Surprisingly, these worms developed an unc-22 null-like phenotype. Further work showed that soaking worms in dsRNA was also able to induce silencing. These strategies, whereby large numbers of nematodes are exposed to dsRNA, have enabled large-scale screens to select for RNAi-defective C. elegans mutants and have led to large numbers of gene knockout studies within this organism. Thus, one embodiment of the present disclosure provides siRNAs comprising a sense strand and an anti-sense strand, wherein the sense strand comprises at least a partial sequence of a target mRNA.

RNAi has also been observed in Drosophila melanogaster. Although a strategy in which yeast were engineered to produce dsRNA and then fed to fruit flies failed to work, microinjecting Drosophila embryos with dsRNA does induce silencing. Silencing can also be induced by biolistic techniques in which dsRNA is “shot” into Drosophila embryos, or by engineering flies to carry DNA containing an inverted repeat of the gene to be silenced. Over the last few years, these RNAi strategies have been used as reverse genetics tools in Drosophila organisms, embryo lysates, and cells to characterize various loss-of-function phenotypes. Zamore and colleagues found that dsRNA added to Drosophila embryo lysates was processed to 21-23 nucleotide species. They also found that the homologous endogenous mRNA was cleaved only in the region corresponding to the introduced dsRNA and that cleavage occurred at 21-23 nucleotide intervals.

Current models of RNAi divide the process of inhibition into broad “initiation” and “effector” stages. In the initiation step, input dsRNA is digested into 21-23 nucleotide small interfering RNAs (siRNAs), which have also been called “guide RNAs.” Evidence indicates that siRNAs are produced when the enzyme Dicer, a member of the RNase III family of dsRNA-specific ribonucleases, processively cleaves dsRNA in an ATP-dependent, processive manner. Successive cleavage events degrade the RNA to 19-21 bp duplexes (siRNAs), each with 2-nucleotide 3′ overhangs. Inhibitory nucleic acids of the present disclosure can be enzymatically cleaved, for example in vivo, to produce siRNAs from 10 to about 30 nucleotides, typically about 19 to about 23 nucleotides.

In the effector step, the siRNA duplexes bind to a nuclease complex to form what is known as the RNA-induced silencing complex, or RISC. An ATP-depending unwinding of the siRNA duplex is required for activation of the RISC. The active RISC then targets the homologous transcript by base pairing interactions and cleaves the mRNA˜12 nucleotides from the 3′ terminus of the siRNA. Although the mechanism of cleavage is at this date unclear, research indicates that each RISC contains a single siRNA and an RNase that appears to be distinct from Dicer. Because of the remarkable potency of RNAi in some organisms, an amplification step within the RNAi pathway has also been proposed. Amplification could occur by copying of the input dsRNAs, which would generate more siRNAs, or by replication of the siRNAs themselves. Alternatively or in addition, amplification could be effected by multiple turnover events of the RISC. One embodiment encompasses the in vivo amplification of the siRNAs disclosed herein. Additionally, the siRNAs described herein can form a complex with additional proteins and/or cofactors to enzymatically cleave a target mRNA.

### Androgens and the Androgen Receptor

Androgens, specifically testosterone (T) and dihydroxytestosterone (DHT), mediate hair loss including AGA. The present disclosure provides siRNAs that inhibit the expression of androgen receptors or 5α0 reductase thereby preventing or reducing hair loss or stimulating hair growth. Testosterone taken up by hair cells in the scalp is converted to DHT by the enzyme 5-α reductase. Inhibiting the expression of 5α reductase with the siRNAs of the present disclosure reduces the levels of DHT. In one embodiment, DHT can be reduced locally or systemically using the inhibitory nucleic acids described herein.

DHT binds to its receptor, the Androgen Receptor (AR), which acts to alter nuclear gene expression. The AR is an intracellular receptor residing in the nucleus or cytoplasm and is a member of the steroid hormone receptor family. The change in nuclear gene expression of androgen responsive genes in response to androgen forces the hair cell to progressively shorten the duration of anagen with successive hair cycles, causing reduced hair renewal and hair loss.

The AR is typically bound to heat shock protein 90 that maintains the AR inactive state and the AR hormone binding affinity (Fang, Y. et al. (1996). J Biol Chem. 271 (45), 28697-28702). Upon binding however, direct androgen action is initiated as inhibitory heat shock proteins are released from the androgen receptor. The AR is then phosphorylated and undergoes a conformational change necessary for translocation and dimerization (Grino, P. B. et al. (1987). Endocrinol. 120, 1914-1920). Although in the wild-type receptor, this ligand binding is necessary for transcriptional activity, one in vivo receptor with a deleted ligand binding domain does possess transcriptional activity. This may indicate that the unliganded binding domain is actually a repressor of receptor action due to conformational constraints in the unbound receptor possessing the ligand binding domain (Jenster, G. et al. Mol Endocrinol. (1991). 5, 1396-1404).

Once in the nucleus (either by direct binding there or by translocation), the phosphorylated receptor is dimerized and binds to a DNA androgen response element (ARE). The hormone response element, which is also bound by other hormone receptors from this family, is a 15 base pair sequence responsible for transcription initiation. Once bound, other transcription regulating proteins or co-activators may also bind the AR-ARE complex to stabilize the promoter of the regulated gene (Shibata, H. (1997). Recent Progress Horm Research. 52, 141-164; Kang, H. Y. (1999). J. Biol. Chem. 274 (13), 8570-8576). Such co-activators include proteins such as ARA 54, ARA 55, ARA 70, ARA 160 (Yeh, S. et al. (1996). Proc. Natl. Acad. Sci. USA. 93 (11), 5517-5521; Hsiao, P. W. (1999). J. Biol. Chem. 32, 22373-22379). This binding of such co-factors ultimately results in the regulation of transcription rate. Thus, one embodiment provides inhibitory nucleic acids, for example siRNAs, comprising at least a partial sequence of a target mRNA encoding a protein that associates with the androgen receptor, for example the AR-ARE. These siRNAs can inhibit or interfere with the expression of the AR associated proteins and thereby inhibit or interfere with the transcription acitivity of the AR. Exemplary AR associated proteins include but are not limited to ARA 54, ARA 55, ARA 70, ARA 160. The resultant mRNA from androgen dependent transcription is then processed and transported to ribosomes where it is translated into proteins that can alter cellular function. Thus, exemplary embodiments of the present disclosure encompass siRNAs that inhibit or interfere with the translation of mRNA from androgen dependent transcription.

In some tissues there is evidence for a ligand-independent dependent activation of transcriptional activity via the AR. An unliganded receptor with a deletion of the ligand binding domain may possess activity. This indicates activity in the absence of ligand binding. In addition, growth factors (insulin-like growth factor, keratinocyte growth factor, and epidermial growth factor) as well as protein kinase A activators might be able to induce a transcriptionally active AR in the absence of ligand binding. Some of these ligand independent transcription activators may act via influencing the AR phosphorylation state. Thus, one embodiment discloses siRNAs targeted to ligand independent transcription activators of AR, for example growth factors and protein kinas A activators for the treatment of androgen related diseases such as hair loss.

Ligand binding, for example DHT, alters the protein conformation of the AR to allow binding of coactivator molecules that can amplify the hormone signal and mediate transcriptional initiation of androgen responsive genes. The AR can also undergo intramolecular interactions that influence its activity and interaction with cofactors. The AR protein has several domains or motifs similar to the modular structure of other steroid hormone receptors. There are two characterized forms of the androgen receptor. The first, and predominant form, is a 110-114 kDa protein of 910-919 amino acids. The second is a smaller 87 kDa protein of about 720-729 amino acids in length that makes up only about 4-26% of the detectible androgen receptors located in varying tissues.

### 5-α Reductase

Exemplary siRNAs of the present disclosure can also be targeted to enzymes that mediate the production or modification of androgens, for ezample 5-α reductase. The enzyme 5-α reductase converts testosterone into DHT and it is DHT that is most potent in promoting AGA. There are at least two 5-α reductase isoenzymes, type I and type II. They are active in different regions of the body, but they both produce DHT that can circulate throughout the body and exert effects in organs other than where DHT was produced. Type II 5-α reductase is the most active enzyme form. It produces 60% to 70% of the total DHT human beings. Type I produces the remaining 30% to 40% of DHT. 5-α reductase inhibitors, promoted as treatments for AGA, should ideally block both types of isoenzyme to have maximum effect. Finasteride is a type II 5-α reductase inhibitor and so reduces DHT levels by at most 70%. However, the use of finesteride has been associated with reduced libido, teratagenic effects and other side effects in certain individuals thus prompting a more specific and safer approach to treating AGA.

Existing Therapies

All androgen-responsive genes are activated by androgen receptor (AR) bound to either T or DHT and it is believed that AR is more transcriptionally active when bound to DHT than T. Thus, therapies for treating androgen related diseases, including alopecia, encompass anti-androgens. The present disclosure encompasses therapies for hair loss, in particular androgen related hair loss, by using the inhibitory nucleic acids disclosed herein, alone, or in combination with existing hair loss therapies. For example the two classes of anti-androgens, presently available, are the steroidal derivatives, all of which possess mixed agonistic and antagonistic activities, and the pure non-steroidal anti-androgens of the class of flutamide and its derivatives. Flutamide has a structure similar to the male sex hormone testosterone. It works by blocking and preventing the binding of testosterone to the receptors on the surface of cells. The non-steroidal flutamide and its derivatives display pure anti-androgenic activity, without exerting agonistic or any other hormonal activity. Flutamide and its derivatives, Casodex and Anandron, are highly effective in the treatment of prostate cancer.

Exemplary anti-androgens with a steroid structure include but are not limited to (17 alpha-acetoxy-6-chloro-2-oxa-4,6-pregnadiene-3,20-dione). This compound inhibits 5-αdihydrotestosterone (5-α-DHT) and testosterone receptors activity by blocking androgen receptors and in this way decreasing the nuclear androgen receptors fraction. Representative examples of steroidal blocking agents include spironolactone, cyproterone acetate, trimethyltrienolone (available from Roussel Uclaf under the designation RU 2956), canrenone and canrenoic acid. Examples of suitable non-steroidal blocking agents include flutamide (α,α,.α.-trifluoro-2-methyl-4′-nitro-m-propionotoluidide) and hydroxy-flutamide (α,α,.α.-trifluoro-2-methyl-4′-nitro-m-lactoluidide), both available from Schering Corp., and RU 23908 (5,5-dimethyl-3-[4-nitro-3(trifluoromethyl)phenyl]-2,4-imidazolidinedione) and RU 22930 (5,6-dihydro-2-methyl-4-[4-nitro-3-(trifluromethyl)phenyl]-2H-1,2,4-oxadio zin-3-(4H)-one), available from Roussel Uclaf.

RU 56187 and RU 58841 are additional examples of non-steroidal anti-androgens. They are N-substituted arylthiohydantoins and inhibit 5 α-dihydrotestosterone (5-α-DHT) and testosterone receptors activity by blocking androgen receptors. RU 58841 and RU 56187 are eliminated from blood stream relatively quickly, but they form a common metabolite, the N-desalkyl derivative, RU 56279 (also anti-androgen), which is eliminated much more slowly and is responsible for general adverse anti-androgenic effects.

Other compounds for the treatment of hair loss are disclosed in U.S. Pat. No. 6,451,777 which is incorporated by reference in its entirety and include but are not limited to lupane triterpenes, derivatives of lupane triterpenes, derivatives of oleanane triterpenes, derivatives of ursane triterpenes, and salts and mixtures thereof.

Additional therapeutic agents for the treatment of hair loss can be used in combination with the present disclosure, including formulations in which a minoxidil solution, typically comprising about 5% minoxidil, is combined with azelaic acid, typically about 5%. Azelaic acid is a topically effective inhibitor of 5-α reductase. Another part of this formulation is the anti-inflammatory agent betamethasone valerate. Additionally, retinoic acid can be added, typically about 0.025%.

Spironolactone is another suitable second therapeutic agent for use with the present disclosure. Spironolactone is a medication that has been used for many years to treat hypertension and fluid retention. Its effect is local at blocking DHT and there are no systemic effects from its use on the scalp. Spironolactone is a potent anti-androgen. It competes with DHT for the receptor sites on the hair follicles

According to another aspect of the present disclosure, a method is provided for hormonal therapy in a patient (i.e., suffering from an androgen-dependent condition) which includes contacting a nucleic acid encoding an 5-α reductase of a patient with an inhibitory RNA under conditions effective to bind the inhibitory RNA compound to the nucleic acid encoding an 5-α reductase and effect a change in an androgen-dependent condition, such as alopecia. Androgen-dependent conditions which may be treated according to the present disclosure include those conditions which are associated hyperandrogenic conditions.

### Pharmaceutical Compositions

Exemplary embodiments of the present disclosure include pharmaceutical compositions that can be manufactured by processes well known in the art, e.g., by means of conventional mixing, dissolving, granulating, dragee-making, levigating, emulsifying, encapsulating, entrapping, lyophilizing processes or spray drying. Moreover, in certain embodiments, the compositions of the present disclosure may be formulated for horticultural or agricultural use. Such formulations include dips, sprays, seed dressings, stem injections, sprays, and mists.

The compositions of the present disclosure can be liquids or lyophilized or otherwise dried formulations and include diluents of various buffer content (e.g., Tris-HCl, acetate, phosphate), pH and ionic strength, additives such as albumin or gelatin to prevent absorption to surfaces, a surfactant such as a polysorbate surfactant (e.g., TWEEN 20, TWEEN 40, TWEEN 60, and TWEEN 80), a pheoxypolyethoxyethanol surfactant (e.g., TRITON X-100, X-301, X-165, X-102, and X-200, and TYLOXAPOL) Pluronic F68, or sodium dodecyl sulfate, solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polglycolic acid, hydrogels, etc, or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance, controlled or sustained release compositions include formulation in lipophilic depots (e.g., fatty acids, waxes, oils).

The present disclosure contemplates formulations that may be employed in pharmaceutical and therapeutic compositions and applications suitable for inhibiting the action of androgens relating to hair loss. Such compositions may be employed to inhibit expression of proteins related to hair growth or loss, interfere with signal transduction mechanisms of the androgen receptor, interfere with the formation, dissemination of androgens, or increase the growth of hair.

For in vivo applications, the compositions can be administered in any effective pharmaceutically acceptable form to warm blooded animals having hair, including human and animal subjects. Generally, this entails preparing compositions that are essentially free of pyrogens, as well as other impurities that could be harmful to humans or animals.

Other embodiments of the disclosure provide particulate compositions coated with polymers (e.g., poloxamers or poloxamines). Still other embodiments of the compositions of the disclosure incorporate particulate forms protective coatings, protease inhibitors or permeation enhancers for various routes of administration, including parenteral, pulmonary, nasal and oral. In one embodiment the pharmaceutical composition is administered buccally, rectally, vaginally, topically, nasally, parenterally, paracancerally, transmucosally, transdermally, intramuscularly, intravenously, intradermally, subcutaneously, intraperitonealy, intraventricularly, intracranially, intratumorally, spray or in any other form effective to deliver active compositions.

For topical applications, the pharmaceutically acceptable carrier may take the form of a liquid, cream, foam, lotion, or gel, and may additionally comprise organic solvents, emulsifiers, gelling agents, moisturizers, stabilizers, surfactants, wetting agents, preservatives, time release agents, and minor amounts of humectants, sequestering agents, dyes, perfumes, and other components commonly employed in pharmaceutical compositions for topical administration.

Further, as used herein “pharmaceutically acceptable carrier” are well known to those skilled in the art and include, but are not limited to, 0.01-0.1M and preferably 0.05M phosphate buffer or 0.8% saline. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media.

Parenteral vehicles include sodium chloride solution, Ringer's dextrose, dextrose and sodium chloride, lactated Ringer's or fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like.

Controlled or sustained release compositions include formulation in lipophilic depots (e.g, fatty acids, waxes, oils). Also comprehended by the disclosure are particulate compositions coated with polymers (e.g. poloxamers or poloxamines) and the compound coupled to antibodies directed against tissue-specific receptors, ligands or antigens or coupled to ligands of tissue-specific receptors.

Tablet and dosage forms of the compositions in which the emulsions are formulated for oral or topical administration include liquid capsules, and suppositories. In solid dosage forms for oral administration, the compositions may be admixed with one or more substantially inert diluent (e.g., sucrose, lactose, or starch, and the like) and may additionally comprise lubricating agents, buffering agents, enteric coatings, and other components well known to those skilled in the art.

Compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline are known to exhibit substantially longer half-lives in blood following intravenous injection than do the corresponding unmodified compounds. Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound.

In yet another embodiment, the pharmaceutical composition can be delivered in a controlled release system. For example, the agent may be administered using intravenous infusion, an implantable osmotic pump, a transdermal patch, liposomes, or other modes of administration. In one embodiment, a pump may be used (Sefton (1987). CRC Crit. Ref. Biomed. Eng. 14:201; Buchwald et al. (1980). Surgery 88:507; Saudek et al. (1989). N. Engl. J. Med. 321:574). In another embodiment, polymeric materials can be used. In yet another embodiment a controlled release system can be placed in proximity of the therapeutic target, i.e., the brain, thus requiring only a fraction of the systemic. Preferably, a controlled release device is introduced into a subject in proximity of the site of inappropriate immune activation or a tumor. Other controlled release systems are discussed in the review by Langer (1990). Science 249:1527-1533.

In other embodiments, the compositions may be impregnated into absorptive materials, such as sutures, bandages, and gauze, or coated onto the surface of solid phase materials, such as surgical staples, zippers and catheters to deliver the compositions to a site for the prevention of microbial infection. Other delivery systems of this type will be readily apparent to those skilled in the art.

Examples of suitable oily vehicles or solvents for use with the present disclosure are vegetable or animal oils such as sunflower oil or fish-liver oil. Preparations can be effected both as dry and as wet granules. For parenteral administration (subcutaneous, intravenous, intra-arterial, or intramuscular injection), the compositions or their physiologically tolerated derivatives such as salts, esters, N-oxides, and the like are converted into a solution, suspension, or emulsion, if desired with the substances customary and suitable for this purpose, for example, solubilizers or other auxiliaries. Examples are: sterile liquids such as water and oils, with or without the addition of a surfactant and other pharmaceutically acceptable adjuvants. Illustrative oils are those of petroleum, animal, vegetable, or synthetic origin, for example, peanut oil, soybean oil, or mineral oil. In general, water, saline, aqueous dextrose and related sugar solutions, and glycols such as propylene glycols or polyethylene glycol are preferred liquid carriers, particularly for injectable solutions.

In addition, if desired, the composition can contain minor amounts of auxiliary substances such as wetting or emulsifying agents, pH buffering agents which enhance the effectiveness of the active ingredient.

An active component can be formulated into the composition as neutralized pharmaceutically acceptable salt forms. Pharmaceutically acceptable salts include the acid addition salts (formed with the free amino groups of the polypeptide or antibody molecule) and which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

For topical administration to body surfaces using, for example, creams, gels, drops, and the like, the inhibitory nucleic acids and there prodrugs or their physiologically tolerated derivatives such as salts, esters, N-oxides, and the like are prepared and applied as solutions, suspensions, or emulsions in a physiologically acceptable diluent with or without a pharmaceutical carrier.

In another embodiment, the active compound can be delivered in a vesicle, in particular a liposome (see Langer (1990). Science, 249:1527-1533; Treat et al. (1989). in Lopez-Berestein and Fidler (eds.), Liposomes in the Therapy of Infectious Disease and Cancer, Liss, N.Y., pp. 353-365).

Suitable salts of the compositions disclosed herein include pharmaceutically acceptable salts. Other salts may, however, be useful, in the preparation of the compounds according to the disclosure or of their pharmaceutically acceptable salts. Suitable pharmaceutically acceptable salts of the compounds of this disclosure include acid addition salts which may, for example, be formed by mixing a solution of the compound according to the disclosure with a solution of a pharmaceutically acceptable acid such as hydrochloric acid, sulphuric acid, methanesulphonic acids fumaric acid, maleic acid, succinic acid, acetic acid, benzoic: acid, oxalic acid, citric acid, tartaric acid, carbonic acid or phosphoric acid.

### Combination Therapy

It is also an aspect of this disclosure that a composition described herein, or its prodrug, salt, or derivative might be combined with other therapeurtic agents for the treatment of the diseases and disorders discussed above. Exemplary second agents include but are not limited to zinc salts of carboxylic acids, saponins, tritapenes, crataegolic acid, celastrol, asiatic acid, inhibitors of 5-α-reductase, 1,4-methyl-4-azasteroids, androgen receptor antagonists, Minoxidil, azelaic acid and its derivatives, cyclosporin, triiodothyronine, diazoxide and potassium channel openers, and combinations thereof.

### Dosage

Compositions disclosed here can be administered in a therapuetically acceptable amount. Generally, a therapeutically effective amount means an amount of compound effective to prevent, alleviate or ameliorate symptoms of disease such as inducing hair growth or preventing hair loss. Therapeutically effective amounts of compositions described herein can be approximately 10 mg/m² to 400 mg/m², preferably 50 mg/m² to 300 mg/m², more preferably 100 mg/m² to 220 mg/m², even more preferably 195 mg/m². For any compound used in the methods of the disclosure, the therapeutically effective amount or dose can be estimated initially from cell culture assays. Then, the dosage can be formulated for use in animal models so as to achieve a circulating concentration range that includes the IC₅₀ as determined in cell culture (i.e., the concentration of the test compound which achieves a half-maximal inhibition of the androgen signal transduction pathway). Such information can then be used to more accurately determine useful doses in humans.

Toxicity and therapeutic efficacy of the compounds described herein can be determined by standard pharmaceutical procedures in cell cultures or experimental animals, e.g., by determining the IC₅₀ and the LD₅₀ for a subject composition. The data obtained from these cell culture assays and animal studies can be used in formulating a range of dosage for use in humans. The dosage may vary depending upon the dosage form employed and the route of administration utilized. The exact formulation, route of administration and dosage can be chosen by the individual physician in view of the patient's condition. (See e.g., Hardman (2001) in The Pharmacological Basis of Therapeutics (10th edition) McGraw Hill).

Dosage amount and interval may be adjusted individually to provide plasma levels of the active species which are sufficient regulate the androgen signal transduction pathway to achieve the desired result, for example induction of hair growth or prevention of hair loss. The minimal effective plasma concentration levels will vary for each compound but can be estimated from in vitro data, e.g., the concentration necessary to achieve 50-90% inhibition of an androgen signal transduction pathway may be ascertained using the assays described herein. Preferably, the dosages necessary to achieve the minimal effective plasma concentration levels will depend on individual characteristics and route of administration. HPLC assays or bioassays can be used to determine plasma concentrations.

Dosage intervals can also be determined using minimal effective plasma concentration levels. Compounds should be administered using a regimen that maintains plasma levels above the minimal effective plasma concentration level for 10-90% of the time, preferably between 30-90% and most preferably between 50-90%. In cases of local administration or selective uptake, the effective local concentration of the compositions may not be related to plasma concentration and other procedures known in the art may be employed to determine the correct dosage amount and interval.

The amount of a composition administered will, of course, be dependent on the subject being treated, the severity of the affliction, the manner of administration, the judgment of the prescribing physician, etc.

### Packaging

The compositions may, if desired, be presented in a pack or dispenser device, such as an FDA approved kit, which may contain one or more unit dosage forms containing the compositions described herein. The pack may for example comprise metal or plastic foil, such as a blister pack. The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions or of human or veterinary administration. Such notice, for example, may be of the labeling approved by the U.S. Food and Drug Administration for prescription drugs or of an approved product insert. Compositions comprising a compound of the disclosure formulated in a compatible pharmaceutical carrier may also be prepared, placed in an appropriate container, and labeled for treatment of an indicated condition. Suitable conditions indicated on the label may include treatment of a tumor, inhibition of angiogenesis, treatment of fibrosis, diabetes, and the like.

### In Vivo Assays

The effect of an inhibitory nucleic acid can be tested by assaying the telogen period in C3H mice (Harlan Sprague Dawley, Inc., Indianapolis, Ind.) from approximately 40 days of age until about 75 days of age, when hair growth is synchronized. It is believed that after 75 days of age, hair growth is no longer synchronized. Wherein about 40 day-old mice with dark fur (brown or black) are used in hair growth experiments, melanogenesis occurs along with hair (fur) growth wherein the topical application of hair growth promoters are evaluated. The Telogen Conversion Assay herein below can be used to screen compounds for potential hair growth by measuring melanogenesis.

For example, three groups of 44 day-old C3H mice can be utilized: a vehicle control group, a positive control group, and a test compound group, wherein the test compound group is administered a compound of the present disclosure. The length of the assay is at least 19 days with 15 treatment days (wherein the treatment days occur Mondays through Fridays). Day 1 is the first day of treatment. Most studies will end on Day 19, but a few may be carried out to Day 24 if the melanogenesis response looks positive, but occurs slowly.

Typically, the mice are treated topically Monday through Friday on their lower back (base of tail to the lower rib). A pipettor and tip are used to deliver 100-400 μL to each mouse's back: The 400 μL application is applied slowly while moving hair on the mouse to allow the application to reach the skin.

While each treatment is being applied to the mouse topically, a visual grade of from 0 to 4 can be given to the skin color in the application area of each animal. As the mice convert from telogen to anagen their skin color will become more bluish-black. The grades 0 to 4 represent the following visual observations as the skin progresses from white to bluish-black: Whitish Skin Color-0; Skin is light gray (indication of initiation of anagen)-1; Appearance of Blue Spots-2; Blue Spots are aggregating to form one large blue area-3; Skin is dark blue (almost black) with color covering majority of treatment area (indication of mouse in full anagen)-4.

### EXAMPLES

### EXAMPLE 1

### Synthesis of siRNA

Single-stranded, gene-specific sense and antisense RNA oligomers optionally with overhanging 3′ deoxynucleotides are prepared and purified by PAGE using the sequences listed in Tables 1-9 below. The two oligomers, 40 μM each, are annealed in 250 μl of buffer containing 50 mM Tris-HCl, pH 8.0 and 100 mM NaCl, by heating to 94° C. for 2 minutes, cooling to 90° C. for 1 minute, then cooling to 20° C. at a rate of 1° C. per minute. The resulting siRNA is stored at −20° C. prior to use.

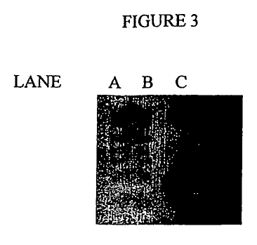

The steroid 5-apha-reductase (SRD5a Locus Link id: 6715) was PCR amplified from the full-length cDNA clone (clone MGC: 12396 IMAGE: 3683274). Primers were designed to generate the full-length clone for TA-cloning into pcDNA 3.1 CT-GFP. As such, the reverse primer lacked a stop codon (Forward Primer: atgcaggttcagtgccagca [Seq ID No.: 1401]; Reverse Primer: ttaaaaagatgaatggaataag [Seq ID No.: 1402]) The 800 base pair product is shown in FIG. 3.

### EXAMPLE 2

### In Vitro Assays

To determine the inhibition of androgen signal transduction proteins with siRNAs prepared above, the siRNAs are administered to cell culture cells expressing androgen signal transduction pathway proteins such as the androgen receptor or 5α reductase. Exemplary cell lines expressing androgen signal transduction pathway proteins are found in the catalogue for American Tissue Type Culture which is incorporated herein in its entirety. Briefly, cell lines are maintained in RPMI 1640 media (GibcoBRL, Gaithersburg, Md.) containing 10% BCS. Varying amounts (150-350 μg/ml) of siRNA were added to the culture media. Cells are incubated under the same conditions, at 37° C., in 5% CO₂ for 1-4 days. At the end of the incubation period, the cells are washed with PBS (phosphate buffered saline) and detached from the culture vessels using eversene. The cells are then assayed for expression of androgen signal transduction proteins such as androgen receptor and or 5α reductase.

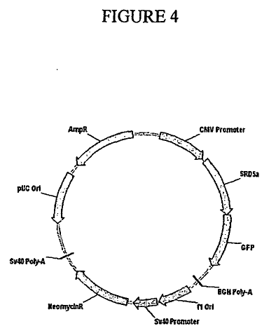

FIG. 4 shows the PCR amplicon that was TA cloned (Invitrogen) according to manufacture's instructions to generate pcDNA 3.1/SRD5a-GFP. This plasmid generates the SRD5a protein linked to the GFP (green fluorescent protein) reporter in mammalian cells. The pcDNA 3.1/SRD5a-GFP was transfected into Sy5y cells and confocal images were taken 24 hours later.

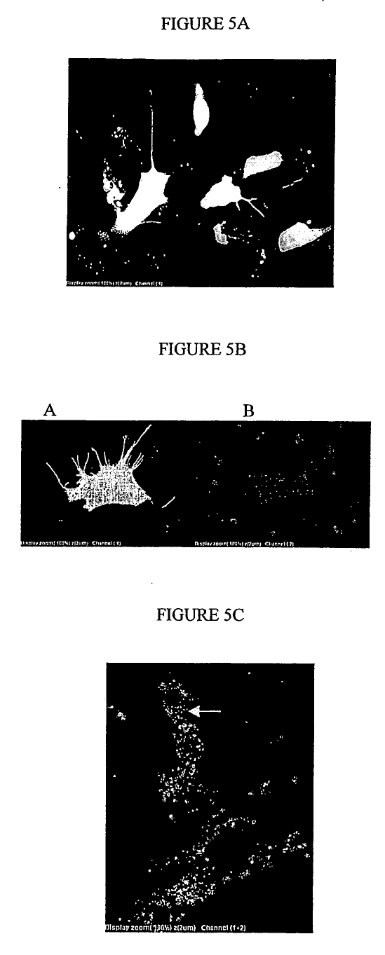

FIG. 5A shows abundant reporter gene expression. The SRD5a amplicon was ligated 5′ and 3′ with T7 primers. An in vitro transcription reaction (Ambion) was conducted according to manufacturer's instructions to generate full-length double stranded RNA (dsRNA) encoding for the SRD5a protein. RNase III digestion was carried out according to manufacturer's (Ambion) directions to generate several 19-21 nucleotide dsRNAs. These dsRNA species were rhodamine labeled (Molecular Probes) and transfected (Qiagen) into Sy5y cells expressing the SRD5a-GFP protein. Procedures were preformed according to the respective manufacturer's instructions.

FIG. 5B shows confocal micrographs of GFP reporter cells containing rhodamine labeled dsRNA for knockdown of the SRD5a protein 4 hours post-transfection of Rhodamine labeled dsRNA. Panel A shows green fluorescent protein fluorescence in transfected cells, and panel B shows the same cells with rhodamine labeled dsRNA fluorescence.

FIG. 5C is a confocal fluorescence micrograph taken 24 hrs after transfection with the dsRNA. The arrow indicates puntate green fluoresence in a field of predominant rhodamine fluorescence. Thus, the data show that transfection with dsRNA inhibits the expression of the the SRD5a-GFP protein.

The present disclosure is not to be limited in scope by the exemplified embodiments which are intended as illustrations of single aspects of the disclosure, and any clones, nucleic or amino acid sequences which are functionally equivalent are within the scope of the disclosure. Indeed, various modifications of the disclosure in addition to those described herein will become apparent to those skilled in the art from the foregoing description and accompanying drawings. Such modifications are intended to fall within the scope of the appended claims.

All references cited herein are hereby incorporated by reference in their entirety.

TABLE 1
5-alpha reductase Type I

5-alpha reductase Type I - P18405 (gi: 134151) [SEQ ID NO. 1]

| | |
|---|---|
| 1 | atggcaacgg cgacgggggt ggcggaggag cgcctgctgg ccgcgctcgc ctacctgcag |
| 61 | tgcgccgtgg gctgcgcggt cttcccgcgt ctccgcagcg cgcccaactg catcctcctg |
| 121 | gccatgttcc tcgtccacta cgggcatcgg tgcttaattt acccatttct gatgcgagga |
| 181 | ggaaagccta tgccactgtt ggcgtgtaca atggcgatta tgttctgtac ctgtaacggc |
| 241 | tatttgcaaa gcagatactt gagccattgt gcagtgtatg ctgatgactg ggtaacagat |
| 301 | ccccgttttc taataggttt tggcttgtgg ttaacaggca tgttgataaa catccattca |
| 361 | gatcatatcc taaggaatct cagaaaacca ggagatactg gatacaaaat accaagggga |
| 421 | ggcttatttg aatacgtaac tgcagccaac tattttggag aaatcatgga gtggtgtggc |
| 481 | tatgccctgg ccagctggtc tgtccaaggc gcggcttttg ctttcttcac gttttgtttt |
| 541 | ttatctggta gagcaaaaga gcatcatgag tggtacctcc ggaaatttga agagtatcca |
| 601 | aagttcagaa aaattataat tccatttttg ttttaa |

| | |
|---|---|
| Target sequence 1: AACGGCGACGGGGGTGGCGGA | [SEQ ID NO. 2] |
| Position in gene sequence: 6 | |
| GC content: 76.2% | |
| Sense strand siRNA: CGGCGACGGGGGUGGCGGAtt | [SEQ ID NO. 3] |
| Antisense strand siRNA: UCCGCCACCCCCGUCGCCGtt | [SEQ ID NO. 4] |
| Target sequence 2: AACTGCATCCTCCTG121GCC | [SEQ ID NO. 5] |
| Position in gene sequence: 108 | |
| GC content: 52.4% | |
| Sense strand siRNA: CUGCAUCCUCCUG121GCCtt | [SEQ ID NO. 6] |
| Antisense strand siRNA: GGC121CAGGAGGAUGCAGtt | [SEQ ID NO. 7] |
| Target sequence 3: AATTTACCCATTTCTGATGCG | [SEQ ID NO. 8] |
| Position in gene sequence: 161 | |
| GC content: 38.1% | |
| Sense strand siRNA: UUUACCCAUUUCUGAUGCGtt | [SEQ ID NO. 9] |
| Antisense strand siRNA: CGCAUCAGAAAUGGGUAAAtt | [SEQ ID NO. 10] |
| Target sequence 4: AAAGCCTATGCCACTGTTGGC | [SEQ ID NO. 11] |
| Position in gene sequence: 191 | |
| GC content: 52.4% | |
| Sense strand siRNA: AGCCUAUGCCACUGUUGGCtt | [SEQ ID NO. 12] |
| Antisense strand siRNA: GCCAACAGUGGCAUAGGCUtt | [SEQ ID NO. 13] |
| Target sequence 5: AATGGCGATIATGTTCTGTAC | [SEQ ID NO. 14] |
| Position in gene sequence: 218 | |
| GC content: 38.1% | |
| Sense strand siRNA: UGGCGAUUAUGUUCUGUACtt | [SEQ ID NO. 15] |
| Antisense strand siRNA: GUACAGAACAUAAUCGCCAtt | [SEQ ID NO. 16] |
| Target sequence 6: AACGGC241TATTTGCAAAGC | [SEQ ID NO. 17] |
| Position in gene sequence: 243 | |
| GC content: 38.1% | |
| Sense strand siRNA: CGGC241UAUUUGCAAAGCtt | [SEQ ID NO. 18] |
| Antisense strand siRNA: GCUUUGCAAAUA142GCCGtt | [SEQ ID NO. 19] |
| Target sequence 7: AAAGCAGATAC1TGAGCCATT | [SEQ ID NO. 20] |
| Position in gene sequence: 259 | |
| GC content: 38.1% | |
| Sense strand siRNA: AGCAGAUACUUGAGCCAUUtt | [SEQ ID NO. 21] |
| Antisense strand siRNA: AAUGGCUCAAGUAUCUGCUtt | [SEQ ID NO. 22] |
| Target sequence 8: AACAGAT301CCCCGTTTTCT | [SEQ ID NO. 23] |
| Position in gene sequence: 305 | |
| GC content: 38.1% | |
| Sense strand siRNA: CAGAU301CCCCGUUUUCUtt | [SEQ ID NO. 24] |
| Antisense strand siRNA: AGAAAACGGGG103AUCUGtt | [SEQ ID NO. 25] |
| Target sequence 9: AATAGGTTTTGGCTTGTGGTT | [SEQ ID NO. 26] |
| Position in gene sequence: 326 | |
| GC content: 38.1% | |
| Sense strand siRNA: UAGGUUUUGGCUUGUGGUUtt | [SEQ ID NO. 27] |
| Antisense strand siRNA: AACCACAAGCCAAAACCUAtt | [SEQ ID NO. 28] |
| Target sequence 10: AACAGGCATGTTGATAAACAT | [SEQ ID NO. 29] |
| Position in gene sequence: 347 | |
| GC content: 33.3% | |
| Sense strand siRNA: CAGGCAUGUUGAUAAACAUtt | [SEQ ID NO. 30] |
| Antisense strand siRNA: AUGUUUAUCAACAUGCCUGtt | [SEQ ID NO. 31] |
| Target sequence 11: AAACATCCATTCA361GATCA | [SEQ ID NO. 32] |
| Position in gene sequence: 362 | |
| GC content: 28.6% | |
| Sense strand siRNA: ACAUCCAUUCA361GAUCAtt | [SEQ ID NO. 33] |
| Antisense strand siRNA: UGAUC163UGAAUGGAUGUtt | [SEQ ID NO. 34] |
| Target sequence 12: AAGGAATCTCAGAAAACCAGG | [SEQ ID NO. 35] |
| Position in gene sequence: 389 | |
| GC content: 42.9% | |
| Sense strand siRNA: GGAAUCUCAGAAAACCAGGtt | [SEQ ID NO. 36] |
| Antisense strand siRNA: CCUGGUUUUCUGAGAUUCCtt | [SEQ ID NO. 37] |
| Target sequence 13: AATCTCAGAAAACCAGGAGAT | [SEQ ID NO. 38] |
| Position in gene sequence: 393 | |
| GC content: 38.1% | |
| Sense strand siRNA: UCUCAGAAAACCAGGAGAUtt | [SEQ ID NO. 39] |
| Antisense strand siRNA: AUCUCCUGGUUUUCUGAGAtt | [SEQ ID NO. 40] |
| Target sequence 14: AAAACCAGGAGATACTGGATA | [SEQ ID NO. 41] |
| Position in gene sequence: 401 | |
| GC content: 38.1% | |
| Sense strand siRNA: AACCAGGAGAUACUGGAUAtt | [SEQ ID NO. 42] |
| Antisense strand siRNA: UAUCCAGUAUCUCCUGGUUtt | [SEQ ID NO. 43] |
| Target sequence 15: AACCAGGAGATACTGGATACA | [SEQ ID NO. 44] |
| Position in gene sequence: 403 | |
| GC content: 42.9% | |
| Sense strand siRNA: CCAGGAGAUACUGGAUACAtt | [SEQ ID NO. 45] |
| Antisense strand siRNA: UGUAUCCAGUAUCUCCUGGtt | [SEQ ID NO. 46] |
| Target sequence 16: AAAATACCAAGGGGA421GGC | [SEQ ID NO. 47] |
| Position in gene sequence: 423 | |
| GC content: 42.9% | |
| Sense strand siRNA: AAUACCAAGGGGA421GGCtt | [SEQ ID NO. 48] |
| Antisense strand siRNA: GCC124UCCCCUUGGUAUUtt | [SEQ ID NO. 49] |
| Target sequence 17: AATACCAAGGGGA421GGCTT | [SEQ ID NO. 50] |
| Position in gene sequence: 425 | |
| GC content: 42.9% | |
| Sense strand siRNA: UACCAAGGGGA421GGCUUtt | [SEQ ID NO. 51] |
| Antisense strand siRNA: AAGCC124UCCCCUUGGUAtt | [SEQ ID NO. 52] |
| Target sequence 18: AAGGGGA421GGCTTATTTGA | [SEQ ID NO. 53] |
| Position in gene sequence: 431 | |
| GC content: 38.1% | |
| Sense strand siRNA: GGGGA421GGCUUAUUUGAtt | [SEQ ID NO. 54] |
| Antisense strand siRNA: UCAAAUAAGCC124UCCCCtt | [SEQ ID NO. 55] |
| Target sequence 19: AATACGTAACTGCAGCCAACT | [SEQ ID NO. 56] |
| Position in gene sequence: 451 | |
| GC content: 42.9% | |
| Sense strand siRNA: UACGUAACUGCAGCCAACUtt | [SEQ ID NO. 57] |
| Antisense strand siRNA: AGUUGGCUGCAGUUACGUAtt | [SEQ ID NO. 58] |
| Target sequence 20: AACTGCAGCCAACTATTTTGG | [SEQ ID NO. 59] |
| Position in gene sequence: 458 | |
| GC content: 42.9% | |
| Sense strand siRNA: CUGCAGCCAACUAUUUUGGtt | [SEQ ID NO. 60] |
| Antisense strand siRNA: CCAAAAUAGUUGGCUGCAGtt | [SEQ ID NO. 61] |
| Target sequence 21: AACTATTTTGGAGAAATCATG | [SEQ ID NO. 62] |
| Position in gene sequence: 468 | |
| GC content: 28.6% | |
| Sense strand siRNA: CUAUUUUGGAGAAAUCAUGtt | [SEQ ID NO. 63] |
| Antisense strand siRNA: CAUGAUUUCUCCAAAAUAGtt | [SEQ ID NO. 64] |
| Target sequence 22: AAATCATGGAGTGGTGTGGC4 | [SEQ ID NO. 65] |
| Position in gene sequence: 481 | |
| GC content: 47.6% | |
| Sense strand siRNA: AUCAUGGAGUGGUGUGGC4tt | [SEQ ID NO. 66] |
| Antisense strand siRNA: 4GCCACACCACUCCAUGAUtt | [SEQ ID NO. 67] |
| Target sequence 23: AAGGCGCGGCTTTTGCTTTCT | [SEQ ID NO. 68] |
| Position in gene sequence: 529 | |
| GC content: 52.4% | |
| Sense strand siRNA: GGCGCGGCUUUUGCUUUCUtt | [SEQ ID NO. 69] |
| Antisense strand siRNA: AGAAAGCAAAAGCCGCGCCtt | [SEQ ID NO. 70] |
| Target sequence 24: AAAAGAGCATCATGAGTGGTA | [SEQ ID NO. 71] |
| Position in gene sequence: 581 | |
| GC content: 38.1% | |
| Sense strand siRNA: AAGAGCAUCAUGAGUGGUAtt | [SEQ ID NO. 72] |
| Antisense strand siRNA: UACCACUCAUGAUGCUCUUtt | [SEQ ID NO. 73] |
| Target sequence 25: AAGAGCATCATGAGTGGTACC | [SEQ ID NO. 74] |
| Position in gene sequence: 583 | |
| GC content: 47.6% | |
| Sense strand siRNA: GAGCAUCAUGAGUGGUACCtt | [SEQ ID NO. 75] |
| Antisense strand siRNA: GGUACCACUCAUGAUGCUCtt | [SEQ ID NO. 76] |
| Target sequence 26: AAATTTGAAGAGTATCCA601 | [SEQ ID NO. 77] |
| Position in gene sequence: 609 | |
| GC content: 23.8% | |
| Sense strand siRNA: AUUUGAAGAGUAUCCA601tt | [SEQ ID NO. 78] |
| Antisense strand siRNA: 106UGGAUACUCUUCAAAUtt | [SEQ ID NO. 79] |
| Target sequence 27: AAGAGTATCCA601AAGTTCA | [SEQ ID NO. 80] |
| Position in gene sequence: 616 | |
| GC content: 28.6% | |
| Sense strand siRNA: GAGUAUCCA601AAGUUCAtt | [SEQ ID NO. 81] |
| Antisense strand siRNA: UGAACUU106UGGAUACUCtt | [SEQ ID NO. 82] |
| Target sequence 28: AAGTTCAGAAAAATTATAATT | [SEQ ID NO. 83] |
| Position in gene sequence: 630 | |
| GC content: 14.3% | |
| Sense strand siRNA: GUUCAGAAAAAUUAUAAUUtt | [SEQ ID NO. 84] |
| Antisense strand siRNA: AAUUAUAAUUUUUCUGAACtt | [SEQ ID NO. 85] |
| Target sequence 29: AAAAATTATAATTCCATTTTT | [SEQ ID NO. 86] |
| Position in gene sequence: 638 | |
| GC content: 9.5% | |
| Sense strand siRNA: AAAUUAUAAUUCCAUUUUUtt | [SEQ ID NO. 87] |
| Antisense strand siRNA: AAAAAUGGAAUUAUAAUUUtt | [SEQ ID NO. 88] |
| Target sequence 30: AAATTATAATTCCATTTTTGT | [SEQ ID NO. 89] |
| Position in gene sequence: 640 | |
| GC content: 14.3% | |
| Sense strand siRNA: AUUAUAAUUCCAUUUUUGUtt | [SEQ ID NO. 90] |
| Antisense strand siRNA: ACAAAAAUGGAAUUAUAAUtt | [SEQ ID NO. 91] |

TABLE 2
5-alpha reductase Type II

5-alpha reductase Type II - Q28892 (gi: 2498888) [SEQ ID NO. 92]

| | |
|---|---|
| 1 | atgcaggttc agtgccagca gagcccagtg ctggcaggca gcgccacttt ggtcgccctt |
| 61 | ggggcactgg tcttgtacgt cgcgaagccc tccggctacg ggaagcacac ggagagcctg |
| 121 | aagcccgcgg ctacccgcct gccagcccgc gcagcctggt tcctgcagga gctgccctcc |
| 181 | ttcgcggtgc ccgcggggat cctcgctcgg cagcccctct ccctcttcgg gccacctggg |
| 241 | acggtgcttc tgggcctctt ctgcgtacat tacttccaca ggacatttgt gtactcactg |
| 301 | ctcaatcgag ggaggcctta tccagctgta ctcattttcc gaggcattgc cttctgcgct |
| 361 | ggaaatggat tccttcaaag ctactatctg atttactgtg ctgaataccc tgatgggtgg |
| 421 | tacacagaca tacggttttg cttgggtgtc ttcttattta ttttgggaat gggagtcaac |
| 481 | atccatagtg actatatatt gcgccagctc aggaagcctg gagaaatcac ctacaggatt |
| 541 | ccaaaaggtg gcttgtttac gtatgtttct ggagccaatt tccttggtga gatcattgaa |
| 601 | tggatcggct atgcgctggc cacttggtcc ctcccagcac ttgcatttgc atttttctca |
| 661 | gtttgtttcc ttgggctgcg agcttttcac caccataggt tctacctcaa gatgtttgag |
| 721 | gactacccca aatctcggaa agcccttatt ccattcatct tttaa |

| | |
|---|---|
| Target sequence 1: AAGCCCTCCGGCTACGGGAAG | [SEQ ID NO. 93] |
| Position in gene sequence: 88 | |
| GC content: 66.7% | |
| Sense strand siRNA: GCCCUCCGGCUACGGGAAGtt | [SEQ ID NO. 94] |
| Antisense strand siRNA: CUUCCCGUAGCCGGAGGGCtt | [SEQ ID NO. 95] |
| Target sequence 2: AAGCACACGGAGAGCCTG121 | [SEQ ID NO. 96] |
| Position in gene sequence: 106 | |
| GC content: 52.4% | |
| Sense strand siRNA: GCACACGGAGAGCCUG121tt | [SEQ ID NO. 97] |
| Antisense strand siRNA: 121CAGGCUCUCCGUGUGCtt | [SEQ ID NO. 98] |
| Target sequence 3: AAGCCCGCGGCTACCCGCCTG | [SEQ ID NO. 99] |
| Position in gene sequence: 127 | |
| GC content: 76.2% | |
| Sense strand siRNA: GCCCGCGGCUACCCGCCUGtt | [SEQ ID NO. 100] |
| Antisense strand siRNA: CAGGCGGGUAGCCGCGGGCtt | [SEQ ID NO. 101] |
| Target sequence 4: AATCGAGGGAGGCCTTATCCA | [SEQ ID NO. 102] |
| Position in gene sequence: 319 | |
| GC content: 52.4% | |
| Sense strand siRNA: UCGAGGGAGGCCUUAUCCAtt | [SEQ ID NO. 103] |
| Antisense strand siRNA: UGGAUAAGGCCUCCCUCGAtt | [SEQ ID NO. 104] |
| Target sequence 5: AAATGGATTCCTTCAAAGCTA | [SEQ ID NO. 105] |
| Position in gene sequence: 381 | |
| GC content: 33.3% | |
| Sense strand siRNA: AUGGAUUCCUUCAAAGCUAtt | [SEQ ID NO: 106] |
| Antisense strand siRNA: UAGCUUUGAAGGAAUCCAUtt | [SEQ ID NO. 107] |
| Target sequence 6: AAAGCTACTATCTGATTTACT | [SEQ ID NO. 108] |
| Position in gene sequence: 395 | |
| GC content: 28.6% | |
| Sense strand siRNA: AGCUACUAUCUGAUUUACUtt | [SEQ ID NO. 109] |
| Antisense strand siRNA: AGUAAAUCAGAUAGUAGCUtt | [SEQ ID NO. 110] |
| Target sequence 7: AATACCCTGATGGGTGG421T | [SEQ ID NO. 111] |
| Position in gene sequence: 422 | |
| GC content: 42.9% | |
| Sense strand siRNA: UACCCUGAUGGGUGG421Utt | [SEQ ID NO. 112] |
| Antisense strand siRNA: A124CCACCCAUCAGGGUAtt | [SEQ ID NO. 113] |
| Target sequence 8: AATGGGAGTCAAC481ATCCA | [SEQ ID NO. 114] |
| Position in gene sequence: 489 | |
| GC content: 38.1% | |
| Sense strand siRNA: UGGGAGUCAAC481AUCCAtt | [SEQ ID NO. 115] |
| Antisense strand siRNA: UGGAU184GUUGACUCCCAtt | [SEQ ID NO. 116] |
| Target sequence 9: AAC481ATCCATAGTGACTAT | [SEQ ID NO. 117] |
| Position in gene sequence: 499 | |
| GC content: 28.6% | |
| Sense strand siRNA: C481AUCCAUAGUGACUAUtt | [SEQ ID NO. 118] |
| Antisense strand siRNA: AUAGUCACUAUGGAU184Gtt | [SEQ ID NO. 119] |
| Target sequence 10: AAGCCTGGAGAAATCACCTAC | [SEQ ID NO. 120] |
| Position in gene sequence: 538 | |
| GC content: 47.6% | |
| Sense strand siRNA: GCCUGGAGAAAUCACCUACtt | [SEQ ID NO. 121] |
| Antisense strand siRNA: GUAGGUGAUUUCUCCAGGCtt | [SEQ ID NO. 122] |
| Target sequence 11: AAATCACCTACAGGATT541C | [SEQ ID NO. 123] |
| Position in gene sequence: 548 | |
| GC content: 33.3% | |
| Sense strand siRNA: AUCACCUACAGGAUU541Ctt | [SEQ ID NO. 124] |
| Antisense strand siRNA: G145AAUCCUGUAGGUGAUtt | [SEQ ID NO. 125] |
| Target sequence 12: AAAAGGTGGCTTGTTTACGTA | [SEQ ID NO. 126] |
| Position in gene sequence: 570 | |
| GC content: 38.1% | |
| Sense strand siRNA: AAGGUGGCUUGUUUACGUAtt | [SEQ ID NO. 127] |
| Antisense strand siRNA: UACGUAAACAAGCCACCUUtt | [SEQ ID NO. 128] |
| Target sequence 13: AAGGTGGCTTGTTTACGTATG | [SEQ ID NO. 129] |
| Position in gene sequence: 572 | |
| GC content: 42.9% | |
| Sense strand siRNA: GGUGGCUUGUUUACGUAUGtt | [SEQ ID NO. 130] |
| Antisense strand siRNA: CAUACGUAAACAAGCCACCtt | [SEQ ID NO. 131] |
| Target sequence 14: AATTTCCTTGGTGAGATCATT | [SEQ ID NO. 132] |
| Position in gene sequence: 604 | |
| GC content: 33.3% | |
| Sense strand siRNA: UUUCCUUGGUGAGAUCAUUtt | [SEQ ID NO. 133] |
| Antisense strand siRNA: AAUGAUCUCACCAAGGAAAtt | [SEQ ID NO. 134] |
| Target sequence 15: AA601TGGATCGGCTATGCGC | [SEQ ID NO. 135] |
| Position in gene sequence: 626 | |
| GC content: 47.6% | |
| Sense strand siRNA: 601UGGAUCGGCUAUGCGCtt | [SEQ ID NO. 136] |
| Antisense strand siRNA: GCGCAUAGCCGAUCCA106tt | [SEQ ID NO. 137] |
| Target sequence 16: AAGATGTTTGAG721GACTAC | [SEQ ID NO. 138] |
| Position in gene sequence: 742 | |
| GC content: 33.3% | |
| Sense strand siRNA: GAUGUUUGAG721GACUACtt | [SEQ ID NO. 139] |
| Antisense strand siRNA: GUAGUC127CUCAAACAUCtt | [SEQ ID NO. 140] |
| Target sequence 17: AAATCTCGGAAAGCCCTTATT | [SEQ ID NO. 141] |
| Position in gene sequence: 766 | |
| GC content: 38.1% | |
| Sense strand siRNA: AUCUCGGAAAGCCCUUAUUtt | [SEQ ID NO. 142] |
| Antisense strand siRNA: AAUAAGGGCUUUCCGAGAUtt | [SEQ ID NO. 143] |
| Target sequence 18: AAAGCCCTTATTCCATTCATC | [SEQ ID NO. 144] |
| Position in gene sequence: 775 | |
| GC content: 38.1% | |
| Sense strand siRNA: AGCCCUUAUUCCAUUCAUCtt | [SEQ ID NO. 145] |
| Antisense strand siRNA: GAUGAAUGGAAUAAGGGCUtt | [SEQ ID NO. 146] |

TABLE 3
Androgen Receptor

Androgen Receptor - P10275 (gi: 21322251) [SEQ ID NO. 147]

| | |
|---|---|
| 1 | atggaagtgc agttagggct gggaagggtc taccctcggc cgccgtccaa gacctaccga |
| 61 | ggagctttcc agaatctgtt ccagagcgtg cgcgaagtga tccagaaccc gggccccagg |
| 121 | cacccagagg ccgcgagcgc agcacctccc ggcgccagtt tgctgctgct gcagcagcag |
| 181 | cagcagcagc agcagcagca gcagcagcag cagcagcagc agcagcagca gcagcagcaa |
| 241 | gagactagcc ccaggcagca gcagcagcag cagggtgagg atggttctcc ccaagcccat |
| 301 | cgtagaggcc ccacaggcta cctggtcctg gatgaggaac agcaaccttc acagccgcag |
| 361 | tcggccctgg agtgccaccc cgagagaggt tgcgtcccag agcctggagc cgccgtggcc |
| 421 | gccagcaagg ggctgccgca gcagctgcca gcacctccgg acgaggatga ctcagctgcc |
| 481 | ccatccacgt tgtccctgct gggccccact ttccccggct taagcagctg ctccgctgac |
| 541 | cttaaagaca tcctgagcga ggccagcacc atgcaactcc ttcagcaaca gcagcaggaa |
| 601 | gcagtatccg aaggcagcag cagcgggaga gcgagggagg cctcgggggc tcccacttcc |
| 661 | tccaaggaca attacttagg gggcacttcg accatttctg acaacgccaa ggagttgtgt |
| 721 | aaggcagtgt cggtgtccat gggcctgggt gtggaggcgt tggagcatct gagtccaggg |
| 781 | gaacagcttc ggggggattg catgtacgcc ccacttttgg gagttccacc cgctgtgcgt |
| 841 | cccactcctt gtgccccatt ggccgaatgc aaaggttctc tgctagacga cagcgcaggc |
| 901 | aagagcactg aagatactgc tgagtattcc cctttcaagg gaggttacac caaagggcta |
| 961 | gaaggcgaga gcctaggctg ctctggcagc gctgcagcag ggagctccgg gacacttgaa |
| 1021 | ctgccgtcta ccctgtctct ctacaagtcc ggagcactgg acgaggcagc tgcgtaccag |
| 1081 | agtcgcgact actacaactt tccactggct ctggccggac cgccgccccc tccgccgcct |
| 1141 | ccccatcccc acgctcgcat caagctggag aacccgctgg actacggcag cgcctgggcg |
| 1201 | gctgcggcgg cgcagtgccg ctatggggac ctggcgagcc tgcatggcgc gggtgcagcg |
| 1261 | ggacccggtt ctgggtcacc ctcagccgcc gcttcctcat cctggcacac tctcttcaca |
| 1321 | gccgaagaag gccagttgta tggaccgtgt ggtggtggtg ggggtggtgg cggcggcggc |
| 1381 | ggcggcggcg gcggcggcgg cggcggcggc ggcggcggcg aggcgggagc tgtagccccc |
| 1441 | tacggctaca ctcggccccc tcaggggctg gcgggccagg aaagcgactt caccgcacct |
| 1501 | gatgtgtggt accctggcgg catggtgagc agagtgccct atcccagtcc cacttgtgtc |
| 1561 | aaaagcgaaa tgggcccctg gatggatagc tactccggac cttacgggga catgcgtttg |
| 1621 | gagactgcca gggaccatgt tttgcccatt gactattact ttccacccca gaagacctgc |
| 1681 | ctgatctgtg gagatgaagc ttctgggtgt cactatggag ctctcacatg tggaagctgc |
| 1741 | aaggtcttct tcaaaagagc cgctgaaggg aaacagaagt acctgtgcgc cagcagaaat |
| 1801 | gattgcacta ttgataaatt ccgaaggaaa aattgtccat cttgtcgtct tcggaaatgt |
| 1861 | tatgaagcag ggatgactct gggagcccgg aagctgaaga aacttggtaa tctgaaacta |
| 1921 | caggaggaag gagaggcttc cagcaccacc agccccactg aggagacaac ccagaagctg |
| 1981 | acagtgtcac acattgaagg ctatgaatgt cagcccatct ttctgaatgt cctggaagcc |
| 2041 | attgagccag gtgtagtgtg tgctggacac gacaacaacc agcccgactc ctttgcagcc |
| 2101 | ttgctctcta gcctcaatga actgggagag agacagcttg tacacgtggt caagtgggcc |
| 2161 | aaggccttgc ctggcttccg caacttacac gtggacgacc agatggctgt cattcagtac |
| 2221 | tcctggatgg ggctcatggt gtttgccatg ggctggcgat ccttcaccaa tgtcaactcc |
| 2281 | aggatgctct acttcgcccc tgatctggtt ttcaatgagt accgcatgca caagtcccgg |
| 2341 | atgtacagcc agtgtgtccg aatgaggcac ctctctcaag agtttggatg gctccaaatc |
| 2401 | accccccagg aattcctgtg catgaaagca ctgctactct tcagcattat tccagtggat |
| 2461 | gggctgaaaa atcaaaaatt ctttgatgaa cttcgaatga actacatcaa ggaactcgat |
| 2521 | cgtatcattg catgcaaaag aaaaaatccc acatcctgct caagacgctt ctaccagctc |
| 2581 | accaagctcc tggactccgt gcagcctatt gcgagagagc tgcatcagtt cacttttgac |
| 2641 | ctgctaatca agtcacacat ggtgagcgtg gactttccgg aaatgatggc agagatcatc |
| 2701 | tctgtgcaag tgcccaagat cctttctggg aaagtcaagc ccatctattt ccacacccag |
| 2761 | tga |

| | |
|---|---|
| Target sequence 1: AAGTGCAGTTAGGGCTGGGAA | [SEQ IN NO. 148] |
| Position in gene sequence: 5 | |
| GC content: 52.4% | |
| Sense strand siRNA: GUGCAGUUAGGGCUGGGAAtt | [SEQ ID NO. 149] |
| Antisense strand siRNA: UUCCCAGCCCUAACUGCACtt | [SEQ ID NO. 150] |
| Target sequence 2: AAGGGTCTACCCTCGGCCGCC | [SEQ ID NO. 151] |
| Position in gene sequence: 24 | |
| GC content: 71.4% | |
| Sense strand siRNA: GGGUCUACCCUCGGCCGCCtt | [SEQ ID NO. 152] |
| Antisense strand siRNA: GGCGGCCGAGGGUAGACCCtt | [SEQ ID NO. 153] |
| Target sequence 3: AAGACCTACCGA61GGAGCTT | [SEQ ID NO. 154] |
| Position in gene sequence: 49 | |
| GC content: 47.6% | |
| Sense strand siRNA: GACCUACCGA61GGAGCUUtt | [SEQ ID NO. 155] |
| Antisense strand siRNA: AAGCUCC16UCGGUAGGUCtt | [SEQ ID NO. 156] |
| Target sequence 4: AATCTGTTCCAGAGCGTGCGC | [SEQ ID NO. 157] |
| Position in gene sequence: 75 | |
| GC content: 57.1% | |
| Sense strand siRNA: UCUGUUCCAGAGCGUGCGCtt | [SEQ ID NO. 158] |
| Antisense strand siRNA: GCGCACGCUCUGGAACAGAtt | [SEQ ID NO. 159] |
| Target sequence 5: AAGTGATCCAGAACCCGGGCC | [SEQ ID NO. 160] |
| Position in gene sequence: 97 | |
| GC content: 61.9% | |
| Sense strand siRNA: GUGAUCCAGAACCCGGGCCtt | [SEQ ID NO. 161] |
| Antisense strand siRNA: GGCCCGGGUUCUGGAUCACtt | [SEQ ID NO. 162] |
| Target sequence 6: AACCCGGGCCCCAGG121CAC | [SEQ ID NO. 163] |
| Position in gene sequence: 108 | |
| GC content: 66.7% | |
| Sense strand siRNA: CCCGGGCCCCAGG121CACtt | [SEQ ID NO. 164] |
| Antisense strand siRNA: GUG121CCUGGGGCCCGGGtt | [SEQ ID NO. 165] |
| Target sequence 7: AA241GAGACTAGCCCCAGGC | [SEQ ID NO. 166] |
| Position in gene sequence: 247 | |
| GC content: 52.4% | |
| Sense strand siRNA: 241GAGACUAGCCCCAGGCtt | [SEQ ID NO. 167] |
| Antisense strand siRNA: GCCUGGGGCUAGUCUC142tt | [SEQ ID NO. 168] |
| Target sequence 8: AAGCCCAT301CGTAGAGGCC | [SEQ ID NO. 169] |
| Position in gene sequence: 304 | |
| GC content: 52.4% | |
| Sense strand siRNA: GCCCAU301CGUAGAGGCCtt | [SEQ ID NO. 170] |
| Antisense strand siRNA: GGCCUCUACG103AUGGGCtt | [SEQ ID NO. 171] |
| Target sequence 9: AACAGCAACCTTCACAGCCGC | [SEQ ID NO. 172] |
| Position in gene sequence: 352 | |
| GC content: 57.1% | |
| Sense strand siRNA: CAGCAACCUUCACAGCCGCtt | [SEQ ID NO. 173] |
| Antisense strand siRNA: GCGGCUGUGAAGGUUGCUGtt | [SEQ ID NO. 174] |
| Target sequence 10: AACCTTCACAGCCGCAG361T | [SEQ ID NO. 175] |
| Position in gene sequence: 358 | |
| GC content: 47.6% | |
| Sense strand siRNA: CCUUCACAGCCGCAG361Utt | [SEQ ID NO. 176] |
| Antisense strand siRNA: A163CUGCGGCUGUGAAGGtt | [SEQ ID NO. 177] |
| Target sequence 11: AAGGGGCTGCCGCAGCAGCTG | [SEQ ID NO. 178] |
| Position in gene sequence: 447 | |
| GC content: 71.4% | |
| Sense strand siRNA: GGGGCUGCCGCAGCAGCUGtt | [SEQ ID NO. 179] |
| Antisense strand siRNA: CAGCUGCUGCGGCAGCCCCtt | [SEQ ID NO. 180] |
| Target sequence 12: AAGCAGCTGCTCCGCTGAC54 | [SEQ ID NO. 181] |
| Position in gene sequence: 545 | |
| GC content: 57.1% | |
| Sense strand siRNA: GCAGCUGCUCCGCUGAC54tt | [SEQ ID NO. 182] |
| Antisense strand siRNA: 45GUCAGCGGAGCAGCUGCtt | [SEQ ID NO. 183] |
| Target sequence 13: AAAGACATCCTGAGCGAGGCC | [SEQ ID NO. 184] |
| Position in gene sequence: 570 | |
| GC content: 57.1% | |
| Sense strand siRNA: AGACAUCCUGAGCGAGGCCtt | [SEQ ID NO. 185] |
| Antisense strand siRNA: GGCCUCGCUCAGGAUGUCUtt | [SEQ ID NO. 186] |
| Target sequence 14: AACTCCTTCAGCAACAGCAGC | [SEQ ID NO. 187] |
| Position in gene sequence: 601 | |
| GC content: 52.4% | |
| Sense strand siRNA: CUCCUUCAGCAACAGCAGCtt | [SEQ ID NO. 188] |
| Antisense strand siRNA: GCUGCUGUUGCUGAAGGAGtt | [SEQ ID NO. 189] |
| Target sequence 15: AACAGCAGCAGGAA601GCAG | [SEQ ID NO. 190] |
| Position in gene sequence: 613 | |
| GC content: 47.6% | |
| Sense strand siRNA: CAGCAGCAGGAA601GCAGtt | [SEQ ID NO. 191] |
| Antisense strand siRNA: CUGC106UUCCUGCUGCUGtt | [SEQ ID NO. 192] |
| Target sequence 16: AA601GCAGTATCCGAAGGCA | [SEQ ID NO. 193] |
| Position in gene sequence: 625 | |
| GC content: 42.9% | |
| Sense strand siRNA: 601GCAGUAUCCGAAGGCAtt | [SEQ ID NO. 194] |
| Antisense strand siRNA: UGCCUUCGGAUACUGC106tt | [SEQ ID NO. 195] |
| Target sequence 17: AAGGCAGCAGCAGCGGGAGAG | [SEQ ID NO. 196] |
| Position in gene sequence: 640 | |
| GC content: 66.7% | |
| Sense strand siRNA: GGCAGCAGCAGCGGGAGAGtt | [SEQ ID NO. 197] |
| Antisense strand siRNA: CUCUCCCGCUGCUGCUGCCtt | [SEQ ID NO. 198] |
| Target sequence 18: AAGGACAATTACTTAGGGGGC | [SEQ ID NO. 199] |
| Position in gene sequence: 696 | |
| GC content: 47.6% | |
| Sense strand siRNA: GGACAAUUACUUAGGGGGCtt | [SEQ ID NO. 200] |
| Antisense strand siRNA: GCCCCCUAAGUAAUUGUCCtt | [SEQ ID NO. 201] |
| Target sequence 19: AATTACTTAGGGGGCACTTCG | [SEQ ID NO. 202] |
| Position in gene sequence: 702 | |
| GC content: 47.6% | |
| Sense strand siRNA: UUACUUAGGGGGCACUUCGtt | [SEQ ID NO. 203] |
| Antisense strand siRNA: CGAAGUGCCCCCUAAGUAAtt | [SEQ ID NO. 204] |
| Target sequence 20: AACGCCAAGGAGTTGTGT721 | [SEQ ID NO. 205] |
| Position in gene sequence: 735 | |
| GC content: 42.9% | |
| Sense strand siRNA: CGCCAAGGAGUUGUGU721tt | [SEQ ID NO. 206] |
| Antisense strand siRNA: 127ACACAACUCCUUGGCGtt | [SEQ ID NO. 207] |
| Target sequence 21: AAGGAGTTGTGT721AAGGCA | [SEQ ID NO. 208] |
| Position in gene sequence: 741 | |
| GC content: 38.1% | |
| Sense strand siRNA: GGAGUUGUGU721AAGGCAtt | [SEQ ID NO. 209] |
| Antisense strand siRNA: UGCCUU127ACACAACUCCtt | [SEQ ID NO. 210] |
| Target sequence 22: AAGGCAGTGTCGGTGTCCATG | [SEQ ID NO. 211] |
| Position in gene sequence: 756 | |
| GC content: 57.1% | |
| Sense strand siRNA: GGCAGUGUCGGUGUCCAUGtt | [SEQ ID NO. 212] |
| Antisense strand siRNA: CAUGGACACCGACACUGCCtt | [SEQ ID NO. 213] |
| Target sequence 23: AACAGCTTCGGGGGGATTGCA | [SEQ ID NO. 214] |
| Position in gene sequence: 820 | |
| GC content: 57.1% | |
| Sense strand siRNA: CAGCUUCGGGGGGAUUGCAtt | [SEQ ID NO. 215] |
| Antisense strand siRNA: UGCAAUCCCCCCGAAGCUGtt | [SEQ ID NO. 216] |
| Target sequence 24: AATGCAAAGGTTCTCTGCTAG | [SEQ ID NO. 217] |
| Position in gene sequence: 907 | |
| GC content: 42.9% | |
| Sense strand siRNA: UGCAAAGGUUCUCUGCUAGtt | [SEQ ID NO. 218] |
| Antisense strand siRNA: CUAGCAGAGAACCUUUGCAtt | [SEQ ID NO. 219] |
| Target sequence 25: AAAGGTTCTCTGCTAGACGAC | [SEQ ID NO. 220] |
| Position in gene sequence: 912 | |
| GC content: 47.6% | |
| Sense strand siRNA: AGGUUCUCUGCUAGACGACtt | [SEQ ID NO. 221] |
| Antisense strand siRNA: GUCGUCUAGCAGAGAACCUtt | [SEQ ID NO. 222] |
| Target sequence 26: AAGAGCACTGAAGATACTGCT | [SEQ ID NO. 223] |
| Position in gene sequence: 945 | |
| GC content: 42.9% | |
| Sense strand siRNA: GAGCACUGAAGAUACUGCUtt | [SEQ ID NO. 224] |
| Antisense strand siRNA: AGCAGUAUCUUCAGUGCUCtt | [SEQ ID NO. 225] |
| Target sequence 27: AAGATACTGCTGAGTATTCCC | [SEQ ID NO. 226] |
| Position in gene sequence: 955 | |
| GC content: 42.9% | |
| Sense strand siRNA: GAUACUGCUGAGUAUUCCCtt | [SEQ ID NO. 227] |
| Antisense strand siRNA: GGGAAUACUCAGCAGUAUCtt | [SEQ ID NO. 228] |
| Target sequence 28: AAGGGAGGTTACACCAAAGGG | [SEQ ID NO. 229] |
| Position in gene sequence: 981 | |
| GC content: 52.4% | |
| Sense strand siRNA: GGGAGGUUACACCAAAGGGtt | [SEQ ID NO. 230] |
| Antisense strand siRNA: CCCUUUGGUGUAACCUCCCtt | [SEQ ID NO. 231] |
| Target sequence 29: AAAGGGCTA961GAAGGCGAG | [SEQ ID NO. 232] |
| Position in gene sequence: 996 | |
| GC content: 47.6% | |
| Sense strand siRNA: AGGGCUA961GAAGGCGAGtt | [SEQ ID NO. 233] |
| Antisense strand siRNA: CUCGCCUUC169UAGCCCUtt | [SEQ ID NO. 234] |
| Target sequence 30: AAGGCGAGAGCCTAGGCTGCT | [SEQ ID NO. 235] |
| Position in gene sequence: 1009 | |
| GC content: 61.9% | |
| Sense strand siRNA: GGCGAGAGCCUAGGCUGCUtt | [SEQ ID NO. 236] |
| Antisense strand siRNA: AGCAGCCUAGGCUCUCGCCtt | [SEQ ID NO. 237] |
| Target sequence 31: AA1021CTGCCGTCTACCCTG | [SEQ ID NO. 238] |
| Position in gene sequence: 1066 | |
| GC content: 47.6% | |
| Sense strand siRNA: 1021CUGCCGUCUACCCUGtt | [SEQ ID NO. 239] |
| Antisense strand siRNA: CAGGGUAGACGGCAG1201tt | [SEQ ID NO. 240] |
| Target sequence 32: AAGTCCGGAGCACTGGACGAG | [SEQ ID NO. 241] |
| Position in gene sequence: 1096 | |
| GC content: 61.9% | |
| Sense strand siRNA: GUCCGGAGCACUGGACGAGtt | [SEQ ID NO. 242] |
| Antisense strand siRNA: CUCGUCCAGUGCUCCGGACtt | [SEQ ID NO. 243] |
| Target sequence 33: AACTTTCCACTGGCTCTGGCC | [SEQ ID NO. 244] |
| Position in gene sequence: 1151 | |
| GC content: 57.1% | |
| Sense strand siRNA: CUUUCCACUGGCUCUGGCCtt | [SEQ ID NO. 245] |
| Antisense strand siRNA: GGCCAGAGCCAGUGGAAAGtt | [SEQ ID NO. 246] |
| Target sequence 34: AAGCTGGAGAACCCGCTGGAC | [SEQ ID NO. 247] |
| Position in gene sequence: 1221 | |
| GC content: 61.9% | |
| Sense strand siRNA: GCUGGAGAACCCGCUGGACtt | [SEQ ID NO. 248] |
| Antisense strand siRNA: GUCCAGCGGGUUCUCCAGCtt | [SEQ ID NO. 249] |
| Target sequence 35: AACCCGCTGGACTACGGCAGC | [SEQ ID NO. 250] |
| Position in gene sequence: 1230 | |
| GC content: 66.7% | |
| Sense strand siRNA: CCCGCUGGACUACGGCAGCtt | [SEQ ID NO. 251] |
| Antisense strand siRNA: GCUGCCGUAGUCCAGCGGGtt | [SEQ ID NO. 252] |
| Target sequence 36: AAGAAGGCCAGTTGTATGGAC | [SEQ ID NO. 253] |
| Position in gene sequence: 1396 | |
| GC content: 47.6% | |
| Sense strand siRNA: GAAGGCCAGUUGUAUGGACtt | [SEQ ID NO. 254] |
| Antisense strand siRNA: GUCCAUACAACUGGCCUUCtt | [SEQ ID NO. 255] |
| Target sequence 37: AAGGCCAGTTGTATGGACCGT | [SEQ ID NO. 256] |
| Position in gene sequence: 1399 | |
| GC content: 52.4% | |
| Sense strand siRNA: GGCCAGUUGUAUGGACCGUtt | [SEQ ID NO. 257] |
| Antisense strand siRNA: ACGGUCCAUACAACUGGCCtt | [SEQ ID NO. 258] |
| Target sequence 38: AAAGCGACTTCACCGCACCT1 | [SEQ ID NO. 259] |
| Position in gene sequence: 1560 | |
| GC content: 52.4% | |
| Sense strand siRNA: AGCGACUUCACCGCACCU1tt | [SEQ ID NO. 260] |
| Antisense strand siRNA: 1AGGUGCGGUGAAGUCGCUtt | [SEQ ID NO. 261] |
| Target sequence 39: AAAAGCGAAATGGGCCCCTGG | [SEQ ID NO. 262] |
| Position in gene sequence: 1648 | |
| GC content: 57.1% | |
| Sense strand siRNA: AAGCGAAAUGGGCCCCUGGtt | [SEQ ID NO. 263] |
| Antisense strand siRNA: CCAGGGGCCCAUUUCGCUUtt | [SEQ ID NO. 264] |
| Target sequence 40: AAGCGAAATGGGCCCCTGGAT | [SEQ ID NO. 265] |
| Position in gene sequence: 1650 | |
| GC content: 57.1% | |
| Sense strand siRNA: GCGAAAUGGGCCCCUGGAUtt | [SEQ ID NO. 266] |
| Antisense strand siRNA: AUCCAGGGGCCCAUUUCGCtt | [SEQ ID NO. 267] |
| Target sequence 41: AAATGGGCCCCTGGATGGATA | [SEQ ID NO. 268] |
| Position in gene sequence: 1655 | |
| GC content: 52.4% | |
| Sense strand siRNA: AUGGGCCCCUGGAUGGAUAtt | [SEQ ID NO. 269] |
| Antisense strand siRNA: UAUCCAUCCAGGGGCCCAUtt | [SEQ ID NO. 270] |
| Target sequence 42: AAGACCTGC1681CTGATCTG | [SEQ ID NO. 271] |
| Position in gene sequence: 1763 | |
| GC content: 42.9% | |
| Sense strand siRNA: GACCUGC1681CUGAUCUGtt | [SEQ ID NO. 272] |
| Antisense strand siRNA: CAGAUCAG1861GCAGGUCtt | [SEQ ID NO. 273] |
| Target sequence 43: AAGCTTCTGGGTGTCACTATG | [SEQ ID NO. 274] |
| Position in gene sequence: 1792 | |
| GC content: 47.6% | |
| Sense strand siRNA: GCUUCUGGGUGUCACUAUGtt | [SEQ ID NO. 275] |
| Antisense strand siRNA: CAUAGUGACACCCAGAAGCtt | [SEQ ID NO. 276] |
| Target sequence 44: AAGCTGC1741AAGGTCTTCT | [SEQ ID NO. 277] |
| Position in gene sequence: 1829 | |
| GC content: 38.1% | |
| Sense strand siRNA: GCUGC1741AAGGUCUUCUtt | [SEQ ID NO. 278] |
| Antisense strand siRNA: AGAAGACCUU1471GCAGCtt | [SEQ ID NO. 279] |
| Target sequence 45: AAGGTCTTCTTCAAAAGAGCC | [SEQ ID NO. 280] |
| Position in gene sequence: 1840 | |
| GC content: 42.9% | |
| Sense strand siRNA: GGUCUUCUUCAAAAGAGCCtt | [SEQ ID NO. 281] |
| Antisense strand siRNA: GGCUCUUUUGAAGAAGACCtt | [SEQ ID NO. 282] |
| Target sequence 46: AAAAGAGCCGCTGAAGGGAAA | [SEQ ID NO. 283] |
| Position in gene sequence: 1852 | |
| GC content: 47.6% | |
| Sense strand siRNA: AAGAGCCGCUGAAGGGAAAtt | [SEQ ID NO. 284] |
| Antisense strand siRNA: UUUCCCUUCAGCGGCUCUUtt | [SEQ ID NO. 285] |
| Target sequence 47: AAGAGCCGCTGAAGGGAAACA | [SEQ ID NO. 286] |
| Position in gene sequence: 1854 | |
| GC content: 52.4% | |
| Sense strand siRNA: GAGCCGCUGAAGGGAAACAtt | [SEQ ID NO. 287] |
| Antisense strand siRNA: UGUUUCCCUUCAGCGGCUCtt | [SEQ ID NO. 288] |
| Target sequence 48: AAGGGAAACAGAAGTACCTGT | [SEQ ID NO. 289] |
| Position in gene sequence: 1865 | |
| GC content: 42.9% | |
| Sense strand siRNA: GGGAAACAGAAGUACCUGUtt | [SEQ ID NO. 290] |
| Antisense strand siRNA: ACAGGUACUUCUGUUUCCCtt | [SEQ ID NO. 291] |
| Target sequence 49: AAACAGAAGTACCTGTGCGCC | [SEQ ID NO. 292] |
| Position in gene sequence: 1870 | |
| GC content: 52.4% | |
| Sense strand siRNA: ACAGAAGUACCUGUGCGCCtt | [SEQ ID NO. 293] |
| Antisense strand siRNA: GGCGCACAGGUACUUCUGUtt | [SEQ ID NO. 294] |
| Target sequence 50: AAGTACCTGTGCGCCAGCAGA | [SEQ ID NO. 295] |
| Position in gene sequence: 1876 | |
| GC content: 57.1% | |
| Sense strand siRNA: GUACCUGUGCGCCAGCAGAtt | [SEQ ID NO. 296] |
| Antisense strand siRNA: UCUGCUGGCGCACAGGUACtt | [SEQ ID NO. 297] |
| Target sequence 51: AAAT1801GATTGCACTATTG | [SEQ ID NO. 298] |
| Position in gene sequence: 1896 | |
| GC content: 23.8% | |
| Sense strand siRNA: AU1801GAUUGCACUAUUGtt | [SEQ ID NO. 299] |
| Antisense strand siRNA: CAAUAGUGCAAUC1081Autt | [SEQ ID NO. 300] |
| Target sequence 52: AAATTCCGAAGGAAAAATTGT | [SEQ ID NO. 301] |
| Position in gene sequence: 1919 | |
| GC content: 28.6% | |
| Sense strand siRNA: AUUCCGAAGGAAAAAUUGUtt | [SEQ ID NO. 302] |
| Antisense strand siRNA: ACAAUUUUUCCUUCGGAAUtt | [SEQ ID NO. 303] |
| Target sequence 53: AAGGAAAAATTGTCCATCTTG | [SEQ ID NO. 304] |
| Position in gene sequence: 1927 | |
| GC content: 33.3% | |
| Sense strand siRNA: GGAAAAAUUGUCCAUCUUGtt | [SEQ ID NO. 305] |
| Antisense strand siRNA: CAAGAUGGACAAUUUUUCCtt | [SEQ ID NO. 306] |
| Target sequence 54: AAAAATTGTCCATCTTGTCGT | [SEQ ID NO. 307] |
| Position in gene sequence: 1931 | |
| GC content: 33.3% | |
| Sense strand siRNA: AAAUUGUCCAUCUUGUCGUtt | [SEQ ID NO. 308] |
| Antisense strand siRNA: ACGACAAGAUGGACAAUUUtt | [SEQ ID NO. 309] |
| Target sequence 55: AAATTGTCCATCTTGTCGTCT | [SEQ ID NO. 310] |
| Position in gene sequence: 1933 | |
| GC content: 38.1% | |
| Sense strand siRNA: AUUGUCCAUCUUGUCGUCUtt | [SEQ ID NO. 311] |
| Antisense strand siRNA: AGACGACAAGAUGGACAAUtt | [SEQ ID NO. 312] |
| Target sequence 56: AAATGT1861TATGAAGCAGG | [SEQ ID NO. 313] |
| Position in gene sequence: 1958 | |
| GC content: 28.6% | |
| Sense strand siRNA: AUGU1861UAUGAAGCAGGtt | [SEQ ID NO. 314] |
| Antisense strand siRNA: CCUGCUUCAUA1681ACAUtt | [SEQ ID NO. 315] |
| Target sequence 57: AAGCAGGGATGACTCTGGGAG | [SEQ ID NO. 316] |
| Position in gene sequence: 1972 | |
| GC content: 57.1% | |
| Sense strand siRNA: GCAGGGAUGACUCUGGGAGtt | [SEQ ID NO. 317] |
| Antisense strand siRNA: CUCCCAGAGUCAUCCCUGCtt | [SEQ ID NO. 318] |
| Target sequence 58: AAGCTGAAGAAACTTGGTAAT | [SEQ ID NO. 319] |
| Position in gene sequence: 1998 | |
| GC content: 33.3% | |
| Sense strand siRNA: GCUGAAGAAACUUGGUAAUtt | [SEQ ID NO. 320] |
| Antisense strand siRNA: AUUACCAAGUUUCUUCAGCtt | [SEQ ID NO. 321] |
| Target sequence 59: AAGAAACTTGGTAATCTGAAA | [SEQ ID NO. 322] |
| Position in gene sequence: 2004 | |
| GC content: 28.6% | |
| Sense strand siRNA: GAAACUUGGUAAUCUGAAAtt | [SEQ ID NO. 323] |
| Antisense strand siRNA: UUUCAGAUUACCAAGUUUCtt | [SEQ ID NO. 324] |
| Target sequence 60: AAACTTGGTAATCTGAAACTA | [SEQ ID NO. 325] |
| Position in gene sequence: 2007 | |
| GC content: 28.6% | |
| Sense strand siRNA: ACUUGGUAAUCUGAAACUAtt | [SEQ ID NO. 326] |
| Antisense strand siRNA: UAGUUUCAGAUUACCAAGUtt | [SEQ ID NO. 327] |
| Target sequence 61: AATCTGAAACTA1921CAGGA | [SEQ ID NO. 328] |
| Position in gene sequence: 2016 | |
| GC content: 28.6% | |
| Sense strand siRNA: UCUGAAACUA1921CAGGAtt | [SEQ ID NO. 329] |
| Antisense strand siRNA: UCCUG1291UAGUUUCAGAtt | [SEQ ID NO. 330] |
| Target sequence 62: AAACTA1921CAGGAGGAAGG | [SEQ ID NO. 331] |
| Position in gene sequence: 2022 | |
| GC content: 38.1% | |
| Sense strand siRNA: ACUA1921CAGGAGGAAGGtt | [SEQ ID NO. 332] |
| Antisense strand siRNA: CCUUCCUCCUG1291UAGUtt | [SEQ ID NO. 333] |
| Target sequence 63: AAGGAGAGGCTTCCAGCACCA | [SEQ ID NO. 334] |
| Position in gene sequence: 2039 | |
| GC content: 57.1% | |
| Sense strand siRNA: GGAGAGGCUUCCAGCACCAtt | [SEQ ID NO. 335] |
| Antisense strand siRNA: UGGUGCUGGAAGCCUCUCCtt | [SEQ ID NO. 336] |
| Target sequence 64: AACCCAGAAGCTG1981ACAG | [SEQ ID NO. 337] |
| Position in gene sequence: 2079 | |
| GC content: 42.9% | |
| Sense strand siRNA: CCCAGAAGCUG1981ACAGtt | [SEQ ID NO. 338] |
| Antisense strand siRNA: CUGU1891CAGCUUCUGGGtt | [SEQ ID NO. 339] |
| Target sequence 65: AAGCTG1981ACAGTGTCACA | [SEQ ID NO. 340] |
| Position in gene sequence: 2086 | |
| GC content: 38.1% | |
| Sense strand siRNA: GCUG1981ACAGUGUCACAtt | [SEQ ID NO. 341] |
| Antisense strand siRNA: UGUGACACUGU1891CAGCtt | [SEQ ID NO. 342] |
| Target sequence 66: AAGGCTATGAATGTCAGCCCA | [SEQ ID NO. 343] |
| Position in gene sequence: 2112 | |
| GC content: 47.6% | |
| Sense strand siRNA: GGCUAUGAAUGUCAGCCCAtt | [SEQ ID NO. 344] |
| Antisense strand siRNA: UGGGCUGACAUUCAUAGCCtt | [SEQ ID NO. 345] |
| Target sequence 67: AATGTCAGCCCATCTTTCTGA | [SEQ ID NO. 346] |
| Position in gene sequence: 2121 | |
| GC content: 42.9% | |
| Sense strand siRNA: UGUCAGCCCAUCUUUCUGAtt | [SEQ ID NO. 347] |
| Antisense strand siRNA: UCAGAAAGAUGGGCUGACAtt | [SEQ ID NO. 348] |
| Target sequence 68: AATGTCCTGGAAGCC2041AT | [SEQ ID NO. 349] |
| Position in gene sequence: 2141 | |
| GC content: 38.1% | |
| Sense strand siRNA: UGUCCUGGAAGCC2041Autt | [SEQ ID NO. 350] |
| Antisense strand siRNA: AU1402GGCUUCCAGGACAtt | [SEQ ID NO. 351] |
| Target sequence 69: AAGCC2041ATTGAGCCAGGT | [SEQ ID NO. 352] |
| Position in gene sequence: 2151 | |
| GC content: 42.9% | |
| Sense strand siRNA: GCC2041AUUGAGCCAGGUtt | [SEQ ID NO. 353] |
| Antisense strand siRNA: ACCUGGCUCAAU1402GGCtt | [SEQ ID NO. 354] |
| Target sequence 70: AACAACCAGCCCGACTCCTTT | [SEQ ID NO. 355] |
| Position in gene sequence: 2193 | |
| GC content: 52.4% | |
| Sense strand siRNA: CAACCAGCCCGACUCCUUUtt | [SEQ ID NO. 356] |
| Antisense strand siRNA: AAAGGAGUCGGGCUGGUUGtt | [SEQ ID NO. 357] |
| Target sequence 71: AACCAGCCCGACTCCTTTGCA | [SEQ ID NO. 358] |
| Position in gene sequence: 2196 | |
| GC content: 57.1% | |
| Sense strand siRNA: CCAGCCCGACUCCUUUGCAtt | [SEQ ID NO. 359] |
| Antisense strand siRNA: UGCAAAGGAGUCGGGCUGGtt | [SEQ ID NO. 360] |
| Target sequence 72: AATGAACTGGGAGAGAGACAG | [SEQ ID NO. 361] |
| Position in gene sequence: 2239 | |
| GC content: 47.6% | |
| Sense strand siRNA: UGAACUGGGAGAGAGACAGtt | [SEQ ID NO. 362] |
| Antisense strand siRNA: CUGUCUCUCUCCCAGUUCAtt | [SEQ ID NO. 363] |
| Target sequence 73: AACTGGGAGAGAGACAGCTTG | [SEQ ID NO. 364] |
| Position in gene sequence: 2243 | |
| GC content: 52.4% | |
| Sense strand siRNA: CUGGGAGAGAGACAGCUUGtt | [SEQ ID NO. 365] |
| Antisense strand siRNA: CAAGCUGUCUCUCUCCCAGtt | [SEQ ID NO. 366] |
| Target sequence 74: AAGTGGGCC2161AAGGCCTT | [SEQ ID NO. 367] |
| Position in gene sequence: 2275 | |
| GC content: 47.6% | |
| Sense strand siRNA: GUGGGCC2161AAGGCCUUtt | [SEQ ID NO. 368] |
| Antisense strand siRNA: AAGGCCUU1612GGCCCACtt | [SEQ ID NO. 369] |
| Target sequence 75: AAGGCCTTGCCTGGCTTCCGC | [SEQ ID NO. 370] |
| Position in gene sequence: 2288 | |
| GC content: 66.7% | |
| Sense strand siRNA: GGCCUUGCCUGGCUUCCGCtt | [SEQ ID NO. 371] |
| Antisense strand siRNA: GCGGAAGCCAGGCAAGGCCtt | [SEQ ID NO. 372] |
| Target sequence 76: AACTTACACGTGGACGACCAG | [SEQ ID NO. 373] |
| Position in gene sequence: 2309 | |
| GC content: 52.4% | |
| Sense strand siRNA: CUUACACGUGGACGACCAGtt | [SEQ ID NO. 374] |
| Antisense strand siRNA: CUGGUCGUCCACGUGUAAGtt | [SEQ ID NO. 375] |
| Target sequence 77: AATGTCAACTCC2281AGGAT | [SEQ ID NO. 376] |
| Position in gene sequence: 2400 | |
| GC content: 33.3% | |
| Sense strand siRNA: UGUCAACUCC2281AGGAUtt | [SEQ ID NO. 377] |
| Antisense strand siRNA: AUCCU1822GGAGUUGACAtt | [SEQ ID NO. 378] |
| Target sequence 78: AACTCC2281AGGATGCTCTA | [SEQ ID NO. 379] |
| Position in gene sequence: 2406 | |
| GC content: 38.1% | |
| Sense strand siRNA: CUCC2281AGGAUGCUCUAtt | [SEQ ID NO. 380] |
| Antisense strand siRNA: UAGAGCAUCCU1822GGAGtt | [SEQ ID NO. 381] |
| Target sequence 79: AATGAGTACCGCATGCACAAG | [SEQ ID NO. 382] |
| Position in gene sequence: 2449 | |
| GC content: 47.6% | |
| Sense strand siRNA: UGAGUACCGCAUGCACAAGtt | [SEQ ID NO. 383] |
| Antisense strand siRNA: CUUGUGCAUGCGGUACUCAtt | [SEQ ID NO. 384] |
| Target sequence 80: AAGTCCCGG2341ATGTACAG | [SEQ ID NO. 385] |
| Position in gene sequence: 2467 | |
| GC content: 42.9% | |
| Sense strand siRNA: GUCCCGG2341AUGUACAGtt | [SEQ ID NO. 386] |
| Antisense strand siRNA: CUGUACAU1432CCGGGACtt | [SEQ ID NO. 387] |
| Target sequence 81: AATGAGGCACCTCTCTCAAGA | [SEQ ID NO. 388] |
| Position in gene sequence: 2500 | |
| GC content: 47.6% | |
| Sense strand siRNA: UGAGGCACCUCUCUCAAGAtt | [SEQ ID NO. 389] |
| Antisense strand siRNA: UCUUGAGAGAGGUGCCUCAtt | [SEQ ID NO. 390] |
| Target sequence 82: AAGAGTTTGGATGGCTCCAAA | [SEQ ID NO. 391] |
| Position in gene sequence: 2517 | |
| GC content: 42.9% | |
| Sense strand siRNA: GAGUUUGGAUGGCUCCAAAtt | [SEQ ID NO. 392] |
| Antisense strand siRNA: UUUGGAGCCAUCCAAACUCtt | [SEQ ID NO. 393] |
| Target sequence 83: AAATC2401ACCCCCCAGGAA | [SEQ ID NO. 394] |
| Position in gene sequence: 2535 | |
| GC content: 42.9% | |
| Sense strand siRNA: AUC2401ACCCCCCAGGAAtt | [SEQ ID NO. 395] |
| Antisense strand siRNA: UUCCUGGGGGGU1042GAUtt | [SEQ ID NO. 396] |
| Target sequence 84: AATTCCTGTGCATGAAAGCAC | [SEQ ID NO. 397] |
| Position in gene sequence: 2554 | |
| GC content: 42.9% | |
| Sense strand siRNA: UUCCUGUGCAUGAAAGCACtt | [SEQ ID NO. 398] |
| Antisense strand siRNA: GUGCUUUCAUGCACAGGAAtt | [SEQ ID NO. 399] |
| Target sequence 85: AAAGCACTGCTACTCTTCAGC | [SEQ ID NO. 400] |
| Position in gene sequence: 2568 | |
| GC content: 47.6% | |
| Sense strand siRNA: AGCACUGCUACUCUUCAGCtt | [SEQ ID NO. 401] |
| Antisense strand siRNA: GCUGAAGAGUAGCAGUGCUtt | [SEQ ID NO. 402] |
| Target sequence 86: AAAAATCAAAAATTCTTTGAT | [SEQ ID NO. 403] |
| Position in gene sequence: 2614 | |
| GC content: 14.3% | |
| Sense strand siRNA: AAAUCAAAAAUUCUUUGAUtt | [SEQ ID NO. 404] |
| Antisense strand siRNA: AUCAAAGAAUUUUUGAUUUtt | [SEQ ID NO. 405] |
| Target sequence 87: AAATCAAAAATTCTTTGATGA | [SEQ ID NO. 406] |
| Position in gene sequence: 2616 | |
| GC content: 19% | |
| Sense strand siRNA: AUCAAAAAUUCUUUGAUGAtt | [SEQ ID NO. 407] |
| Antisense strand siRNA: UCAUCAAAGAAUUUUUGAUtt | [SEQ ID NO. 408] |
| Target sequence 88: AAAAATTCTTTGATGAACTTC | [SEQ ID NO. 409] |
| Position in gene sequence: 2621 | |
| GC content: 23.8% | |
| Sense strand siRNA: AAAUUCUUUGAUGAACUUCtt | [SEQ ID NO. 410] |
| Antisense strand siRNA: GAAGUUCAUCAAAGAAUUUtt | [SEQ ID NO. 411] |
| Target sequence 89: AAATTCTTTGATGAACTTCGA | [SEQ ID NO. 412] |
| Position in gene sequence: 2623 | |
| GC content: 28.6% | |
| Sense strand siRNA: AUUCUUUGAUGAACUUCGAtt | [SEQ ID NO. 413] |
| Antisense strand siRNA: UCGAAGUUCAUCAAAGAAUtt | [SEQ ID NO. 414] |
| Target sequence 90: AACTTCGAATGAACTACATCA | [SEQ ID NO. 415] |
| Position in gene sequence: 2636 | |
| GC content: 33.3% | |
| Sense strand siRNA: CUUCGAAUGAACUACAUCAtt | [SEQ ID NO. 416] |
| Antisense strand siRNA: UGAUGUAGUUCAUUCGAAGtt | [SEQ ID NO. 417] |
| Target sequence 91: AATGAACTACATCAAGGAACT | [SEQ ID NO. 418] |
| Position in gene sequence: 2643 | |
| GC content: 33.3% | |
| Sense strand siRNA: UGAACUACAUCAAGGAACUtt | [SEQ ID NO. 419] |
| Antisense strand siRNA: AGUUCCUUGAUGUAGUUCAtt | [SEQ ID NO. 420] |
| Target sequence 92: AACTACATCAAGGAACTCGAT | [SEQ ID NO. 421] |
| Position in gene sequence: 2647 | |
| GC content: 38.1% | |
| Sense strand siRNA: CUACAUCAAGGAACUCGAUtt | [SEQ ID NO. 422] |
| Antisense strand siRNA: AUCGAGUUCCUUGAUGUAGtt | [SEQ ID NO. 423] |
| Target sequence 93: AAGGAACTCGAT2521CGTAT | [SEQ ID NO. 424] |
| Position in gene sequence: 2656 | |
| GC content: 33.3% | |
| Sense strand siRNA: GGAACUCGAU2521CGUAUtt | [SEQ ID NO. 425] |
| Antisense strand siRNA: AUACG1252AUCGAGUUCCtt | [SEQ ID NO. 426] |
| Target sequence 94: AACTCGAT2521CGTATCATT | [SEQ ID NO. 427] |
| Position in gene sequence: 2660 | |
| GC content: 28.6% | |
| Sense strand siRNA: CUCGAU2521CGUAUCAUUtt | [SEQ ID NO. 428] |
| Antisense strand siRNA: AAUGAUACG1252AUCGAGtt | [SEQ ID NO. 429] |
| Target sequence 95: AAAAGAAAAAATCCCACATCC | [SEQ ID NO. 430] |
| Position in gene sequence: 2687 | |
| GC content: 33.3% | |
| Sense strand siRNA: AAGAAAAAAUCCCACAUCCtt | [SEQ ID NO. 431] |
| Antisense strand siRNA: GGAUGUGGGAUUUUUUCUUtt | [SEQ ID NO. 432] |
| Target sequence 96: AAGAAAAAATCCCACATCCTG | [SEQ ID NO. 433] |
| Position in gene sequence: 2689 | |
| GC content: 38.1% | |
| Sense strand siRNA: GAAAAAAUCCCACAUCCUGtt | [SEQ ID NO. 434] |
| Antisense strand siRNA: CAGGAUGUGGGAUUUUUUCtt | [SEQ ID NO. 435] |
| Target sequence 97: AAAAAATCCCACATCCTGCTC | [SEQ ID NO. 436] |
| Position in gene sequence: 2692 | |
| GC content: 42.9% | |
| Sense strand siRNA: AAAAUCCCACAUCCUGCUCtt | [SEQ ID NO. 437] |
| Antisense strand siRNA: GAGCAGGAUGUGGGAUUUUtt | [SEQ ID NO. 438] |
| Target sequence 98: AAAATCCCACATCCTGCTCAA | [SEQ ID NO. 439] |
| Position in gene sequence: 2694 | |
| GC content: 42.9% | |
| Sense strand siRNA: AAUCCCACAUCCUGCUCAAtt | [SEQ ID NO. 440] |
| Antisense strand siRNA: UUGAGCAGGAUGUGGGAUUtt | [SEQ ID NO. 441] |
| Target sequence 99: AATCCCACATCCTGCTCAAGA | [SEQ ID NO. 442] |
| Position in gene sequence: 2696 | |
| GC content: 47.6% | |
| Sense strand siRNA: UCCCACAUCCUGCUCAAGAtt | [SEQ ID NO. 443] |
| Antisense strand siRNA: UCUUGAGCAGGAUGUGGGAtt | [SEQ ID NO. 444] |
| Target sequence 100: AAGACGCTTCTACCAGCTC25 | [SEQ ID NO. 445] |
| Position in gene sequence: 2713 | |
| GC content 47.6% | |
| Sense strand siRNA: GACGCUUCUACCAGCUC25tt | [SEQ ID NO. 446] |
| Antisense strand siRNA: 52GAGCUGGUAGAAGCGUCtt | [SEQ ID NO. 447] |
| Target sequence 101: AAGCTCCTGGACTCCGTGCAG | [SEQ ID NO. 448] |
| Position in gene sequence: 2739 | |
| GC content: 61.9% | |
| Sense strand siRNA: GCUCCUGGACUCCGUGCAGtt | [SEQ ID NO. 449] |
| Antisense strand siRNA: CUGCACGGAGUCCAGGAGCtt | [SEQ ID NO. 450] |
| Target sequence 102: AATCAAGTCACACATGGTGAG | [SEQ ID NO. 451] |
| Position in gene sequence: 2805 | |
| GC content: 42.9% | |
| Sense strand siRNA: UCAAGUCACACAUGGUGAGtt | [SEQ ID NO. 452] |
| Antisense strand siRNA: CUCACCAUGUGUGACUUGAtt | [SEQ ID NO. 453] |
| Target sequence 103: AAGTCACACATGGTGAGCGTG | [SEQ ID NO. 454] |
| Position in gene sequence: 2809 | |
| GC content: 52.4% | |
| Sense strand siRNA: GUCACACAUGGUGAGCGUGtt | [SEQ ID NO. 455] |
| Antisense strand siRNA: CACGCUCACCAUGUGUGACtt | [SEQ ID NO. 456] |
| Target sequence 104: AAATGATGGCAGAGATCATC2 | [SEQ ID NO. 457] |
| Position in gene sequence: 2840 | |
| GC content: 38.1% | |
| Sense strand siRNA: AUGAUGGCAGAGAUCAUC2tt | [SEQ ID NO. 458] |
| Antisense strand siRNA: 2GAUGAUCUCUGCCAUCAUtt | [SEQ ID NO. 459] |
| Target sequence 105: AAGTGCCCAAGATCCTTTCTG | [SEQ ID NO. 460] |
| Position in gene sequence: 2871 | |
| GC content: 47.6% | |
| Sense strand siRNA: GUGCCCAAGAUCCUUUCUGtt | [SEQ ID NO. 461] |
| Antisense strand siRNA: CAGAAAGGAUCUUGGGCACtt | [SEQ ID NO. 462] |
| Target sequence 106: AAGATCCTTTCTGGGAAAGTC | [SEQ ID NO. 463] |
| Position in gene sequence: 2879 | |
| GC content: 42.9% | |
| Sense strand siRNA: GAUCCUUUCUGGGAAAGUCtt | [SEQ ID NO. 464] |
| Antisense strand siRNA: GACUUUCCCAGAAAGGAUCtt | [SEQ ID NO. 465] |
| Target sequence 107: AAAGTCAAGCCCATCTATTTC | [SEQ ID NO. 466] |
| Position in gene sequence: 2894 | |
| GC content: 38.1% | |
| Sense strand siRNA: AGUCAAGCCCAUCUAUUUCtt | [SEQ ID NO. 467] |
| Antisense strand siRNA: GAAAUAGAUGGGCUUGACUtt | [SEQ ID NO. 468] |
| Target sequence 108: AAGCCCATCTATTTCCACACC | [SEQ ID NO. 469] |
| Position in gene sequence: 2900 | |
| GC content: 47.6% | |
| Sense strand siRNA: GCCCAUCUAUUUCCACACCtt | [SEQ ID NO. 470] |
| Antisense strand siRNA: GGUGUGGAAAUAGAUGGGCtt | [SEQ ID NO. 471] |

TABLE 4
Aromatase

Aromatase - P11511 (gi: 117293) [SEQ ID NO. 472]

| | |
|---|---|
| 1 | atggttttgg aaatgctgaa cccgatacat tataacatca ccagcatcgt gcctgaagcc |
| 61 | atgcctgctg ccaccatgcc agtcctgctc ctcactggcc tttttctctt ggtgtggaat |
| 121 | tatgagggca catcctcaat accaggtcct ggctactgca tgggaattgg acccctcatc |
| 181 | tcccacggca gattcctgtg gatggggatc ggcagtgcct gcaactacta caaccgggta |
| 241 | tatggagaat tcatgcgagt ctggatctct ggagaggaaa cactcattat cagcaagtcc |
| 301 | tcaagtatgt tccacataat gaagcacaat cattacagct ctcgattcgg cagcaaactt |
| 361 | gggctgcagt gcatcggtat gcatgagaaa ggcatcatat ttaacaacaa tccagagctc |
| 421 | tggaaaacaa ctcgaccctt ctttatgaaa gctctgtcag gccccggcct tgttcgtatg |
| 481 | gtcacagtct gtgctgaatc cctcaaaaca catctggaca ggttggagga ggtgaccaat |
| 541 | gaatcgggct atgtggacgt gttgaccctt ctgcgtcgtg tcatgctgga cacctctaac |
| 601 | acgctcttct tgaggatccc tttggacgaa agtgctatcg tggttaaaat ccaaggttat |
| 661 | tttgatgcat ggcaagctct cctcatcaaa ccagacatct tctttaagat ttcttggcta |
| 721 | tacaaaaagt atgagaagtc tgtcaaggat ttgaaagatg ccatagaagt tctgatagca |
| 781 | gaaaaaagac gcaggatttc cacagaagag aaactggaag aatgtatgga ctttgccact |
| 841 | gagttgattt tagcagagaa acgtggtgac ctgacaagag agaatgtgaa ccagtgcata |
| 901 | ttggaaatgc tgatcgcagc tcctgacacc atgtctgtct ctttgttctt catgctattt |
| 961 | ctcattgcaa agcaccctaa tgttgaagag gcaataataa aggaaatcca gactgttatt |
| 1021 | ggtgagagag acataaagat tgatgatata caaaaattaa aagtgatgga aaacttcatt |
| 1081 | tatgagagca tgcggtacca gcctgtcgtg gacttggtca tgcgcaaagc cttagaagat |
| 1141 | gatgtaatcg atggctaccc agtgaaaaag gggacaaaca ttatcctgaa tattggaagg |
| 1201 | atgcacagac tcgagttttt ccccaaaccc aatgaattta ctcttgaaaa ttttgcaaag |
| 1261 | aatgttcctt ataggtactt tcagccattt ggctttgggc cccgtggctg tgcaggaaag |
| 1321 | tacatcgcca tggtgatgat gaaagccatc ctcgttacac ttctgagacg attccacgtg |
| 1381 | aagacattgc aaggacagtg tgttgagagc atacagaaga tacacgactt gtccttgcac |
| 1441 | ccagatgaga ctaaaaacat gctggaaatg atctttaccc caagaagctc agacaggtgt |
| 1501 | ctggaacact ag |

| | |
|---|---|
| Target sequence 1: AAATGCTGAACCCGATACATT | [SEQ ID NO. 473] |
| Position in gene sequence: 12 | |
| GC content: 38.1% | |
| Sense strand siRNA: AUGCUGAACCCGAUACAUUtt | [SEQ ID NO. 474] |
| Antisense strand siRNA: AAUGUAUCGGGUUCAGCAUtt | [SEQ ID NO. 475] |
| Target sequence 2: AACCCGATACATTATAACATC | [SEQ ID NO. 476] |
| Position in gene sequence: 20 | |
| GC content: 33.3% | |
| Sense strand siRNA: CCCGAUACAUUAUAACAUCtt | [SEQ ID NO. 477] |
| Antisense strand siRNA: GAUGUUAUAAUGUAUCGGGtt | [SEQ ID NO. 478] |
| Target sequence 3: AACATCACCAGCATCGTGCCT | [SEQ ID NO. 479] |
| Position in gene sequence: 35 | |
| GC content: 52.4% | |
| Sense strand siRNA: CAUCACCAGCAUCGUGCCUtt | [SEQ ID NO. 480] |
| Antisense strand siRNA: AGGCACGAUGCUGGUGAUGtt | [SEQ ID NO. 481] |
| Target sequence 4: AAGCC61ATGCCTGCTGCCAC | [SEQ ID NO. 482] |
| Position in gene sequence: 57 | |
| GC content: 57.1% | |
| Sense strand siRNA: GCC61AUGCCUGCUGCCACtt | [SEQ ID NO. 483] |
| Antisense strand siRNA: GUGGCAGCAGGCAU16GGCtt | [SEQ ID NO. 484] |
| Target sequence 5: AAT121TATGAGGGCACATCC | [SEQ ID NO. 485] |
| Position in gene sequence: 121 | |
| GC content: 38.1% | |
| Sense strand siRNA: U121UAUGAGGGCACAUCCtt | [SEQ ID NO. 486] |
| Antisense strand siRNA: GGAUGUGCCCUCAUA121Att | [SEQ ID NO. 487] |
| Target sequence 6: AATACCAGGTCCTGGCTACTG | [SEQ ID NO. 488] |
| Position in gene sequence: 144 | |
| GC content: 52.4% | |
| Sense strand siRNA: UACCAGGUCCUGGCUACUGtt | [SEQ ID NO. 489] |
| Antisense strand siRNA: CAGUAGCCAGGACCUGGUAtt | [SEQ ID NO. 490] |
| Target sequence 7: AATTGGACCCCTCATC181TC | [SEQ ID NO. 491] |
| Position in gene sequence: 171 | |
| GC content: 42.9% | |
| Sense strand siRNA: UUGGACCCCUCAUC181Uctt | [SEQ ID NO. 492] |
| Antisense strand siRNA: GA181GAUGAGGGGUCCAAtt | [SEQ ID NO. 493] |
| Target sequence 8: AACTACTACAACCGGGTA241 | [SEQ ID NO. 494] |
| Position in gene sequence: 232 | |
| GC content: 38.1% | |
| Sense strand siRNA: CUACUACAACCGGGUA241tt | [SEQ ID NO. 495] |
| Antisense strand siRNA: 142UACCCGGUUGUAGUAGtt | [SEQ ID NO. 496] |
| Target sequence 9: AACCGGGTA241TATGGAGAA | [SEQ ID NO. 497] |
| Position in gene sequence: 241 | |
| GC content: 38.1% | |
| Sense strand siRNA: CCGGGUA241UAUGGAGAAtt | [SEQ ID NO. 498] |
| Antisense strand siRNA: UUCUCCAUA142UACCCGGtt | [SEQ ID NO. 499] |
| Target sequence 10: AATTCATGCGAGTCTGGATCT | [SEQ ID NO. 500] |
| Position in gene sequence: 260 | |
| GC content: 42.9% | |
| Sense strand siRNA: UUCAUGCGAGUCUGGAUCUtt | [SEQ ID NO. 501] |
| Antisense strand siRNA: AGAUCCAGACUCGCAUGAAtt | [SEQ ID NO. 502] |
| Target sequence 11: AAACACTCATTATCAGCAAGT | [SEQ ID NO. 503] |
| Position in gene sequence: 290 | |
| GC content: 33.3% | |
| Sense strand siRNA: ACACUCAUUAUCAGCAAGUtt | [SEQ ID NO. 504] |
| Antisense strand siRNA: ACUUGCUGAUAAUGAGUGUtt | [SEQ ID NO. 505] |
| Target sequence 12: AAGTCC301TCAAGTATGTTC | [SEQ ID NO. 506] |
| Position in gene sequence: 307 | |
| GC content: 33.3% | |
| Sense strand siRNA: GUCC301UCAAGUAUGUUCtt | [SEQ ID NO. 507] |
| Antisense strand siRNA: GAACAUACUUGA103GGACtt | [SEQ ID NO. 508] |
| Target sequence 13: AAGTATGTTCCACATAATGAA | [SEQ ID NO. 509] |
| Position in gene sequence: 318 | |
| GC content: 28.6% | |
| Sense strand siRNA: GUAUGUUCCACAUAAUGAAtt | [SEQ ID NO. 510] |
| Antisense strand siRNA: UUCAUUAUGUGGAACAUACtt | [SEQ ID NO. 511] |
| Target sequence 14: AATGAAGCACAATCATTACAG | [SEQ ID NO. 512] |
| Position in gene sequence: 333 | |
| GC content: 33.3% | |
| Sense strand siRNA: UGAAGCACAAUCAUUACAGtt | [SEQ ID NO. 513] |
| Antisense strand siRNA: CUGUAAUGAUUGUGCUUCAtt | [SEQ ID NO. 514] |
| Target sequence 15: AAGCACAATCATTACAGCTCT | [SEQ ID NO. 515] |
| Position in gene sequence: 337 | |
| GC content: 38.1% | |
| Sense strand siRNA: GCACAAUCAUUACAGCUCUtt | [SEQ ID NO. 516] |
| Antisense strand siRNA: AGAGCUGUAAUGAUUGUGCtt | [SEQ ID NO. 517] |
| Target sequence 16: AATCATTACAGCTCTCGATTC | [SEQ ID NO. 518] |
| Position in gene sequence: 343 | |
| GC content: 38.1% | |
| Sense strand siRNA: UCAUUACAGCUCUCGAUUCtt | [SEQ ID NO. 519] |
| Antisense strand siRNA: GAAUCGAGAGCUGUAAUGAtt | [SEQ ID NO. 520] |
| Target sequence 17: AAACTT361GGGCTGCAGTGC | [SEQ ID NO. 521] |
| Position in gene sequence: 370 | |
| GC content: 47.6% | |
| Sense strand siRNA: ACUU361GGGCUGCAGUGCtt | [SEQ ID NO. 522] |
| Antisense strand siRNA: GCACUGCAGCCC163AAGUtt | [SEQ ID NO. 523] |
| Target sequence 18: AAAGGCATCATATTTAACAAC | [SEQ ID NO. 524] |
| Position in gene sequence: 406 | |
| GC content: 28.6% | |
| Sense strand siRNA: AGGCAUCAUAUUUAACAACtt | [SEQ ID NO. 525] |
| Antisense strand siRNA: GUUGUUAAAUAUGAUGCCUtt | [SEQ ID NO. 526] |
| Target sequence 19: AACAACAATCCAGAGCTC421 | [SEQ ID NO. 527] |
| Position in gene sequence: 421 | |
| GC content: 38.1% | |
| Sense strand siRNA: CAACAAUCCAGAGCUC421tt | [SEQ ID NO. 528] |
| Antisense strand siRNA: 124GAGCUCUGGAUUGUUGtt | [SEQ ID NO. 529] |
| Target sequence 20: AACAATCCAGAGCTC421TGG | [SEQ ID NO. 530] |
| Position in gene sequence: 424 | |
| GC content: 42.9% | |
| Sense strand siRNA: CAAUCCAGAGCUC421UGGtt | [SEQ ID NO. 531] |
| Antisense strand siRNA: CCA124GAGCUCUGGAUUGtt | [SEQ ID NO. 532] |
| Target sequence 21: AATCCAGAGCTC421TGGAAA | [SEQ ID NO. 533] |
| Position in gene sequence: 427 | |
| GC content: 38.1% | |
| Sense strand siRNA: UCCAGAGCUC421UGGAAAtt | [SEQ ID NO. 534] |
| Antisense strand siRNA: UUUCCA124GAGCUCUGGAtt | [SEQ ID NO. 535] |
| Target sequence 22: AAAACAACTCGACCCTTCTTT | [SEQ ID NO. 536] |
| Position in gene sequence: 445 | |
| GC content: 38.1% | |
| Sense strand siRNA: AACAACUCGACCCUUCUUUtt | [SEQ ID NO. 537] |
| Antisense strand siRNA: AAAGAAGGGUCGAGUUGUUtt | [SEQ ID NO. 538] |
| Target sequence 23: AACAACTCGACCCTTCTTTAT | [SEQ ID NO. 539] |
| Position in gene sequence: 447 | |
| GC content: 38.1% | |
| Sense strand siRNA: CAACUCGACCCUUCUUUAUtt | [SEQ ID NO. 540] |
| Antisense strand siRNA: AUAAAGAAGGGUCGAGUUGtt | [SEQ ID NO. 541] |
| Target sequence 24: AACTCGACCCTTCTTTATGAA | [SEQ ID NO. 542] |
| Position in gene sequence: 450 | |
| GC content: 38.1% | |
| Sense strand siRNA: CUCGACCCUUCUUUAUGAAtt | [SEQ ID NO. 543] |
| Antisense strand siRNA: UUCAUAAAGAAGGGUCGAGtt | [SEQ ID NO. 544] |
| Target sequence 25: AAAGCTCTGTCAGGCCCCGGC | [SEQ ID NO. 545] |
| Position in gene sequence: 469 | |
| GC content: 66.7% | |
| Sense strand siRNA: AGCUCUGUCAGGCCCCGGCtt | [SEQ ID NO. 546] |
| Antisense strand siRNA: GCCGGGGCCUGACAGAGCUtt | [SEQ ID NO. 547] |
| Target sequence 26: AATCCCTCAAAACACATCTGG | [SEQ ID NO. 548] |
| Position in gene sequence: 521 | |
| GC content: 42.9% | |
| Sense strand siRNA: UCCCUCAAAACACAUCUGGtt | [SEQ ID NO. 549] |
| Antisense strand siRNA: CCAGAUGUGUUUUGAGGGAtt | [SEQ ID NO. 550] |
| Target sequence 27: AAAACACATCTGGACAGGTTG | [SEQ ID NO. 551] |
| Position in gene sequence: 529 | |
| GC content: 42.9% | |
| Sense strand siRNA: AACACAUCUGGACAGGUUGtt | [SEQ ID NO. 552] |
| Antisense strand siRNA: CAACCUGUCCAGAUGUGUUtt | [SEQ ID NO. 553] |
| Target sequence 28: AACACATCTGGACAGGTTGGA | [SEQ ID NO. 554] |
| Position in gene sequence: 531 | |
| GC content: 47.6% | |
| Sense strand siRNA: CACAUCUGGACAGGUUGGAtt | [SEQ ID NO. 555] |
| Antisense strand siRNA: UCCAACCUGUCCAGAUGUGtt | [SEQ ID NO. 556] |
| Target sequence 29: AAT541GAATCGGGCTATGTG | [SEQ ID NO. 557] |
| Position in gene sequence: 562 | |
| GC content: 38.1% | |
| Sense strand siRNA: U541GAAUCGGGCUAUGUGtt | [SEQ ID NO. 558] |
| Antisense strand siRNA: CACAUAGCCCGAUUC145Att | [SEQ ID NO. 559] |
| Target sequence 30: AATCGGGCTATGTGGACGTGT | [SEQ ID NO. 560] |
| Position in gene sequence: 569 | |
| GC content: 52.4% | |
| Sense strand siRNA: UCGGGCUAUGUGGACGUGUtt | [SEQ ID NO. 561] |
| Antisense strand siRNA: ACACGUCCACAUAGCCCGAtt | [SEQ ID NO. 562] |
| Target sequence 31: AAC601ACGCTCTTCTTGAGG | [SEQ ID NO. 563] |
| Position in gene sequence: 625 | |
| GC content: 42.9% | |
| Sense strand siRNA: C601ACGCUCUUCUUGAGGtt | [SEQ ID NO. 564] |
| Antisense strand siRNA: CCUCAAGAAGAGCGU106Gtt | [SEQ ID NO. 565] |
| Target sequence 32: AAAGTGCTATCGTGGTTAAAA | [SEQ ID NO. 566] |
| Position in gene sequence: 659 | |
| GC content: 33.3% | |
| Sense strand siRNA: AGUGCUAUCGUGGUUAAAAtt | [SEQ ID NO. 567] |
| Antisense strand siRNA: UUUUAACCACGAUAGCACUtt | [SEQ ID NO. 568] |
| Target sequence 33: AAAATCCAAGGTTAT661TTT | [SEQ ID NO. 569] |
| Position in gene sequence: 676 | |
| GC content: 19% | |
| Sense strand siRNA: AAUCCAAGGUUAU661UUUtt | [SEQ ID NO. 570] |
| Antisense strand siRNA: AAA166AUAACCUUGGAUUtt | [SEQ ID NO. 571] |
| Target sequence 34: AATCCAAGGTTAT661TTTGA | [SEQ ID NO. 572] |
| Position in gene sequence: 678 | |
| GC content: 23.8% | |
| Sense strand siRNA: UCCAAGGUUAU661UUUGAtt | [SEQ ID NO. 573] |
| Antisense strand siRNA: UCAAA166AUAACCUUGGAtt | [SEQ ID NO. 574] |
| Target sequence 35: AAGGTTAT661TTTGATGCAT | [SEQ ID NO. 575] |
| Position in gene sequence: 683 | |
| GC content: 23.8% | |
| Sense strand siRNA: GGUUAU661UUUGAUGCAUtt | [SEQ ID NO. 576] |
| Antisense strand siRNA: AUGCAUCAAA166AUAACCtt | [SEQ ID NO. 577] |
| Target sequence 36: AAGCTCTCCTCATCAAACCAG | [SEQ ID NO. 578] |
| Position in gene sequence: 707 | |
| GC content: 47.6% | |
| Sense strand siRNA: GCUCUCCUCAUCAAACCAGtt | [SEQ ID NO. 579] |
| Antisense strand siRNA: CUGGUUUGAUGAGGAGAGCtt | [SEQ ID NO. 580] |
| Target sequence 37: AAACCAGACATCTTCTTTAAG | [SEQ ID NO. 581] |
| Position in gene sequence: 721 | |
| GC content: 33.3% | |
| Sense strand siRNA: ACCAGACAUCUUCUUUAAGtt | [SEQ ID NO. 582] |
| Antisense strand siRNA: CUUAAAGAAGAUGUCUGGUtt | [SEQ ID NO. 583] |
| Target sequence 38: AAGATTTCTTGGCTA721TAC | [SEQ ID NO. 584] |
| Position in gene sequence: 739 | |
| GC content: 28.6% | |
| Sense strand siRNA: GAUUUCUUGGCUA721UACtt | [SEQ ID NO. 585] |
| Antisense strand siRNA: GUA127UAGCCAAGAAAUCtt | [SEQ ID NO. 586] |
| Target sequence 39: AAAAAGTATGAGAAGTCTGTC | [SEQ ID NO. 587] |
| Position in gene sequence: 760 | |
| GC content: 33.3% | |
| Sense strand siRNA: AAAGUAUGAGAAGUCUGUCtt | [SEQ ID NO. 588] |
| Antisense strand siRNA: GACAGACUUCUCAUACUUUtt | [SEQ ID NO. 589] |
| Target sequence 40: AAAGTATGAGAAGTCTGTCAA | [SEQ ID NO. 590] |
| Position in gene sequence: 762 | |
| GC content: 33.3% | |
| Sense strand siRNA: AGUAUGAGAAGUCUGUCAAtt | [SEQ ID NO. 591] |
| Antisense strand siRNA: UUGACAGACUUCUCAUACUtt | [SEQ ID NO. 592] |
| Target sequence 41: AAGTCTGTCAAGGATTTGAAA | [SEQ ID NO. 593] |
| Position in gene sequence: 772 | |
| GC content: 33.3% | |
| Sense strand siRNA: GUCUGUCAAGGAUUUGAAAtt | [SEQ ID NO. 594] |
| Antisense strand siRNA: UUUCAAAUCCUUGACAGACtt | [SEQ ID NO. 595] |
| Target sequence 42: AAGGATTTGAAAGATGCCATA | [SEQ ID NO. 596] |
| Position in gene sequence: 781 | |
| GC content: 33.3% | |
| Sense strand siRNA: GGAUUUGAAAGAUGCCAUAtt | [SEQ ID NO. 597] |
| Antisense strand siRNA: UAUGGCAUCUUUCAAAUCCtt | [SEQ ID NO. 598] |
| Target sequence 43: AAAGATGCCATAGAAGTTCTG | [SEQ ID NO. 599] |
| Position in gene sequence: 790 | |
| GC content: 38.1% | |
| Sense strand siRNA: AGAUGCCAUAGAAGUUCUGtt | [SEQ ID NO. 600] |
| Antisense strand siRNA: CAGAACUUCUAUGGCAUCUtt | [SEQ ID NO. 601] |
| Target sequence 44: AAGTTCTGATAGCA781GAAA | [SEQ ID NO. 602] |
| Position in gene sequence: 803 | |
| GC content: 28.6% | |
| Sense strand siRNA: GUUCUGAUAGCA781GAAAtt | [SEQ ID NO. 693] |
| Antisense strand siRNA: UUUC187UGCUAUCAGAACtt | [SEQ ID NO. 604] |
| Target sequence 45: AAAAAAGACGCAGGATTTCCA | [SEQ ID NO. 605] |
| Position in gene sequence: 821 | |
| GC content: 38.1% | |
| Sense strand siRNA: AAAAGACGCAGGAUUUCCAtt | [SEQ ID NO. 606] |
| Antisense strand siRNA: UGGAAAUCCUGCGUCUUUUtt | [SEQ ID NO. 607] |
| Target sequence 46: AAAAGACGCAGGATTTCCACA | [SEQ ID NO. 608] |
| Position in gene sequence: 823 | |
| GC content: 42.9% | |
| Sense strand siRNA: AAGACGCAGGAUUUCCACAtt | [SEQ ID NO. 609] |
| Antisense strand siRNA: UGUGGAAAUCCUGCGUCUUtt | [SEQ ID NO. 610] |
| Target sequence 47: AAGACGCAGGATTTCCACAGA | [SEQ ID NO. 611] |
| Position in gene sequence: 825 | |
| GC content: 47.6% | |
| Sense strand siRNA: GACGCAGGAUUUCCACAGAtt | [SEQ ID NO. 612] |
| Antisense strand siRNA: UCUGUGGAAAUCCUGCGUCtt | [SEQ ID NO. 613] |
| Target sequence 48: AAGAGAAACTGGAAGAATGTA | [SEQ ID NO. 614] |
| Position in gene sequence: 845 | |
| GC content: 33.3% | |
| Sense strand siRNA: GAGAAACUGGAAGAAUGUAtt | [SEQ ID NO. 615] |
| Antisense strand siRNA: UACAUUCUUCCAGUUUCUCtt | [SEQ ID NO. 616] |
| Target sequence 49: AAACTGGAAGAATGTATGGAC | [SEQ ID NO. 617] |
| Position in gene sequence: 850 | |
| GC content: 38.1% | |
| Sense strand siRNA: ACUGGAAGAAUGUAUGGACtt | [SEQ ID NO. 618] |
| Antisense strand siRNA: GUCCAUACAUUCUUCCAGUtt | [SEQ ID NO. 619] |
| Target sequence 50: AAGAATGTATGGACTTTGCCA | [SEQ ID NO. 620] |
| Position in gene sequence: 857 | |
| GC content: 38.1% | |
| Sense strand siRNA: GAAUGUAUGGACUUUGCCAtt | [SEQ ID NO. 621] |
| Antisense strand siRNA: UGGCAAAGUCCAUACAUUCtt | [SEQ ID NO. 622] |
| Target sequence 51: AATGTATGGACTTTGCCACT8 | [SEQ ID NO. 623] |
| Position in gene sequence: 860 | |
| GC content: 38.1% | |
| Sense strand siRNA: UGUAUGGACUUUGCCACU8tt | [SEQ ID NO. 624] |
| Antisense strand siRNA: 8AGUGGCAAAGUCCAUACAtt | [SEQ ID NO. 625] |
| Target sequence 52: AAACGTGGTGACCTGACAAGA | [SEQ ID NO. 626] |
| Position in gene sequence: 901 | |
| GC content: 47.6% | |
| Sense strand siRNA: ACGUGGUGACCUGACAAGAtt | [SEQ ID NO. 627] |
| Antisense strand siRNA: UCUUGUCAGGUCACCACGUtt | [SEQ ID NO. 628] |
| Target sequence 53: AAGAGAGAATGTGAACCAGTG | [SEQ ID NO. 629] |
| Position in gene sequence: 918 | |
| GC content: 42.9% | |
| Sense strand siRNA: GAGAGAAUGUGAACCAGUGtt | [SEQ ID NO. 630] |
| Antisense strand siRNA: CACUGGUUCACAUUCUCUCtt | [SEQ ID NO. 631] |
| Target sequence 54: AATGTGAACCAGTGCATA901 | [SEQ ID NO. 632] |
| Position in gene sequence: 925 | |
| GC content: 33.3% | |
| Sense strand siRNA: UGUGAACCAGUGCAUA901tt | [SEQ ID NO. 633] |
| Antisense strand siRNA: 109UAUGCACUGGUUCACAtt | [SEQ ID NO. 634] |
| Target sequence 55: AACCAGTGCATA901TTGGAA | [SEQ ID NO. 635] |
| Position in gene sequence: 931 | |
| GC content: 33.3% | |
| Sense strand siRNA: CCAGUGCAUA901UUGGAAtt | [SEQ ID NO. 636] |
| Antisense strand siRNA: UUCCAA109UAUGCACUGGtt | [SEQ ID NO. 637] |
| Target sequence 56: AAATGCTGATCGCAGCTCCTG | [SEQ ID NO. 638] |
| Position in gene sequence: 950 | |
| GC content: 52.4% | |
| Sense strand siRNA: AUGCUGAUCGCAGCUCCUGtt | [SEQ ID NO. 639] |
| Antisense strand siRNA: CAGGAGCUGCGAUCAGCAUtt | [SEQ ID NO. 640] |
| Target sequence 57: AAAGCACCCTAATGTTGAAGA | [SEQ ID NO. 641] |
| Position in gene sequence: 1017 | |
| GC content: 38.1% | |
| Sense strand siRNA: AGCACCCUAAUGUUGAAGAtt | [SEQ ID NO. 642] |
| Antisense strand siRNA: UCUUCAACAUUAGGGUGCUtt | [SEQ ID NO. 643] |
| Target sequence 58: AATGTTGAAGAGGCAATAATA | [SEQ ID NO. 644] |
| Position in gene sequence: 1027 | |
| GC content: 28.6% | |
| Sense strand siRNA: UGUUGAAGAGGCAAUAAUAtt | [SEQ ID NO. 645] |
| Antisense strand siRNA: UAUUAUUGCCUCUUCAACAtt | [SEQ ID NO. 646] |
| Target sequence 59: AAGAGGCAATAATAAAGGAAA | [SEQ ID NO. 647] |
| Position in gene sequence: 1034 | |
| GC content: 28.6% | |
| Sense strand siRNA: GAGGCAAUAAUAAAGGAAAtt | [SEQ ID NO. 648] |
| Antisense strand siRNA: UUUCCUUUAUUAUUGCCUCtt | [SEQ ID NO. 649] |
| Target sequence 60: AATAATAAAGGAAATCCAGAC | [SEQ ID NO. 650] |
| Position in gene sequence: 1041 | |
| GC content: 28.6% | |
| Sense strand siRNA: UAAUAAAGGAAAUCCAGACtt | [SEQ ID NO. 651] |
| Antisense strand siRNA: GUCUGGAUUUCCUUUAUUAtt | [SEQ ID NO. 652] |
| Target sequence 61: AATAAAGGAAATCCAGACTGT | [SEQ ID NO. 653] |
| Position in gene sequence: 1044 | |
| GC content: 33.3% | |
| Sense strand siRNA: UAAAGGAAAUCCAGACUGUtt | [SEQ ID NO. 654] |
| Antisense strand siRNA: ACAGUCUGGAUUUCCUUUAtt | [SEQ ID NO. 655] |
| Target sequence 62: AAAGGAAATCCAGACTGTTAT | [SEQ ID NO. 656] |
| Position in gene sequence: 1047 | |
| GC content: 33.3% | |
| Sense strand siRNA: AGGAAAUCCAGACUGUUAUtt | [SEQ ID NO. 657] |
| Antisense strand siRNA: AUAACAGUCUGGAUUUCCUtt | [SEQ ID NO. 658] |
| Target sequence 63: AAATCCAGACTGTTATT1021 | [SEQ ID NO. 659] |
| Position in gene sequence: 1052 | |
| GC content: 23.8% | |
| Sense strand siRNA: AUCCAGACUGUUAUU1021tt | [SEQ ID NO. 660] |
| Antisense strand siRNA: 1201AAUAACAGUCUGGAUtt | [SEQ ID NO. 661] |
| Target sequence 64: AAAGATTGATGATATACAAAA | [SEQ ID NO. 662] |
| Position in gene sequence: 1087 | |
| GC content: 19% | |
| Sense strand siRNA: AGAUUGAUGAUAUACAAAAtt | [SEQ ID NO. 663] |
| Antisense strand siRNA: UUUUGUAUAUCAUCAAUCUtt | [SEQ ID NO. 664] |
| Target sequence 65: AAAAATTAAAAGTGATGGAAA | [SEQ ID NO. 665] |
| Position in gene sequence: 1104 | |
| GC content: 19% | |
| Sense strand siRNA: AAAUUAAAAGUGAUGGAAAtt | [SEQ ID NO. 666] |
| Antisense strand siRNA: UUUCCAUCACUUUUAAUUUtt | [SEQ ID NO. 667] |
| Target sequence 66: AAATTAAAAGTGATGGAAAAC | [SEQ ID NO. 668] |
| Position in gene sequence: 1106 | |
| GC content: 23.8% | |
| Sense strand siRNA: AUUAAAAGUGAUGGAAAACtt | [SEQ ID NO. 669] |
| Antisense strand siRNA: GUUUUCCAUCACUUUUAAUtt | [SEQ ID NO. 670] |
| Target sequence 67: AAAAGTGATGGAAAACTTCAT | [SEQ ID NO. 671] |
| Position in gene sequence: 1111 | |
| GC content: 28.6% | |
| Sense strand siRNA: AAGUGAUGGAAAACUUCAUtt | [SEQ ID NO. 672] |
| Antisense strand siRNA: AUGAAGUUUUCCAUCACUUtt | [SEQ ID NO. 673] |
| Target sequence 68: AAGTGATGGAAAACTTCATT1 | [SEQ ID NO. 674] |
| Position in gene sequence: 1113 | |
| GC content: 28.6% | |
| Sense strand siRNA: GUGAUGGAAAACUUCAUU1tt | [SEQ ID NO. 675] |
| Antisense strand siRNA: 1AAUGAAGUUUUCCAUCACtt | [SEQ ID NO. 676] |
| Target sequence 69: AAAACTTCATT1081TATGAG | [SEQ ID NO. 677] |
| Position in gene sequence: 1122 | |
| GC content: 19% | |
| Sense strand siRNA: AACUUCAUU1081UAUGAGtt | [SEQ ID NO. 678] |
| Antisense strand siRNA: CUCAUA1801AAUGAAGUUtt | [SEQ ID NO. 679] |
| Target sequence 70: AACTTCATT1081TATGAGAG | [SEQ ID NO. 680] |
| Position in gene sequence: 1124 | |
| GC content: 23.8% | |
| Sense strand siRNA: CUUCAUU1081UAUGAGAGtt | [SEQ ID NO. 681] |
| Antisense strand siRNA: CUCUCAUA1801AAUGAAGtt | [SEQ ID NO. 682] |
| Target sequence 71: AAAGCCTTAGAAGAT1141GA | [SEQ ID NO. 683] |
| Position in gene sequence: 1182 | |
| GC content: 28.6% | |
| Sense strand siRNA: AGCCUUAGAAGAU1141Gatt | [SEQ ID NO. 684] |
| Antisense strand siRNA: UC1411AUCUUCUAAGGCUtt | [SEQ ID NO. 685] |
| Target sequence 72: AAGAT1141GATGTAATCGAT | [SEQ ID NO. 686] |
| Position in gene sequence: 1192 | |
| GC content: 23.8% | |
| Sense strand siRNA: GAU1141GAUGUAAUCGAUtt | [SEQ ID NO. 687] |
| Antisense strand siRNA: AUCGAUUACAUC1411AUCtt | [SEQ ID NO. 688] |
| Target sequence 73: AATCGATGGCTACCCAGTGAA | [SEQ ID NO. 689] |
| Position in gene sequence: 1206 | |
| GC content: 47.6% | |
| Sense strand siRNA: UCGAUGGCUACCCAGUGAAtt | [SEQ ID NO. 690] |
| Antisense strand siRNA: UUCACUGGGUAGCCAUCGAtt | [SEQ ID NO. 691] |
| Target sequence 74: AAAAAGGGGACAAACATTATC | [SEQ ID NO. 692] |
| Position in gene sequence: 1225 | |
| GC content: 33.3% | |
| Sense strand siRNA: AAAGGGGACAAACAUUAUCtt | [SEQ ID NO. 693] |
| Antisense strand siRNA: GAUAAUGUUUGUCCCCUUUtt | [SEQ ID NO. 694] |
| Target sequence 75: AAAGGGGACAAACATTATCCT | [SEQ ID NO. 695] |
| Position in gene sequence: 1227 | |
| GC content: 38.1% | |
| Sense strand siRNA: AGGGGACAAACAUUAUCCUtt | [SEQ ID NO. 696] |
| Antisense strand siRNA: AGGAUAAUGUUUGUCCCCUtt | [SEQ ID NO. 697] |
| Target sequence 76: AAACATTATCCTGAATATTGG | [SEQ ID NO. 698] |
| Position in gene sequence: 1236 | |
| GC content: 28.6% | |
| Sense strand siRNA: ACAUUAUCCUGAAUAUUGGtt | [SEQ ID NO. 699] |
| Antisense strand siRNA: CCAAUAUUCAGGAUAAUGUtt | [SEQ ID NO. 700] |
| Target sequence 77: AATATTGGAAGG1201ATGCA | [SEQ ID NO. 701] |
| Position in gene sequence: 1249 | |
| GC content: 28.6% | |
| Sense strand siRNA: UAUUGGAAGG1201AUGCAtt | [SEQ ID NO. 702] |
| Antisense strand siRNA: UGCAU1021CCUUCCAAUAtt | [SEQ ID NO. 703] |
| Target sequence 78: AAGG1201ATGCACAGACTCG | [SEQ ID NO. 704] |
| Position in gene sequence: 1257 | |
| GC content: 42.9% | |
| Sense strand siRNA: GG1201AUGCACAGACUCGtt | [SEQ ID NO. 705] |
| Antisense strand siRNA: CGAGUCUGUGCAU1021CCtt | [SEQ ID NO. 706] |
| Target sequence 79: AAACCCAATGAATTTACTCTT | [SEQ ID NO. 707] |
| Position in gene sequence: 1289 | |
| GC content: 28.6% | |
| Sense strand siRNA: ACCCAAUGAAUUUACUCUUtt | [SEQ ID NO. 708] |
| Antisense strand siRNA: AAGAGUAAAUUCAUUGGGUtt | [SEQ ID NO. 709] |
| Target sequence 80: AATGAATTTACTCTTGAAAAT | [SEQ ID NO. 710] |
| Position in gene sequence: 1295 | |
| GC content: 19% | |
| Sense strand siRNA: UGAAUUUACUCUUGAAAAUtt | [SEQ ID NO. 711] |
| Antisense strand siRNA: AUUUUCAAGAGUAAAUUCAtt | [SEQ ID NO. 712] |
| Target sequence 81: AATTTACTCTTGAAAATTTTG | [SEQ ID NO. 713] |
| Position in gene sequence: 1299 | |
| GC content: 19% | |
| Sense strand siRNA: UUUACUCUUGAAAAUUUUGtt | [SEQ ID NO. 714] |
| Antisense strand siRNA: CAAAAUUUUCAAGAGUAAAtt | [SEQ ID NO. 715] |
| Target sequence 82: AAAATTTTGCAAAG1261AAT | [SEQ ID NO. 716] |
| Position in gene sequence: 1311 | |
| GC content: 14.3% | |
| Sense strand siRNA: AAUUUUGCAAAG1261AAUtt | [SEQ ID NO. 717] |
| Antisense strand siRNA: AUU1621CUUUGCAAAAUUtt | [SEQ ID NO. 718] |
| Target sequence 83: AATTTTGCAAAG1261AATGT | [SEQ ID NO. 719] |
| Position in gene sequence: 1313 | |
| GC content: 19% | |
| Sense strand siRNA: UUUUGCAAAG1261AAUGUtt | [SEQ ID NO. 720] |
| Antisense strand siRNA: ACAUU1621CUUUGCAAAAtt | [SEQ ID NO. 721] |
| Target sequence 84: AAAG1261AATGTTCCTTATA | [SEQ ID NO. 722] |
| Position in gene sequence: 1321 | |
| GC content: 19% | |
| Sense strand siRNA: AG1261AAUGUUCCUUAUAtt | [SEQ ID NO. 723] |
| Antisense strand siRNA: UAUAAGGAACAUU1621Cutt | [SEQ ID NO. 724] |
| Target sequence 85: AATGTTCCTTATAGGTACTTT | [SEQ ID NO. 725] |
| Position in gene sequence: 1329 | |
| GC content: 28.6% | |
| Sense strand siRNA: UGUUCCUUAUAGGUACUUUtt | [SEQ ID NO. 726] |
| Antisense strand siRNA: AAAGUACCUAUAAGGAACAtt | [SEQ ID NO. 727] |
| Target sequence 86: AAAG1321TACATCGCCATGG | [SEQ ID NO. 728] |
| Position in gene sequence: 1385 | |
| GC content: 38.1% | |
| Sense strand siRNA: AG1321UACAUCGCCAUGGtt | [SEQ ID NO. 729] |
| Antisense strand siRNA: CCAUGGCGAUGUA1231CUtt | [SEQ ID NO. 730] |
| Target sequence 87: AAAGCCATCCTCGTTACACTT | [SEQ ID NO. 731] |
| Position in gene sequence: 1414 | |
| GC content: 42.9% | |
| Sense strand siRNA: AGCCAUCCUCGUUACACUUtt | [SEQ ID NO. 732] |
| Antisense strand siRNA: AAGUGUAACGAGGAUGGCUtt | [SEQ ID NO. 733] |
| Target sequence 88: AAGACATTGCAAGGACAGTGT | [SEQ ID NO. 734] |
| Position in gene sequence: 1457 | |
| GC content: 42.9% | |
| Sense strand siRNA: GACAUUGCAAGGACAGUGUtt | [SEQ ID NO. 735] |
| Antisense strand siRNA: ACACUGUCCUUGCAAUGUCtt | [SEQ ID NO. 736] |
| Target sequence 89: AAGGACAGTGTGTTGAGAGCA | [SEQ ID NO. 737] |
| Position in gene sequence: 1467 | |
| GC content: 47.6% | |
| Sense strand siRNA: GGACAGUGUGUUGAGAGCAtt | [SEQ ID NO. 738] |
| Antisense strand siRNA: UGCUCUCAACACACUGUCCtt | [SEQ ID NO. 739] |
| Target sequence 90: AAGATACACGACTTGTCCTTG | [SEQ ID NO. 740] |
| Position in gene sequence: 1493 | |
| GC content: 42.9% | |
| Sense strand siRNA: GAUACACGACUUGUCCUUGtt | [SEQ ID NO. 741] |
| Antisense strand siRNA: CAAGGACAAGUCGUGUAUCtt | [SEQ ID NO. 742] |
| Target sequence 91: AAAAACATGCTGGAAATGATC | [SEQ ID NO. 743] |
| Position in gene sequence: 1533 | |
| GC content: 33.3% | |
| Sense strand siRNA: AAACAUGCUGGAAAUGAUCtt | [SEQ ID NO. 744] |
| Antisense strand siRNA: GAUCAUUUCCAGCAUGUUUtt | [SEQ ID NO. 745] |
| Target sequence 92: AAACATGCTGGAAATGATCTT | [SEQ ID NO. 746] |
| Position in gene sequence: 1535 | |
| GC content: 33.3% | |
| Sense strand siRNA: ACAUGCUGGAAAUGAUCUUtt | [SEQ ID NO. 747] |
| Antisense strand siRNA: AAGAUCAUUUCCAGCAUGUtt | [SEQ ID NO. 748] |
| Target sequence 93: AAATGATCTTTACCCCAAGAA | [SEQ ID NO. 749] |
| Position in gene sequence: 1546 | |
| GC content: 33.3% | |
| Sense strand siRNA: AUGAUCUUUACCCCAAGAAtt | [SEQ ID NO. 750] |
| Antisense strand siRNA: UUCUUGGGGUAAAGAUCAUtt | [SEQ ID NO. 751] |
| Target sequence 94: AAGAAGCTCAGACAGGTGT15 | [SEQ ID NO. 752] |
| Position in gene sequence: 1562 | |
| GC content: 42.9% | |
| Sense strand siRNA: GAAGCUCAGACAGGUGU15tt | [SEQ ID NO. 753] |
| Antisense strand siRNA: 51ACACCUGUCUGAGCUUCtt | [SEQ ID NO. 754] |
| Target sequence 95: AAGCTCAGACAGGTGT1501C | [SEQ ID NO. 755] |
| Position in gene sequence: 1565 | |
| GC content: 42.9% | |
| Sense strand siRNA: GCUCAGACAGGUGU1501Ctt | [SEQ ID NO. 756] |
| Antisense strand siRNA: G1051ACACCUGUCUGAGCtt | [SEQ ID NO. 757] |

TABLE 5
3-alpha-Hydroxysteroiddehydrogenase

3-alpha-Hydroxysteroiddebydrogenase - BAA99542 (gi: 9186908)
[SEQ ID NO. 758]

| | |
|---|---|
| 1 | atggatccca aatatcagcg tgtagagcta aatgatggtc atttcatgcc cgtattggga |
| 61 | tttggcacct atgcacctcc agaggttccg aggaacagag ctgtagaggt caccaaatta |
| 121 | gcaatagaag ctggcttccg ccatattgat tctgcttatt tatacaataa tgaggagcag |
| 181 | gttggactgg ccatccgaag caagattgca gatggcagtg tgaagagaga agacatattc |
| 241 | tacacttcaa agctttggtg cactttcttt caaccacaga tggtccaacc agccttggaa |
| 301 | agctcactga aaaaacttca actggactat gttgacctct atcttcttca tttcccaatg |
| 361 | gctctcaagc caggtgagac gccactacca aaagatgaaa atggaaaagt aatattcgac |
| 421 | acagtggatc tctgtgccac atgggaggtc atggagaagt gtaaggatgc aggattggcc |
| 481 | aagtccatcg gggtgtcaaa cttcaactgc aggcagctgg agatgatcct caacaagcca |
| 541 | ggactcaagt acaagcctgt ctgcaaccag gtagaatgtc atccttacct caaccagagc |
| 601 | aaactgctgg atttctgcaa gtcaaaagac attgttctgg ttgcccacag tgctctggga |
| 661 | acccaacgac ataaactatg ggtggaccca aactccccag ttcttttgga ggacccagtt |
| 721 | ctttgtgcct tagcaaagaa acacaaacga accccagccc tgattgccct gcgctaccag |
| 781 | ctgcagcgtg gggttgtggt cctggccaag agctacaatg agcagcggat cagagagaac |
| 841 | atccaggttt ttgaattcca gttgacatca gaggatatga aagttctaga tggtctaaac |
| 901 | agaaattatc gatatgttgt catggatttt gttatggacc atcctgatta tccattttca |
| 961 | gatgaatatt ag |

| | |
|---|---|
| Target sequence 1: AAATATCAGCGTGTAGAGCTA | [SEQ ID NO. 759] |
| Position in gene sequence: 11 | |
| GC content: 38.1% | |
| Sense strand siRNA: AUAUCAGCGUGUAGAGCUAtt | [SEQ ID NO. 760] |
| Antisense strand siRNA: UAGCUCUACACGCUGAUAUtt | [SEQ ID NO. 761] |
| Target sequence 2: AAATGATGGTCATTTCATGCC | [SEQ ID NO. 762] |
| Position in gene sequence: 31 | |
| GC content: 38.1% | |
| Sense strand siRNA: AUGAUGGUCAUUUCAUGCCtt | [SEQ ID NO. 763] |
| Antisense strand siRNA: GGCAUGAAAUGACCAUCAUtt | [SEQ ID NO. 764] |
| Target sequence 3: AACAGAGCTGTAGAGGTCACC | [SEQ ID NO. 765] |
| Position in gene sequence: 97 | |
| GC content: 52.4% | |
| Sense strand siRNA: CAGAGCUGUAGAGGUCACCtt | [SEQ ID NO. 766] |
| Antisense strand siRNA: GGUGACCUCUACAGCUCUGtt | [SEQ ID NO. 767] |
| Target sequence 4: AAATTA121GCAATAGAAGCT | [SEQ ID NO. 768] |
| Position in gene sequence: 118 | |
| GC content: 23.8% | |
| Sense strand siRNA: AUUA121GCAAUAGAAGCUtt | [SEQ ID NO. 769] |
| Antisense strand siRNA: AGCUUCUAUUGC121UAAUtt | [SEQ ID NO. 770] |
| Target sequence 5: AATAGAAGCTGGCTTCCGCCA | [SEQ ID NO. 771] |
| Position in gene sequence: 129 | |
| GC content: 52.4% | |
| Sense strand siRNA: UAGAAGCUGGCUUCCGCCAtt | [SEQ ID NO. 772] |
| Antisense strand siRNA: UGGCGGAAGCCAGCUUCUAtt | [SEQ ID NO. 773] |
| Target sequence 6: AAGCTGGCTTCCGCCATATTG | [SEQ ID NO. 774] |
| Position in gene sequence: 134 | |
| GC content: 52.4% | |
| Sense strand siRNA: GCUGGCUUCCGCCAUAUUGtt | [SEQ ID NO. 775] |
| Antisense strand siRNA: CAAUAUGGCGGAAGCCAGCtt | [SEQ ID NO. 776] |
| Target sequence 7: AATAATGAGGAGCAG181GTT | [SEQ ID NO. 777] |
| Position in gene sequence: 172 | |
| GC content: 33.3% | |
| Sense strand siRNA: UAAUGAGGAGCAG181GUUtt | [SEQ ID NO. 778] |
| Antisense strand siRNA: AAC181CUGCUCCUCAUUAtt | [SEQ ID NO. 779] |
| Target sequence 8: AATGAGGAGCAG181GTTGGA | [SEQ ID NO. 780] |
| Position in gene sequence: 175 | |
| GC content: 42.9% | |
| Sense strand siRNA: UGAGGAGCAG181GUUGGAtt | [SEQ ID NO. 781] |
| Antisense strand siRNA: UCCAAC181CUGCUCCUCAtt | [SEQ ID NO. 782] |
| Target sequence 9: AAGCAAGATTGCAGATGGCAG | [SEQ ID NO. 783] |
| Position in gene sequence: 207 | |
| GC content: 47.6% | |
| Sense strand siRNA: GCAAGAUUGCAGAUGGCAGtt | [SEQ ID NO. 784] |
| Antisense strand siRNA: CUGCCAUCUGCAAUCUUGCtt | [SEQ ID NO. 785] |
| Target sequence 10: AAGATTGCAGATGGCAGTGTG | [SEQ ID NO. 786] |
| Position in gene sequence: 211 | |
| GC content: 47.6% | |
| Sense strand siRNA: GAUUGCAGAUGGCAGUGUGtt | [SEQ ID NO. 787] |
| Antisense strand siRNA: CACACUGCCAUCUGCAAUCtt | [SEQ ID NO. 788] |
| Target sequence 11: AAGAGAGAAGACATATTC241 | [SEQ ID NO. 789] |
| Position in gene sequence: 232 | |
| GC content: 28.6% | |
| Sense strand siRNA: GAGAGAAGACAUAUUC241tt | [SEQ ID NO. 790] |
| Antisense strand siRNA: 142GAAUAUGUCUUCUCUCtt | [SEQ ID NO. 791] |
| Target sequence 12: AAGACATATTC241TACACTT | [SEQ ID NO. 792] |
| Position in gene sequence: 239 | |
| GC content: 23.8% | |
| Sense strand siRNA: GACAUAUUC241UACACUUtt | [SEQ ID NO. 793] |
| Antisense strand siRNA: AAGUGUA142GAAUAUGUCtt | [SEQ ID NO. 794] |
| Target sequence 13: AAAGCTTTGGTGCACTTTCTT | [SEQ ID NO. 795] |
| Position in gene sequence: 261 | |
| GC content: 38.1% | |
| Sense strand siRNA: AGCUUUGGUGCACUUUCUUtt | [SEQ ID NO. 796] |
| Antisense strand siRNA: AAGAAAGUGCACCAAAGCUtt | [SEQ ID NO. 797] |
| Target sequence 14: AACCACAGATGGTCCAACCAG | [SEQ ID NO. 798] |
| Position in gene sequence: 284 | |
| GC content: 52.4% | |
| Sense strand siRNA: CCACAGAUGGUCCAACCAGtt | [SEQ ID NO. 799] |
| Antisense strand siRNA: CUGGUUGGACCAUCUGUGGtt | [SEQ ID NO. 800] |
| Target sequence 15: AACCAGCCTTGGAA301AGCT | [SEQ ID NO. 801] |
| Position in gene sequence: 299 | |
| GC content: 42.9% | |
| Sense strand siRNA: CCAGCCUUGGAA301AGCUtt | [SEQ ID NO. 802] |
| Antisense strand siRNA: AGCU103UUCCAAGGCUGGtt | [SEQ ID NO. 803] |
| Target sequence 16: AA301AGCTCACTGAAAAAAC | [SEQ ID NO. 804] |
| Position in gene sequence: 311 | |
| GC content: 28.6% | |
| Sense strand siRNA: 301AGCUCACUGAAAAAACtt | [SEQ ID NO. 805] |
| Antisense strand siRNA: GUUUUUUCAGUGAGCU103tt | [SEQ ID NO. 806] |
| Target sequence 17: AAAAAACTTCAACTGGACTAT | [SEQ ID NO. 807] |
| Position in gene sequence: 325 | |
| GC content: 28.6% | |
| Sense strand siRNA: AAAACUUCAACUGGACUAUtt | [SEQ ID NO. 808] |
| Antisense strand siRNA: AUAGUCCAGUUGAAGUUUUtt | [SEQ ID NO. 809] |
| Target sequence 18: AAAACTTCAACTGGACTATGT | [SEQ ID NO. 810] |
| Position in gene sequence: 327 | |
| GC content: 33.3% | |
| Sense strand siRNA: AACUUCAACUGGACUAUGUtt | [SEQ ID NO. 811] |
| Antisense strand siRNA: ACAUAGUCCAGUUGAAGUUtt | [SEQ ID NO. 812] |
| Target sequence 19: AACTTCAACTGGACTATGTTG | [SEQ ID NO. 813] |
| Position in gene sequence: 329 | |
| GC content: 38.1% | |
| Sense strand siRNA: CUUCAACUGGACUAUGUUGtt | [SEQ ID NO. 814] |
| Antisense strand siRNA: CAACAUAGUCCAGUUGAAGtt | [SEQ ID NO. 815] |
| Target sequence 20: AACTGGACTATGTTGACCTCT | [SEQ ID NO. 816] |
| Position in gene sequence: 335 | |
| GC content: 42.9% | |
| Sense strand siRNA: CUGGACUAUGUUGACCUCUtt | [SEQ ID NO. 817] |
| Antisense strand siRNA: AGAGGUCAACAUAGUCCAGtt | [SEQ ID NO. 818] |
| Target sequence 21: AATG361GCTCTCAAGCCAGG | [SEQ ID NO. 819] |
| Position in gene sequence: 372 | |
| GC content: 47.6% | |
| Sense strand siRINA: UG361GCUCUCAAGCCAGGtt | [SEQ ID NO. 820] |
| Antisense strand siRNA: CCUGGCUUGAGAGC163Catt | [SEQ ID NO. 821] |
| Target sequence 22: AAGCCAGGTGAGACGCCACTA | [SEQ ID NO. 822] |
| Position in gene sequence: 385 | |
| GC content: 57.1% | |
| Sense strand siRNA: GCCAGGUGAGACGCCACUAtt | [SEQ ID NO. 823] |
| Antisense strand siRNA: UAGUGGCGUCUCACCUGGCtt | [SEQ ID NO. 824] |
| Target sequence 23: AAAAGATGAAAATGGAAAAGT | [SEQ ID NO. 825] |
| Position in gene sequence: 408 | |
| GC content: 23.8% | |
| Sense strand siRNA: AAGAUGAAAAUGGAAAAGUtt | [SEQ ID NO. 826] |
| Antisense strand siRNA: ACUUUUCCAUUUUCAUCUUtt | [SEQ ID NO. 827] |
| Target sequence 24: AAGATGAAAATGGAAAAGTAA | [SEQ ID NO. 828] |
| Position in gene sequence: 410 | |
| GC content: 23.8% | |
| Sense strand siRNA: GAUGAAAAUGGAAAAGUAAtt | [SEQ ID NO. 829] |
| Antisense strand siRNA: UUACUUUUCCAUUUUCAUCtt | [SEQ ID NO. 830] |
| Target sequence 25: AAAATGGAAAAGTAATATTCG | [SEQ ID NO. 831] |
| Position in gene sequence: 416 | |
| GC content: 23.8% | |
| Sense strand siRNA: AAUGGAAAAGUAAUAUUCGtt | [SEQ ID NO. 832] |
| Antisense strand siRNA: CGAAUAUUACUUUUCCAUUtt | [SEQ ID NO. 833] |
| Target sequence 26: AATGGAAAAGTAATATTCGAC | [SEQ ID NO. 834] |
| Position in gene sequence: 418 | |
| GC content: 28.6% | |
| Sense strand siRNA: UGGAAAAGUAAUAUUCGACtt | [SEQ ID NO. 835] |
| Antisense strand siRNA: GUCGAAUAUUACUUUUCCAtt | [SEQ ID NO. 836] |
| Target sequence 27: AAAAGTAATATTCGAC421AC | [SEQ ID NO. 837] |
| Position in gene sequence: 423 | |
| GC content: 23.8% | |
| Sense strand siRNA: AAGUAAUAUUCGAC421ACtt | [SEQ ID NO. 838] |
| Antisense strand siRNA: GU124GUCGAAUAUUACUUtt | [SEQ ID NO. 839] |
| Target sequence 28: AAGTAATATTCGAC421ACAG | [SEQ ID NO. 840] |
| Position in gene sequence: 425 | |
| GC content: 28.6% | |
| Sense strand siRNA: GUAAUAUUCGAC421ACAGtt | [SEQ ID NO. 841] |
| Antisense strand siRNA: CUGU124GUCGAAUAUUACtt | [SEQ ID NO. 842] |
| Target sequence 29: AATATTCGAC421ACAGTGGA | [SEQ ID NO. 843] |
| Position in gene sequence: 429 | |
| GC content: 33.3% | |
| Sense strand siRNA: UAUUCGAC421ACAGUGGAtt | [SEQ ID NO. 844] |
| Antisense strand siRNA: UCCACUGU124GUCGAAUAtt | [SEQ ID NO. 845] |
| Target sequence 30: AAGTGTAAGGATGCAGGATTG | [SEQ ID NO. 846] |
| Position in gene sequence: 478 | |
| GC content: 42.9% | |
| Sense strand siRNA: GUGUAAGGAUGCAGGAUUGtt | [SEQ ID NO. 847] |
| Antisense strand siRNA: CAAUCCUGCAUCCUUACACtt | [SEQ ID NO. 848] |
| Target sequence 31: AAGGATGCAGGATTGGCC481 | [SEQ ID NO. 849] |
| Position in gene sequence: 484 | |
| GC content: 47.6% | |
| Sense strand siRNA: GGAUGCAGGAUUGGCC481tt | [SEQ ID NO. 850] |
| Antisense strand siRNA: 184GGCCAAUCCUGCAUCCtt | [SEQ ID NO. 851] |
| Target sequence 32: AAGTCCATCGGGGTGTCAAAC | [SEQ ID NO. 852] |
| Position in gene sequence: 505 | |
| GC content: 52.4% | |
| Sense strand siRNA: GUCCAUCGGGGUGUCAAACtt | [SEQ ID NO. 853] |
| Antisense strand siRNA: GUUUGACACCCCGAUGGACtt | [SEQ ID NO. 854] |
| Target sequence 33: AAACTTCAACTGCAGGCAGCT | [SEQ ID NO. 855] |
| Position in gene sequence: 522 | |
| GC content: 47.6% | |
| Sense strand siRNA: ACUUCAACUGCAGGCAGCUtt | [SEQ ID NO. 856] |
| Antisense strand siRNA: AGCUGCCUGCAGUUGAAGUtt | [SEQ ID NO. 857] |
| Target sequence 34: AACTGCAGGCAGCTGGAGATG | [SEQ ID NO. 858] |
| Position in gene sequence: 529 | |
| GC content: 57.1% | |
| Sense strand siRNA: CUGCAGGCAGCUGGAGAUGtt | [SEQ ID NO. 859] |
| Antisense strand siRNA: CAUCUCCAGCUGCCUGCAGtt | [SEQ ID NO. 560] |
| Target sequence 35: AACAAGCCA541GGACTCAAG | [SEQ ID NO. 861] |
| Position in gene sequence: 556 | |
| GC content: 42.9% | |
| Sense strand siRNA: CAAGCCA541GGACUCAAGtt | [SEQ ID NO. 862] |
| Antisense strand siRNA: CUUGAGUCC145UGGCUUGtt | [SEQ ID NO. 863] |
| Target sequence 36: AAGCCA541GGACTCAAGTAC | [SEQ ID NO. 864] |
| Position in gene sequence: 559 | |
| GC content: 42.9% | |
| Sense strand siRNA: GCCA541GGACUCAAGUACtt | [SEQ ID NO. 865] |
| Antisense strand siRNA: GUACUUGAGUCC145UGGCtt | [SEQ ID NO. 866] |
| Target sequence 37: AAGTACAAGCCTGTCTGCAAC | [SEQ ID NO. 867] |
| Position in gene sequence: 574 | |
| GC content: 47.6% | |
| Sense strand siRNA: GUACAAGCCUGUCUGCAACtt | [SEQ ID NO. 868] |
| Antisense strand siRNA: GUUGCAGACAGGCUUGUACtt | [SEQ ID NO. 869] |
| Target sequence 38: AAGCCTGTCTGCAACCAGGTA | [SEQ ID NO. 870] |
| Position in gene sequence: 580 | |
| GC content: 52.4% | |
| Sense strand siRNA: GCCUGUCUGCAACCAGGUAtt | [SEQ ID NO. 871] |
| Antisense strand siRNA: UACCUGGUUGCAGACAGGCtt | [SEQ ID NO. 872] |
| Target sequence 39: AACCAGGTAGAATGTCATCCT | [SEQ ID NO. 873] |
| Position in gene sequence: 592 | |
| GC content: 42.9% | |
| Sense strand siRNA: CCAGGUAGAAUGUCAUCCUtt | [SEQ ID NO. 874] |
| Antisense strand siRNA: AGGAUGACAUUCUACCUGGtt | [SEQ ID NO. 875] |
| Target sequence 40: AATGTCATCCTTACCTCAACC | [SEQ ID NO. 876] |
| Position in gene sequence: 602 | |
| GC content: 42.9% | |
| Sense strand siRNA: UGUCAUCCUUACCUCAACCtt | [SEQ ID NO. 877] |
| Antisense strand siRNA: GGUUGAGGUAAGGAUGACAtt | [SEQ ID NO. 878] |
| Target sequence 41: AACCAGAGC601AAACTGCTG | [SEQ ID NO. 879] |
| Position in gene sequence: 619 | |
| GC content: 42.9% | |
| Sense strand siRNA: CCAGAGC601AAACUGCUGtt | [SEQ ID NO. 880] |
| Antisense strand siRNA: CAGCAGUUU106GCUCUGGtt | [SEQ ID NO. 881] |
| Target sequence 42: AAACTGCTGGATTTCTGCAAG | [SEQ ID NO. 882] |
| Position in gene sequence: 631 | |
| GC content: 42.9% | |
| Sense strand siRNA: ACUGCUGGAUUUCUGCAAGtt | [SEQ ID NO. 883] |
| Antisense strand siRNA: CUUGCAGAAAUCCAGCAGUtt | [SEQ ID NO. 884] |
| Target sequence 43: AAGTCAAAAGACATTGTTCTG | [SEQ ID NO. 885] |
| Position in gene sequence: 649 | |
| GC content: 33.3% | |
| Sense strand siRNA: GUCAAAAGACAUUGUUCUGtt | [SEQ ID NO. 886] |
| Antisense strand siRNA: CAGAACAAUGUCUUUUGACtt | [SEQ ID NO. 887] |
| Target sequence 44: AAAAGACATTGTTCTGGTTGC | [SEQ ID NO. 888] |
| Position in gene sequence: 654 | |
| GC content: 38.1% | |
| Sense strand siRNA: AAGACAUUGUUCUGGUUGCtt | [SEQ ID NO. 889] |
| Antisense strand siRNA: GCAACCAGAACAAUGUCUUtt | [SEQ ID NO. 890] |
| Target sequence 45: AAGACATTGTTCTGGTTGCCC | [SEQ ID NO. 891] |
| Position in gene sequence: 656 | |
| GC content: 47.6% | |
| Sense strand siRNA: GACAUUGUUCUGGUUGCCCtt | [SEQ ID NO. 892] |
| Antisense strand siRNA: GGGCAACCAGAACAAUGUCtt | [SEQ ID NO. 893] |
| Target sequence 46: AACGACATAAACTATGGGTGG | [SEQ ID NO. 894] |
| Position in gene sequence: 698 | |
| GC content: 42.9% | |
| Sense strand siRNA: CGACAUAAACUAUGGGUGGtt | [SEQ ID NO. 895] |
| Antisense strand siRNA: CCACCCAUAGUUUAUGUCGtt | [SEQ ID NO. 896] |
| Target sequence 47: AAACTATGGGTGGACCCAAAC | [SEQ ID NO. 897] |
| Position in gene sequence: 706 | |
| GC content: 47.6% | |
| Sense strand siRNA: ACUAUGGGUGGACCCAAACtt | [SEQ ID NO. 898] |
| Antisense strand siRNA: GUUUGGGUCCACCCAUAGUtt | [SEQ ID NO. 899] |
| Target sequence 48: AAACTCCCCAGTTCTTTTGGA | [SEQ ID NO. 900] |
| Position in gene sequence: 723 | |
| GC content: 42.9% | |
| Sense strand siRNA: ACUCCCCAGUUCUUUUGGAtt | [SEQ ID NO. 901] |
| Antisense strand siRNA: UCCAAAAGAACUGGGGAGUtt | [SEQ ID NO. 902] |
| Target sequence 49: AAAGAAACACAAACGAACCCC | [SEQ ID NO. 903] |
| Position in gene sequence: 771 | |
| GC content: 42.9% | |
| Sense strand siRNA: AGAAACACAAACGAACCCCtt | [SEQ ID NO. 904] |
| Antisense strand siRNA: GGGGUUCGUUUGUGUUUCUtt | [SEQ ID NO. 905] |
| Target sequence 50: AAACACAAACGAACCCCAGCC | [SEQ ID NO. 906] |
| Position in gene sequence: 775 | |
| GC content: 52.4% | |
| Sense strand siRNA: ACACAAACGAACCCCAGCCtt | [SEQ ID NO. 907] |
| Antisense strand siRNA: GGCUGGGGUUCGUUUGUGUtt | [SEQ ID NO. 908] |
| Target sequence 51: AAACGAACCCCAGCCCTGATT | [SEQ ID NO. 909] |
| Position in gene sequence: 781 | |
| GC content: 52.4% | |
| Sense strand siRNA: ACGAACCCCAGCCCUGAUUtt | [SEQ ID NO. 910] |
| Antisense strand siRNA: AAUCAGGGCUGGGGUUCGUtt | [SEQ ID NO. 911] |
| Target sequence 52: AACCCCAGCCCTGATTGCCCT | [SEQ ID NO. 912] |
| Position in gene sequence: 786 | |
| GC content: 61.9% | |
| Sense strand siRNA: CCCCAGCCCUGAUUGCCCUtt | [SEQ ID NO. 913] |
| Antisense strand siRNA: AGGGCAAUCAGGGCUGGGGtt | [SEQ ID NO. 914] |
| Target sequence 53: AAGAGCTACAATGAGCAGCGG | [SEQ ID NO. 915] |
| Position in gene sequence: 847 | |
| GC content: 52.4% | |
| Sense strand siRNA: GAGCUACAAUGAGCAGCGGtt | [SEQ ID NO. 916] |
| Antisense strand siRNA: CCGCUGCUCAUUGUAGCUCtt | [SEQ ID NO. 917] |
| Target sequence 54: AATGAGCAGCGGATCAGAGAG | [SEQ ID NO. 918] |
| Position in gene sequence: 856 | |
| GC content: 52.4% | |
| Sense strand siRNA: UGAGCAGCGGAUCAGAGAGtt | [SEQ ID NO. 919] |
| Antisense strand siRNA: CUCUCUGAUCCGCUGCUCAtt | [SEQ ID NO. 920] |
| Target sequence 55: AAC841ATCCAGGTTTTTGAA | [SEQ ID NO. 921] |
| Position in gene sequence: 877 | |
| GC content: 28.6% | |
| Sense strand siRNA: C841AUCCAGGUUUUUGAAtt | [SEQ ID NO. 922] |
| Antisense strand siRNA: UUCAAAAACCUGGAU148Gtt | [SEQ ID NO. 923] |
| Target sequence 56: AATTCCAGTTGACATCAGAGG | [SEQ ID NO. 924] |
| Position in gene sequence: 896 | |
| GC content: 42.9% | |
| Sense strand siRNA: UUCCAGUUGACAUCAGAGGtt | [SEQ ID NO. 925] |
| Antisense strand siRNA: CCUCUGAUGUCAACUGGAAtt | [SEQ ID NO. 926] |
| Target sequence 57: AAAGTTCTAGATGGTCTAAAC | [SEQ ID NO. 927] |
| Position in gene sequence: 922 | |
| GC content: 33.3% | |
| Sense strand siRNA: AGUUCUAGAUGGUCUAAACtt | [SEQ ID NO. 928] |
| Antisense strand siRNA: GUUUAGACCAUCUAGAACUtt | [SEQ ID NO. 929] |
| Target sequence 58: AAAC901AGAAATTATCGATA | [SEQ ID NO. 930] |
| Position in gene sequence: 939 | |
| GC content: 19% | |
| Sense strand siRNA: AC901AGAAAUUAUCGAUAtt | [SEQ ID NO. 931] |
| Antisense strand siRNA: UAUCGAUAAUUUCU109GUtt | [SEQ ID NO. 932] |
| Target sequence 59: AAATTATCGATATGTTGTCAT | [SEQ ID NO. 933] |
| Position in gene sequence: 948 | |
| GC content: 23.8% | |
| Sense strand siRNA: AUUAUCGAUAUGUUGUCAUtt | [SEQ ID NO. 934] |
| Antisense strand siRNA: AUGACAACAUAUCGAUAAUtt | [SEQ ID NO. 935] |

TABLE 6
3-beta-Hydroxysteroiddehydrogenase

3-beta-Hydroxysteroiddehydrogeoase - DEHUHS (gi: 65959)
[SEQ ID NO. 936]

| | |
|---|---|
| 1 | atgacgggct ggagctgcct tgtgacagga gcaggagggt ttctgggaca gaggatcatc |
| 61 | cgcctcttgg tgaaggagaa ggagctgaag gagatcaggg tcttggacaa ggccttcgga |
| 121 | ccagaattga gagaggaatt ttctaaactc cagaacaaga ccaagctgac agtgctggaa |
| 181 | ggagacattc tggatgagcc attcctgaag agagcctgcc aggacgtctc ggtcatcatc |
| 241 | cacaccgcct gtatcattga tgtcttcggt gtcactcaca gagagtctat catgaatgtc |
| 301 | aatgtgaaag gtacccagct cctgttagag gcctgtgtcc aagctagtgt gccagtcttc |
| 361 | atctacacca gtagcataga ggtagccggg cccaactcct acaaggaaat catccagaat |
| 421 | ggccatgaag aagagcctct ggaaaacaca tggcccgctc catacccaca cagcaaaaag |
| 481 | cttgctgaga aggctgtact ggcggctaac gggtggaatc tgaaaaacgg cggcaccctg |
| 541 | tacacttgtg ccttacgacc catgtatatc tatggggaag gaagccgatt cctttctgct |
| 601 | agtataaacg aggccctgaa caacaatggg atcctgtcaa gtgttggaaa gttctccact |
| 661 | gttaacccag tctatgttgg caatgtggcc tgggcccaca ttctggcctt gagggccctg |
| 721 | caggacccca agaaggcccc aagcatccga ggacagttct actatatctc agatgacacg |
| 781 | cctcaccaaa gctatgataa ccttaattac accctgagca aagagttcgg cctccgcctt |
| 841 | gattccagat ggagctttcc tttatccctg atgtattgga ttggcttcct gctggaaata |
| 901 | gtgagcttcc tactcaggcc aatttacacc tatcgaccgc ccttcaaccg ccacatagtc |
| 961 | acattgtcaa atagcgtatt caccttctct tataagaagg ctcagcgaga tctggcgtat |
| 1021 | aagccactct acagctggga ggaagccaag cagaaaacgg tggagtgggt tggttccctt |
| 1081 | gtggaccggc acaaggagac cctgaagtcc aagactcagt ga |

| | |
|---|---|
| Target sequence 1: AAGGAGAAGGAGCTGAAGGAG | [SEQ ID NO. 937] |
| Position in gene sequence: 75 | |
| GC content: 52.4% | |
| Sense strand siRNA: GGAGAAGGAGCUGAAGGAGtt | [SEQ ID NO. 938] |
| Antisense strand siRNA: CUCCUUCAGCUCCUUCUCCtt | [SEQ ID NO. 939] |
| Target sequence 2: AAGGAGCTGAAGGAGATCAGG | [SEQ ID NO. 940] |
| Position in gene sequence: 81 | |
| GC content: 52.4% | |
| Sense strand siRNA: GGAGCUGAAGGAGAUCAGGtt | [SEQ ID NO. 941] |
| Antisense strand siRNA: CCUGAUCUCCUUCAGCUCCtt | [SEQ ID NO. 942] |
| Target sequence 3: AAGGAGATCAGGGTCTTGGAC | [SEQ ID NO. 943] |
| Position in gene sequence: 90 | |
| GC content: 52.4% | |
| Sense strand siRNA: GGAGAUCAGGGUCUUGGACtt | [SEQ ID NO. 944] |
| Antisense strand siRNA: GUCCAAGACCCUGAUCUCCtt | [SEQ ID NO. 945] |
| Target sequence 4: AAGGCCTTCGGA121CCAGAA | [SEQ ID NO. 946] |
| Position in gene sequence: 111 | |
| GC content: 47.6% | |
| Sense strand siRNA: GGCCUUCGGA121CCAGAAtt | [SEQ ID NO. 947] |
| Antisense strand siRNA: UUCUGG121UCCGAAGGCCtt | [SEQ ID NO. 948] |
| Target sequence 5: AATTGAGAGAGGAATTTTCTA | [SEQ ID NO. 949] |
| Position in gene sequence: 130 | |
| GC content: 28.6% | |
| Sense strand siRNA: UUGAGAGAGGAAUUUUCUAtt | [SEQ ID NO. 950] |
| Antisense strand siRNA: UAGAAAAUUCCUCUCUCAAtt | [SEQ ID NO. 951] |
| Target sequence 6: AATTTTCTAAACTCCAGAACA | [SEQ ID NO. 952] |
| Position in gene sequence: 142 | |
| GC content: 28.6% | |
| Sense strand siRNA: UUUUCUAAACUCCAGAACAtt | [SEQ ID NO. 953] |
| Antisense strand siRNA: UGUUCUGGAGUUUAGAAAAtt | [SEQ ID NO. 954] |
| Target sequence 7: AAACTCCAGAACAAGACCAAG | [SEQ ID NO. 955] |
| Position in gene sequence: 150 | |
| GC content: 42.9% | |
| Sense strand siRNA: ACUCCAGAACAAGACCAAGtt | [SEQ ID NO. 956] |
| Antisense strand siRNA: CUUGGUCUUGUUCUGGAGUtt | [SEQ ID NO. 957] |
| Target sequence 8: AACAAGACCAAGCTGACAGTG | [SEQ ID NO. 958] |
| Position in gene sequence: 159 | |
| GC content: 47.6% | |
| Sense strand siRNA: CAAGACCAAGCUGACAGUGtt | [SEQ ID NO. 959] |
| Antisense strand siRNA: CACUGUCAGCUUGGUCUUGtt | [SEQ ID NO. 960] |
| Target sequence 9: AAGACCAAGCTGACAGTGCTG | [SEQ ID NO. 961] |
| Position in gene sequence: 162 | |
| GC content: 52.4% | |
| Sense strand siRNA: GACCAAGCUGACAGUGCUGtt | [SEQ ID NO. 962] |
| Antisense strand siRNA: CAGCACUGUCAGCUUGGUCtt | [SEQ ID NO. 963] |
| Target sequence 10: AAGCTGACAGTGCTGGAA181 | [SEQ ID NO. 964] |
| Position in gene sequence: 168 | |
| GC content: 42.9% | |
| Sense strand siRNA: GCUGACAGUGCUGGAA181tt | [SEQ ID NO. 965] |
| Antisense strand siRNA: 181UUCCAGCACUGUCAGCtt | [SEQ ID NO. 966] |
| Target sequence 11: AA181GGAGACATTCTGGATG | [SEQ ID NO. 967] |
| Position in gene sequence: 184 | |
| GC content: 38.1% | |
| Sense strand siRNA: 181GGAGACAUUCUGGAUGtt | [SEQ ID NO. 968] |
| Antisense strand siRNA: CAUCCAGAAUGUCUCC181tt | [SEQ ID NO. 969] |
| Target sequence 12: AAGAGAGCCTGCCAGGACGTC | [SEQ ID NO. 970] |
| Position in gene sequence: 216 | |
| GC content: 61.9% | |
| Sense strand siRNA: GAGAGCCUGCCAGGACGUCtt | [SEQ ID NO. 971] |
| Antisense strand siRNA: GACGUCCUGGCAGGCUCUCtt | [SEQ ID NO. 972] |
| Target sequence 13: AATGTC301AATGTGAAAGGT | [SEQ ID NO. 973] |
| Position in gene sequence: 306 | |
| GC content: 28.6% | |
| Sense strand siRNA: UGUC301AAUGUGAAAGGUtt | [SEQ ID NO. 974] |
| Antisense strand siRNA: ACCUUUCACAUU103GACAtt | [SEQ ID NO. 975] |
| Target sequence 14: AATGTGAAAGGTACCCAGCTC | [SEQ ID NO. 976] |
| Position in gene sequence: 315 | |
| GC content: 47.6% | |
| Sense strand siRNA: UGUGAAAGGUACCCAGCUCtt | [SEQ ID NO. 977] |
| Antisense strand siRNA: GAGCUGGGUACCUUUCACAtt | [SEQ ID NO. 978] |
| Target sequence 15: AAAGGTACCCAGCTCCTGTTA | [SEQ ID NO. 979] |
| Position in gene sequence: 321 | |
| GC content 47.6% | |
| Sense strand siRNA: AGGUACCCAGCUCCUGUUAtt | [SEQ ID NO. 980] |
| Antisense strand siRNA: UAACAGGAGCUGGGUACCUtt | [SEQ ID NO. 981] |
| Target sequence 16: AAGCTAGTGTGCCAGTCTTC3 | [SEQ ID NO. 982] |
| Position in gene sequence: 355 | |
| GC content: 47.6% | |
| Sense strand siRNA: GCUAGUGUGCCAGUCUUC3tt | [SEQ ID NO. 983] |
| Antisense strand siRNA: 3GAAGACUGGCACACUAGCtt | [SEQ ID NO. 984] |
| Target sequence 17: AACTCCTACAAGGAAATCATC | [SEQ ID NO. 985] |
| Position in gene sequence: 411 | |
| GC content: 38.1% | |
| Sense strand siRNA: CUCCUACAAGGAAAUCAUCtt | [SEQ ID NO. 986] |
| Antisense strand siRNA: GAUGAUUUCCUUGUAGGAGtt | [SEQ ID NO. 987] |
| Target sequence 18: AAGGAAATCATCCAGAAT421 | [SEQ ID NO. 988] |
| Position in gene sequence: 420 | |
| GC content: 28.6% | |
| Sense strand siRNA: GGAAAUCAUCCAGAAU421tt | [SEQ ID NO. 989] |
| Antisense strand siRNA: 124AUUCUGGAUGAUUUCCtt | [SEQ ID NO. 990] |
| Target sequence 19: AAATCATCCAGAAT421GGCC | [SEQ ID NO. 991] |
| Position in gene sequence: 424 | |
| GC content: 38.1% | |
| Sense strand siRNA: AUCAUCCAGAAU421GGCCtt | [SEQ ID NO. 992] |
| Antisense strand siRNA: GGCC124AUUCUGGAUGAUtt | [SEQ ID NO. 993] |
| Target sequence 20: AAT421GGCCATGAAGAAGAG | [SEQ ID NO. 994] |
| Position in gene sequence: 435 | |
| GC content: 38.1% | |
| Sense strand siRNA: U421GGCCAUGAAGAAGAGtt | [SEQ ID NO. 995] |
| Antisense strand siRNA: CUCUUCUUCAUGGCC124Att | [SEQ ID NO. 996] |
| Target sequence 21: AAGAAGAGCCTCTGGAAAACA | [SEQ ID NO. 997] |
| Position in gene sequence: 448 | |
| GC content: 42.9% | |
| Sense strand siRNA: GAAGAGCCUCUGGAAAACAtt | [SEQ ID NO. 998] |
| Antisense strand siRNA: UGUUUUCCAGAGGCUCUUCtt | [SEQ ID NO. 999] |
| Target sequence 22: AAGAGCCTCTGGAAAACACAT | [SEQ ID NO. 1000] |
| Position in gene sequence: 451 | |
| GC content: 42.9% | |
| Sense strand siRNA: GAGCCUCUGGAAAACACAUtt | [SEQ ID NO. 1001] |
| Antisense strand siRNA: AUGUGUUUUCCAGAGGCUCtt | [SEQ ID NO. 1002] |
| Target sequence 23: AAAACACATGGCCCGCTCCAT | [SEQ ID NO. 1003] |
| Position in gene sequence: 463 | |
| GC content: 52.4% | |
| Sense strand siRNA: AACACAUGGCCCGCUCCAUtt | [SEQ ID NO. 1004] |
| Antisense strand siRNA: AUGGAGCGGGCCAUGUGUUtt | [SEQ ID NO. 1005] |
| Target sequence 24: AACACATGGCCCGCTCCATAC | [SEQ ID NO. 1006] |
| Position in gene sequence: 465 | |
| GC content: 57.1% | |
| Sense strand siRNA: CACAUGGCCCGCUCCAUACtt | [SEQ ID NO. 1007] |
| Antisense strand siRNA: GUAUGGAGCGGGCCAUGUGtt | [SEQ ID NO. 1008] |
| Target sequence 25: AAAAAG481CTTGCTGAGAAG | [SEQ ID NO. 1009] |
| Position in gene sequence: 495 | |
| GC content: 33.3% | |
| Sense strand siRNA: AAAG481CUUGCUGAGAAGtt | [SEQ ID NO. 1010] |
| Antisense strand siRNA: CUUCUCAGCAAG184CUUUtt | [SEQ ID NO. 1011] |
| Target sequence 26: AAAG481CTTGCTGAGAAGGC | [SEQ ID NO. 1012] |
| Position in gene sequence: 497 | |
| GC content: 42.9% | |
| Sense strand siRNA: AG481CUUGCUGAGAAGGCtt | [SEQ ID NO. 1013] |
| Antisense strand siRNA: GCCUUCUCAGCAAG184CUtt | [SEQ ID NO. 1014] |
| Target sequence 27: AAGGCTGTACTGGCGGCTAAC | [SEQ ID NO. 1015] |
| Position in gene sequence: 513 | |
| GC content: 57.1% | |
| Sense strand siRNA: GGCUGUACUGGCGGCUAACtt | [SEQ ID NO. 1016] |
| Antisense strand siRNA: GUUAGCCGCCAGUACAGCCtt | [SEQ ID NO. 1017] |
| Target sequence 28: AACGGGTGGAATCTGAAAAAC | [SEQ ID NO. 1018] |
| Position in gene sequence: 531 | |
| GC content: 42.9% | |
| Sense strand siRNA: CGGGUGGAAUCUGAAAAACtt | [SEQ ID NO. 1019] |
| Antisense strand siRNA: GUUUUUCAGAUUCCACCCGtt | [SEQ ID NO. 1020] |
| Target sequence 29: AATCTGAAAAACGGCGGCACC | [SEQ ID NO. 1021] |
| Position in gene sequence: 540 | |
| GC content: 52.4% | |
| Sense strand siRNA: UCUGAAAAACGGCGGCACCtt | [SEQ ID NO. 1022] |
| Antisense strand siRNA: GGUGCCGCCGUUUUUCAGAtt | [SEQ ID NO. 1023] |
| Target sequence 30: AAAAACGGCGGCACCCTG541 | [SEQ ID NO. 1024] |
| Position in gene sequence: 546 | |
| GC content: 52.4% | |
| Sense strand siRNA: AAACGGCGGCACCCUG541tt | [SEQ ID NO. 1025] |
| Antisense strand siRNA: 145CAGGGUGCCGCCGUUUtt | [SEQ ID NO. 1026] |
| Target sequence 31: AAACGGCGGCACCCTG541TA | [SEQ ID NO. 1027] |
| Position in gene sequence: 548 | |
| GC content: 52.4% | |
| Sense strand siRNA: ACGGCGGCACCCUG541UAtt | [SEQ ID NO. 1028] |
| Antisense strand siRNA: UA145CAGGGUGCCGCCGUtt | [SEQ ID NO. 1029] |
| Target sequence 32: AAGGAAGCCGATTCCTTTCTG | [SEQ ID NO. 1030] |
| Position in gene sequence: 604 | |
| GC content: 47.6% | |
| Sense strand siRNA: GGAAGCCGAUUCCUUUCUGtt | [SEQ ID NO. 1031] |
| Antisense strand siRNA: CAGAAAGGAAUCGGCUUCCtt | [SEQ ID NO. 1032] |
| Target sequence 33: AAGCCGATTCCTTTCTGCT60 | [SEQ ID NO. 1033] |
| Position in gene sequence: 608 | |
| GC content: 42.9% | |
| Sense strand siRNA: GCCGAUUCCUUUCUGCU60tt | [SEQ ID NO. 1034] |
| Antisense strand siRNA: 06AGCAGAAAGGAAUCGGCtt | [SEQ ID NO. 1035] |
| Target sequence 34: AAACGAGGCCCTGAACAACAA | [SEQ ID NO. 1036] |
| Position in gene sequence: 635 | |
| GC content: 47.6% | |
| Sense strand siRNA: ACGAGGCCCUGAACAACAAtt | [SEQ ID NO. 1037] |
| Antisense strand siRNA: UUGUUGUUCAGGGCCUCGUtt | [SEQ ID NO. 1038] |
| Target sequence 35: AACAACAATGGGATCCTGTCA | [SEQ ID NO. 1039] |
| Position in gene sequence: 648 | |
| GC content: 42.9% | |
| Sense strand siRNA: CAACAAUGGGAUCCUGUCAtt | [SEQ ID NO. 1040] |
| Antisense strand siRNA: UGACAGGAUCCCAUUGUUGtt | [SEQ ID NO. 1041] |
| Target sequence 36: AACAATGGGATCCTGTCAAGT | [SEQ ID NO. 1042] |
| Position in gene sequence: 651 | |
| GC content: 42.9% | |
| Sense strand siRNA: CAAUGGGAUCCUGUCAAGUtt | [SEQ ID NO. 1043] |
| Antisense strand siRNA: ACUUGACAGGAUCCCAUUGtt | [SEQ ID NO. 1044] |
| Target sequence 37: AATGGGATCCTGTCAAGTGTT | [SEQ ID NO. 1045] |
| Position in gene sequence: 654 | |
| GC content: 42.9% | |
| Sense strand siRNA: UGGGAUCCUGUCAAGUGUUtt | [SEQ ID NO. 1046] |
| Antisense strand siRNA: AACACUUGACAGGAUCCCAtt | [SEQ ID NO. 1047] |
| Target sequence 38: AAGTGTTGGAAAGTTCTCCAC | [SEQ ID NO. 1048] |
| Position in gene sequence: 668 | |
| UC content: 42.9% | |
| Sense strand siRNA: GUGUUGGAAAGUUCUCCACtt | [SEQ ID NO. 1049] |
| Antisense strand siRNA: GUGGAGAACUUUCCAACACtt | [SEQ ID NO. 1050] |
| Target sequence 39: AAAGTTCTCCACT661GTTAA | [SEQ ID NO. 1051] |
| Position in gene sequence: 677 | |
| GC content: 28.6% | |
| Sense strand siRNA: AGUUCUCCACU661GUUAAtt | [SEQ ID NO. 1052] |
| Antisense strand siRNA: UUAAC166AGUGGAGAACUtt | [SEQ ID NO. 1053] |
| Target sequence 40: AACCCAGTCTATGTTGGCAAT | [SEQ ID NO. 1054] |
| Position in gene sequence: 696 | |
| GC content: 42.9% | |
| Sense strand siRNA: CCCAGUCUAUGUUGGCAAUtt | [SEQ ID NO. 1055] |
| Antisense strand siRNA: AUUGCCAACAUAGACUGGGtt | [SEQ ID NO. 1056] |
| Target sequence 41: AATGTGGCCTGGGCCCACATT | [SEQ ID NO. 1057] |
| Position in gene sequence: 714 | |
| GC content: 57.1% | |
| Sense strand siRNA: UGUGGCCUGGGCCCACAUUtt | [SEQ ID NO. 1058] |
| Antisense strand siRNA: AAUGUGGGCCCAGGCCACAtt | [SEQ ID NO. 1059] |
| Target sequence 42: AAGAAGGCCCCAAGCATCCGA | [SEQ ID NO. 1060] |
| Position in gene sequence: 765 | |
| GC content: 57.1% | |
| Sense strand siRNA: GAAGGCCCCAAGCAUCCGAtt | [SEQ ID NO. 1061] |
| Antisense strand siRNA: UCGGAUGCUUGGGGCCUUCtt | [SEQ ID NO. 1062] |
| Target sequence 43: AAGGCCCCAAGCATCCGAGGA | [SEQ ID NO. 1063] |
| Position in gene sequence: 768 | |
| GC content: 61.9% | |
| Sense strand siRNA: GGCCCCAAGCAUCCGAGGAtt | [SEQ ID NO. 1064] |
| Antisense strand siRNA: UCCUCGGAUGCUUGGGGCCtt | [SEQ ID NO. 1065] |
| Target sequence 44: AAGCATCCGAGGACAGTTCTA | [SEQ ID NO. 1066] |
| Position in gene sequence: 776 | |
| GC content: 47.6% | |
| Sense strand siRNA: GCAUCCGAGGACAGUUCUAtt | [SEQ ID NO. 1067] |
| Antisense strand siRNA: UAGAACUGUCCUCGGAUGCtt | [SEQ ID NO. 1068] |
| Target sequence 45: AAAGCTATGATAACCTTAATT | [SEQ ID NO. 1069] |
| Position in gene sequence: 826 | |
| GC content: 23.8% | |
| Sense strand siRNA: AGCUAUGAUAACCUUAAUUtt | [SEQ ID NO. 1070] |
| Antisense strand siRNA: AAUUAAGGUUAUCAUAGCUtt | [SEQ ID NO. 1071] |
| Target sequence 46: AACCTTAATTACACCCTGAGC | [SEQ ID NO. 1072] |
| Position in gene sequence: 837 | |
| GC content: 42.9% | |
| Sense strand siRNA: CCUUAAUUACACCCUGAGCtt | [SEQ ID NO. 1073] |
| Antisense strand siRNA: GCUCAGGGUGUAAUUAAGGtt | [SEQ ID NO. 1074] |
| Target sequence 47: AATTACACCCTGAGCAAAGAG | [SEQ ID NO. 1075] |
| Position in gene sequence: 843 | |
| GC content: 42.9% | |
| Sense strand siRNA: UUACACCCUGAGCAAAGAGtt | [SEQ ID NO. 1076] |
| Antisense strand siRNA: CUCUUUGCUCAGGGUGUAAtt | [SEQ ID NO. 1077] |
| Target sequence 48: AAAGAGTTCGGCCTCCGCCTT | [SEQ ID NO. 1078] |
| Position in gene sequence: 858 | |
| GC content: 57.1% | |
| Sense strand siRNA: AGAGUUCGGCCUCCGCCUUtt | [SEQ ID NO. 1079] |
| Antisense strand siRNA: AAGGCGGAGGCCGAACUCUtt | [SEQ ID NO. 1080] |
| Target sequence 49: AAATA901GTGAGCTTCCTAC | [SEQ ID NO. 1081] |
| Position in gene sequence: 937 | |
| GC content: 33.3% | |
| Sense strand siRNA: AUA901GUGAGCUUCCUACtt | [SEQ ID NO. 1082] |
| Antisense strand siRNA: GUAGGAAGCUCAC109UAUtt | [SEQ ID NO. 1083] |
| Target sequence 50: AATTTACACCTATCGACCGCC | [SEQ ID NO. 1084] |
| Position in gene sequence: 965 | |
| GC content: 47.6% | |
| Sense strand siRNA: UUUACACCUAUCGACCGCCtt | [SEQ ID NO. 1085] |
| Antisense strand siRNA: GGCGGUCGAUAGGUGUAAAtt | [SEQ ID NO. 1086] |
| Target sequence 51: AACCGCCACATAGTC961ACA | [SEQ ID NO. 1087] |
| Position in gene sequence: 990 | |
| GC content: 42.9% | |
| Sense strand siRNA: CCGCCACAUAGUC961ACAtt | [SEQ ID NO. 1088] |
| Antisense strand siRNA: UGU169GACUAUGUGGCGGtt | [SEQ ID NO. 1089] |
| Target sequence 52: AAATAGCGTATTCACCTTCTC | [SEQ ID NO. 1090] |
| Position in gene sequence: 1016 | |
| GC content: 38.1% | |
| Sense strand siRNA: AUAGCGUAUUCACCUUCUCtt | [SEQ ID NO. 1091] |
| Antisense strand siRNA: GAGAAGGUGAAUACGCUAUtt | [SEQ ID NO. 1092] |
| Target sequence 53: AAGAAGGCTCAGCGAGATCTG | [SEQ ID NO. 1093] |
| Position in gene sequence: 1041 | |
| GC content: 52.4% | |
| Sense strand siRNA: GAAGGCUCAGCGAGAUCUGtt | [SEQ ID NO. 1094] |
| Antisense strand siRNA: CAGAUCUCGCUGAGCCUUCtt | [SEQ ID NO. 1095] |
| Target sequence 54: AAGGCTCAGCGAGATCTGGCG | [SEQ ID NO. 1096] |
| Position in gene sequence: 1044 | |
| GC content: 61.9% | |
| Sense strand siRNA: GGCUCAGCGAGAUCUGGCGtt | [SEQ ID NO. 1097] |
| Antisense strand siRNA: CGCCAGAUCUCGCUGAGCCtt | SEQ ID NO. 1098] |
| Target sequence 55: AAGCCACTCTACAGCTGGGAG | [SEQ ID NO. 1099] |
| Position in gene sequence: 1072 | |
| GC content: 57.1% | |
| Sense strand siRNA: GCCACUCUACAGCUGGGAGtt | [SEQ ID NO. 1100] |
| Antisense strand siRNA: CUCCCAGCUGUAGAGUGGCtt | [SEQ ID NO. 1101] |
| Target sequence 56: AAGCCAAGCAGAAAACGGTGG | [SEQ ID NO. 1102] |
| Position in gene sequence: 1094 | |
| UC content: 52.4% | |
| Sense strand siRNA: GCCAAGCAGAAAACGGUGGtt | [SEQ ID NO. 1103] |
| Antisense strand siRNA: CCACCGUUUUCUGCUUGGCtt | [SEQ ID NO. 1104] |
| Target sequence 57: AAGCAGAAAACGGTGGAGTGG | [SEQ ID NO. 1105] |
| Position in gene sequence: 1099 | |
| GC content: 52.4% | |
| Sense strand siRNA: GCAGAAAACGGUGGAGUGGtt | [SEQ ID NO. 1106] |
| Antisense strand siRNA: CCACUCCACCGUUUUCUGCtt | [SEQ ID NO. 1107] |
| Target sequence 58: AAAACGGTGGAGTGGGTTGGT | [SEQ ID NO. 1108] |
| Position in gene sequence: 1105 | |
| GC content: 52.4% | |
| Sense strand siRNA: AACGGUGGAGUGGGUUGGUtt | [SEQ ID NO. 1109] |
| Antisense strand siRNA: ACCAACCCACUCCACCGUUtt | [SEQ ID NO. 1110] |
| Target sequence 59: AACGGTGGAGTGGGTTGGTTC | [SEQ ID NO. 1111] |
| Position in gene sequence: 1107 | |
| GC content: 57.1% | |
| Sense strand siRNA: CGGUGGAGUGGGUUGGUUCtt | [SEQ ID NO. 1112] |
| Antisense strand siRNA: GAACCAACCCACUCCACCGtt | [SEQ ID NO. 1113] |
| Target sequence 60: AAGGAGACCCTGAAGTCCAAG | [SEQ ID NO. 1114] |
| Position in gene sequence: 1148 | |
| GC content: 52.4% | |
| Sense strand siRNA: GGAGACCCUGAAGUCCAAGtt | [SEQ ID NO. 1115] |
| Antisense strand siRNA: CUUGGACUUCAGGGUCUCCtt | [SEQ ID NO. 1116] |

TABLE 7
3-beta-Hydroxysteroiddehydrogenase-4-5-isomerase

3-beta-Hydroxysteroiddehydrogenase-4-5-isomerase - S45679 (gi: 257052)
[SEQ ID NO. 1117]

| | |
|---|---|
| 1 | atgacgggct ggagctgcct tgtgacagga gcaggagggt ttctgggaca gaggatcatc |
| 61 | cgcctcttgg tgaaggagaa ggagctgaag gagatcaggg tcttggacaa ggccttcgga |
| 121 | ccagaattga gagaggaatt ttctaaactc cagaacaaga ccaagctgac agtgctggaa |
| 181 | ggagacattc tggatgagcc attcctgaag agagcctgcc aggacgtctc ggtcatcatc |
| 241 | cacaccgcct gtatcattga tgtcttcggt gtcactcaca gagagtctat catgaatgtc |
| 301 | aatgtgaaag gtacccagct cctgttagag gcctgtgtcc aagctagtgt gccagtcttc |
| 361 | atctacacca gtagcataga ggtagccggg cccaactcct acaaggaaat catccagaat |
| 421 | ggccatgaag aagagcctct ggaaaacaca tggcccgctc catacccaca cagcaaaaag |
| 481 | cttgctgaga aggctgtact ggcggctaac gggtggaatc tgaaaaacgg cggcaccctg |
| 541 | tacacttgtg ccttacgacc catgtatatc tatggggaag gaagccgatt cctttctgct |
| 601 | agtataaacg aggccctgaa caacaatggg atcctgtcaa gtgttggaaa gttctccact |
| 661 | gttaacccag tctatgttgg caatgtggcc tgggcccaca ttctggcctt gagggccctg |
| 721 | caggacccca agaaggcccc aagcatccga ggacagttct actatatctc agatgacacg |
| 781 | cctcaccaaa gctatgataa ccttaattac accctgagca aagagttcgg cctccgcctt |
| 841 | gattccagat ggagctttcc tttatccctg atgtattgga ttggcttcct gctggaaata |
| 901 | gtgagcttcc tactcaggcc aatttacacc tatcgaccgc ccttcaaccg ccacatagtc |
| 961 | acattgtcaa atagcgtatt caccttctct tataagaagg ctcagcgaga tctggcgtat |
| 1021 | aagccactct acagctggga ggaagccaag cagaaaacgg tggagtgggt tggttccctt |
| 1081 | gtggaccggc acaaggagac cctgaagtcc aagactcagt ga |

| | |
|---|---|
| Target sequence 1: AAGGAGAAGGAGCTGAAGGAG | [SEQ ID NO. 1118] |
| Position in gene sequence: 76 | |
| GC content: 52.4% | |
| Sense strand siRNA: GGAGAAGGAGCUGAAGGAGtt | [SEQ ID NO. 1119] |
| Antisense strand siRNA: CUCCUUCAGCUCCUUCUCCtt | [SEQ ID NO. 1120] |
| Target sequence 2: AAGGAGCTGAAGGAGATCAGG | [SEQ ID NO. 1121] |
| Position in gene sequence: 82 | |
| GC content: 52.4% | |
| Sense strand siRNA: GGAGCUGAAGGAGAUCAGGtt | [SEQ ID NO. 1122] |
| Antisense strand siRNA: CCUGAUCUCCUUCAGCUCCtt | [SEQ ID NO. 1123] |
| Target sequence 3: AAGGAGATCAGGGTCTTGGAC | [SEQ ID NO. 1124] |
| Position in gene sequence: 91 | |
| GC content: 52.4% | |
| Sense strand siRNA: GGAGAUCAGGGUCUUGGACtt | [SEQ ID NO. 1125] |
| Antisense strand siRNA: GUCCAAGACCCUGAUCUCCtt | [SEQ ID NO. 1126] |
| Target sequence 4: AAGGCCTTCGGA121CCAGAA | [SEQ ID NO. 1127] |
| Position in gene sequence: 112 | |
| GC content: 47.6% | |
| Sense strand siRNA: GGCCUUCGGA121CCAGAAtt | [SEQ ID NO. 1128] |
| Antisense strand siRNA: UUCUGG121UCCGAAGGCCtt | [SEQ ID NO. 1129] |
| Target sequence 5: AATTGAGAGAGGAATTTTCTA | [SEQ ID NO. 1130] |
| Position in gene sequence: 131 | |
| GC content: 28.6% | |
| Sense strand siRNA: UUGAGAGAGGAAUUUUCUAtt | [SEQ ID NO. 1131] |
| Antisense strand siRNA: UAGAAAAUUCCUCUCUCAAtt | [SEQ ID NO. 1132] |
| Target sequence 6: AATTTTCTAAACTCCAGAACA | [SEQ ID NO. 1133] |
| Position in gene sequence: 143 | |
| GC content: 28.6% | |
| Sense strand siRNA: UUUUCUAAACUCCAGAACAtt | [SEQ ID NO. 1134] |
| Antisense strand siRNA: UGUUCUGGAGUUUAGAAAAtt | [SEQ ID NO. 1135] |
| Target sequence 7: AAACTCCAGAACAAGACCAAG | [SEQ ID NO. 1136] |
| Position in gene sequence: 151 | |
| GC content: 42.9% | |
| Sense strand siRNA: ACUCCAGAACAAGACCAAGtt | [SEQ ID NO. 1137] |
| Antisense strand siRNA: CUUGGUCUUGUUCUGGAGUtt | [SEQ ID NO. 1138] |
| Target sequence 8: AACAAGACCAAGCTGACAGTG | [SEQ ID NO. 1139] |
| Position in gene sequence: 160 | |
| GC content: 47.6% | |
| Sense strand siRNA: CAAGACCAAGCUGACAGUGtt | [SEQ ID NO. 1140] |
| Antisense strand siRNA: CACUGUCAGCUUGGUCUUGtt | [SEQ ID NO. 1141] |
| Target sequence 9: AAGACCAAGCTGACAGTGCTG | [SEQ ID NO. 1142] |
| Position in gene sequence: 163 | |
| GC content: 52.4% | |
| Sense strand siRNA: GACCAAGCUGACAGUGCUGtt | [SEQ ID NO. 1143] |
| Antisense strand siRNA: CAGCACUGUCAGCUUGGUCtt | [SEQ ID NO. 1144] |
| Target sequence 10: AAGCTGACAGTGCTGGAA181 | [SEQ ID NO. 1145] |
| Position in gene sequence: 169 | |
| GC content: 42.9% | |
| Sense strand siRNA: GCUGACAGUGCUGGAA181tt | [SEQ ID NO. 1146] |
| Antisense strand siRNA: 181UUCCAGCACUGUCAGCtt | [SEQ ID NO. 1147] |
| Target sequence 11: AA181GGAGACATTCTGGATG | [SEQ ID NO. 1148] |
| Position in gene sequence: 185 | |
| GC content: 38.1% | |
| Sense strand siRNA: 181GGAGACAUUCUGGAUGtt | [SEQ ID NO. 1149] |
| Antisense strand siRNA: CAUCCAGAAUGUCUCC181tt | [SEQ ID NO. 1150] |
| Target sequence 12: AAGAGAGCCTGCCAGGACGTC | [SEQ ID NO. 1151] |
| Position in gene sequence: 217 | |
| GC content: 61.9% | |
| Sense strand siRNA: GAGAGCCUGCCAGGACGUCtt | [SEQ ID NO. 1152] |
| Antisense strand siRNA: GACGUCCUGGCAGGCUCUCtt | [SEQ ID NO. 1153] |
| Target sequence 13: AATGTC301AATGTGAAAGGT | [SEQ ID NO. 1154] |
| Position in gene sequence: 307 | |
| GC content: 28.6% | |
| Sense strand siRNA: UGUC301AAUGUGAAAGGUtt | [SEQ ID NO. 1155] |
| Antisense strand siRNA: ACCUUUCACAUU103GACAtt | [SEQ ID NO. 1156] |
| Target sequence 14: AATGTGAAAGGTACCCAGCTC | [SEQ ID NO. 1157] |
| Position in gene sequence: 316 | |
| GC content: 47.6% | |
| Sense strand siRNA: UGUGAAAGGUACCCAGCUCtt | [SEQ ID NO. 1158] |
| Antisense strand siRNA: GAGCUGGGUACCUUUCACAtt | [SEQ ID NO. 1159] |
| Target sequence 15: AAAGGTACCCAGCTCCTGTTA | [SEQ ID NO. 1160] |
| Position in gene sequence: 322 | |
| GC content: 47.6% | |
| Sense strand siRNA: AGGUACCCAGCUCCUGUUAtt | [SEQ ID NO. 1161] |
| Antisense strand siRNA: UAACAGGAGCUGGGUACCUtt | [SEQ ID NO. 1162] |
| Target sequence 16: AAGCTAGTGTGCCAGTCTTC3 | [SEQ ID NO. 1163] |
| Position in gene sequence: 356 | |
| GC content: 47.6% | |
| Sense strand siRNA: GCUAGUGUGCCAGUCUUC3tt | [SEQ ID NO. 1164] |
| Antisense strand siRNA: 3GAAGACUGGCACACUAGCtt | [SEQ ID NO. 1165] |
| Target sequence 17: AACTCCTACAAGGAAATCATC | [SEQ ID NO. 1166] |
| Position in gene sequence: 412 | |
| GC content: 38.1% | |
| Sense strand siRNA: CUCCUACAAGGAAAUCAUCtt | [SEQ ID NO. 1167] |
| Antisense strand siRNA: GAUGAUUUCCUUGUAGGAGtt | [SEQ ID NO. 1168] |
| Target sequence 18: AAGGAAATCATCCAGAAT421 | [SEQ ID NO. 1169] |
| Position in gene sequence: 421 | |
| GC content: 28.6% | |
| Sense strand siRNA: GGAAAUCAUCCAGAAU421tt | [SEQ ID NO. 1170] |
| Antisense strand siRNA: 124AUUCUGGAUGAUUUCCtt | [SEQ ID NO. 1171] |
| Target sequence 19: AAATCATCCAGAAT421GGCC | [SEQ ID NO. 1172] |
| Position in gene sequence: 425 | |
| GC content: 38.1% | |
| Sense strand siRNA: AUCAUCCAGAAU421GGCCtt | [SEQ ID NO. 1173] |
| Antisense strand siRNA: GGCC124AUUCUGGAUGAUtt | [SEQ ID NO. 1174] |
| Target sequence 20: AAT421GGCCATGAAGAAGAG | [SEQ ID NO. 1175] |
| Position in gene sequence: 436 | |
| GC content: 38.1% | |
| Sense strand siRNA: U421GGCCAUGAAGAAGAGtt | [SEQ ID NO. 1176] |
| Antisense strand siRNA: CUCUUCUUCAUGGCC124Att | [SEQ ID NO. 1177] |
| Target sequence 21: AAGAAGAGCCTCTGGAAAACA | [SEQ ID NO. 1178] |
| Position in gene sequence: 449 | |
| GC content: 42.9% | |
| Sense strand siRNA: GAAGAGCCUCUGGAAAACAtt | [SEQ ID NO. 1179] |
| Antisense strand siRNA: UGUUUUCCAGAGGCUCUUCtt | [SEQ ID NO. 1180] |
| Target sequence 22: AAGAGCCTCTGGAAAACACAT | [SEQ ID NO. 1181] |
| Position in gene sequence: 452 | |
| GC content: 42.9% | |
| Sense strand siRNA: GAGCCUCUGGAAAACACAUtt | [SEQ ID NO. 1182] |
| Antisense strand siRNA: AUGUGUUUUCCAGAGGCUCtt | [SEQ ID NO. 1183] |
| Target sequence 23: AAAACACATGGCCCGCTCCAT | [SEQ ID NO. 1184] |
| Position in gene sequence: 464 | |
| GC content: 52.4% | |
| Sense strand siRNA: AACACAUGGCCCGCUCCAUtt | [SEQ ID NO. 1185] |
| Antisense strand siRNA: AUGGAGCGGGCCAUGUGUUtt | [SEQ ID NO. 1186] |
| Target sequence 24: AACACATGGCCCGCTCCATAC | [SEQ ID NO. 1187] |
| Position in gene sequence: 466 | |
| GC content: 57.1% | |
| Sense strand siRNA: CACAUGGCCCGCUCCAUACtt | [SEQ ID NO. 1188] |
| Antisense strand siRNA: GUAUGGAGCGGGCCAUGUGtt | [SEQ ID NO. 1189] |
| Target sequence 25: AAAAAG481CTTGCTGAGAAG | [SEQ ID NO. 1190] |
| Position in gene sequence: 496 | |
| GC content: 33.3% | |
| Sense strand siRNA: AAAG481CUUGCUGAGAAGtt | [SEQ ID NO. 1191] |
| Antisense strand siRNA: CUUCUCAGCAAG184CUUUtt | [SEQ ID NO. 1192] |
| Target sequence 26: AAAG481CTTGCTGAGAAGGC | [SEQ ID NO. 1193] |
| Position in gene sequence: 498 | |
| GC content: 42.9% | |
| Sense strand siRNA: AG481CUUGCUGAGAAGGCtt | [SEQ ID NO. 1194] |
| Antisense strand siRNA: GCCUUCUCAGCAAG184Cutt | [SEQ ID NO. 1195] |
| Target sequence 27: AAGGCTGTACTGGCGGCTAAC | [SEQ ID NO. 1196] |
| Position in gene sequence: 514 | |
| GC content: 57.1% | |
| Sense strand siRNA: GGCUGUACUGGCGGCUAACtt | [SEQ ID NO. 1197] |
| Antisense strand siRNA: GUUAGCCGCCAGUACAGCCtt | [SEQ ID NO. 1198] |
| Target sequence 28: AACGGGTGGAATCTGAAAAAC | [SEQ ID NO. 1199] |
| Position in gene sequence: 532 | |
| GC content: 42.9% | |
| Sense strand siRNA: CGGGUGGAAUCUGAAAAACtt | [SEQ ID NO. 1200] |
| Antisense strand siRNA: GUUUUUCAGAUUCCACCCGtt | [SEQ ID NO. 1201] |
| Target sequence 29: AATCTGAAAAACGGCGGCACC | [SEQ ID NO. 1202] |
| Position in gene sequence: 541 | |
| GC content: 52.4% | |
| Sense strand siRNA: UCUGAAAAACGGCGGCACCtt | [SEQ ID NO. 1203] |
| Antisense strand siRNA: GGUGCCGCCGUUUUUCAGAtt | [SEQ ID NO. 1204] |
| Target sequence 30: AAAAACGGCGGCACCCTG541 | [SEQ ID NO. 1205] |
| Position in gene sequence: 547 | |
| GC content: 52.4% | |
| Sense strand siRNA: AAACGGCGGCACCCUG541tt | [SEQ ID NO. 1206] |
| Antisense strand siRNA: 145CAGGGUGCCGCCGUUUtt | [SEQ ID NO. 1207] |
| Target sequence 31: AAACGGCGGCACCCTG541TA | [SEQ ID NO. 1208] |
| Position in gene sequence: 549 | |
| GC content: 52.4% | |
| Sense strand siRNA: ACGGCGGCACCCUG541Uatt | [SEQ ID NO. 1209] |
| Antisense strand siRNA: UA145CAGGGUGCCGCCGUtt | [SEQ ID NO. 1210] |
| Target sequence 32: AAGGAAGCCGATTCCTTTCTG | [SEQ ID NO. 1211] |
| Position in gene sequence: 605 | |
| GC content: 47.6% | |
| Sense strand siRNA: GGAAGCCGAUUCCUUUCUGtt | [SEQ ID NO. 1212] |
| Antisense strand siRNA: CAGAAAGGAAUCGGCUUCCtt | [SEQ ID NO. 1213] |
| Target sequence 33: AAGCCGATTCCTTTCTGCT60 | [SEQ ID NO. 1214] |
| Position in gene sequence: 609 | |
| GC content: 42.9% | |
| Sense strand siRNA: GCCGAUUCCUUUCUGCU60tt | [SEQ ID NO. 1215] |
| Antisense strand siRNA: 06AGCAGAAAGGAAUCGGCtt | [SEQ ID NO. 1216] |
| Target sequence 34: AAACGAGGCCCTGAACAACAA | [SEQ ID NO. 1217] |
| Position in gene sequence: 636 | |
| GC content: 47.6% | |
| Sense strand siRNA: ACGAGGCCCUGAACAACAAtt | [SEQ ID NO. 1218] |
| Antisense strand siRNA: UUGUUGUUCAGGGCCUCGUtt | [SEQ ID NO. 1219] |
| Target sequence 35: AACAACAATGGGATCCTGTCA | [SEQ ID NO. 1220] |
| Position in gene sequence: 649 | |
| GC content: 42.9% | |
| Sense strand siRNA: CAACAAUGGGAUCCUGUCAtt | [SEQ ID NO. 1221] |
| Antisense strand siRNA: UGACAGGAUCCCAUUGUUGtt | [SEQ ID NO. 1222] |
| Target sequence 36: AACAATGGGATCCTGTCAAGT | [SEQ ID NO. 1223] |
| Position in gene sequence: 652 | |
| GC content: 42.9% | |
| Sense strand siRNA: CAAUGGGAUCCUGUCAAGUtt | [SEQ ID NO. 1224] |
| Antisense strand siRNA: ACUUGACAGGAUCCCAUUGtt | [SEQ ID NO. 1225] |
| Target sequence 37: AATGGGATCCTGTCAAGTGTT | [SEQ ID NO. 1226] |
| Position in gene sequence: 655 | |
| GC content: 42.9% | |
| Sense strand siRNA: UGGGAUCCUGUCAAGUGUUtt | [SEQ ID NO. 1227] |
| Antisense strand siRNA: AACACUUGACAGGAUCCCAtt | [SEQ ID NO. 1228] |
| Target sequence 38: AAGTGTTGGAAAGTTCTCCAC | [SEQ ID NO. 1229] |
| Position in gene sequence: 669 | |
| GC content: 42.9% | |
| Sense strand siRNA: GUGUUGGAAAGUUCUCCACtt | [SEQ ID NO. 1230] |
| Antisense strand siRNA: GUGGAGAACUUUCCAACACtt | [SEQ ID NO. 1231] |
| Target sequence 39: AAAGTTCTCCACT661GTTAA | [SEQ ID NO. 1232] |
| Position in gene sequence: 678 | |
| GC content: 28.6% | |
| Sense strand siRNA: AGUUCUCCACU661GUUAAtt | [SEQ ID NO. 1233] |
| Antisense strand siRNA: UUAAC166AGUGGAGAACUtt | [SEQ ID NO. 1234] |
| Target sequence 40: AACCCAGTCTATGTTGGCAAT | [SEQ ID NO. 1235] |
| Position in gene sequence: 697 | |
| GC content: 42.9% | |
| Sense strand siRNA: CCCAGUCUAUGUUGGCAAUtt | [SEQ ID NO. 1236] |
| Antisense strand siRNA: AUUGCCAACAUAGACUGGGtt | [SEQ ID NO. 1237] |
| Target sequence 41: AATGTGGCCTGGGCCCACATT | [SEQ ID NO. 1238] |
| Position in gene sequence: 715 | |
| GC content: 57.1% | |
| Sense strand siRNA: UGUGGCCUGGGCCCACAUUtt | [SEQ ID NO. 1239] |
| Antisense strand siRNA: AAUGUGGGCCCAGGCCACAtt | [SEQ ID NO. 1240] |
| Target sequence 42: AAGAAGGCCCCAAGCATCCGA | [SEQ ID NO. 1241] |
| Position in gene sequence: 766 | |
| GC content: 57.1% | |
| Sense strand siRNA: GAAGGCCCCAAGCAUCCGAtt | [SEQ ID NO. 1242] |
| Antisense strand siRNA: UCGGAUGCUUGGGGCCUUCtt | [SEQ ID NO. 1243] |
| Target sequence 43: AAGGCCCCAAGCATCCGAGGA | [SEQ ID NO. 1244] |
| Position in gene sequence: 769 | |
| GC content: 61.9% | |
| Sense strand siRNA: GGCCCCAAGCAUCCGAGGAtt | [SEQ ID NO. 1245] |
| Antisense strand siRNA: UCCUCGGAUGCUUGGGGCCtt | [SEQ ID NO. 1246] |
| Target sequence 44: AAGCATCCGAGGACAGTTCTA | [SEQ ID NO. 1247] |
| Position in gene sequence: 777 | |
| GC content: 47.6% | |
| Sense strand siRNA: GCAUCCGAGGACAGUUCUAtt | [SEQ ID NO. 1248] |
| Antisense strand siRNA: UAGAACUGUCCUCGGAUGCtt | [SEQ ID NO. 1249] |
| Target sequence 45: AAAGCTATGATAACCTTAATT | [SEQ ID NO. 1250] |
| Position in gene sequence: 827 | |
| GC content: 23.8% | |
| Sense strand siRNA: AGCUAUGAUAACCUUAAUUtt | [SEQ ID NO. 1251] |
| Antisense strand siRNA: AAUUAAGGUUAUCAUAGCUtt | [SEQ ID NO. 1252] |
| Target sequence 46: AACCTTAATTACACCCTGAGC | [SEQ ID NO. 1253] |
| Position in gene sequence: 838 | |
| GC content: 42.9% | |
| Sense strand siRNA: CCUUAAUUACACCCUGAGCtt | [SEQ ID NO. 1254] |
| Antisense strand siRNA: GCUCAGGGUGUAAUUAAGGtt | [SEQ ID NO. 1255] |
| Target sequence 47: AATTACACCCTGAGCAAAGAG | [SEQ ID NO. 1256] |
| Position in gene sequence: 844 | |
| GC content: 42.9% | |
| Sense strand siRNA: UUACACCCUGAGCAAAGAGtt | [SEQ ID NO. 1257] |
| Antisense strand siRNA: CUCUUUGCUCAGGGUGUAAtt | [SEQ ID NO. 1258] |
| Target sequence 48: AAAGAGTTCGGCCTCCGCCTT | [SEQ ID NO. 1259] |
| Position in gene sequence: 859 | |
| GC content: 57.1% | |
| Sense strand siRNA: AGAGUUCGGCCUCCGCCUUtt | [SEQ ID NO. 1260] |
| Antisense strand siRNA: AAGGCGGAGGCCGAACUCUtt | [SEQ ID NO. 1261] |
| Target sequence 49: AAATA901GTGAGCTTCCTAC | [SEQ ID NO. 1262] |
| Position in gene sequence: 938 | |
| GC content: 33.3% | |
| Sense strand siRNA: AUA901GUGAGCUUCCUACtt | [SEQ ID NO. 1263] |
| Antisense strand siRNA: GUAGGAAGCUCAC109UAUtt | [SEQ ID NO. 1264] |
| Target sequence 50: AATTTACACCTATCGACCGCC | [SEQ ID NO. 1265] |
| Position in gene sequence: 966 | |
| GC content: 47.6% | |
| Sense strand siRNA: UUUACACCUAUCGACCGCCtt | [SEQ ID NO. 1266] |
| Antisense strand siRNA: GGCGGUCGAUAGGUGUAAAtt | [SEQ ID NO. 1267] |
| Target sequence 51: AACCGCCACATAGTC961ACA | [SEQ ID NO. 1268] |
| Position in gene sequence: 991 | |
| GC content: 42.9% | |
| Sense strand siRNA: CCGCCACAUAGUC961ACAtt | [SEQ ID NO. 1269] |
| Antisense strand siRNA: UGU169GACUAUGUGGCGGtt | [SEQ ID NO. 1270] |
| Target sequence 52: AAATAGCGTATTCACCTTCTC | [SEQ ID NO. 1271] |
| Position in gene sequence: 1017 | |
| GC content: 38.1% | |
| Sense strand siRNA: AUAGCGUAUUCACCUUCUCtt | [SEQ ID NO. 1272] |
| Antisense strand siRNA: GAGAAGGUGAAUACGCUAUtt | [SEQ ID NO. 1273] |
| Target sequence 53: AAGAAGGCTCAGCGAGATCTG | [SEQ ID NO. 1274] |
| Position in gene sequence: 1042 | |
| GC content: 52.4% | |
| Sense strand siRNA: GAAGGCUCAGCGAGAUCUGtt | [SEQ ID NO. 1275] |
| Antisense strand siRNA: CAGAUCUCGCUGAGCCUUCtt | [SEQ ID NO. 1276] |
| Target sequence 54: AAGGCTCAGCGAGATCTGGCG | [SEQ ID NO. 1277] |
| Position in gene sequence: 1045 | |
| GC content: 61.9% | |
| Sense strand siRNA: GGCUCAGCGAGAUCUGGCGtt | [SEQ ID NO. 1278] |
| Antisense strand siRNA: CGCCAGAUCUCGCUGAGCCtt | [SEQ ID NO. 1279] |
| Target sequence 55: AAGCCACTCTACAGCTGGGAG | [SEQ ID NO. 1280] |
| Position in gene sequence: 1073 | |
| GC content: 57.1% | |
| Sense strand siRNA: GCCACUCUACAGCUGGGAGtt | [SEQ ID NO. 1281] |
| Antisense strand siRNA: CUCCCAGCUGUAGAGUGGCtt | [SEQ ID NO. 1282] |
| Target sequence 56: AAGCCAAGCAGAAAACGGTGG | [SEQ ID NO. 1283] |
| Position in gene sequence: 1095 | |
| GC content: 52.4% | |
| Sense strand siRNA: GCCAAGCAGAAAACGGUGGtt | [SEQ ID NO. 1284] |
| Antisense strand siRNA: CCACCGUUUUCUGCUUGGCtt | [SEQ ID NO. 1285] |
| Target sequence 57: AAGCAGAAAACGGTGGAGTGG | [SEQ ID NO. 1286] |
| Position in gene sequence: 1100 | |
| GC content: 52.4% | |
| Sense strand siRNA: GCAGAAAACGGUGGAGUGGtt | [SEQ ID NO. 1287] |
| Antisense strand siRNA: CCACUCCACCGUUUUCUGCtt | [SEQ ID NO. 1288] |
| Target sequence 58: AAAACGGTGGAGTGGGTTGGT | [SEQ ID NO. 1289] |
| Position in gene sequence: 1106 | |
| GC content: 52.4% | |
| Sense strand siRNA: AACGGUGGAGUGGGUUGGUtt | [SEQ ID NO. 1290] |
| Antisense strand siRNA: ACCAACCCACUCCACCGUUtt | [SEQ ID NO. 1291] |
| Target sequence 59: AACGGTGGAGTGGGTTGGTTC | [SEQ ID NO. 1292] |
| Position in gene sequence: 1108 | |
| GC content: 57.1% | |
| Sense strand siRNA: CGGUGGAGUGGGUUGGUUCtt | [SEQ ID NO. 1293] |
| Antisense strand siRNA: GAACCAACCCACUCCACCGtt | [SEQ ID NO. 1294] |
| Target sequence 60: AAGGAGACCCTGAAGTCCAAG | [SEQ ID NO. 1295] |
| Position in gene sequence: 1149 | |
| GC content: 52.4% | |
| Sense strand siRNA: GGAGACCCUGAAGUCCAAGtt | [SEQ ID NO. 1296] |
| Antisense strand siRNA: CUUGGACUUCAGGGUCUCCtt | [SEQ ID NO. 1297] |

TABLE 8
17-beta-Hydroxysteroidoxidoreductase

17-beta-Hydroxysteroidoxidoreductase - P14061 (gi: 118554)
[SEQ ID NO. 1298]

| | |
|---|---|
| 1 | atggcccgca ccgtggtgct catcaccggc tgttcctcgg gcatcggcct gcacttggcc |
| 61 | gtacgtctgg cttcagatcc atcccagagc ttcaaagtgt atgccacgtt gagggacctg |
| 121 | aaaacacagg gccggctgtg ggaggcggcc cgggccctgg catgccctcc gggatccctg |
| 181 | gagacgttgc agctggacgt aagggactca aaatccgtgg ccgctgcccg ggaacgcgtg |
| 241 | actgagggcc gcgtggacgt gctggtgtgt aacgcaggcc tgggcctgct ggggccgctg |
| 301 | gaggcgctgg gggaggacgc cgtggcctct gtgctggacg tgaatgtagt agggactgtg |
| 361 | cggatgctgc aggccttcct gccagacatg aagaggcgcg gttcgggacg cgtgttggtg |
| 421 | accgggagcg tgggaggatt gatggggctg cctttcaatg acgtttattg cgccagcaag |
| 481 | ttcgcgctcg aaggcttatg cgagagtctg gcggttctgc tgctgccctt tggggtccac |
| 541 | ttgagcctga tcgagtgcgg cccagtgcac accgccttca tggagaaggt gttgggcagc |
| 601 | ccagaggagg tgctggaccg cacggacatc cacaccttcc accgcttcta ccaatacctc |
| 661 | gcccacagca agcaagtctt tcgcgaggcg gcgcagaacc ctgaggaggt ggcggaggtc |
| 721 | ttcctcaccg ctttgcgcgc cccgaagccg accctgcgct acttcaccac cgagcgcttc |
| 781 | ctgcccctgc tgcggatgcg cctggacgac cccagcggct ccaactacgt caccgccatg |
| 841 | caccgggaag tgttcggcga cgttccggca aaggccgagg ctggggccga ggctgggggc |
| 901 | ggggccgggc ctggggcaga ggacgaggcc gggcgcagtg cggtggggga ccctgagctc |
| 961 | ggcgatcctc cggccgcccc gcagtaa |

| | |
|---|---|
| Target sequence 1: AAAGTGTATGCCACGTTGAGG | [SEQ ID NO. 1299] |
| Position in gene sequence: 97 | |
| GC content: 47.6% | |
| Sense strand siRNA: AGUGUAUGCCACGUUGAGGtt | [SEQ ID NO. 1300] |
| Antisense strand siRNA: CCUCAACGUGGCAUACACUtt | [SEQ ID NO. 1301] |
| Target sequence 2: AAAACACAGGGCCGGCTGTGG | [SEQ ID NO. 1302] |
| Position in gene sequence: 127 | |
| GC content: 61.9% | |
| Sense strand siRNA: AACACAGGGCCGGCUGUGGtt | [SEQ ID NO. 1303] |
| Antisense strand siRNA: CCACAGCCGGCCCUGUGUUtt | [SEQ ID NO. 1304] |
| Target sequence 3: AACACAGGGCCGGCTGTGGGA | [SEQ ID NO. 1305] |
| Position in gene sequence: 129 | |
| GC content: 66.7% | |
| Sense strand siRNA: CACAGGGCCGGCUGUGGGAtt | [SEQ ID NO. 1306] |
| Antisense strand siRNA: UCCCACAGCCGGCCCUGUGtt | [SEQ ID NO. 1307] |
| Target sequence 4: AAGGGACTCAAAATCCGTGGC | [SEQ ID NO. 1308] |
| Position in gene sequence: 210 | |
| GC content: 52.4% | |
| Sense strand siRNA: GGGACUCAAAAUCCGUGGCtt | [SEQ ID NO. 1309] |
| Antisense strand siRNA: GCCACGGAUUUUGAGUCCCtt | [SEQ ID NO. 1310] |
| Target sequence 5: AAAATCCGTGGCCGCTGCCCG | [SEQ ID NO. 1311] |
| Position in gene sequence: 219 | |
| GC content: 66.7% | |
| Sense strand siRNA: AAUCCGUGGCCGCUGCCCGtt | [SEQ ID NO. 1312] |
| Antisense strand siRNA: CGGGCAGCGGCCACGGAUUtt | [SEQ ID NO. 1313] |
| Target sequence 6: AATCCGTGGCCGCTGCCCGGG | [SEQ ID NO. 1314] |
| Position in gene sequence: 221 | |
| GC content: 76.2% | |
| Sense strand siRNA: UCCGUGGCCGCUGCCCGGGtt | [SEQ ID NO. 1315] |
| Antisense strand siRNA: CCCGGGCAGCGGCCACGGAtt | [SEQ ID NO. 1316] |
| Target sequence 7: AACGCGTG241ACTGAGGGCC | [SEQ ID NO. 1317] |
| Position in gene sequence: 242 | |
| GC content: 57.1% | |
| Sense strand siRNA: CGCGUG241ACUGAGGGCCtt | [SEQ ID NO. 1318] |
| Antisense strand siRNA: GGCCCUCAGU142CACGCGtt | [SEQ ID NO. 1319] |
| Target sequence 8: AACGCAGGCCTGGGCCTGCTG | [SEQ ID NO. 1320] |
| Position in gene sequence: 283 | |
| GC content: 71.4% | |
| Sense strand siRNA: CGCAGGCCUGGGCCUGCUGtt | [SEQ ID NO. 1321] |
| Antisense strand siRNA: CAGCAGGCCCAGGCCUGCGtt | [SEQ ID NO. 1322] |
| Target sequence 9: AATGTAGTAGGGACTGTG361 | [SEQ ID NO. 1323] |
| Position in gene sequence: 358 | |
| GC content: 38.1% | |
| Sense strand siRNA: UGUAGUAGGGACUGUG361tt | [SEQ ID NO. 1324] |
| Antisense strand siRNA: 163CACAGUCCCUACUACAtt | [SEQ ID NO. 1325] |
| Target sequence 10: AAGAGGCGCGGTTCGGGACGC | [SEQ ID NO. 1326] |
| Position in gene sequence: 409 | |
| GC content: 71.4% | |
| Sense strand siRNA: GAGGCGCGGUUCGGGACGCtt | [SEQ ID NO. 1327] |
| Antisense strand siRNA: GCGUCCCGAACCGCGCCUCtt | [SEQ ID NO. 1328] |
| Target sequence 11: AATGACGTTTATTGCGCCAGC | [SEQ ID NO. 1329] |
| Position in gene sequence: 478 | |
| GC content: 47.6% | |
| Sense strand siRNA: UGACGUUUAUUGCGCCAGCtt | [SEQ ID NO. 1330] |
| Antisense strand siRNA: GCUGGCGCAAUAAACGUCAtt | [SEQ ID NO. 1331] |
| Target sequence 12: AAG481TTCGCGCTCGAAGGC | [SEQ ID NO. 1332] |
| Position in gene sequence: 499 | |
| GC content: 52.4% | |
| Sense strand siRNA: G481UUCGCGCUCGAAGGCtt | [SEQ ID NO. 1333] |
| Antisense strand siRNA: GCCUUCGAGCGCGAA184Ctt | [SEQ ID NO. 1334] |
| Target sequence 13: AAGGCTTATGCGAGAGTCTGG | [SEQ ID NO. 1335] |
| Position in gene sequence: 515 | |
| GC content: 52.4% | |
| Sense strand siRNA: GGCUUAUGCGAGAGUCUGGtt | [SEQ ID NO. 1336] |
| Antisense strand siRNA: CCAGACUCUCGCAUAAGCCtt | [SEQ ID NO. 1337] |
| Target sequence 14: AAGGTGTTGGGCAGC601CCA | [SEQ ID NO. 1338] |
| Position in gene sequence: 613 | |
| GC content: 52.4% | |
| Sense strand siRNA: GGUGUUGGGCAGC601CCAtt | [SEQ ID NO. 1339] |
| Antisense strand siRNA: UGG106GCUGCCCAACACCtt | [SEQ ID NO. 1340] |
| Target sequence 15: AATACCTC661GCCCACAGCA | [SEQ ID NO. 1341] |
| Position in gene sequence: 683 | |
| GC content: 47.6% | |
| Sense strand siRNA: UACCUC661GCCCACAGCAtt | [SEQ ID NO. 1342] |
| Antisense strand siRNA: UGCUGUGGGC166GAGGUAtt | [SEQ ID NO. 1343] |
| Target sequence 16: AAGCAAGTCTTTCGCGAGGCG | [SEQ ID NO. 1344] |
| Position in gene sequence: 703 | |
| GC content: 57.1% | |
| Sense strand siRNA: GCAAGUCUUUCGCGAGGCGtt | [SEQ ID NO. 1345] |
| Antisense strand siRNA: CGCCUCGCGAAAGACUUGCtt | [SEQ ID NO. 1346] |
| Target sequence 17: AAGTCTTTCGCGAGGCGGCGC | [SEQ ID NO. 1347] |
| Position in gene sequence: 707 | |
| GC content: 66.7% | |
| Sense strand siRNA: GUCUUUCGCGAGGCGGCGCtt | [SEQ ID NO. 1348] |
| Antisense strand siRNA: GCGCCGCCUCGCGAAAGACtt | [SEQ ID NO. 1349] |
| Target sequence 18: AACCCTGAGGAGGTGGCGGAG | [SEQ ID NO. 1350] |
| Position in gene sequence: 730 | |
| GC content: 66.7% | |
| Sense strand siRNA: CCCUGAGGAGGUGGCGGAGtt | [SEQ ID NO. 1351] |
| Antisense strand siRNA: CUCCGCCACCUCCUCAGGGtt | [SEQ ID NO. 1352] |
| Target sequence 19: AAGCCGACCCTGCGCTACTTC | [SEQ ID NO. 1353] |
| Position in gene sequence: 781 | |
| GC content: 61.9% | |
| Sense strand siRNA: GCCGACCCUGCGCUACUUCtt | [SEQ ID NO. 1354] |
| Antisense strand siRNA: GAAGUAGCGCAGGGUCGGCtt | [SEQ ID NO. 1355] |
| Target sequence 20: AACTACGTCACCGCCATG841 | [SEQ ID NO. 1356] |
| Position in gene sequence: 862 | |
| GC content: 47.6% | |
| Sense strand siRNA: CUACGUCACCGCCAUG841tt | [SEQ ID NO. 1357] |
| Antisense strand siRNA: 148CAUGGCGGUGACGUAGtt | [SEQ ID NO. 1358] |
| Target sequence 21: AAGTGTTCGGCGACGTTCCGG | [SEQ ID NO. 1359] |
| Position in gene sequence: 890 | |
| GC content: 61.9% | |
| Sense strand siRNA: GUGUUCGGCGACGUUCCGGtt | [SEQ ID NO. 1360] |
| Antisense strand siRNA: CCGGAACGUCGCCGAACACtt | [SEQ ID NO. 1361] |
| Target sequence 22: AAAGGCCGAGGCTGGGGCCGA | [SEQ ID NO. 1362] |
| Position in gene sequence: 912 | |
| GC content: 71.4% | |
| Sense strand siRNA: AGGCCGAGGCUGGGGCCGAtt | [SEQ ID NO. 1363] |
| Antisense strand siRNA: UCGGCCCCAGCCUCGGCCUtt | [SEQ ID NO. 1364] |

TABLE 9
Steroid Sulfatase

Steroid Sulfatase - AAA60598 (gi: 338607) [SEQ ID NO. 1365]

| | |
|---|---|
| 1 | ggaaatggca ccttgggatg agctgtcaca gcaagactga cttctgtcac caccctttac |
| 61 | atcacggctt caattatttc tatgggatct ctttgaccaa tctgagagac tgcaagcccg |
| 121 | gagagggcag tgtcttcacc acgggcttca agaggctggt cttcctcccc ctgcagatcg |
| 181 | tcggggtcac cctccttacc cttgctgcac tcaattgtct ggggctactc cacgtgcctc |
| 241 | taggcgtttt tttcagcctt ctcttcctag cagccctaat cctgaccctt ttcttgggct |
| 301 | tccttcatta cttccggccc ctgaactgct tcatgatgag gaactacgag atcattcagc |
| 361 | agcccatgtc ctatgacaat ctcacccaga ggctaacggt ggaggcggcc cagttcatac |
| 421 | agcg |

| | |
|---|---|
| Target sequence 1: AAATGGCACCTTGGGATGAGC | [SEQ ID NO. 1366] |
| Position in gene sequence: 4 | |
| GC content: 52.4% | |
| Sense strand siRNA: AUGGCACCUUGGGAUGAGCtt | [SEQ ID NO. 1367] |
| Antisense strand siRNA: GCUCAUCCCAAGGUGCCAUtt | [SEQ ID NO. 1368] |
| Target sequence 2: AAGACTGACTTCTGTCACCAC | [SEQ ID NO. 1369] |
| Position in gene sequence: 34 | |
| GC content: 47.6% | |
| Sense strand siRNA: GACUGACUUCUGUCACCACtt | [SEQ ID NO. 1370] |
| Antisense strand siRNA: GUGGUGACAGAAGUCAGUCtt | [SEQ ID NO. 1371] |
| Target sequence 3: AATTATTTCTATGGGATCTCT | [SEQ ID NO. 1372] |
| Position in gene sequence: 75 | |
| GC content: 28.6% | |
| Sense strand siRNA: UUAUUUCUAUGGGAUCUCUtt | [SEQ ID NO. 1373] |
| Antisense strand siRNA: AGAGAUCCCAUAGAAAUAAtt | [SEQ ID NO. 1374] |
| Target sequence 4: AATCTGAGAGACTGCAAGCCC | [SEQ ID NO. 1375] |
| Position in gene sequence: 102 | |
| GC content: 52.4% | |
| Sense strand siRNA: UCUGAGAGACUGCAAGCCCtt | [SEQ ID NO. 1376] |
| Antisense strand siRNA: GGGCUUGCAGUCUCUCAGAtt | [SEQ ID NO. 1377] |
| Target sequence 5: AAGCCCG121GAGAGGGCAGT | [SEQ ID NO. 1378] |
| Position in gene sequence: 117 | |
| GC content: 57.1% | |
| Sense strand siRNA: GCCCG121GAGAGGGCAGUtt | [SEQ ID NO. 1379] |
| Antisense strand siRNA: ACUGCCCUCUC121CGGGCtt | [SEQ ID NO. 1380] |
| Target sequence 6: AAGAGGCTGGTCTTCCTCCCC | [SEQ ID NO. 1381] |
| Position in gene sequence: 156 | |
| GC content: 61.9% | |
| Sense strand siRNA: GAGGCUGGUCUUCCUCCCCtt | [SEQ ID NO. 1382] |
| Antisense strand siRNA: GGGGAGGAAGACCAGCCUCtt | [SEQ ID NO. 1383] |
| Target sequence 7: AATTGTCTGGGGCTACTCCAC | [SEQ ID NO. 1384] |
| Position in gene sequence: 222 | |
| GC content: 52.4% | |
| Sense strand siRNA: UUGUCUGGGGCUACUCCACtt | [SEQ ID NO. 1385] |
| Antisense strand siRNA: GUGGAGUAGCCCCAGACAAtt | [SEQ ID NO. 1386] |
| Target sequence 8: AATCCTGACCCTTTTCTTGGG | [SEQ ID NO. 1387] |
| Position in gene sequence: 290 | |
| GC content: 47.6% | |
| Sense strand siRNA: UCCUGACCCUUUUCUUGGGtt | [SEQ ID NO. 1388] |
| Antisense strand siRNA: CCCAAGAAAAGGGUCAGGAtt | [SEQ ID NO. 1389] |
| Target sequence 9: AACTGCTTCATGATGAGGAAC | [SEQ ID NO. 1390] |
| Position in gene sequence: 339 | |
| GC content: 42.9% | |
| Sense strand siRNA: CUGCUUCAUGAUGAGGAACtt | [SEQ ID NO. 1391] |
| Antisense strand siRNA: GUUCCUCAUCAUGAAGCAGtt | [SEQ ID NO. 1392] |
| Target sequence 10: AACTACGAGATCATTCAGC36 | [SEQ ID NO. 1393] |
| Position in gene sequence: 357 | |
| GC content: 38.1% | |
| Sense strand siRNA: CUACGAGAUCAUUCAGC36tt | [SEQ ID NO. 1394] |
| Antisense strand siRNA: 63GCUGAAUGAUCUCGUAGtt | [SEQ ID NO. 1395] |
| Target sequence 11: AATCTCACCCAGAGGCTAACG | [SEQ ID NO. 1396] |
| Position in gene sequence: 396 | |
| GC content: 52.4% | |
| Sense strand siRNA: UCUCACCCAGAGGCUAACGtt | [SEQ ID NO. 1397] |
| Antisense strand siRNA: CGUUAGCCUCUGGGUGAGAtt | [SEQ ID NO. 1398] |
| Target sequence 12: AACGGTGGAGGCGGCCCAGTT | [SEQ ID NO. 1399] |
| Position in gene sequence: 413 | |
| GC content: 66.7% | |
| Sense strand siRNA: CGGUGGAGGCGGCCCAGUUtt | [SEQ ID NO. 1400] |
| Antisense strand siRNA: AACUGGGCCGCCUCCACCGtt | [SEQ ID NO. 1401] |

1. A method of reducing hair loss in a mammal comprising the step of: contacting a plurality of the mammal's hair cells with double-stranded ribonucleic acid, wherein one strand comprises at least a partial nucleic acid sequence of an androgen signal transduction pathway protein, and wherein the double-stranded ribonucleic acid interferes with the translation of mRNA of the androgen signal transduction protein.

2. A method of reducing hair loss in mammal comprising the step of: administering to the mammal a composition comprising a double-stranded ribonucleic acid, wherein one strand comprises at least a partial nucleic acid sequence of an androgen signal transduction pathway protein, and wherein the double-stranded ribonucleic acid interferes with the translation of mRNA of the androgen signal transduction protein; and a pharmaceutically acceptable carrier.

3. A method of treating alopecia comprising the step of: administering to a mammal a composition comprising a double-stranded ribonucleic acid, wherein one strand comprises at least a partial nucleic acid sequence of an androgen signal transduction pathway protein, and wherein the double-stranded ribonucleic acid interferes with the translation of mRNA of the androgen signal transduction protein; and a pharmaceutically acceptable carrier.

4. A method of inducing hair growth comprising the step of: increasing the duration of anagen of a hair cell of a mammal by contacting the hair cell with a double-stranded ribonucleic acid, wherein one strand comprises at least a partial nucleic acid sequence of an androgen signal transduction pathway protein.

5. A method of promoting hair growth or decreasing hair loss, said method comprising the steps of: a) providing an agent comprising at least one member selected from the group consisting of a small inhibitory ribonucleic acid comprising at least a partial nucleic acid sequence of an androgen signal transduction pathway protein and physiologically compatible salts thereof; and b) applying said agent to hair and scalp in an amount sufficient for said promoting of said hair growth or said decreasing of said hair loss.

6. The method of claim 1, further comprising the step of c) leaving said agent on said hair and scalp for at least 24 hours.

7. An inhibitory nucleic acid comprising: a double-stranded ribonucleic acid, wherein one strand comprises at least a partial nucleic acid sequence of an androgen signal transduction pathway protein, and wherein the double-stranded ribonucleic acid inhibits the translation of a mRNA encoding the nucleic acid sequence of the androgen signal transduction pathway protein.

8. A vector comprising: a double-stranded ribonucleic acid, wherein one strand comprises at least a partial nucleic acid sequence of all androgen signal transduction pathway protein, and wherein the double-stranded ribonucleic acid inhibits the translation of a mRNA encoding the nucleic acid sequence of the androgen signal transduction pathway protein.

9. A cell comprising: a double-stranded ribonucleic acid, wherein one strand comprises at least a partial nucleic acid sequence of an androgen signal transduction pathway protein, and wherein the double-stranded ribonucleic acid inhibits the translation of a mRNA encoding the nucleic acid sequence of the androgen signal transduction pathway protein.

10. Claim 1, wherein the androgen signal transduction pathway protein is selected from the group consisting of isozymes I and II of 5-a reductase, the androgen receptor, aromatase, 3-a-hydroxysteroiddehydrogenase, 3-(3-hydroxysteroiddehydrogenase, 3-β-hydroxysteroiddehydrogenase-4-5-isomerase, 17-β-hydroxysteroidoxidoreductase, and steroid sulfatase.

11. Claim 1, wherein the partial nucleic acid sequence is selected from SEQ. ID Nos 1-1400 in Tables 1-9.

12. Claim 1, further comprising the step of: administering a second therapeutic agent.

13. The method of claim 12, wherein the second therapeutic agent is selected from the group consisting of 17β-N-t butylcarbamoyl-4-aza-5a-androst-1-en-3-one, 6-amino-1,2-dihydro-1-hydroxy-2-imino-4-piperodinopyrimidine, a combination thereof, and a prodrug thereof.

14. A method of treating hair loss in a mammal comprising reducing the levels of dihydroxy testosterone in the mammal by contacting the mammal with an effective amount of inhibitory nucleic acid specific for 5-a-reductase, wherein the effective amount of inhibitory nucleic acid specific for 5-a-reductase inhibits the expression of 5-a reductase protein.

15. A method of reducing testosterone metabolites in a cell comprising contacting the cell with an amount of inhibitory nucleic acid specific for 5-a-reductase sufficient to reduce the levels of 5-a reductase protein in the cell.

16. The method of claim 15, wherein the testosterone metabolites comprise dihydroxy testosterone.

17. The method of claim 15, wherein the cell is a hair cell.

18. The method of claim 15, wherein the inhibitory nucleic acid comprises a nucelotide sequence selected from SEQ. ID. Nos 1-91.


SEQUENCE LISTING (1403 sequences)

SEQ ID NO: 1 - DNA, 636 bp, Homo sapiens
atggcaacggcgacgggggtggcggaggagcgcctgctggccgcgctcgcctacctgcagtgcgccgtgggctgcgcggtcttcccgcgtctccgcagcgcgcccaactgcatcctcctggccatgttcctcgtccactacgggcatcggtgcttaatttacccatttctgatgcgaggaggaaagcctatgccactgttggcgtgtacaatggcgattatgttctgtacctgtaacggctatttgcaaagcagatacttgagccattgtgcagtgtatgctgatgactgggtaacagatccccgttttctaataggttttggcttgtggttaacaggcatgttgataaacatccattcagatcatatcctaaggaatctcagaaaaccaggagatactggatacaaaataccaaggggaggcttatttgaatacgtaactgcagccaactattttggagaaatcatggagtggtgtggctatgccctggccagctggtctgtccaaggcgcggcttttgctttcttcacgttttgttttttatctggtagagcaaaagagcatcatgagtggtacctccggaaatttgaagagtatccaaagttcagaaaaattataattccatttttgttttaa

SEQ ID NO: 2 - DNA, 21 bp, Homo sapiens
aacggcgacgggggtggcgga

SEQ ID NO: 3 - DNA, 21 bp, Homo sapiens
cggcgacggggguggcggatt

SEQ ID NO: 4 - DNA, 21 bp, Homo sapiens
uccgccacccccgucgccgtt

SEQ ID NO: 5 - DNA, 18 bp, Homo sapiens
aactgcatcctcctggcc

SEQ ID NO: 6 - DNA, 18 bp, Homo sapiens
cugcauccuccuggcctt

SEQ ID NO: 7 - DNA, 18 bp, Homo sapiens
ggccaggaggaugcagtt

SEQ ID NO: 8 - DNA, 21 bp, Homo sapiens
aatttacccatttctgatgcg

SEQ ID NO: 9 - DNA, 21 bp, Homo sapiens
uuuacccauuucugaugcgtt

SEQ ID NO: 10 - DNA, 21 bp, Homo sapiens
cgcaucagaaauggguaaatt

SEQ ID NO: 11 - DNA, 21 bp, Homo sapiens
aaagcctatgccactgttggc

SEQ ID NO: 12 - DNA, 21 bp, Homo sapiens
agccuaugccacuguuggctt

SEQ ID NO: 13 - DNA, 21 bp, Homo sapiens
gccaacaguggcauaggcutt

SEQ ID NO: 14 - DNA, 21 bp, Homo sapiens
aatggcgattatgttctgtac

SEQ ID NO: 15 - DNA, 21 bp, Homo sapiens
uggcgauuauguucuguactt

SEQ ID NO: 16 - DNA, 21 bp, Homo sapiens
guacagaacauaaucgccatt

SEQ ID NO: 17 - DNA, 18 bp, Homo sapiens
aacggctatttgcaaagc

SEQ ID NO: 18 - DNA, 18 bp, Homo sapiens
cggcuauuugcaaagctt

SEQ ID NO: 19 - DNA, 18 bp, Homo sapiens
gcuuugcaaauagccgtt

SEQ ID NO: 20 - DNA, 21 bp, Homo sapiens
aaagcagatacttgagccatt

SEQ ID NO: 21 - DNA, 21 bp, Homo sapiens
agcagauacuugagccauutt

SEQ ID NO: 22 - DNA, 21 bp, Homo sapiens
aauggcucaaguaucugcutt

SEQ ID NO: 23 - DNA, 18 bp, Homo sapiens
aacagatccccgttttct

SEQ ID NO: 24 - DNA, 18 bp, Homo sapiens
cagauccccguuuucutt

SEQ ID NO: 25 - DNA, 18 bp, Homo sapiens
agaaaacggggaucugtt

SEQ ID NO: 26 - DNA, 21 bp, Homo sapiens
aataggttttggcttgtggtt

SEQ ID NO: 27 - DNA, 21 bp, Homo sapiens
uagguuuuggcuugugguutt

SEQ ID NO: 28 - DNA, 21 bp, Homo sapiens
aaccacaagccaaaaccuatt

SEQ ID NO: 29 - DNA, 21 bp, Homo sapiens
aacaggcatgttgataaacat

SEQ ID NO: 30 - DNA, 21 bp, Homo sapiens
caggcauguugauaaacautt

SEQ ID NO: 31 - DNA, 21 bp, Homo sapiens
auguuuaucaacaugccugtt

SEQ ID NO: 32 - DNA, 18 bp, Homo sapiens
aaacatccattcagatca

SEQ ID NO: 33 - DNA, 18 bp, Homo sapiens
acauccauucagaucatt

SEQ ID NO: 34 - DNA, 18 bp, Homo sapiens
ugaucugaauggaugutt

SEQ ID NO: 35 - DNA, 21 bp, Homo sapiens
aaggaatctcagaaaaccagg

SEQ ID NO: 36 - DNA, 21 bp, Homo sapiens
ggaaucucagaaaaccaggtt

SEQ ID NO: 37 - DNA, 21 bp, Homo sapiens
ccugguuuucugagauucctt

SEQ ID NO: 38 - DNA, 21 bp, Homo sapiens
aatctcagaaaaccaggagat

SEQ ID NO: 39 - DNA, 21 bp, Homo sapiens
ucucagaaaaccaggagautt

SEQ ID NO: 40 - DNA, 21 bp, Homo sapiens
aucuccugguuuucugagatt

SEQ ID NO: 41 - DNA, 21 bp, Homo sapiens
aaaaccaggagatactggata

SEQ ID NO: 42 - DNA, 21 bp, Homo sapiens
aaccaggagauacuggauatt

SEQ ID NO: 43 - DNA, 21 bp, Homo sapiens
uauccaguaucuccugguutt

SEQ ID NO: 44 - DNA, 21 bp, Homo sapiens
aaccaggagatactggataca

SEQ ID NO: 45 - DNA, 21 bp, Homo sapiens
ccaggagauacuggauacatt

SEQ ID NO: 46 - DNA, 21 bp, Homo sapiens
uguauccaguaucuccuggtt

SEQ ID NO: 47 - DNA, 18 bp, Homo sapiens
aaaataccaaggggaggc

SEQ ID NO: 48 - DNA, 18 bp, Homo sapiens
aauaccaaggggaggctt

SEQ ID NO: 49 - DNA, 18 bp, Homo sapiens
gccuccccuugguauutt

SEQ ID NO: 50 - DNA, 18 bp, Homo sapiens
aataccaaggggaggctt

SEQ ID NO: 51 - DNA, 18 bp, Homo sapiens
uaccaaggggaggcuutt

SEQ ID NO: 52 - DNA, 18 bp, Homo sapiens
aagccuccccuugguatt

SEQ ID NO: 53 - DNA, 18 bp, Homo sapiens
aaggggaggcttatttga

SEQ ID NO: 54 - DNA, 18 bp, Homo sapiens
ggggaggcuuauuugatt

SEQ ID NO: 55 - DNA, 18 bp, Homo sapiens
ucaaauaagccucccctt

SEQ ID NO: 56 - DNA, 21 bp, Homo sapiens
aatacgtaactgcagccaact

SEQ ID NO: 57 - DNA, 21 bp, Homo sapiens
uacguaacugcagccaacutt

SEQ ID NO: 58 - DNA, 21 bp, Homo sapiens
aguuggcugcaguuacguatt

SEQ ID NO: 59 - DNA, 21 bp, Homo sapiens
aactgcagccaactattttgg

SEQ ID NO: 60 - DNA, 21 bp, Homo sapiens
cugcagccaacuauuuuggtt

SEQ ID NO: 61 - DNA, 21 bp, Homo sapiens
ccaaaauaguuggcugcagtt

SEQ ID NO: 62 - DNA, 21 bp, Homo sapiens
aactattttggagaaatcatg

SEQ ID NO: 63 - DNA, 21 bp, Homo sapiens
cuauuuuggagaaaucaugtt

SEQ ID NO: 64 - DNA, 21 bp, Homo sapiens
caugauuucuccaaaauagtt

SEQ ID NO: 65 - DNA, 20 bp, Homo sapiens
aaatcatggagtggtgtggc

SEQ ID NO: 66 - DNA, 20 bp, Homo sapiens
aucauggagugguguggctt

SEQ ID NO: 67 - DNA, 20 bp, Homo sapiens
gccacaccacuccaugautt

SEQ ID NO: 68 - DNA, 21 bp, Homo sapiens
aaggcgcggcttttgctttct

SEQ ID NO: 69 - DNA, 21 bp, Homo sapiens
ggcgcggcuuuugcuuucutt

SEQ ID NO: 70 - DNA, 21 bp, Homo sapiens
agaaagcaaaagccgcgcctt

SEQ ID NO: 71 - DNA, 21 bp, Homo sapiens
aaaagagcatcatgagtggta

SEQ ID NO: 72 - DNA, 21 bp, Homo sapiens
aagagcaucaugagugguatt

SEQ ID NO: 73 - DNA, 21 bp, Homo sapiens
uaccacucaugaugcucuutt

SEQ ID NO: 74 - DNA, 21 bp, Homo sapiens
aagagcatcatgagtggtacc

SEQ ID NO: 75 - DNA, 21 bp, Homo sapiens
gagcaucaugagugguacctt

SEQ ID NO: 76 - DNA, 21 bp, Homo sapiens
gguaccacucaugaugcuctt

SEQ ID NO: 77 - DNA, 18 bp, Homo sapiens
aaatttgaagagtatcca

SEQ ID NO: 78 - DNA, 18 bp, Homo sapiens
auuugaagaguauccatt

SEQ ID NO: 79 - DNA, 18 bp, Homo sapiens
uggauacucuucaaautt

SEQ ID NO: 80 - DNA, 18 bp, Homo sapiens
aagagtatccaaagttca

SEQ ID NO: 81 - DNA, 18 bp, Homo sapiens
gaguauccaaaguucatt

SEQ ID NO: 82 - DNA, 18 bp, Homo sapiens
ugaacuuuggauacuctt

SEQ ID NO: 83 - DNA, 21 bp, Homo sapiens
aagttcagaaaaattataatt

SEQ ID NO: 84 - DNA, 21 bp, Homo sapiens
guucagaaaaauuauaauutt

SEQ ID NO: 85 - DNA, 21 bp, Homo sapiens
aauuauaauuuuucugaactt

SEQ ID NO: 86 - DNA, 21 bp, Homo sapiens
aaaaattataattccattttt

SEQ ID NO: 87 - DNA, 21 bp, Homo sapiens
aaauuauaauuccauuuuutt

SEQ ID NO: 88 - DNA, 21 bp, Homo sapiens
aaaaauggaauuauaauuutt

SEQ ID NO: 89 - DNA, 21 bp, Homo sapiens
aaattataattccatttttgt

SEQ ID NO: 90 - DNA, 21 bp, Homo sapiens
auuauaauuccauuuuugutt

SEQ ID NO: 91 - DNA, 21 bp, Homo sapiens
acaaaaauggaauuauaautt

SEQ ID NO: 92 - DNA, 765 bp, Homo sapiens
atgcaggttcagtgccagcagagcccagtgctggcaggcagcgccactttggtcgcccttggggcactggtcttgtacgtcgcgaagccctccggctacgggaagcacacggagagcctgaagcccgcggctacccgcctgccagcccgcgcagcctggttcctgcaggagctgccctccttcgcggtgcccgcggggatcctcgctcggcagcccctctccctcttcgggccacctgggacggtgcttctgggcctcttctgcgtacattacttccacaggacatttgtgtactcactgctcaatcgagggaggccttatccagctgtactcattttccgaggcattgccttctgcgctggaaatggattccttcaaagctactatctgatttactgtgctgaataccctgatgggtggtacacagacatacggttttgcttgggtgtcttcttatttattttgggaatgggagtcaacatccatagtgactatatattgcgccagctcaggaagcctggagaaatcacctacaggattccaaaaggtggcttgtttacgtatgtttctggagccaatttccttggtgagatcattgaatggatcggctatgcgctggccacttggtccctcccagcacttgcatttgcatttttctcagtttgtttccttgggctgcgagcttttcaccaccataggttctacctcaagatgtttgaggactaccccaaatctcggaaagcccttattccattcatcttttaa

SEQ ID NO: 93 - DNA, 21 bp, Homo sapiens
aagccctccggctacgggaag

SEQ ID NO: 94 - DNA, 21 bp, Homo sapiens
gcccuccggcuacgggaagtt

SEQ ID NO: 95 - DNA, 21 bp, Homo sapiens
cuucccguagccggagggctt

SEQ ID NO: 96 - DNA, 18 bp, Homo sapiens
aagcacacggagagcctg

SEQ ID NO: 97 - DNA, 18 bp, Homo sapiens
gcacacggagagccugtt

SEQ ID NO: 98 - DNA, 18 bp, Homo sapiens
caggcucuccgugugctt

SEQ ID NO: 99 - DNA, 21 bp, Homo sapiens
aagcccgcggctacccgcctg

SEQ ID NO: 100 - DNA, 21 bp, Homo sapiens
gcccgcggcuacccgccugtt

SEQ ID NO: 101 - DNA, 21 bp, Homo sapiens
caggcggguagccgcgggctt

SEQ ID NO: 102 - DNA, 21 bp, Homo sapiens
aatcgagggaggccttatcca

SEQ ID NO: 103 - DNA, 21 bp, Homo sapiens
ucgagggaggccuuauccatt

SEQ ID NO: 104 - DNA, 21 bp, Homo sapiens
uggauaaggccucccucgatt

SEQ ID NO: 105 - DNA, 21 bp, Homo sapiens
aaatggattccttcaaagcta

SEQ ID NO: 106 - DNA, 21 bp, Homo sapiens
auggauuccuucaaagcuatt

SEQ ID NO: 107 - DNA, 21 bp, Homo sapiens
uagcuuugaaggaauccautt

SEQ ID NO: 108 - DNA, 21 bp, Homo sapiens
aaagctactatctgatttact

SEQ ID NO: 109 - DNA, 21 bp, Homo sapiens
agcuacuaucugauuuacutt

SEQ ID NO: 110 - DNA, 21 bp, Homo sapiens
aguaaaucagauaguagcutt

SEQ ID NO: 111 - DNA, 18 bp, Homo sapiens
aataccctgatgggtggt

SEQ ID NO: 112 - DNA, 18 bp, Homo sapiens
uacccugauggguggutt

SEQ ID NO: 113 - DNA, 18 bp, Homo sapiens
accacccaucaggguatt

SEQ ID NO: 114 - DNA, 18 bp, Homo sapiens
aatgggagtcaacatcca

SEQ ID NO: 115 - DNA, 18 bp, Homo sapiens
ugggagucaacauccatt

SEQ ID NO: 116 - DNA, 18 bp, Homo sapiens
uggauguugacucccatt

SEQ ID NO: 117 - DNA, 18 bp, Homo sapiens
aacatccatagtgactat

SEQ ID NO: 118 - DNA, 18 bp, Homo sapiens
cauccauagugacuautt

SEQ ID NO: 119 - DNA, 18 bp, Homo sapiens
auagucacuauggaugtt

SEQ ID NO: 120 - DNA, 21 bp, Homo sapiens
aagcctggagaaatcacctac

SEQ ID NO: 121 - DNA, 21 bp, Homo sapiens
gccuggagaaaucaccuactt

SEQ ID NO: 122 - DNA, 21 bp, Homo sapiens
guaggugauuucuccaggctt

SEQ ID NO: 123 - DNA, 18 bp, Homo sapiens
aaatcacctacaggattc

SEQ ID NO: 124 - DNA, 18 bp, Homo sapiens
aucaccuacaggauuctt

SEQ ID NO: 125 - DNA, 18 bp, Homo sapiens
gaauccuguaggugautt

SEQ ID NO: 126 - DNA, 21 bp, Homo sapiens
aaaaggtggcttgtttacgta

SEQ ID NO: 127 - DNA, 21 bp, Homo sapiens
aagguggcuuguuuacguatt

SEQ ID NO: 128 - DNA, 21 bp, Homo sapiens
uacguaaacaagccaccuutt

SEQ ID NO: 129 - DNA, 21 bp, Homo sapiens
aaggtggcttgtttacgtatg

SEQ ID NO: 130 - DNA, 21 bp, Homo sapiens
gguggcuuguuuacguaugtt

SEQ ID NO: 131 - DNA, 21 bp, Homo sapiens
cauacguaaacaagccacctt

SEQ ID NO: 132 - DNA, 21 bp, Homo sapiens
aatttccttggtgagatcatt

SEQ ID NO: 133 - DNA, 21 bp, Homo sapiens
uuuccuuggugagaucauutt

SEQ ID NO: 134 - DNA, 21 bp, Homo sapiens
aaugaucucaccaaggaaatt

SEQ ID NO: 135 - DNA, 18 bp, Homo sapiens
aatggatcggctatgcgc

SEQ ID NO: 136 - DNA, 18 bp, Homo sapiens
uggaucggcuaugcgctt

SEQ ID NO: 137 - DNA, 18 bp, Homo sapiens
gcgcauagccgauccatt

SEQ ID NO: 138 - DNA, 18 bp, Homo sapiens
aagatgtttgaggactac

SEQ ID NO: 139 - DNA, 18 bp, Homo sapiens
gauguuugaggacuactt

SEQ ID NO: 140 - DNA, 18 bp, Homo sapiens
guaguccucaaacauctt

SEQ ID NO: 141 - DNA, 21 bp, Homo sapiens
aaatctcggaaagcccttatt

SEQ ID NO: 142 - DNA, 21 bp, Homo sapiens
aucucggaaagcccuuauutt

SEQ ID NO: 143 - DNA, 21 bp, Homo sapiens
aauaagggcuuuccgagautt

SEQ ID NO: 144 - DNA, 21 bp, Homo sapiens
aaagcccttattccattcatc

SEQ ID NO: 145 - DNA, 21 bp, Homo sapiens
agcccuuauuccauucauctt

SEQ ID NO: 146 - DNA, 21 bp, Homo sapiens
gaugaauggaauaagggcutt

SEQ ID NO: 147 - DNA, 2763 bp, Homo sapiens
atggaagtgcagttagggctgggaagggtctaccctcggccgccgtccaagacctaccgaggagctttccagaatctgttccagagcgtgcgcgaagtgatccagaacccgggccccaggcacccagaggccgcgagcgcagcacctcccggcgccagtttgctgctgctgcagcagcagcagcagcagcagcagcagcagcagcagcagcagcagcagcagcagcagcagcagcagcaagagactagccccaggcagcagcagcagcagcagggtgaggatggttctccccaagcccatcgtagaggccccacaggctacctggtcctggatgaggaacagcaaccttcacagccgcagtcggccctggagtgccaccccgagagaggttgcgtcccagagcctggagccgccgtggccgccagcaaggggctgccgcagcagctgccagcacctccggacgaggatgactcagctgccccatccacgttgtccctgctgggccccactttccccggcttaagcagctgctccgctgaccttaaagacatcctgagcgaggccagcaccatgcaactccttcagcaacagcagcaggaagcagtatccgaaggcagcagcagcgggagagcgagggaggcctcgggggctcccacttcctccaaggacaattacttagggggcacttcgaccatttctgacaacgccaaggagttgtgtaaggcagtgtcggtgtccatgggcctgggtgtggaggcgttggagcatctgagtccaggggaacagcttcggggggattgcatgtacgccccacttttgggagttccacccgctgtgcgtcccactccttgtgccccattggccgaatgcaaaggttctctgctagacgacagcgcaggcaagagcactgaagatactgctgagtattcccctttcaagggaggttacaccaaagggctagaaggcgagagcctaggctgctctggcagcgctgcagcagggagctccgggacacttgaactgccgtctaccctgtctctctacaagtccggagcactggacgaggcagctgcgtaccagagtcgcgactactacaactttccactggctctggccggaccgccgccccctccgccgcctccccatccccacgctcgcatcaagctggagaacccgctggactacggcagcgcctgggcggctgcggcggcgcagtgccgctatggggacctggcgagcctgcatggcgcgggtgcagcgggacccggttctgggtcaccctcagccgccgcttcctcatcctggcacactctcttcacagccgaagaaggccagttgtatggaccgtgtggtggtggtgggggtggtggcggcggcggcggcggcggcggcggcggcggcggcggcggcggcggcggcgaggcgggagctgtagccccctacggctacactcggccccctcaggggctggcgggccaggaaagcgacttcaccgcacctgatgtgtggtaccctggcggcatggtgagcagagtgccctatcccagtcccacttgtgtcaaaagcgaaatgggcccctggatggatagctactccggaccttacggggacatgcgtttggagactgccagggaccatgttttgcccattgactattactttccaccccagaagacctgcctgatctgtggagatgaagcttctgggtgtcactatggagctctcacatgtggaagctgcaaggtcttcttcaaaagagccgctgaagggaaacagaagtacctgtgcgccagcagaaatgattgcactattgataaattccgaaggaaaaattgtccatcttgtcgtcttcggaaatgttatgaagcagggatgactctgggagcccggaagctgaagaaacttggtaatctgaaactacaggaggaaggagaggcttccagcaccaccagccccactgaggagacaacccagaagctgacagtgtcacacattgaaggctatgaatgtcagcccatctttctgaatgtcctggaagccattgagccaggtgtagtgtgtgctggacacgacaacaaccagcccgactcctttgcagccttgctctctagcctcaatgaactgggagagagacagcttgtacacgtggtcaagtgggccaaggccttgcctggcttccgcaacttacacgtggacgaccagatggctgtcattcagtactcctggatggggctcatggtgtttgccatgggctggcgatccttcaccaatgtcaactccaggatgctctacttcgcccctgatctggttttcaatgagtaccgcatgcacaagtcccggatgtacagccagtgtgtccgaatgaggcacctctctcaagagtttggatggctccaaatcaccccccaggaattcctgtgcatgaaagcactgctactcttcagcattattccagtggatgggctgaaaaatcaaaaattctttgatgaacttcgaatgaactacatcaaggaactcgatcgtatcattgcatgcaaaagaaaaaatcccacatcctgctcaagacgcttctaccagctcaccaagctcctggactccgtgcagcctattgcgagagagctgcatcagttcacttttgacctgctaatcaagtcacacatggtgagcgtggactttccggaaatgatggcagagatcatctctgtgcaagtgcccaagatcctttctgggaaagtcaagcccatctatttccacacccagtga

SEQ ID NO: 148 - DNA, 21 bp, Homo sapiens
aagtgcagttagggctgggaa

SEQ ID NO: 149 - DNA, 21 bp, Homo sapiens
gugcaguuagggcugggaatt

SEQ ID NO: 150 - DNA, 21 bp, Homo sapiens
uucccagcccuaacugcactt

SEQ ID NO: 151 - DNA, 21 bp, Homo sapiens
aagggtctaccctcggccgcc

SEQ ID NO: 152 - DNA, 21 bp, Homo sapiens
gggucuacccucggccgcctt

SEQ ID NO: 153 - DNA, 21 bp, Homo sapiens
ggcggccgaggguagaccctt

SEQ ID NO: 154 - DNA, 19 bp, Homo sapiens
aagacctaccgaggagctt

SEQ ID NO: 155 - DNA, 19 bp, Homo sapiens
gaccuaccgaggagcuutt

SEQ ID NO: 156 - DNA, 19 bp, Homo sapiens
aagcuccucgguagguctt

SEQ ID NO: 157 - DNA, 21 bp, Homo sapiens
aatctgttccagagcgtgcgc

SEQ ID NO: 158 - DNA, 21 bp, Homo sapiens
ucuguuccagagcgugcgctt

SEQ ID NO: 159 - DNA, 21 bp, Homo sapiens
gcgcacgcucuggaacagatt

SEQ ID NO: 160 - DNA, 21 bp, Homo sapiens
aagtgatccagaacccgggcc

SEQ ID NO: 161 - DNA, 21 bp, Homo sapiens
gugauccagaacccgggcctt

SEQ ID NO: 162 - DNA, 21 bp, Homo sapiens
ggcccggguucuggaucactt

SEQ ID NO: 163 - DNA, 18 bp, Homo sapiens
aacccgggccccaggcac

SEQ ID NO: 164 - DNA, 18 bp, Homo sapiens
cccgggccccaggcactt

SEQ ID NO: 165 - DNA, 18 bp, Homo sapiens
gugccuggggcccgggtt

SEQ ID NO: 166 - DNA, 18 bp, Homo sapiens
aagagactagccccaggc

SEQ ID NO: 167 - DNA, 18 bp, Homo sapiens
gagacuagccccaggctt

SEQ ID NO: 168 - DNA, 18 bp, Homo sapiens
gccuggggcuagucuctt

SEQ ID NO: 169 - DNA, 18 bp, Homo sapiens
aagcccatcgtagaggcc

SEQ ID NO: 170 - DNA, 18 bp, Homo sapiens
gcccaucguagaggcctt

SEQ ID NO: 171 - DNA, 18 bp, Homo sapiens
ggccucuacgaugggctt

SEQ ID NO: 172 - DNA, 21 bp, Homo sapiens
aacagcaaccttcacagccgc

SEQ ID NO: 173 - DNA, 21 bp, Homo sapiens
cagcaaccuucacagccgctt

SEQ ID NO: 174 - DNA, 21 bp, Homo sapiens
gcggcugugaagguugcugtt

SEQ ID NO: 175 - DNA, 18 bp, Homo sapiens
aaccttcacagccgcagt

SEQ ID NO: 176 - DNA, 18 bp, Homo sapiens
ccuucacagccgcagutt

SEQ ID NO: 177 - DNA, 18 bp, Homo sapiens
acugcggcugugaaggtt

SEQ ID NO: 178 - DNA, 21 bp, Homo sapiens
aaggggctgccgcagcagctg

SEQ ID NO: 179 - DNA, 21 bp, Homo sapiens
ggggcugccgcagcagcugtt

SEQ ID NO: 180 - DNA, 21 bp, Homo sapiens
cagcugcugcggcagcccctt

SEQ ID NO: 181 - DNA, 19 bp, Homo sapiens
aagcagctgctccgctgac

SEQ ID NO: 182 - DNA, 19 bp, Homo sapiens
gcagcugcuccgcugactt

SEQ ID NO: 183 - DNA, 19 bp, Homo sapiens
gucagcggagcagcugctt

SEQ ID NO: 184 - DNA, 21 bp, Homo sapiens
aaagacatcctgagcgaggcc

SEQ ID NO: 185 - DNA, 21 bp, Homo sapiens
agacauccugagcgaggcctt

SEQ ID NO: 186 - DNA, 21 bp, Homo sapiens
ggccucgcucaggaugucutt

SEQ ID NO: 187 - DNA, 21 bp, Homo sapiens
aactccttcagcaacagcagc

SEQ ID NO: 188 - DNA, 21 bp, Homo sapiens
cuccuucagcaacagcagctt

SEQ ID NO: 189 - DNA, 21 bp, Homo sapiens
gcugcuguugcugaaggagtt

SEQ ID NO: 190 - DNA, 18 bp, Homo sapiens
aacagcagcaggaagcag

SEQ ID NO: 191 - DNA, 18 bp, Homo sapiens
cagcagcaggaagcagtt

SEQ ID NO: 192 - DNA, 18 bp, Homo sapiens
cugcuuccugcugcugtt

SEQ ID NO: 193 - DNA, 18 bp, Homo sapiens
aagcagtatccgaaggca

SEQ ID NO: 194 - DNA, 18 bp, Homo sapiens
gcaguauccgaaggcatt

SEQ ID NO: 195 - DNA, 18 bp, Homo sapiens
ugccuucggauacugctt

SEQ ID NO: 196 - DNA, 21 bp, Homo sapiens
aaggcagcagcagcgggagag

SEQ ID NO: 197 - DNA, 21 bp, Homo sapiens
ggcagcagcagcgggagagtt

SEQ ID NO: 198 - DNA, 21 bp, Homo sapiens
cucucccgcugcugcugcctt

SEQ ID NO: 199 - DNA, 21 bp, Homo sapiens
aaggacaattacttagggggc

SEQ ID NO: 200 - DNA, 21 bp, Homo sapiens
ggacaauuacuuagggggctt

SEQ ID NO: 201 - DNA, 21 bp, Homo sapiens
gcccccuaaguaauugucctt

SEQ ID NO: 202 - DNA, 21 bp, Homo sapiens
aattacttagggggcacttcg

SEQ ID NO: 203 - DNA, 21 bp, Homo sapiens
uuacuuagggggcacuucgtt

SEQ ID NO: 204 - DNA, 21 bp, Homo sapiens
cgaagugcccccuaaguaatt

SEQ ID NO: 205 - DNA, 18 bp, Homo sapiens
aacgccaaggagttgtgt

SEQ ID NO: 206 - DNA, 18 bp, Homo sapiens
cgccaaggaguugugutt

SEQ ID NO: 207 - DNA, 18 bp, Homo sapiens
acacaacuccuuggcgtt

SEQ ID NO: 208 - DNA, 18 bp, Homo sapiens
aaggagttgtgtaaggca

SEQ ID NO: 209 - DNA, 18 bp, Homo sapiens
ggaguuguguaaggcatt

SEQ ID NO: 210 - DNA, 18 bp, Homo sapiens
ugccuuacacaacucctt

SEQ ID NO: 211 - DNA, 21 bp, Homo sapiens
aaggcagtgtcggtgtccatg

SEQ ID NO: 212 - DNA, 21 bp, Homo sapiens
ggcagugucgguguccaugtt

SEQ ID NO: 213 - DNA, 21 bp, Homo sapiens
cauggacaccgacacugcctt

SEQ ID NO: 214 - DNA, 21 bp, Homo sapiens
aacagcttcggggggattgca

SEQ ID NO: 215 - DNA, 21 bp, Homo sapiens
cagcuucggggggauugcatt

SEQ ID NO: 216 - DNA, 21 bp, Homo sapiens
ugcaauccccccgaagcugtt

SEQ ID NO: 217 - DNA, 21 bp, Homo sapiens
aatgcaaaggttctctgctag

SEQ ID NO: 218 - DNA, 21 bp, Homo sapiens
ugcaaagguucucugcuagtt

SEQ ID NO: 219 - DNA, 21 bp, Homo sapiens
cuagcagagaaccuuugcatt

SEQ ID NO: 220 - DNA, 21 bp, Homo sapiens
aaaggttctctgctagacgac

SEQ ID NO: 221 - DNA, 21 bp, Homo sapiens
agguucucugcuagacgactt

SEQ ID NO: 222 - DNA, 21 bp, Homo sapiens
gucgucuagcagagaaccutt

SEQ ID NO: 223 - DNA, 21 bp, Homo sapiens
aagagcactgaagatactgct

SEQ ID NO: 224 - DNA, 21 bp, Homo sapiens
gagcacugaagauacugcutt

SEQ ID NO: 225 - DNA, 21 bp, Homo sapiens
agcaguaucuucagugcuctt

SEQ ID NO: 226 - DNA, 21 bp, Homo sapiens
aagatactgctgagtattccc

SEQ ID NO: 227 - DNA, 21 bp, Homo sapiens
gauacugcugaguauuccctt

SEQ ID NO: 228 - DNA, 21 bp, Homo sapiens
gggaauacucagcaguauctt

SEQ ID NO: 229 - DNA, 21 bp, Homo sapiens
aagggaggttacaccaaaggg

SEQ ID NO: 230 - DNA, 21 bp, Homo sapiens
gggagguuacaccaaagggtt

SEQ ID NO: 231 - DNA, 21 bp, Homo sapiens
cccuuugguguaaccuccctt

SEQ ID NO: 232 - DNA, 18 bp, Homo sapiens
aaagggctagaaggcgag

SEQ ID NO: 233 - DNA, 18 bp, Homo sapiens
agggcuagaaggcgagtt

SEQ ID NO: 234 - DNA, 18 bp, Homo sapiens
cucgccuucuagcccutt

SEQ ID NO: 235 - DNA, 21 bp, Homo sapiens
aaggcgagagcctaggctgct

SEQ ID NO: 236 - DNA, 21 bp, Homo sapiens
ggcgagagccuaggcugcutt

SEQ ID NO: 237 - DNA, 21 bp, Homo sapiens
agcagccuaggcucucgcctt

SEQ ID NO: 238 - DNA, 17 bp, Homo sapiens
aactgccgtctaccctg

SEQ ID NO: 239 - DNA, 17 bp, Homo sapiens
cugccgucuacccugtt

SEQ ID NO: 240 - DNA, 17 bp, Homo sapiens
caggguagacggcagtt

SEQ ID NO: 241 - DNA, 21 bp, Homo sapiens
aagtccggagcactggacgag

SEQ ID NO: 242 - DNA, 21 bp, Homo sapiens
guccggagcacuggacgagtt

SEQ ID NO: 243 - DNA, 21 bp, Homo sapiens
cucguccagugcuccggactt

SEQ ID NO: 244 - DNA, 21 bp, Homo sapiens
aactttccactggctctggcc

SEQ ID NO: 245 - DNA, 21 bp, Homo sapiens
cuuuccacuggcucuggcctt

SEQ ID NO: 246 - DNA, 21 bp, Homo sapiens
ggccagagccaguggaaagtt

SEQ ID NO: 247 - DNA, 21 bp, Homo sapiens
aagctggagaacccgctggac

SEQ ID NO: 248 - DNA, 21 bp, Homo sapiens
gcuggagaacccgcuggactt

SEQ ID NO: 249 - DNA, 21 bp, Homo sapiens
guccagcggguucuccagctt

SEQ ID NO: 250 - DNA, 21 bp, Homo sapiens
aacccgctggactacggcagc

SEQ ID NO: 251 - DNA, 21 bp, Homo sapiens
cccgcuggacuacggcagctt

SEQ ID NO: 252 - DNA, 21 bp, Homo sapiens
gcugccguaguccagcgggtt

SEQ ID NO: 253 - DNA, 21 bp, Homo sapiens
aagaaggccagttgtatggac

SEQ ID NO: 254 - DNA, 21 bp, Homo sapiens
gaaggccaguuguauggactt

SEQ ID NO: 255 - DNA, 21 bp, Homo sapiens
guccauacaacuggccuuctt

SEQ ID NO: 256 - DNA, 21 bp, Homo sapiens
aaggccagttgtatggaccgt

SEQ ID NO: 257 - DNA, 21 bp, Homo sapiens
ggccaguuguauggaccgutt

SEQ ID NO: 258 - DNA, 21 bp, Homo sapiens
acgguccauacaacuggcctt

SEQ ID NO: 259 - DNA, 20 bp, Homo sapiens
aaagcgacttcaccgcacct

SEQ ID NO: 260 - DNA, 20 bp, Homo sapiens
agcgacuucaccgcaccutt

SEQ ID NO: 261 - DNA, 20 bp, Homo sapiens
aggugcggugaagucgcutt

SEQ ID NO: 262 - DNA, 21 bp, Homo sapiens
aaaagcgaaatgggcccctgg

SEQ ID NO: 263 - DNA, 21 bp, Homo sapiens
aagcgaaaugggccccuggtt

SEQ ID NO: 264 - DNA, 21 bp, Homo sapiens
ccaggggcccauuucgcuutt

SEQ ID NO: 265 - DNA, 21 bp, Homo sapiens
aagcgaaatgggcccctggat

SEQ ID NO: 266 - DNA, 21 bp, Homo sapiens
gcgaaaugggccccuggautt

SEQ ID NO: 267 - DNA, 21 bp, Homo sapiens
auccaggggcccauuucgctt

SEQ ID NO: 268 - DNA, 21 bp, Homo sapiens
aaatgggcccctggatggata

SEQ ID NO: 269 - DNA, 21 bp, Homo sapiens
augggccccuggauggauatt

SEQ ID NO: 270 - DNA, 21 bp, Homo sapiens
uauccauccaggggcccautt

SEQ ID NO: 271 - DNA, 17 bp, Homo sapiens
aagacctgcctgatctg

SEQ ID NO: 272 - DNA, 17 bp, Homo sapiens
gaccugccugaucugtt

SEQ ID NO: 273 - DNA, 17 bp, Homo sapiens
cagaucaggcagguctt

SEQ ID NO: 274 - DNA, 21 bp, Homo sapiens
aagcttctgggtgtcactatg

SEQ ID NO: 275 - DNA, 21 bp, Homo sapiens
gcuucugggugucacuaugtt

SEQ ID NO: 276 - DNA, 21 bp, Homo sapiens
cauagugacacccagaagctt

SEQ ID NO: 277 - DNA, 17 bp, Homo sapiens
aagctgcaaggtcttct

SEQ ID NO: 278 - DNA, 17 bp, Homo sapiens
gcugcaaggucuucutt

SEQ ID NO: 279 - DNA, 17 bp, Homo sapiens
agaagaccuugcagctt

SEQ ID NO: 280 - DNA, 21 bp, Homo sapiens
aaggtcttcttcaaaagagcc

SEQ ID NO: 281 - DNA, 21 bp, Homo sapiens
ggucuucuucaaaagagcctt

SEQ ID NO: 282 - DNA, 21 bp, Homo sapiens
ggcucuuuugaagaagacctt

SEQ ID NO: 283 - DNA, 21 bp, Homo sapiens
aaaagagccgctgaagggaaa

SEQ ID NO: 284 - DNA, 21 bp, Homo sapiens
aagagccgcugaagggaaatt

SEQ ID NO: 285 - DNA, 21 bp, Homo sapiens
uuucccuucagcggcucuutt

SEQ ID NO: 286 - DNA, 21 bp, Homo sapiens
aagagccgctgaagggaaaca

SEQ ID NO: 287 - DNA, 21 bp, Homo sapiens
gagccgcugaagggaaacatt

SEQ ID NO: 288 - DNA, 21 bp, Homo sapiens
uguuucccuucagcggcuctt

SEQ ID NO: 289 - DNA, 21 bp, Homo sapiens
aagggaaacagaagtacctgt

SEQ ID NO: 290 - DNA, 21 bp, Homo sapiens
gggaaacagaaguaccugutt

SEQ ID NO: 291 - DNA, 21 bp, Homo sapiens
acagguacuucuguuuccctt

SEQ ID NO: 292 - DNA, 21 bp, Homo sapiens
aaacagaagtacctgtgcgcc

SEQ ID NO: 293 - DNA, 21 bp, Homo sapiens
acagaaguaccugugcgcctt

SEQ ID NO: 294 - DNA, 21 bp, Homo sapiens
ggcgcacagguacuucugutt

SEQ ID NO: 295 - DNA, 21 bp, Homo sapiens
aagtacctgtgcgccagcaga

SEQ ID NO: 296 - DNA, 21 bp, Homo sapiens
guaccugugcgccagcagatt

SEQ ID NO: 297 - DNA, 21 bp, Homo sapiens
ucugcuggcgcacagguactt

SEQ ID NO: 298 - DNA, 17 bp, Homo sapiens
aaatgattgcactattg

SEQ ID NO: 299 - DNA, 17 bp, Homo sapiens
augauugcacuauugtt

SEQ ID NO: 300 - DNA, 17 bp, Homo sapiens
caauagugcaaucautt

SEQ ID NO: 301 - DNA, 21 bp, Homo sapiens
aaattccgaaggaaaaattgt

SEQ ID NO: 302 - DNA, 21 bp, Homo sapiens
auuccgaaggaaaaauugutt

SEQ ID NO: 303 - DNA, 21 bp, Homo sapiens
acaauuuuuccuucggaautt

SEQ ID NO: 304 - DNA, 21 bp, Homo sapiens
aaggaaaaattgtccatcttg

SEQ ID NO: 305 - DNA, 21 bp, Homo sapiens
ggaaaaauuguccaucuugtt

SEQ ID NO: 306 - DNA, 21 bp, Homo sapiens
caagauggacaauuuuucctt

SEQ ID NO: 307 - DNA, 21 bp, Homo sapiens
aaaaattgtccatcttgtcgt

SEQ ID NO: 308 - DNA, 21 bp, Homo sapiens
aaauuguccaucuugucgutt

SEQ ID NO: 309 - DNA, 21 bp, Homo sapiens
acgacaagauggacaauuutt

SEQ ID NO: 310 - DNA, 21 bp, Homo sapiens
aaattgtccatcttgtcgtct

SEQ ID NO: 311 - DNA, 21 bp, Homo sapiens
auuguccaucuugucgucutt

SEQ ID NO: 312 - DNA, 21 bp, Homo sapiens
agacgacaagauggacaautt

SEQ ID NO: 313 - DNA, 17 bp, Homo sapiens
aaatgttatgaagcagg

SEQ ID NO: 314 - DNA, 17 bp, Homo sapiens
auguuaugaagcaggtt

SEQ ID NO: 315 - DNA, 17 bp, Homo sapiens
ccugcuucauaacautt

SEQ ID NO: 316 - DNA, 21 bp, Homo sapiens
aagcagggatgactctgggag

SEQ ID NO: 317 - DNA, 21 bp, Homo sapiens
gcagggaugacucugggagtt

SEQ ID NO: 318 - DNA, 21 bp, Homo sapiens
cucccagagucaucccugctt

SEQ ID NO: 319 - DNA, 21 bp, Homo sapiens
aagctgaagaaacttggtaat

SEQ ID NO: 320 - DNA, 21 bp, Homo sapiens
gcugaagaaacuugguaautt

SEQ ID NO: 321 - DNA, 21 bp, Homo sapiens
auuaccaaguuucuucagctt

SEQ ID NO: 322 - DNA, 21 bp, Homo sapiens
aagaaacttggtaatctgaaa

SEQ ID NO: 323 - DNA, 21 bp, Homo sapiens
gaaacuugguaaucugaaatt

SEQ ID NO: 324 - DNA, 21 bp, Homo sapiens
uuucagauuaccaaguuuctt

SEQ ID NO: 325 - DNA, 21 bp, Homo sapiens
aaacttggtaatctgaaacta

SEQ ID NO: 326 - DNA, 21 bp, Homo sapiens
acuugguaaucugaaacuatt

SEQ ID NO: 327 - DNA, 21 bp, Homo sapiens
uaguuucagauuaccaagutt

SEQ ID NO: 328 - DNA, 17 bp, Homo sapiens
aatctgaaactacagga

SEQ ID NO: 329 - DNA, 17 bp, Homo sapiens
ucugaaacuacaggatt

SEQ ID NO: 330 - DNA, 17 bp, Homo sapiens
uccuguaguuucagatt

SEQ ID NO: 331 - DNA, 17 bp, Homo sapiens
aaactacaggaggaagg

SEQ ID NO: 332 - DNA, 17 bp, Homo sapiens
acuacaggaggaaggtt

SEQ ID NO: 333 - DNA, 17 bp, Homo sapiens
ccuuccuccuguagutt

SEQ ID NO: 334 - DNA, 21 bp, Homo sapiens
aaggagaggcttccagcacca

SEQ ID NO: 335 - DNA, 21 bp, Homo sapiens
ggagaggcuuccagcaccatt

SEQ ID NO: 336 - DNA, 21 bp, Homo sapiens
uggugcuggaagccucucctt

SEQ ID NO: 337 - DNA, 17 bp, Homo sapiens
aacccagaagctgacag

SEQ ID NO: 338 - DNA, 17 bp, Homo sapiens
cccagaagcugacagtt

SEQ ID NO: 339 - DNA, 17 bp, Homo sapiens
cugucagcuucugggtt

SEQ ID NO: 340 - DNA, 17 bp, Homo sapiens
aagctgacagtgtcaca

SEQ ID NO: 341 - DNA, 17 bp, Homo sapiens
gcugacagugucacatt

SEQ ID NO: 342 - DNA, 17 bp, Homo sapiens
ugugacacugucagctt

SEQ ID NO: 343 - DNA, 21 bp, Homo sapiens
aaggctatgaatgtcagccca

SEQ ID NO: 344 - DNA, 21 bp, Homo sapiens
ggcuaugaaugucagcccatt

SEQ ID NO: 345 - DNA, 21 bp, Homo sapiens
ugggcugacauucauagcctt

SEQ ID NO: 346 - DNA, 21 bp, Homo sapiens
aatgtcagcccatctttctga

SEQ ID NO: 347 - DNA, 21 bp, Homo sapiens
ugucagcccaucuuucugatt

SEQ ID NO: 348 - DNA, 21 bp, Homo sapiens
ucagaaagaugggcugacatt

SEQ ID NO: 349 - DNA, 17 bp, Homo sapiens
aatgtcctggaagccat

SEQ ID NO: 350 - DNA, 17 bp, Homo sapiens
uguccuggaagccautt

SEQ ID NO: 351 - DNA, 17 bp, Homo sapiens
auggcuuccaggacatt

SEQ ID NO: 352 - DNA, 17 bp, Homo sapiens
aagccattgagccaggt

SEQ ID NO: 353 - DNA, 17 bp, Homo sapiens
gccauugagccaggutt

SEQ ID NO: 354 - DNA, 17 bp, Homo sapiens
accuggcucaauggctt

SEQ ID NO: 355 - DNA, 21 bp, Homo sapiens
aacaaccagcccgactccttt

SEQ ID NO: 356 - DNA, 21 bp, Homo sapiens
caaccagcccgacuccuuutt

SEQ ID NO: 357 - DNA, 21 bp, Homo sapiens
aaaggagucgggcugguugtt

SEQ ID NO: 358 - DNA, 21 bp, Homo sapiens
aaccagcccgactcctttgca

SEQ ID NO: 359 - DNA, 21 bp, Homo sapiens
ccagcccgacuccuuugcatt

SEQ ID NO: 360 - DNA, 21 bp, Homo sapiens
ugcaaaggagucgggcuggtt

SEQ ID NO: 361 - DNA, 21 bp, Homo sapiens
aatgaactgggagagagacag

SEQ ID NO: 362 - DNA, 21 bp, Homo sapiens
ugaacugggagagagacagtt

SEQ ID NO: 363 - DNA, 21 bp, Homo sapiens
cugucucucucccaguucatt

SEQ ID NO: 364 - DNA, 21 bp, Homo sapiens
aactgggagagagacagcttg

SEQ ID NO: 365 - DNA, 21 bp, Homo sapiens
cugggagagagacagcuugtt

SEQ ID NO: 366 - DNA, 21 bp, Homo sapiens
caagcugucucucucccagtt

SEQ ID NO: 367 - DNA, 17 bp, Homo sapiens
aagtgggccaaggcctt

SEQ ID NO: 368 - DNA, 17 bp, Homo sapiens
gugggccaaggccuutt

SEQ ID NO: 369 - DNA, 17 bp, Homo sapiens
aaggccuuggcccactt

SEQ ID NO: 370 - DNA, 21 bp, Homo sapiens
aaggccttgcctggcttccgc

SEQ ID NO: 371 - DNA, 21 bp, Homo sapiens
ggccuugccuggcuuccgctt

SEQ ID NO: 372 - DNA, 21 bp, Homo sapiens
gcggaagccaggcaaggcctt

SEQ ID NO: 373 - DNA, 21 bp, Homo sapiens
aacttacacgtggacgaccag

SEQ ID NO: 374 - DNA, 21 bp, Homo sapiens
cuuacacguggacgaccagtt

SEQ ID NO: 375 - DNA, 21 bp, Homo sapiens
cuggucguccacguguaagtt

SEQ ID NO: 376 - DNA, 17 bp, Homo sapiens
aatgtcaactccaggat

SEQ ID NO: 377 - DNA, 17 bp, Homo sapiens
ugucaacuccaggautt

SEQ ID NO: 378 - DNA, 17 bp, Homo sapiens
auccuggaguugacatt

SEQ ID NO: 379 - DNA, 17 bp, Homo sapiens
aactccaggatgctcta

SEQ ID NO: 380 - DNA, 17 bp, Homo sapiens
cuccaggaugcucuatt

SEQ ID NO: 381 - DNA, 17 bp, Homo sapiens
uagagcauccuggagtt

SEQ ID NO: 382 - DNA, 21 bp, Homo sapiens
aatgagtaccgcatgcacaag

SEQ ID NO: 383 - DNA, 21 bp, Homo sapiens
ugaguaccgcaugcacaagtt

SEQ ID NO: 384 - DNA, 21 bp, Homo sapiens
cuugugcaugcgguacucatt

SEQ ID NO: 385 - DNA, 17 bp, Homo sapiens
aagtcccggatgtacag

SEQ ID NO: 386 - DNA, 17 bp, Homo sapiens
gucccggauguacagtt

SEQ ID NO: 387 - DNA, 17 bp, Homo sapiens
cuguacauccgggactt

SEQ ID NO: 388 - DNA, 21 bp, Homo sapiens
aatgaggcacctctctcaaga

SEQ ID NO: 389 - DNA, 21 bp, Homo sapiens
ugaggcaccucucucaagatt

SEQ ID NO: 390 - DNA, 21 bp, Homo sapiens
ucuugagagaggugccucatt

SEQ ID NO: 391 - DNA, 21 bp, Homo sapiens
aagagtttggatggctccaaa

SEQ ID NO: 392 - DNA, 21 bp, Homo sapiens
gaguuuggauggcuccaaatt

SEQ ID NO: 393 - DNA, 21 bp, Homo sapiens
uuuggagccauccaaacuctt

SEQ ID NO: 394 - DNA, 17 bp, Homo sapiens
aaatcaccccccaggaa

SEQ ID NO: 395 - DNA, 17 bp, Homo sapiens
aucaccccccaggaatt

SEQ ID NO: 396 - DNA, 17 bp, Homo sapiens
uuccuggggggugautt

SEQ ID NO: 397 - DNA, 21 bp, Homo sapiens
aattcctgtgcatgaaagcac

SEQ ID NO: 398 - DNA, 21 bp, Homo sapiens
uuccugugcaugaaagcactt

SEQ ID NO: 399 - DNA, 21 bp, Homo sapiens
gugcuuucaugcacaggaatt

SEQ ID NO: 400 - DNA, 21 bp, Homo sapiens
aaagcactgctactcttcagc

SEQ ID NO: 401 - DNA, 21 bp, Homo sapiens
agcacugcuacucuucagctt

SEQ ID NO: 402 - DNA, 21 bp, Homo sapiens
gcugaagaguagcagugcutt

SEQ ID NO: 403 - DNA, 21 bp, Homo sapiens
aaaaatcaaaaattctttgat

SEQ ID NO: 404 - DNA, 21 bp, Homo sapiens
aaaucaaaaauucuuugautt

SEQ ID NO: 405 - DNA, 21 bp, Homo sapiens
aucaaagaauuuuugauuutt

SEQ ID NO: 406 - DNA, 21 bp, Homo sapiens
aaatcaaaaattctttgatga

SEQ ID NO: 407 - DNA, 21 bp, Homo sapiens
aucaaaaauucuuugaugatt

SEQ ID NO: 408 - DNA, 21 bp, Homo sapiens
ucaucaaagaauuuuugautt

SEQ ID NO: 409 - DNA, 21 bp, Homo sapiens
aaaaattctttgatgaacttc

SEQ ID NO: 410 - DNA, 21 bp, Homo sapiens
aaauucuuugaugaacuuctt

SEQ ID NO: 411 - DNA, 21 bp, Homo sapiens
gaaguucaucaaagaauuutt

SEQ ID NO: 412 - DNA, 21 bp, Homo sapiens
aaattctttgatgaacttcga

SEQ ID NO: 413 - DNA, 21 bp, Homo sapiens
auucuuugaugaacuucgatt

SEQ ID NO: 414 - DNA, 21 bp, Homo sapiens
ucgaaguucaucaaagaautt

SEQ ID NO: 415 - DNA, 21 bp, Homo sapiens
aacttcgaatgaactacatca

SEQ ID NO: 416 - DNA, 21 bp, Homo sapiens
cuucgaaugaacuacaucatt

SEQ ID NO: 417 - DNA, 21 bp, Homo sapiens
ugauguaguucauucgaagtt

SEQ ID NO: 418 - DNA, 21 bp, Homo sapiens
aatgaactacatcaaggaact

SEQ ID NO: 419 - DNA, 21 bp, Homo sapiens
ugaacuacaucaaggaacutt

SEQ ID NO: 420 - DNA, 21 bp, Homo sapiens
aguuccuugauguaguucatt

SEQ ID NO: 421 - DNA, 21 bp, Homo sapiens
aactacatcaaggaactcgat

SEQ ID NO: 422 - DNA, 21 bp, Homo sapiens
cuacaucaaggaacucgautt

SEQ ID NO: 423 - DNA, 21 bp, Homo sapiens
aucgaguuccuugauguagtt

SEQ ID NO: 424 - DNA, 17 bp, Homo sapiens
aaggaactcgatcgtat

SEQ ID NO: 425 - DNA, 17 bp, Homo sapiens
ggaacucgaucguautt

SEQ ID NO: 426 - DNA, 17 bp, Homo sapiens
auacgaucgaguucctt

SEQ ID NO: 427 - DNA, 17 bp, Homo sapiens
aactcgatcgtatcatt

SEQ ID NO: 428 - DNA, 17 bp, Homo sapiens
cucgaucguaucauutt

SEQ ID NO: 429 - DNA, 17 bp, Homo sapiens
aaugauacgaucgagtt

SEQ ID NO: 430 - DNA, 21 bp, Homo sapiens
aaaagaaaaaatcccacatcc

SEQ ID NO: 431 - DNA, 21 bp, Homo sapiens
aagaaaaaaucccacaucctt

SEQ ID NO: 432 - DNA, 21 bp, Homo sapiens
ggaugugggauuuuuucuutt

SEQ ID NO: 433 - DNA, 21 bp, Homo sapiens
aagaaaaaatcccacatcctg

SEQ ID NO: 434 - DNA, 21 bp, Homo sapiens
gaaaaaaucccacauccugtt

SEQ ID NO: 435 - DNA, 21 bp, Homo sapiens
caggaugugggauuuuuuctt

SEQ ID NO: 436 - DNA, 21 bp, Homo sapiens
aaaaaatcccacatcctgctc

SEQ ID NO: 437 - DNA, 21 bp, Homo sapiens
aaaaucccacauccugcuctt

SEQ ID NO: 438 - DNA, 21 bp, Homo sapiens
gagcaggaugugggauuuutt

SEQ ID NO: 439 - DNA, 21 bp, Homo sapiens
aaaatcccacatcctgctcaa

SEQ ID NO: 440 - DNA, 21 bp, Homo sapiens
aaucccacauccugcucaatt

SEQ ID NO: 441 - DNA, 21 bp, Homo sapiens
uugagcaggaugugggauutt

SEQ ID NO: 442 - DNA, 21 bp, Homo sapiens
aatcccacatcctgctcaaga

SEQ ID NO: 443 - DNA, 21 bp, Homo sapiens
ucccacauccugcucaagatt

SEQ ID NO: 444 - DNA, 21 bp, Homo sapiens
ucuugagcaggaugugggatt

SEQ ID NO: 445 - DNA, 19 bp, Homo sapiens
aagacgcttctaccagctc

SEQ ID NO: 446 - DNA, 19 bp, Homo sapiens
gacgcuucuaccagcuctt

SEQ ID NO: 447 - DNA, 19 bp, Homo sapiens
gagcugguagaagcguctt

SEQ ID NO: 448 - DNA, 21 bp, Homo sapiens
aagctcctggactccgtgcag

SEQ ID NO: 449 - DNA, 21 bp, Homo sapiens
gcuccuggacuccgugcagtt

SEQ ID NO: 450 - DNA, 21 bp, Homo sapiens
cugcacggaguccaggagctt

SEQ ID NO: 451 - DNA, 21 bp, Homo sapiens
aatcaagtcacacatggtgag

SEQ ID NO: 452 - DNA, 21 bp, Homo sapiens
ucaagucacacauggugagtt

SEQ ID NO: 453 - DNA, 21 bp, Homo sapiens
cucaccaugugugacuugatt

SEQ ID NO: 454 - DNA, 21 bp, Homo sapiens
aagtcacacatggtgagcgtg

SEQ ID NO: 455 - DNA, 21 bp, Homo sapiens
gucacacauggugagcgugtt

SEQ ID NO: 456 - DNA, 21 bp, Homo sapiens
cacgcucaccaugugugactt

SEQ ID NO: 457 - DNA, 20 bp, Homo sapiens
aaatgatggcagagatcatc

SEQ ID NO: 458 - DNA, 20 bp, Homo sapiens
augauggcagagaucauctt

SEQ ID NO: 459 - DNA, 20 bp, Homo sapiens
gaugaucucugccaucautt

SEQ ID NO: 460 - DNA, 21 bp, Homo sapiens
aagtgcccaagatcctttctg

SEQ ID NO: 461 - DNA, 21 bp, Homo sapiens
gugcccaagauccuuucugtt

SEQ ID NO: 462 - DNA, 21 bp, Homo sapiens
cagaaaggaucuugggcactt

SEQ ID NO: 463 - DNA, 21 bp, Homo sapiens
aagatcctttctgggaaagtc

SEQ ID NO: 464 - DNA, 21 bp, Homo sapiens
gauccuuucugggaaaguctt

SEQ ID NO: 465 - DNA, 21 bp, Homo sapiens
gacuuucccagaaaggauctt

SEQ ID NO: 466 - DNA, 21 bp, Homo sapiens
aaagtcaagcccatctatttc

SEQ ID NO: 467 - DNA, 21 bp, Homo sapiens
agucaagcccaucuauuuctt

SEQ ID NO: 468 - DNA, 21 bp, Homo sapiens
gaaauagaugggcuugacutt

SEQ ID NO: 469 - DNA, 21 bp, Homo sapiens
aagcccatctatttccacacc

SEQ ID NO: 470 - DNA, 21 bp, Homo sapiens
gcccaucuauuuccacacctt

SEQ ID NO: 471 - DNA, 21 bp, Homo sapiens
gguguggaaauagaugggctt

SEQ ID NO: 472 - DNA, 1512 bp, Homo sapiens
atggttttggaaatgctgaacccgatacattataacatcaccagcatcgtgcctgaagccatgcctgctgccaccatgccagtcctgctcctcactggcctttttctcttggtgtggaattatgagggcacatcctcaataccaggtcctggctactgcatgggaattggacccctcatctcccacggcagattcctgtggatggggatcggcagtgcctgcaactactacaaccgggtatatggagaattcatgcgagtctggatctctggagaggaaacactcattatcagcaagtcctcaagtatgttccacataatgaagcacaatcattacagctctcgattcggcagcaaacttgggctgcagtgcatcggtatgcatgagaaaggcatcatatttaacaacaatccagagctctggaaaacaactcgacccttctttatgaaagctctgtcaggccccggccttgttcgtatggtcacagtctgtgctgaatccctcaaaacacatctggacaggttggaggaggtgaccaatgaatcgggctatgtggacgtgttgacccttctgcgtcgtgtcatgctggacacctctaacacgctcttcttgaggatccctttggacgaaagtgctatcgtggttaaaatccaaggttattttgatgcatggcaagctctcctcatcaaaccagacatcttctttaagatttcttggctatacaaaaagtatgagaagtctgtcaaggatttgaaagatgccatagaagttctgatagcagaaaaaagacgcaggatttccacagaagagaaactggaagaatgtatggactttgccactgagttgattttagcagagaaacgtggtgacctgacaagagagaatgtgaaccagtgcatattggaaatgctgatcgcagctcctgacaccatgtctgtctctttgttcttcatgctatttctcattgcaaagcaccctaatgttgaagaggcaataataaaggaaatccagactgttattggtgagagagacataaagattgatgatatacaaaaattaaaagtgatggaaaacttcatttatgagagcatgcggtaccagcctgtcgtggacttggtcatgcgcaaagccttagaagatgatgtaatcgatggctacccagtgaaaaaggggacaaacattatcctgaatattggaaggatgcacagactcgagtttttccccaaacccaatgaatttactcttgaaaattttgcaaagaatgttccttataggtactttcagccatttggctttgggccccgtggctgtgcaggaaagtacatcgccatggtgatgatgaaagccatcctcgttacacttctgagacgattccacgtgaagacattgcaaggacagtgtgttgagagcatacagaagatacacgacttgtccttgcacccagatgagactaaaaacatgctggaaatgatctttaccccaagaagctcagacaggtgtctggaacactag

SEQ ID NO: 473 - DNA, 21 bp, Homo sapiens
aaatgctgaacccgatacatt

SEQ ID NO: 474 - DNA, 21 bp, Homo sapiens
augcugaacccgauacauutt

SEQ ID NO: 475 - DNA, 21 bp, Homo sapiens
aauguaucggguucagcautt

SEQ ID NO: 476 - DNA, 21 bp, Homo sapiens
aacccgatacattataacatc

SEQ ID NO: 477 - DNA, 21 bp, Homo sapiens
cccgauacauuauaacauctt

SEQ ID NO: 478 - DNA, 21 bp, Homo sapiens
gauguuauaauguaucgggtt

SEQ ID NO: 479 - DNA, 21 bp, Homo sapiens
aacatcaccagcatcgtgcct

SEQ ID NO: 480 - DNA, 21 bp, Homo sapiens
caucaccagcaucgugccutt

SEQ ID NO: 481 - DNA, 21 bp, Homo sapiens
aggcacgaugcuggugaugtt

SEQ ID NO: 482 - DNA, 19 bp, Homo sapiens
aagccatgcctgctgccac

SEQ ID NO: 483 - DNA, 19 bp, Homo sapiens
gccaugccugcugccactt

SEQ ID NO: 484 - DNA, 19 bp, Homo sapiens
guggcagcaggcauggctt

SEQ ID NO: 485 - DNA, 18 bp, Homo sapiens
aattatgagggcacatcc

SEQ ID NO: 486 - DNA, 18 bp, Homo sapiens
uuaugagggcacaucctt

SEQ ID NO: 487 - DNA, 18 bp, Homo sapiens
ggaugugcccucauaatt

SEQ ID NO: 488 - DNA, 21 bp, Homo sapiens
aataccaggtcctggctactg

SEQ ID NO: 489 - DNA, 21 bp, Homo sapiens
uaccagguccuggcuacugtt

SEQ ID NO: 490 - DNA, 21 bp, Homo sapiens
caguagccaggaccugguatt

SEQ ID NO: 491 - DNA, 18 bp, Homo sapiens
aattggacccctcatctc

SEQ ID NO: 492 - DNA, 18 bp, Homo sapiens
uuggaccccucaucuctt

SEQ ID NO: 493 - DNA, 18 bp, Homo sapiens
gagaugagggguccaatt

SEQ ID NO: 494 - DNA, 18 bp, Homo sapiens
aactactacaaccgggta

SEQ ID NO: 495 - DNA, 18 bp, Homo sapiens
cuacuacaaccggguatt

SEQ ID NO: 496 - DNA, 18 bp, Homo sapiens
uacccgguuguaguagtt

SEQ ID NO: 497 - DNA, 18 bp, Homo sapiens
aaccgggtatatggagaa

SEQ ID NO: 498 - DNA, 18 bp, Homo sapiens
ccggguauauggagaatt

SEQ ID NO: 499 - DNA, 18 bp, Homo sapiens
uucuccauauacccggtt

SEQ ID NO: 500 - DNA, 21 bp, Homo sapiens
aattcatgcgagtctggatct

SEQ ID NO: 501 - DNA, 21 bp, Homo sapiens
uucaugcgagucuggaucutt

SEQ ID NO: 502 - DNA, 21 bp, Homo sapiens
agauccagacucgcaugaatt

SEQ ID NO: 503 - DNA, 21 bp, Homo sapiens
aaacactcattatcagcaagt

SEQ ID NO: 504 - DNA, 21 bp, Homo sapiens
acacucauuaucagcaagutt

SEQ ID NO: 505 - DNA, 21 bp, Homo sapiens
acuugcugauaaugagugutt

SEQ ID NO: 506 - DNA, 18 bp, Homo sapiens
aagtcctcaagtatgttc

SEQ ID NO: 507 - DNA, 18 bp, Homo sapiens
guccucaaguauguuctt

SEQ ID NO: 508 - DNA, 18 bp, Homo sapiens
gaacauacuugaggactt

SEQ ID NO: 509 - DNA, 21 bp, Homo sapiens
aagtatgttccacataatgaa

SEQ ID NO: 510 - DNA, 21 bp, Homo sapiens
guauguuccacauaaugaatt

SEQ ID NO: 511 - DNA, 21 bp, Homo sapiens
uucauuauguggaacauactt

SEQ ID NO: 512 - DNA, 21 bp, Homo sapiens
aatgaagcacaatcattacag

SEQ ID NO: 513 - DNA, 21 bp, Homo sapiens
ugaagcacaaucauuacagtt

SEQ ID NO: 514 - DNA, 21 bp, Homo sapiens
cuguaaugauugugcuucatt

SEQ ID NO: 515 - DNA, 21 bp, Homo sapiens
aagcacaatcattacagctct

SEQ ID NO: 516 - DNA, 21 bp, Homo sapiens
gcacaaucauuacagcucutt

SEQ ID NO: 517 - DNA, 21 bp, Homo sapiens
agagcuguaaugauugugctt

SEQ ID NO: 518 - DNA, 21 bp, Homo sapiens
aatcattacagctctcgattc

SEQ ID NO: 519 - DNA, 21 bp, Homo sapiens
ucauuacagcucucgauuctt

SEQ ID NO: 520 - DNA, 21 bp, Homo sapiens
gaaucgagagcuguaaugatt

SEQ ID NO: 521 - DNA, 18 bp, Homo sapiens
aaacttgggctgcagtgc

SEQ ID NO: 522 - DNA, 18 bp, Homo sapiens
acuugggcugcagugctt

SEQ ID NO: 523 - DNA, 18 bp, Homo sapiens
gcacugcagcccaagutt

SEQ ID NO: 524 - DNA, 21 bp, Homo sapiens
aaaggcatcatatttaacaac

SEQ ID NO: 525 - DNA, 21 bp, Homo sapiens
aggcaucauauuuaacaactt

SEQ ID NO: 526 - DNA, 21 bp, Homo sapiens
guuguuaaauaugaugccutt

SEQ ID NO: 527 - DNA, 18 bp, Homo sapiens
aacaacaatccagagctc

SEQ ID NO: 528 - DNA, 18 bp, Homo sapiens
caacaauccagagcuctt

SEQ ID NO: 529 - DNA, 18 bp, Homo sapiens
gagcucuggauuguugtt

SEQ ID NO: 530 - DNA, 18 bp, Homo sapiens
aacaatccagagctctgg

SEQ ID NO: 531 - DNA, 18 bp, Homo sapiens
caauccagagcucuggtt

SEQ ID NO: 532 - DNA, 18 bp, Homo sapiens
ccagagcucuggauugtt

SEQ ID NO: 533 - DNA, 18 bp, Homo sapiens
aatccagagctctggaaa

SEQ ID NO: 534 - DNA, 18 bp, Homo sapiens
uccagagcucuggaaatt

SEQ ID NO: 535 - DNA, 18 bp, Homo sapiens
uuuccagagcucuggatt

SEQ ID NO: 536 - DNA, 21 bp, Homo sapiens
aaaacaactcgacccttcttt

SEQ ID NO: 537 - DNA, 21 bp, Homo sapiens
aacaacucgacccuucuuutt

SEQ ID NO: 538 - DNA, 21 bp, Homo sapiens
aaagaagggucgaguuguutt

SEQ ID NO: 539 - DNA, 21 bp, Homo sapiens
aacaactcgacccttctttat

SEQ ID NO: 540 - DNA, 21 bp, Homo sapiens
caacucgacccuucuuuautt

SEQ ID NO: 541 - DNA, 21 bp, Homo sapiens
auaaagaagggucgaguugtt

SEQ ID NO: 542 - DNA, 21 bp, Homo sapiens
aactcgacccttctttatgaa

SEQ ID NO: 543 - DNA, 21 bp, Homo sapiens
cucgacccuucuuuaugaatt

SEQ ID NO: 544 - DNA, 21 bp, Homo sapiens
uucauaaagaagggucgagtt

SEQ ID NO: 545 - DNA, 21 bp, Homo sapiens
aaagctctgtcaggccccggc

SEQ ID NO: 546 - DNA, 21 bp, Homo sapiens
agcucugucaggccccggctt

SEQ ID NO: 547 - DNA, 21 bp, Homo sapiens
gccggggccugacagagcutt

SEQ ID NO: 548 - DNA, 21 bp, Homo sapiens
aatccctcaaaacacatctgg

SEQ ID NO: 549 - DNA, 21 bp, Homo sapiens
ucccucaaaacacaucuggtt

SEQ ID NO: 550 - DNA, 21 bp, Homo sapiens
ccagauguguuuugagggatt

SEQ ID NO: 551 - DNA, 21 bp, Homo sapiens
aaaacacatctggacaggttg

SEQ ID NO: 552 - DNA, 21 bp, Homo sapiens
aacacaucuggacagguugtt

SEQ ID NO: 553 - DNA, 21 bp, Homo sapiens
caaccuguccagauguguutt

SEQ ID NO: 554 - DNA, 21 bp, Homo sapiens
aacacatctggacaggttgga

SEQ ID NO: 555 - DNA, 21 bp, Homo sapiens
cacaucuggacagguuggatt

SEQ ID NO: 556 - DNA, 21 bp, Homo sapiens
uccaaccuguccagaugugtt

SEQ ID NO: 557 - DNA, 18 bp, Homo sapiens
aatgaatcgggctatgtg

SEQ ID NO: 558 - DNA, 18 bp, Homo sapiens
ugaaucgggcuaugugtt

SEQ ID NO: 559 - DNA, 18 bp, Homo sapiens
cacauagcccgauucatt

SEQ ID NO: 560 - DNA, 21 bp, Homo sapiens
aatcgggctatgtggacgtgt

SEQ ID NO: 561 - DNA, 21 bp, Homo sapiens
ucgggcuauguggacgugutt

SEQ ID NO: 562 - DNA, 21 bp, Homo sapiens
acacguccacauagcccgatt

SEQ ID NO: 563 - DNA, 18 bp, Homo sapiens
aacacgctcttcttgagg

SEQ ID NO: 564 - DNA, 18 bp, Homo sapiens
cacgcucuucuugaggtt

SEQ ID NO: 565 - DNA, 18 bp, Homo sapiens
ccucaagaagagcgugtt

SEQ ID NO: 566 - DNA, 21 bp, Homo sapiens
aaagtgctatcgtggttaaaa

SEQ ID NO: 567 - DNA, 21 bp, Homo sapiens
agugcuaucgugguuaaaatt

SEQ ID NO: 568 - DNA, 21 bp, Homo sapiens
uuuuaaccacgauagcacutt

SEQ ID NO: 569 - DNA, 18 bp, Homo sapiens
aaaatccaaggttatttt

SEQ ID NO: 570 - DNA, 18 bp, Homo sapiens
aauccaagguuauuuutt

SEQ ID NO: 571 - DNA, 18 bp, Homo sapiens
aaaauaaccuuggauutt

SEQ ID NO: 572 - DNA, 18 bp, Homo sapiens
aatccaaggttattttga

SEQ ID NO: 573 - DNA, 18 bp, Homo sapiens
uccaagguuauuuugatt

SEQ ID NO: 574 - DNA, 18 bp, Homo sapiens
ucaaaauaaccuuggatt

SEQ ID NO: 575 - DNA, 18 bp, Homo sapiens
aaggttattttgatgcat

SEQ ID NO: 576 - DNA, 18 bp, Homo sapiens
gguuauuuugaugcautt

SEQ ID NO: 577 - DNA, 18 bp, Homo sapiens
augcaucaaaauaacctt

SEQ ID NO: 578 - DNA, 21 bp, Homo sapiens
aagctctcctcatcaaaccag

SEQ ID NO: 579 - DNA, 21 bp, Homo sapiens
gcucuccucaucaaaccagtt

SEQ ID NO: 580 - DNA, 21 bp, Homo sapiens
cugguuugaugaggagagctt

SEQ ID NO: 581 - DNA, 21 bp, Homo sapiens
aaaccagacatcttctttaag

SEQ ID NO: 582 - DNA, 21 bp, Homo sapiens
accagacaucuucuuuaagtt

SEQ ID NO: 583 - DNA, 21 bp, Homo sapiens
cuuaaagaagaugucuggutt

SEQ ID NO: 584 - DNA, 18 bp, Homo sapiens
aagatttcttggctatac

SEQ ID NO: 585 - DNA, 18 bp, Homo sapiens
gauuucuuggcuauactt

SEQ ID NO: 586 - DNA, 18 bp, Homo sapiens
guauagccaagaaauctt

SEQ ID NO: 587 - DNA, 21 bp, Homo sapiens
aaaaagtatgagaagtctgtc

SEQ ID NO: 588 - DNA, 21 bp, Homo sapiens
aaaguaugagaagucuguctt

SEQ ID NO: 589 - DNA, 21 bp, Homo sapiens
gacagacuucucauacuuutt

SEQ ID NO: 590 - DNA, 21 bp, Homo sapiens
aaagtatgagaagtctgtcaa

SEQ ID NO: 591 - DNA, 21 bp, Homo sapiens
aguaugagaagucugucaatt

SEQ ID NO: 592 - DNA, 21 bp, Homo sapiens
uugacagacuucucauacutt

SEQ ID NO: 593 - DNA, 21 bp, Homo sapiens
aagtctgtcaaggatttgaaa

SEQ ID NO: 594 - DNA, 21 bp, Homo sapiens
gucugucaaggauuugaaatt

SEQ ID NO: 595 - DNA, 21 bp, Homo sapiens
uuucaaauccuugacagactt

SEQ ID NO: 596 - DNA, 21 bp, Homo sapiens
aaggatttgaaagatgccata

SEQ ID NO: 597 - DNA, 21 bp, Homo sapiens
ggauuugaaagaugccauatt

SEQ ID NO: 598 - DNA, 21 bp, Homo sapiens
uauggcaucuuucaaaucctt

SEQ ID NO: 599 - DNA, 21 bp, Homo sapiens
aaagatgccatagaagttctg

SEQ ID NO: 600 - DNA, 21 bp, Homo sapiens
agaugccauagaaguucugtt

SEQ ID NO: 601 - DNA, 21 bp, Homo sapiens
cagaacuucuauggcaucutt

SEQ ID NO: 602 - DNA, 18 bp, Homo sapiens
aagttctgatagcagaaa

SEQ ID NO: 603 - DNA, 18 bp, Homo sapiens
guucugauagcagaaatt

SEQ ID NO: 604 - DNA, 18 bp, Homo sapiens
uuucugcuaucagaactt

SEQ ID NO: 605 - DNA, 21 bp, Homo sapiens
aaaaaagacgcaggatttcca

SEQ ID NO: 606 - DNA, 21 bp, Homo sapiens
aaaagacgcaggauuuccatt

SEQ ID NO: 607 - DNA, 21 bp, Homo sapiens
uggaaauccugcgucuuuutt

SEQ ID NO: 608 - DNA, 21 bp, Homo sapiens
aaaagacgcaggatttccaca

SEQ ID NO: 609 - DNA, 21 bp, Homo sapiens
aagacgcaggauuuccacatt

SEQ ID NO: 610 - DNA, 21 bp, Homo sapiens
uguggaaauccugcgucuutt

SEQ ID NO: 611 - DNA, 21 bp, Homo sapiens
aagacgcaggatttccacaga

SEQ ID NO: 612 - DNA, 21 bp, Homo sapiens
gacgcaggauuuccacagatt

SEQ ID NO: 613 - DNA, 21 bp, Homo sapiens
ucuguggaaauccugcguctt

SEQ ID NO: 614 - DNA, 21 bp, Homo sapiens
aagagaaactggaagaatgta

SEQ ID NO: 615 - DNA, 21 bp, Homo sapiens
gagaaacuggaagaauguatt

SEQ ID NO: 616 - DNA, 21 bp, Homo sapiens
uacauucuuccaguuucuctt

SEQ ID NO: 617 - DNA, 21 bp, Homo sapiens
aaactggaagaatgtatggac

SEQ ID NO: 618 - DNA, 21 bp, Homo sapiens
acuggaagaauguauggactt

SEQ ID NO: 619 - DNA, 21 bp, Homo sapiens
guccauacauucuuccagutt

SEQ ID NO: 620 - DNA, 21 bp, Homo sapiens
aagaatgtatggactttgcca

SEQ ID NO: 621 - DNA, 21 bp, Homo sapiens
gaauguauggacuuugccatt

SEQ ID NO: 622 - DNA, 21 bp, Homo sapiens
uggcaaaguccauacauuctt

SEQ ID NO: 623 - DNA, 20 bp, Homo sapiens
aatgtatggactttgccact

SEQ ID NO: 624 - DNA, 20 bp, Homo sapiens
uguauggacuuugccacutt

SEQ ID NO: 625 - DNA, 20 bp, Homo sapiens
aguggcaaaguccauacatt

SEQ ID NO: 626 - DNA, 21 bp, Homo sapiens
aaacgtggtgacctgacaaga

SEQ ID NO: 627 - DNA, 21 bp, Homo sapiens
acguggugaccugacaagatt

SEQ ID NO: 628 - DNA, 21 bp, Homo sapiens
ucuugucaggucaccacgutt

SEQ ID NO: 629 - DNA, 21 bp, Homo sapiens
aagagagaatgtgaaccagtg

SEQ ID NO: 630 - DNA, 21 bp, Homo sapiens
gagagaaugugaaccagugtt

SEQ ID NO: 631 - DNA, 21 bp, Homo sapiens
cacugguucacauucucuctt

SEQ ID NO: 632 - DNA, 18 bp, Homo sapiens
aatgtgaaccagtgcata

SEQ ID NO: 633 - DNA, 18 bp, Homo sapiens
ugugaaccagugcauatt

SEQ ID NO: 634 - DNA, 18 bp, Homo sapiens
uaugcacugguucacatt

SEQ ID NO: 635 - DNA, 18 bp, Homo sapiens
aaccagtgcatattggaa

SEQ ID NO: 636 - DNA, 18 bp, Homo sapiens
ccagugcauauuggaatt

SEQ ID NO: 637 - DNA, 18 bp, Homo sapiens
uuccaauaugcacuggtt

SEQ ID NO: 638 - DNA, 21 bp, Homo sapiens
aaatgctgatcgcagctcctg

SEQ ID NO: 639 - DNA, 21 bp, Homo sapiens
augcugaucgcagcuccugtt

SEQ ID NO: 640 - DNA, 21 bp, Homo sapiens
caggagcugcgaucagcautt

SEQ ID NO: 641 - DNA, 21 bp, Homo sapiens
aaagcaccctaatgttgaaga

SEQ ID NO: 642 - DNA, 21 bp, Homo sapiens
agcacccuaauguugaagatt

SEQ ID NO: 643 - DNA, 21 bp, Homo sapiens
ucuucaacauuagggugcutt

SEQ ID NO: 644 - DNA, 21 bp, Homo sapiens
aatgttgaagaggcaataata

SEQ ID NO: 645 - DNA, 21 bp, Homo sapiens
uguugaagaggcaauaauatt

SEQ ID NO: 646 - DNA, 21 bp, Homo sapiens
uauuauugccucuucaacatt

SEQ ID NO: 647 - DNA, 21 bp, Homo sapiens
aagaggcaataataaaggaaa

SEQ ID NO: 648 - DNA, 21 bp, Homo sapiens
gaggcaauaauaaaggaaatt

SEQ ID NO: 649 - DNA, 21 bp, Homo sapiens
uuuccuuuauuauugccuctt

SEQ ID NO: 650 - DNA, 21 bp, Homo sapiens
aataataaaggaaatccagac

SEQ ID NO: 651 - DNA, 21 bp, Homo sapiens
uaauaaaggaaauccagactt

SEQ ID NO: 652 - DNA, 21 bp, Homo sapiens
gucuggauuuccuuuauuatt

SEQ ID NO: 653 - DNA, 21 bp, Homo sapiens
aataaaggaaatccagactgt

SEQ ID NO: 654 - DNA, 21 bp, Homo sapiens
uaaaggaaauccagacugutt

SEQ ID NO: 655 - DNA, 21 bp, Homo sapiens
acagucuggauuuccuuuatt

SEQ ID NO: 656 - DNA, 21 bp, Homo sapiens
aaaggaaatccagactgttat

SEQ ID NO: 657 - DNA, 21 bp, Homo sapiens
aggaaauccagacuguuautt

SEQ ID NO: 658 - DNA, 21 bp, Homo sapiens
auaacagucuggauuuccutt

SEQ ID NO: 659 - DNA, 17 bp, Homo sapiens
aaatccagactgttatt

SEQ ID NO: 660 - DNA, 17 bp, Homo sapiens
auccagacuguuauutt

SEQ ID NO: 661 - DNA, 17 bp, Homo sapiens
aauaacagucuggautt

SEQ ID NO: 662 - DNA, 21 bp, Homo sapiens
aaagattgatgatatacaaaa

SEQ ID NO: 663 - DNA, 21 bp, Homo sapiens
agauugaugauauacaaaatt

SEQ ID NO: 664 - DNA, 21 bp, Homo sapiens
uuuuguauaucaucaaucutt

SEQ ID NO: 665 - DNA, 21 bp, Homo sapiens
aaaaattaaaagtgatggaaa

SEQ ID NO: 666 - DNA, 21 bp, Homo sapiens
aaauuaaaagugauggaaatt

SEQ ID NO: 667 - DNA, 21 bp, Homo sapiens
uuuccaucacuuuuaauuutt

SEQ ID NO: 668 - DNA, 21 bp, Homo sapiens
aaattaaaagtgatggaaaac

SEQ ID NO: 669 - DNA, 21 bp, Homo sapiens
auuaaaagugauggaaaactt

SEQ ID NO: 670 - DNA, 21 bp, Homo sapiens
guuuuccaucacuuuuaautt

SEQ ID NO: 671 - DNA, 21 bp, Homo sapiens
aaaagtgatggaaaacttcat

SEQ ID NO: 672 - DNA, 21 bp, Homo sapiens
aagugauggaaaacuucautt

SEQ ID NO: 673 - DNA, 21 bp, Homo sapiens
augaaguuuuccaucacuutt

SEQ ID NO: 674 - DNA, 20 bp, Homo sapiens
aagtgatggaaaacttcatt

SEQ ID NO: 675 - DNA, 20 bp, Homo sapiens
gugauggaaaacuucauutt

SEQ ID NO: 676 - DNA, 20 bp, Homo sapiens
aaugaaguuuuccaucactt

SEQ ID NO: 677 - DNA, 17 bp, Homo sapiens
aaaacttcatttatgag

SEQ ID NO: 678 - DNA, 17 bp, Homo sapiens
aacuucauuuaugagtt

SEQ ID NO: 679 - DNA, 17 bp, Homo sapiens
cucauaaaugaaguutt

SEQ ID NO: 680 - DNA, 17 bp, Homo sapiens
aacttcatttatgagag

SEQ ID NO: 681 - DNA, 17 bp, Homo sapiens
cuucauuuaugagagtt

SEQ ID NO: 682 - DNA, 17 bp, Homo sapiens
cucucauaaaugaagtt

SEQ ID NO: 683 - DNA, 17 bp, Homo sapiens
aaagccttagaagatga

SEQ ID NO: 684 - DNA, 17 bp, Homo sapiens
agccuuagaagaugatt

SEQ ID NO: 685 - DNA, 17 bp, Homo sapiens
ucaucuucuaaggcutt

SEQ ID NO: 686 - DNA, 17 bp, Homo sapiens
aagatgatgtaatcgat

SEQ ID NO: 687 - DNA, 17 bp, Homo sapiens
gaugauguaaucgautt

SEQ ID NO: 688 - DNA, 17 bp, Homo sapiens
aucgauuacaucauctt

SEQ ID NO: 689 - DNA, 21 bp, Homo sapiens
aatcgatggctacccagtgaa

SEQ ID NO: 690 - DNA, 21 bp, Homo sapiens
ucgauggcuacccagugaatt

SEQ ID NO: 691 - DNA, 21 bp, Homo sapiens
uucacuggguagccaucgatt

SEQ ID NO: 692 - DNA, 21 bp, Homo sapiens
aaaaaggggacaaacattatc

SEQ ID NO: 693 - DNA, 21 bp, Homo sapiens
aaaggggacaaacauuauctt

SEQ ID NO: 694 - DNA, 21 bp, Homo sapiens
gauaauguuuguccccuuutt

SEQ ID NO: 695 - DNA, 21 bp, Homo sapiens
aaaggggacaaacattatcct

SEQ ID NO: 696 - DNA, 21 bp, Homo sapiens
aggggacaaacauuauccutt

SEQ ID NO: 697 - DNA, 21 bp, Homo sapiens
aggauaauguuuguccccutt

SEQ ID NO: 698 - DNA, 21 bp, Homo sapiens
aaacattatcctgaatattgg

SEQ ID NO: 699 - DNA, 21 bp, Homo sapiens
acauuauccugaauauuggtt

SEQ ID NO: 700 - DNA, 21 bp, Homo sapiens
ccaauauucaggauaaugutt

SEQ ID NO: 701 - DNA, 17 bp, Homo sapiens
aatattggaaggatgca

SEQ ID NO: 702 - DNA, 17 bp, Homo sapiens
uauuggaaggaugcatt

SEQ ID NO: 703 - DNA, 17 bp, Homo sapiens
ugcauccuuccaauatt

SEQ ID NO: 704 - DNA, 17 bp, Homo sapiens
aaggatgcacagactcg

SEQ ID NO: 705 - DNA, 17 bp, Homo sapiens
ggaugcacagacucgtt

SEQ ID NO: 706 - DNA, 17 bp, Homo sapiens
cgagucugugcaucctt

SEQ ID NO: 707 - DNA, 21 bp, Homo sapiens
aaacccaatgaatttactctt

SEQ ID NO: 708 - DNA, 21 bp, Homo sapiens
acccaaugaauuuacucuutt

SEQ ID NO: 709 - DNA, 21 bp, Homo sapiens
aagaguaaauucauugggutt

SEQ ID NO: 710 - DNA, 21 bp, Homo sapiens
aatgaatttactcttgaaaat

SEQ ID NO: 711 - DNA, 21 bp, Homo sapiens
ugaauuuacucuugaaaautt

SEQ ID NO: 712 - DNA, 21 bp, Homo sapiens
auuuucaagaguaaauucatt

SEQ ID NO: 713 - DNA, 21 bp, Homo sapiens
aatttactcttgaaaattttg

SEQ ID NO: 714 - DNA, 21 bp, Homo sapiens
uuuacucuugaaaauuuugtt

SEQ ID NO: 715 - DNA, 21 bp, Homo sapiens
caaaauuuucaagaguaaatt

SEQ ID NO: 716 - DNA, 17 bp, Homo sapiens
aaaattttgcaaagaat

SEQ ID NO: 717 - DNA, 17 bp, Homo sapiens
aauuuugcaaagaautt

SEQ ID NO: 718 - DNA, 17 bp, Homo sapiens
auucuuugcaaaauutt

SEQ ID NO: 719 - DNA, 17 bp, Homo sapiens
aattttgcaaagaatgt

SEQ ID NO: 720 - DNA, 17 bp, Homo sapiens
uuuugcaaagaaugutt

SEQ ID NO: 721 - DNA, 17 bp, Homo sapiens
acauucuuugcaaaatt

SEQ ID NO: 722 - DNA, 17 bp, Homo sapiens
aaagaatgttccttata

SEQ ID NO: 723 - DNA, 17 bp, Homo sapiens
agaauguuccuuauatt

SEQ ID NO: 724 - DNA, 17 bp, Homo sapiens
uauaaggaacauucutt

SEQ ID NO: 725 - DNA, 21 bp, Homo sapiens
aatgttccttataggtacttt

SEQ ID NO: 726 - DNA, 21 bp, Homo sapiens
uguuccuuauagguacuuutt

SEQ ID NO: 727 - DNA, 21 bp, Homo sapiens
aaaguaccuauaaggaacatt

SEQ ID NO: 728 - DNA, 17 bp, Homo sapiens
aaagtacatcgccatgg

SEQ ID NO: 729 - DNA, 17 bp, Homo sapiens
aguacaucgccauggtt

SEQ ID NO: 730 - DNA, 17 bp, Homo sapiens
ccauggcgauguacutt

SEQ ID NO: 731 - DNA, 21 bp, Homo sapiens
aaagccatcctcgttacactt

SEQ ID NO: 732 - DNA, 21 bp, Homo sapiens
agccauccucguuacacuutt

SEQ ID NO: 733 - DNA, 21 bp, Homo sapiens
aaguguaacgaggauggcutt

SEQ ID NO: 734 - DNA, 21 bp, Homo sapiens
aagacattgcaaggacagtgt

SEQ ID NO: 735 - DNA, 21 bp, Homo sapiens
gacauugcaaggacagugutt

SEQ ID NO: 736 - DNA, 21 bp, Homo sapiens
acacuguccuugcaauguctt

SEQ ID NO: 737 - DNA, 21 bp, Homo sapiens
aaggacagtgtgttgagagca

SEQ ID NO: 738 - DNA, 21 bp, Homo sapiens
ggacaguguguugagagcatt

SEQ ID NO: 739 - DNA, 21 bp, Homo sapiens
ugcucucaacacacugucctt

SEQ ID NO: 740 - DNA, 21 bp, Homo sapiens
aagatacacgacttgtccttg

SEQ ID NO: 741 - DNA, 21 bp, Homo sapiens
gauacacgacuuguccuugtt

SEQ ID NO: 742 - DNA, 21 bp, Homo sapiens
caaggacaagucguguauctt

SEQ ID NO: 743 - DNA, 21 bp, Homo sapiens
aaaaacatgctggaaatgatc

SEQ ID NO: 744 - DNA, 21 bp, Homo sapiens
aaacaugcuggaaaugauctt

SEQ ID NO: 745 - DNA, 21 bp, Homo sapiens
gaucauuuccagcauguuutt

SEQ ID NO: 746 - DNA, 21 bp, Homo sapiens
aaacatgctggaaatgatctt

SEQ ID NO: 747 - DNA, 21 bp, Homo sapiens
acaugcuggaaaugaucuutt

SEQ ID NO: 748 - DNA, 21 bp, Homo sapiens
aagaucauuuccagcaugutt

SEQ ID NO: 749 - DNA, 21 bp, Homo sapiens
aaatgatctttaccccaagaa

SEQ ID NO: 750 - DNA, 21 bp, Homo sapiens
augaucuuuaccccaagaatt

SEQ ID NO: 751 - DNA, 21 bp, Homo sapiens
uucuugggguaaagaucautt

SEQ ID NO: 752 - DNA, 19 bp, Homo sapiens
aagaagctcagacaggtgt

SEQ ID NO: 753 - DNA, 19 bp, Homo sapiens
gaagcucagacaggugutt

SEQ ID NO: 754 - DNA, 19 bp, Homo sapiens
acaccugucugagcuuctt

SEQ ID NO: 755 - DNA, 17 bp, Homo sapiens
aagctcagacaggtgtc

SEQ ID NO: 756 - DNA, 17 bp, Homo sapiens
gcucagacagguguctt

SEQ ID NO: 757 - DNA, 17 bp, Homo sapiens
gacaccugucugagctt

SEQ ID NO: 758 - DNA, 972 bp, Homo sapiens
atggatcccaaatatcagcgtgtagagctaaatgatggtcatttcatgcccgtattgggatttggcacctatgcacctccagaggttccgaggaacagagctgtagaggtcaccaaattagcaatagaagctggcttccgccatattgattctgcttatttatacaataatgaggagcaggttggactggccatccgaagcaagattgcagatggcagtgtgaagagagaagacatattctacacttcaaagctttggtgcactttctttcaaccacagatggtccaaccagccttggaaagctcactgaaaaaacttcaactggactatgttgacctctatcttcttcatttcccaatggctctcaagccaggtgagacgccactaccaaaagatgaaaatggaaaagtaatattcgacacagtggatctctgtgccacatgggaggtcatggagaagtgtaaggatgcaggattggccaagtccatcggggtgtcaaacttcaactgcaggcagctggagatgatcctcaacaagccaggactcaagtacaagcctgtctgcaaccaggtagaatgtcatccttacctcaaccagagcaaactgctggatttctgcaagtcaaaagacattgttctggttgcccacagtgctctgggaacccaacgacataaactatgggtggacccaaactccccagttcttttggaggacccagttctttgtgccttagcaaagaaacacaaacgaaccccagccctgattgccctgcgctaccagctgcagcgtggggttgtggtcctggccaagagctacaatgagcagcggatcagagagaacatccaggtttttgaattccagttgacatcagaggatatgaaagttctagatggtctaaacagaaattatcgatatgttgtcatggattttgttatggaccatcctgattatccattttcagatgaatattag

SEQ ID NO: 759 - DNA, 21 bp, Homo sapiens
aaatatcagcgtgtagagcta

SEQ ID NO: 760 - DNA, 21 bp, Homo sapiens
auaucagcguguagagcuatt

SEQ ID NO: 761 - DNA, 21 bp, Homo sapiens
uagcucuacacgcugauautt

SEQ ID NO: 762 - DNA, 21 bp, Homo sapiens
aaatgatggtcatttcatgcc

SEQ ID NO: 763 - DNA, 21 bp, Homo sapiens
augauggucauuucaugcctt

SEQ ID NO: 764 - DNA, 21 bp, Homo sapiens
ggcaugaaaugaccaucautt

SEQ ID NO: 765 - DNA, 21 bp, Homo sapiens
aacagagctgtagaggtcacc

SEQ ID NO: 766 - DNA, 21 bp, Homo sapiens
cagagcuguagaggucacctt

SEQ ID NO: 767 - DNA, 21 bp, Homo sapiens
ggugaccucuacagcucugtt

SEQ ID NO: 768 - DNA, 18 bp, Homo sapiens
aaattagcaatagaagct

SEQ ID NO: 769 - DNA, 18 bp, Homo sapiens
auuagcaauagaagcutt

SEQ ID NO: 770 - DNA, 18 bp, Homo sapiens
agcuucuauugcuaautt

SEQ ID NO: 771 - DNA, 21 bp, Homo sapiens
aatagaagctggcttccgcca

SEQ ID NO: 772 - DNA, 21 bp, Homo sapiens
uagaagcuggcuuccgccatt

SEQ ID NO: 773 - DNA, 21 bp, Homo sapiens
uggcggaagccagcuucuatt

SEQ ID NO: 774 - DNA, 21 bp, Homo sapiens
aagctggcttccgccatattg

SEQ ID NO: 775 - DNA, 21 bp, Homo sapiens
gcuggcuuccgccauauugtt

SEQ ID NO: 776 - DNA, 21 bp, Homo sapiens
caauauggcggaagccagctt

SEQ ID NO: 777 - DNA, 18 bp, Homo sapiens
aataatgaggagcaggtt

SEQ ID NO: 778 - DNA, 18 bp, Homo sapiens
uaaugaggagcagguutt

SEQ ID NO: 779 - DNA, 18 bp, Homo sapiens
aaccugcuccucauuatt

SEQ ID NO: 780 - DNA, 18 bp, Homo sapiens
aatgaggagcaggttgga

SEQ ID NO: 781 - DNA, 18 bp, Homo sapiens
ugaggagcagguuggatt

SEQ ID NO: 782 - DNA, 18 bp, Homo sapiens
uccaaccugcuccucatt

SEQ ID NO: 783 - DNA, 21 bp, Homo sapiens
aagcaagattgcagatggcag

SEQ ID NO: 784 - DNA, 21 bp, Homo sapiens
gcaagauugcagauggcagtt

SEQ ID NO: 785 - DNA, 21 bp, Homo sapiens
cugccaucugcaaucuugctt

SEQ ID NO: 786 - DNA, 21 bp, Homo sapiens
aagattgcagatggcagtgtg

SEQ ID NO: 787 - DNA, 21 bp, Homo sapiens
gauugcagauggcagugugtt

SEQ ID NO: 788 - DNA, 21 bp, Homo sapiens
cacacugccaucugcaauctt

SEQ ID NO: 789 - DNA, 18 bp, Homo sapiens
aagagagaagacatattc

SEQ ID NO: 790 - DNA, 18 bp, Homo sapiens
gagagaagacauauuctt

SEQ ID NO: 791 - DNA, 18 bp, Homo sapiens
gaauaugucuucucuctt

SEQ ID NO: 792 - DNA, 18 bp, Homo sapiens
aagacatattctacactt

SEQ ID NO: 793 - DNA, 18 bp, Homo sapiens
gacauauucuacacuutt

SEQ ID NO: 794 - DNA, 18 bp, Homo sapiens
aaguguagaauauguctt

SEQ ID NO: 795 - DNA, 21 bp, Homo sapiens
aaagctttggtgcactttctt

SEQ ID NO: 796 - DNA, 21 bp, Homo sapiens
agcuuuggugcacuuucuutt

SEQ ID NO: 797 - DNA, 21 bp, Homo sapiens
aagaaagugcaccaaagcutt

SEQ ID NO: 798 - DNA, 21 bp, Homo sapiens
aaccacagatggtccaaccag

SEQ ID NO: 799 - DNA, 21 bp, Homo sapiens
ccacagaugguccaaccagtt

SEQ ID NO: 800 - DNA, 21 bp, Homo sapiens
cugguuggaccaucuguggtt

SEQ ID NO: 801 - DNA, 18 bp, Homo sapiens
aaccagccttggaaagct

SEQ ID NO: 802 - DNA, 18 bp, Homo sapiens
ccagccuuggaaagcutt

SEQ ID NO: 803 - DNA, 18 bp, Homo sapiens
agcuuuccaaggcuggtt

SEQ ID NO: 804 - DNA, 18 bp, Homo sapiens
aaagctcactgaaaaaac

SEQ ID NO: 805 - DNA, 18 bp, Homo sapiens
agcucacugaaaaaactt

SEQ ID NO: 806 - DNA, 18 bp, Homo sapiens
guuuuuucagugagcutt

SEQ ID NO: 807 - DNA, 21 bp, Homo sapiens
aaaaaacttcaactggactat

SEQ ID NO: 808 - DNA, 21 bp, Homo sapiens
aaaacuucaacuggacuautt

SEQ ID NO: 809 - DNA, 21 bp, Homo sapiens
auaguccaguugaaguuuutt

SEQ ID NO: 810 - DNA, 21 bp, Homo sapiens
aaaacttcaactggactatgt

SEQ ID NO: 811 - DNA, 21 bp, Homo sapiens
aacuucaacuggacuaugutt

SEQ ID NO: 812 - DNA, 21 bp, Homo sapiens
acauaguccaguugaaguutt

SEQ ID NO: 813 - DNA, 21 bp, Homo sapiens
aacttcaactggactatgttg

SEQ ID NO: 814 - DNA, 21 bp, Homo sapiens
cuucaacuggacuauguugtt

SEQ ID NO: 815 - DNA, 21 bp, Homo sapiens
caacauaguccaguugaagtt

SEQ ID NO: 816 - DNA, 21 bp, Homo sapiens
aactggactatgttgacctct

SEQ ID NO: 817 - DNA, 21 bp, Homo sapiens
cuggacuauguugaccucutt

SEQ ID NO: 818 - DNA, 21 bp, Homo sapiens
agaggucaacauaguccagtt

SEQ ID NO: 819 - DNA, 18 bp, Homo sapiens
aatggctctcaagccagg

SEQ ID NO: 820 - DNA, 18 bp, Homo sapiens
uggcucucaagccaggtt

SEQ ID NO: 821 - DNA, 18 bp, Homo sapiens
ccuggcuugagagccatt

SEQ ID NO: 822 - DNA, 21 bp, Homo sapiens
aagccaggtgagacgccacta

SEQ ID NO: 823 - DNA, 21 bp, Homo sapiens
gccaggugagacgccacuatt

SEQ ID NO: 824 - DNA, 21 bp, Homo sapiens
uaguggcgucucaccuggctt

SEQ ID NO: 825 - DNA, 21 bp, Homo sapiens
aaaagatgaaaatggaaaagt

SEQ ID NO: 826 - DNA, 21 bp, Homo sapiens
aagaugaaaauggaaaagutt

SEQ ID NO: 827 - DNA, 21 bp, Homo sapiens
acuuuuccauuuucaucuutt

SEQ ID NO: 828 - DNA, 21 bp, Homo sapiens
aagatgaaaatggaaaagtaa

SEQ ID NO: 829 - DNA, 21 bp, Homo sapiens
gaugaaaauggaaaaguaatt

SEQ ID NO: 830 - DNA, 21 bp, Homo sapiens
uuacuuuuccauuuucauctt

SEQ ID NO: 831 - DNA, 21 bp, Homo sapiens
aaaatggaaaagtaatattcg

SEQ ID NO: 832 - DNA, 21 bp, Homo sapiens
aauggaaaaguaauauucgtt

SEQ ID NO: 833 - DNA, 21 bp, Homo sapiens
cgaauauuacuuuuccauutt

SEQ ID NO: 834 - DNA, 21 bp, Homo sapiens
aatggaaaagtaatattcgac

SEQ ID NO: 835 - DNA, 21 bp, Homo sapiens
uggaaaaguaauauucgactt

SEQ ID NO: 836 - DNA, 21 bp, Homo sapiens
gucgaauauuacuuuuccatt

SEQ ID NO: 837 - DNA, 18 bp, Homo sapiens
aaaagtaatattcgacac

SEQ ID NO: 838 - DNA, 18 bp, Homo sapiens
aaguaauauucgacactt

SEQ ID NO: 839 - DNA, 18 bp, Homo sapiens
gugucgaauauuacuutt

SEQ ID NO: 840 - DNA, 18 bp, Homo sapiens
aagtaatattcgacacag

SEQ ID NO: 841 - DNA, 18 bp, Homo sapiens
guaauauucgacacagtt

SEQ ID NO: 842 - DNA, 18 bp, Homo sapiens
cugugucgaauauuactt

SEQ ID NO: 843 - DNA, 18 bp, Homo sapiens
aatattcgacacagtgga

SEQ ID NO: 844 - DNA, 18 bp, Homo sapiens
uauucgacacaguggatt

SEQ ID NO: 845 - DNA, 18 bp, Homo sapiens
uccacugugucgaauatt

SEQ ID NO: 846 - DNA, 21 bp, Homo sapiens
aagtgtaaggatgcaggattg

SEQ ID NO: 847 - DNA, 21 bp, Homo sapiens
guguaaggaugcaggauugtt

SEQ ID NO: 848 - DNA, 21 bp, Homo sapiens
caauccugcauccuuacactt

SEQ ID NO: 849 - DNA, 18 bp, Homo sapiens
aaggatgcaggattggcc

SEQ ID NO: 850 - DNA, 18 bp, Homo sapiens
ggaugcaggauuggcctt

SEQ ID NO: 851 - DNA, 18 bp, Homo sapiens
ggccaauccugcaucctt

SEQ ID NO: 852 - DNA, 21 bp, Homo sapiens
aagtccatcggggtgtcaaac

SEQ ID NO: 853 - DNA, 21 bp, Homo sapiens
guccaucggggugucaaactt

SEQ ID NO: 854 - DNA, 21 bp, Homo sapiens
guuugacaccccgauggactt

SEQ ID NO: 855 - DNA, 21 bp, Homo sapiens
aaacttcaactgcaggcagct

SEQ ID NO: 856 - DNA, 21 bp, Homo sapiens
acuucaacugcaggcagcutt

SEQ ID NO: 857 - DNA, 21 bp, Homo sapiens
agcugccugcaguugaagutt

SEQ ID NO: 858 - DNA, 21 bp, Homo sapiens
aactgcaggcagctggagatg

SEQ ID NO: 859 - DNA, 21 bp, Homo sapiens
cugcaggcagcuggagaugtt

SEQ ID NO: 860 - DNA, 21 bp, Homo sapiens
caucuccagcugccugcagtt

SEQ ID NO: 861 - DNA, 18 bp, Homo sapiens
aacaagccaggactcaag

SEQ ID NO: 862 - DNA, 18 bp, Homo sapiens
caagccaggacucaagtt

SEQ ID NO: 863 - DNA, 18 bp, Homo sapiens
cuugaguccuggcuugtt

SEQ ID NO: 864 - DNA, 18 bp, Homo sapiens
aagccaggactcaagtac

SEQ ID NO: 865 - DNA, 18 bp, Homo sapiens
gccaggacucaaguactt

SEQ ID NO: 866 - DNA, 18 bp, Homo sapiens
guacuugaguccuggctt

SEQ ID NO: 867 - DNA, 21 bp, Homo sapiens
aagtacaagcctgtctgcaac

SEQ ID NO: 868 - DNA, 21 bp, Homo sapiens
guacaagccugucugcaactt

SEQ ID NO: 869 - DNA, 21 bp, Homo sapiens
guugcagacaggcuuguactt

SEQ ID NO: 870 - DNA, 21 bp, Homo sapiens
aagcctgtctgcaaccaggta

SEQ ID NO: 871 - DNA, 21 bp, Homo sapiens
gccugucugcaaccagguatt

SEQ ID NO: 872 - DNA, 21 bp, Homo sapiens
uaccugguugcagacaggctt

SEQ ID NO: 873 - DNA, 21 bp, Homo sapiens
aaccaggtagaatgtcatcct

SEQ ID NO: 874 - DNA, 21 bp, Homo sapiens
ccagguagaaugucauccutt

SEQ ID NO: 875 - DNA, 21 bp, Homo sapiens
aggaugacauucuaccuggtt

SEQ ID NO: 876 - DNA, 21 bp, Homo sapiens
aatgtcatccttacctcaacc

SEQ ID NO: 877 - DNA, 21 bp, Homo sapiens
ugucauccuuaccucaacctt

SEQ ID NO: 878 - DNA, 21 bp, Homo sapiens
gguugagguaaggaugacatt

SEQ ID NO: 879 - DNA, 18 bp, Homo sapiens
aaccagagcaaactgctg

SEQ ID NO: 880 - DNA, 18 bp, Homo sapiens
ccagagcaaacugcugtt

SEQ ID NO: 881 - DNA, 18 bp, Homo sapiens
cagcaguuugcucuggtt

SEQ ID NO: 882 - DNA, 21 bp, Homo sapiens
aaactgctggatttctgcaag

SEQ ID NO: 883 - DNA, 21 bp, Homo sapiens
acugcuggauuucugcaagtt

SEQ ID NO: 884 - DNA, 21 bp, Homo sapiens
cuugcagaaauccagcagutt

SEQ ID NO: 885 - DNA, 21 bp, Homo sapiens
aagtcaaaagacattgttctg

SEQ ID NO: 886 - DNA, 21 bp, Homo sapiens
gucaaaagacauuguucugtt

SEQ ID NO: 887 - DNA, 21 bp, Homo sapiens
cagaacaaugucuuuugactt

SEQ ID NO: 888 - DNA, 21 bp, Homo sapiens
aaaagacattgttctggttgc

SEQ ID NO: 889 - DNA, 21 bp, Homo sapiens
aagacauuguucugguugctt

SEQ ID NO: 890 - DNA, 21 bp, Homo sapiens
gcaaccagaacaaugucuutt

SEQ ID NO: 891 - DNA, 21 bp, Homo sapiens
aagacattgttctggttgccc

SEQ ID NO: 892 - DNA, 21 bp, Homo sapiens
gacauuguucugguugccctt

SEQ ID NO: 893 - DNA, 21 bp, Homo sapiens
gggcaaccagaacaauguctt

SEQ ID NO: 894 - DNA, 21 bp, Homo sapiens
aacgacataaactatgggtgg

SEQ ID NO: 895 - DNA, 21 bp, Homo sapiens
cgacauaaacuauggguggtt

SEQ ID NO: 896 - DNA, 21 bp, Homo sapiens
ccacccauaguuuaugucgtt

SEQ ID NO: 897 - DNA, 21 bp, Homo sapiens
aaactatgggtggacccaaac

SEQ ID NO: 898 - DNA, 21 bp, Homo sapiens
acuauggguggacccaaactt

SEQ ID NO: 899 - DNA, 21 bp, Homo sapiens
guuuggguccacccauagutt

SEQ ID NO: 900 - DNA, 21 bp, Homo sapiens
aaactccccagttcttttgga

SEQ ID NO: 901 - DNA, 21 bp, Homo sapiens
acuccccaguucuuuuggatt

SEQ ID NO: 902 - DNA, 21 bp, Homo sapiens
uccaaaagaacuggggagutt

SEQ ID NO: 903 - DNA, 21 bp, Homo sapiens
aaagaaacacaaacgaacccc

SEQ ID NO: 904 - DNA, 21 bp, Homo sapiens
agaaacacaaacgaacccctt

SEQ ID NO: 905 - DNA, 21 bp, Homo sapiens
gggguucguuuguguuucutt

SEQ ID NO: 906 - DNA, 21 bp, Homo sapiens
aaacacaaacgaaccccagcc

SEQ ID NO: 907 - DNA, 21 bp, Homo sapiens
acacaaacgaaccccagcctt

SEQ ID NO: 908 - DNA, 21 bp, Homo sapiens
ggcugggguucguuugugutt

SEQ ID NO: 909 - DNA, 21 bp, Homo sapiens
aaacgaaccccagccctgatt

SEQ ID NO: 910 - DNA, 21 bp, Homo sapiens
acgaaccccagcccugauutt

SEQ ID NO: 911 - DNA, 21 bp, Homo sapiens
aaucagggcugggguucgutt

SEQ ID NO: 912 - DNA, 21 bp, Homo sapiens
aaccccagccctgattgccct

SEQ ID NO: 913 - DNA, 21 bp, Homo sapiens
ccccagcccugauugcccutt

SEQ ID NO: 914 - DNA, 21 bp, Homo sapiens
agggcaaucagggcuggggtt

SEQ ID NO: 915 - DNA, 21 bp, Homo sapiens
aagagctacaatgagcagcgg

SEQ ID NO: 916 - DNA, 21 bp, Homo sapiens
gagcuacaaugagcagcggtt

SEQ ID NO: 917 - DNA, 21 bp, Homo sapiens
ccgcugcucauuguagcuctt

SEQ ID NO: 918 - DNA, 21 bp, Homo sapiens
aatgagcagcggatcagagag

SEQ ID NO: 919 - DNA, 21 bp, Homo sapiens
ugagcagcggaucagagagtt

SEQ ID NO: 920 - DNA, 21 bp, Homo sapiens
cucucugauccgcugcucatt

SEQ ID NO: 921 - DNA, 18 bp, Homo sapiens
aacatccaggtttttgaa

SEQ ID NO: 922 - DNA, 18 bp, Homo sapiens
cauccagguuuuugaatt

SEQ ID NO: 923 - DNA, 18 bp, Homo sapiens
uucaaaaaccuggaugtt

SEQ ID NO: 924 - DNA, 21 bp, Homo sapiens
aattccagttgacatcagagg

SEQ ID NO: 925 - DNA, 21 bp, Homo sapiens
uuccaguugacaucagaggtt

SEQ ID NO: 926 - DNA, 21 bp, Homo sapiens
ccucugaugucaacuggaatt

SEQ ID NO: 927 - DNA, 21 bp, Homo sapiens
aaagttctagatggtctaaac

SEQ ID NO: 928 - DNA, 21 bp, Homo sapiens
aguucuagauggucuaaactt

SEQ ID NO: 929 - DNA, 21 bp, Homo sapiens
guuuagaccaucuagaacutt

SEQ ID NO: 930 - DNA, 18 bp, Homo sapiens
aaacagaaattatcgata

SEQ ID NO: 931 - DNA, 18 bp, Homo sapiens
acagaaauuaucgauatt

SEQ ID NO: 932 - DNA, 18 bp, Homo sapiens
uaucgauaauuucugutt

SEQ ID NO: 933 - DNA, 21 bp, Homo sapiens
aaattatcgatatgttgtcat

SEQ ID NO: 934 - DNA, 21 bp, Homo sapiens
auuaucgauauguugucautt

SEQ ID NO: 935 - DNA, 21 bp, Homo sapiens
augacaacauaucgauaautt

SEQ ID NO: 936 - DNA, 1122 bp, Homo sapiens
atgacgggctggagctgccttgtgacaggagcaggagggtttctgggacagaggatcatccgcctcttggtgaaggagaaggagctgaaggagatcagggtcttggacaaggccttcggaccagaattgagagaggaattttctaaactccagaacaagaccaagctgacagtgctggaaggagacattctggatgagccattcctgaagagagcctgccaggacgtctcggtcatcatccacaccgcctgtatcattgatgtcttcggtgtcactcacagagagtctatcatgaatgtcaatgtgaaaggtacccagctcctgttagaggcctgtgtccaagctagtgtgccagtcttcatctacaccagtagcatagaggtagccgggcccaactcctacaaggaaatcatccagaatggccatgaagaagagcctctggaaaacacatggcccgctccatacccacacagcaaaaagcttgctgagaaggctgtactggcggctaacgggtggaatctgaaaaacggcggcaccctgtacacttgtgccttacgacccatgtatatctatggggaaggaagccgattcctttctgctagtataaacgaggccctgaacaacaatgggatcctgtcaagtgttggaaagttctccactgttaacccagtctatgttggcaatgtggcctgggcccacattctggccttgagggccctgcaggaccccaagaaggccccaagcatccgaggacagttctactatatctcagatgacacgcctcaccaaagctatgataaccttaattacaccctgagcaaagagttcggcctccgccttgattccagatggagctttcctttatccctgatgtattggattggcttcctgctggaaatagtgagcttcctactcaggccaatttacacctatcgaccgcccttcaaccgccacatagtcacattgtcaaatagcgtattcaccttctcttataagaaggctcagcgagatctggcgtataagccactctacagctgggaggaagccaagcagaaaacggtggagtgggttggttcccttgtggaccggcacaaggagaccctgaagtccaagactcagtga

SEQ ID NO: 937 - DNA, 21 bp, Homo sapiens
aaggagaaggagctgaaggag

SEQ ID NO: 938 - DNA, 21 bp, Homo sapiens
ggagaaggagcugaaggagtt

SEQ ID NO: 939 - DNA, 21 bp, Homo sapiens
cuccuucagcuccuucucctt

SEQ ID NO: 940 - DNA, 21 bp, Homo sapiens
aaggagctgaaggagatcagg

SEQ ID NO: 941 - DNA, 21 bp, Homo sapiens
ggagcugaaggagaucaggtt

SEQ ID NO: 942 - DNA, 21 bp, Homo sapiens
ccugaucuccuucagcucctt

SEQ ID NO: 943 - DNA, 21 bp, Homo sapiens
aaggagatcagggtcttggac

SEQ ID NO: 944 - DNA, 21 bp, Homo sapiens
ggagaucagggucuuggactt

SEQ ID NO: 945 - DNA, 21 bp, Homo sapiens
guccaagacccugaucucctt

SEQ ID NO: 946 - DNA, 18 bp, Homo sapiens
aaggccttcggaccagaa

SEQ ID NO: 947 - DNA, 18 bp, Homo sapiens
ggccuucggaccagaatt

SEQ ID NO: 948 - DNA, 18 bp, Homo sapiens
uucugguccgaaggcctt

SEQ ID NO: 949 - DNA, 21 bp, Homo sapiens
aattgagagaggaattttcta

SEQ ID NO: 950 - DNA, 21 bp, Homo sapiens
uugagagaggaauuuucuatt

SEQ ID NO: 951 - DNA, 21 bp, Homo sapiens
uagaaaauuccucucucaatt

SEQ ID NO: 952 - DNA, 21 bp, Homo sapiens
aattttctaaactccagaaca

SEQ ID NO: 953 - DNA, 21 bp, Homo sapiens
uuuucuaaacuccagaacatt

SEQ ID NO: 954 - DNA, 21 bp, Homo sapiens
uguucuggaguuuagaaaatt

SEQ ID NO: 955 - DNA, 21 bp, Homo sapiens
aaactccagaacaagaccaag

SEQ ID NO: 956 - DNA, 21 bp, Homo sapiens
acuccagaacaagaccaagtt

SEQ ID NO: 957 - DNA, 21 bp, Homo sapiens
cuuggucuuguucuggagutt

SEQ ID NO: 958 - DNA, 21 bp, Homo sapiens
aacaagaccaagctgacagtg

SEQ ID NO: 959 - DNA, 21 bp, Homo sapiens
caagaccaagcugacagugtt

SEQ ID NO: 960 - DNA, 21 bp, Homo sapiens
cacugucagcuuggucuugtt

SEQ ID NO: 961 - DNA, 21 bp, Homo sapiens
aagaccaagctgacagtgctg

SEQ ID NO: 962 - DNA, 21 bp, Homo sapiens
gaccaagcugacagugcugtt

SEQ ID NO: 963 - DNA, 21 bp, Homo sapiens
cagcacugucagcuugguctt

SEQ ID NO: 964 - DNA, 18 bp, Homo sapiens
aagctgacagtgctggaa

SEQ ID NO: 965 - DNA, 18 bp, Homo sapiens
gcugacagugcuggaatt

SEQ ID NO: 966 - DNA, 18 bp, Homo sapiens
uuccagcacugucagctt

SEQ ID NO: 967 - DNA, 18 bp, Homo sapiens
aaggagacattctggatg

SEQ ID NO: 968 - DNA, 18 bp, Homo sapiens
ggagacauucuggaugtt

SEQ ID NO: 969 - DNA, 18 bp, Homo sapiens
cauccagaaugucucctt

SEQ ID NO: 970 - DNA, 21 bp, Homo sapiens
aagagagcctgccaggacgtc

SEQ ID NO: 971 - DNA, 21 bp, Homo sapiens
gagagccugccaggacguctt

SEQ ID NO: 972 - DNA, 21 bp, Homo sapiens
gacguccuggcaggcucuctt

SEQ ID NO: 973 - DNA, 18 bp, Homo sapiens
aatgtcaatgtgaaaggt

SEQ ID NO: 974 - DNA, 18 bp, Homo sapiens
ugucaaugugaaaggutt

SEQ ID NO: 975 - DNA, 18 bp, Homo sapiens
accuuucacauugacatt

SEQ ID NO: 976 - DNA, 21 bp, Homo sapiens
aatgtgaaaggtacccagctc

SEQ ID NO: 977 - DNA, 21 bp, Homo sapiens
ugugaaagguacccagcuctt

SEQ ID NO: 978 - DNA, 21 bp, Homo sapiens
gagcuggguaccuuucacatt

SEQ ID NO: 979 - DNA, 21 bp, Homo sapiens
aaaggtacccagctcctgtta

SEQ ID NO: 980 - DNA, 21 bp, Homo sapiens
agguacccagcuccuguuatt

SEQ ID NO: 981 - DNA, 21 bp, Homo sapiens
uaacaggagcuggguaccutt

SEQ ID NO: 982 - DNA, 20 bp, Homo sapiens
aagctagtgtgccagtcttc

SEQ ID NO: 983 - DNA, 20 bp, Homo sapiens
gcuagugugccagucuuctt

SEQ ID NO: 984 - DNA, 20 bp, Homo sapiens
gaagacuggcacacuagctt

SEQ ID NO: 985 - DNA, 21 bp, Homo sapiens
aactcctacaaggaaatcatc

SEQ ID NO: 986 - DNA, 21 bp, Homo sapiens
cuccuacaaggaaaucauctt

SEQ ID NO: 987 - DNA, 21 bp, Homo sapiens
gaugauuuccuuguaggagtt

SEQ ID NO: 988 - DNA, 18 bp, Homo sapiens
aaggaaatcatccagaat

SEQ ID NO: 989 - DNA, 18 bp, Homo sapiens
ggaaaucauccagaautt

SEQ ID NO: 990 - DNA, 18 bp, Homo sapiens
auucuggaugauuucctt

SEQ ID NO: 991 - DNA, 18 bp, Homo sapiens
aaatcatccagaatggcc

SEQ ID NO: 992 - DNA, 18 bp, Homo sapiens
aucauccagaauggcctt

SEQ ID NO: 993 - DNA, 18 bp, Homo sapiens
ggccauucuggaugautt

SEQ ID NO: 994 - DNA, 18 bp, Homo sapiens
aatggccatgaagaagag

SEQ ID NO: 995 - DNA, 18 bp, Homo sapiens
uggccaugaagaagagtt

SEQ ID NO: 996 - DNA, 18 bp, Homo sapiens
cucuucuucauggccatt

SEQ ID NO: 997 - DNA, 21 bp, Homo sapiens
aagaagagcctctggaaaaca

SEQ ID NO: 998 - DNA, 21 bp, Homo sapiens
gaagagccucuggaaaacatt

SEQ ID NO: 999 - DNA, 21 bp, Homo sapiens
uguuuuccagaggcucuuctt

SEQ ID NO: 1000 - DNA, 21 bp, Homo sapiens
aagagcctctggaaaacacat

SEQ ID NO: 1001 - DNA, 21 bp, Homo sapiens
gagccucuggaaaacacautt

SEQ ID NO: 1002 - DNA, 21 bp, Homo sapiens
auguguuuuccagaggcuctt

SEQ ID NO: 1003 - DNA, 21 bp, Homo sapiens
aaaacacatggcccgctccat

SEQ ID NO: 1004 - DNA, 21 bp, Homo sapiens
aacacauggcccgcuccautt

SEQ ID NO: 1005 - DNA, 21 bp, Homo sapiens
auggagcgggccauguguutt

SEQ ID NO: 1006 - DNA, 21 bp, Homo sapiens
aacacatggcccgctccatac

SEQ ID NO: 1007 - DNA, 21 bp, Homo sapiens
cacauggcccgcuccauactt

SEQ ID NO: 1008 - DNA, 21 bp, Homo sapiens
guauggagcgggccaugugtt

SEQ ID NO: 1009 - DNA, 18 bp, Homo sapiens
aaaaagcttgctgagaag

SEQ ID NO: 1010 - DNA, 18 bp, Homo sapiens
aaagcuugcugagaagtt

SEQ ID NO: 1011 - DNA, 18 bp, Homo sapiens
cuucucagcaagcuuutt

SEQ ID NO: 1012 - DNA, 18 bp, Homo sapiens
aaagcttgctgagaaggc

SEQ ID NO: 1013 - DNA, 18 bp, Homo sapiens
agcuugcugagaaggctt

SEQ ID NO: 1014 - DNA, 18 bp, Homo sapiens
gccuucucagcaagcutt

SEQ ID NO: 1015 - DNA, 21 bp, Homo sapiens
aaggctgtactggcggctaac

SEQ ID NO: 1016 - DNA, 21 bp, Homo sapiens
ggcuguacuggcggcuaactt

SEQ ID NO: 1017 - DNA, 21 bp, Homo sapiens
guuagccgccaguacagcctt

SEQ ID NO: 1018 - DNA, 21 bp, Homo sapiens
aacgggtggaatctgaaaaac

SEQ ID NO: 1019 - DNA, 21 bp, Homo sapiens
cggguggaaucugaaaaactt

SEQ ID NO: 1020 - DNA, 21 bp, Homo sapiens
guuuuucagauuccacccgtt

SEQ ID NO: 1021 - DNA, 21 bp, Homo sapiens
aatctgaaaaacggcggcacc

SEQ ID NO: 1022 - DNA, 21 bp, Homo sapiens
ucugaaaaacggcggcacctt

SEQ ID NO: 1023 - DNA, 21 bp, Homo sapiens
ggugccgccguuuuucagatt

SEQ ID NO: 1024 - DNA, 18 bp, Homo sapiens
aaaaacggcggcaccctg

SEQ ID NO: 1025 - DNA, 18 bp, Homo sapiens
aaacggcggcacccugtt

SEQ ID NO: 1026 - DNA, 18 bp, Homo sapiens
cagggugccgccguuutt

SEQ ID NO: 1027 - DNA, 18 bp, Homo sapiens
aaacggcggcaccctgta

SEQ ID NO: 1028 - DNA, 18 bp, Homo sapiens
acggcggcacccuguatt

SEQ ID NO: 1029 - DNA, 18 bp, Homo sapiens
uacagggugccgccgutt

SEQ ID NO: 1030 - DNA, 21 bp, Homo sapiens
aaggaagccgattcctttctg

SEQ ID NO: 1031 - DNA, 21 bp, Homo sapiens
ggaagccgauuccuuucugtt

SEQ ID NO: 1032 - DNA, 21 bp, Homo sapiens
cagaaaggaaucggcuucctt

SEQ ID NO: 1033 - DNA, 19 bp, Homo sapiens
aagccgattcctttctgct

SEQ ID NO: 1034 - DNA, 19 bp, Homo sapiens
gccgauuccuuucugcutt

SEQ ID NO: 1035 - DNA, 19 bp, Homo sapiens
agcagaaaggaaucggctt

SEQ ID NO: 1036 - DNA, 21 bp, Homo sapiens
aaacgaggccctgaacaacaa

SEQ ID NO: 1037 - DNA, 21 bp, Homo sapiens
acgaggcccugaacaacaatt

SEQ ID NO: 1038 - DNA, 21 bp, Homo sapiens
uuguuguucagggccucgutt

SEQ ID NO: 1039 - DNA, 21 bp, Homo sapiens
aacaacaatgggatcctgtca

SEQ ID NO: 1040 - DNA, 21 bp, Homo sapiens
caacaaugggauccugucatt

SEQ ID NO: 1041 - DNA, 21 bp, Homo sapiens
ugacaggaucccauuguugtt

SEQ ID NO: 1042 - DNA, 21 bp, Homo sapiens
aacaatgggatcctgtcaagt

SEQ ID NO: 1043 - DNA, 21 bp, Homo sapiens
caaugggauccugucaagutt

SEQ ID NO: 1044 - DNA, 21 bp, Homo sapiens
acuugacaggaucccauugtt

SEQ ID NO: 1045 - DNA, 21 bp, Homo sapiens
aatgggatcctgtcaagtgtt

SEQ ID NO: 1046 - DNA, 21 bp, Homo sapiens
ugggauccugucaaguguutt

SEQ ID NO: 1047 - DNA, 21 bp, Homo sapiens
aacacuugacaggaucccatt

SEQ ID NO: 1048 - DNA, 21 bp, Homo sapiens
aagtgttggaaagttctccac

SEQ ID NO: 1049 - DNA, 21 bp, Homo sapiens
guguuggaaaguucuccactt

SEQ ID NO: 1050 - DNA, 21 bp, Homo sapiens
guggagaacuuuccaacactt

SEQ ID NO: 1051 - DNA, 18 bp, Homo sapiens
aaagttctccactgttaa

SEQ ID NO: 1052 - DNA, 18 bp, Homo sapiens
aguucuccacuguuaatt

SEQ ID NO: 1053 - DNA, 18 bp, Homo sapiens
uuaacaguggagaacutt

SEQ ID NO: 1054 - DNA, 21 bp, Homo sapiens
aacccagtctatgttggcaat

SEQ ID NO: 1055 - DNA, 21 bp, Homo sapiens
cccagucuauguuggcaautt

SEQ ID NO: 1056 - DNA, 21 bp, Homo sapiens
auugccaacauagacugggtt

SEQ ID NO: 1057 - DNA, 21 bp, Homo sapiens
aatgtggcctgggcccacatt

SEQ ID NO: 1058 - DNA, 21 bp, Homo sapiens
uguggccugggcccacauutt

SEQ ID NO: 1059 - DNA, 21 bp, Homo sapiens
aaugugggcccaggccacatt

SEQ ID NO: 1060 - DNA, 21 bp, Homo sapiens
aagaaggccccaagcatccga

SEQ ID NO: 1061 - DNA, 21 bp, Homo sapiens
gaaggccccaagcauccgatt

SEQ ID NO: 1062 - DNA, 21 bp, Homo sapiens
ucggaugcuuggggccuuctt

SEQ ID NO: 1063 - DNA, 21 bp, Homo sapiens
aaggccccaagcatccgagga

SEQ ID NO: 1064 - DNA, 21 bp, Homo sapiens
ggccccaagcauccgaggatt

SEQ ID NO: 1065 - DNA, 21 bp, Homo sapiens
uccucggaugcuuggggcctt

SEQ ID NO: 1066 - DNA, 21 bp, Homo sapiens
aagcatccgaggacagttcta

SEQ ID NO: 1067 - DNA, 21 bp, Homo sapiens
gcauccgaggacaguucuatt

SEQ ID NO: 1068 - DNA, 21 bp, Homo sapiens
uagaacuguccucggaugctt

SEQ ID NO: 1069 - DNA, 21 bp, Homo sapiens
aaagctatgataaccttaatt

SEQ ID NO: 1070 - DNA, 21 bp, Homo sapiens
agcuaugauaaccuuaauutt

SEQ ID NO: 1071 - DNA, 21 bp, Homo sapiens
aauuaagguuaucauagcutt

SEQ ID NO: 1072 - DNA, 21 bp, Homo sapiens
aaccttaattacaccctgagc

SEQ ID NO: 1073 - DNA, 21 bp, Homo sapiens
ccuuaauuacacccugagctt

SEQ ID NO: 1074 - DNA, 21 bp, Homo sapiens
gcucaggguguaauuaaggtt

SEQ ID NO: 1075 - DNA, 21 bp, Homo sapiens
aattacaccctgagcaaagag

SEQ ID NO: 1076 - DNA, 21 bp, Homo sapiens
uuacacccugagcaaagagtt

SEQ ID NO: 1077 - DNA, 21 bp, Homo sapiens
cucuuugcucaggguguaatt

SEQ ID NO: 1078 - DNA, 21 bp, Homo sapiens
aaagagttcggcctccgcctt

SEQ ID NO: 1079 - DNA, 21 bp, Homo sapiens
agaguucggccuccgccuutt

SEQ ID NO: 1080 - DNA, 21 bp, Homo sapiens
aaggcggaggccgaacucutt

SEQ ID NO: 1081 - DNA, 18 bp, Homo sapiens
aaatagtgagcttcctac

SEQ ID NO: 1082 - DNA, 18 bp, Homo sapiens
auagugagcuuccuactt

SEQ ID NO: 1083 - DNA, 18 bp, Homo sapiens
guaggaagcucacuautt

SEQ ID NO: 1084 - DNA, 21 bp, Homo sapiens
aatttacacctatcgaccgcc

SEQ ID NO: 1085 - DNA, 21 bp, Homo sapiens
uuuacaccuaucgaccgcctt

SEQ ID NO: 1086 - DNA, 21 bp, Homo sapiens
ggcggucgauagguguaaatt

SEQ ID NO: 1087 - DNA, 18 bp, Homo sapiens
aaccgccacatagtcaca

SEQ ID NO: 1088 - DNA, 18 bp, Homo sapiens
ccgccacauagucacatt

SEQ ID NO: 1089 - DNA, 18 bp, Homo sapiens
ugugacuauguggcggtt

SEQ ID NO: 1090 - DNA, 21 bp, Homo sapiens
aaatagcgtattcaccttctc

SEQ ID NO: 1091 - DNA, 21 bp, Homo sapiens
auagcguauucaccuucuctt

SEQ ID NO: 1092 - DNA, 21 bp, Homo sapiens
gagaaggugaauacgcuautt

SEQ ID NO: 1093 - DNA, 21 bp, Homo sapiens
aagaaggctcagcgagatctg

SEQ ID NO: 1094 - DNA, 21 bp, Homo sapiens
gaaggcucagcgagaucugtt

SEQ ID NO: 1095 - DNA, 21 bp, Homo sapiens
cagaucucgcugagccuuctt

SEQ ID NO: 1096 - DNA, 21 bp, Homo sapiens
aaggctcagcgagatctggcg

SEQ ID NO: 1097 - DNA, 21 bp, Homo sapiens
ggcucagcgagaucuggcgtt

SEQ ID NO: 1098 - DNA, 21 bp, Homo sapiens
cgccagaucucgcugagcctt

SEQ ID NO: 1099 - DNA, 21 bp, Homo sapiens
aagccactctacagctgggag

SEQ ID NO: 1100 - DNA, 21 bp, Homo sapiens
gccacucuacagcugggagtt

SEQ ID NO: 1101 - DNA, 21 bp, Homo sapiens
cucccagcuguagaguggctt

SEQ ID NO: 1102 - DNA, 21 bp, Homo sapiens
aagccaagcagaaaacggtgg

SEQ ID NO: 1103 - DNA, 21 bp, Homo sapiens
gccaagcagaaaacgguggtt

SEQ ID NO: 1104 - DNA, 21 bp, Homo sapiens
ccaccguuuucugcuuggctt

SEQ ID NO: 1105 - DNA, 21 bp, Homo sapiens
aagcagaaaacggtggagtgg

SEQ ID NO: 1106 - DNA, 21 bp, Homo sapiens
gcagaaaacgguggaguggtt

SEQ ID NO: 1107 - DNA, 21 bp, Homo sapiens
ccacuccaccguuuucugctt

SEQ ID NO: 1108 - DNA, 21 bp, Homo sapiens
aaaacggtggagtgggttggt

SEQ ID NO: 1109 - DNA, 21 bp, Homo sapiens
aacgguggaguggguuggutt

SEQ ID NO: 1110 - DNA, 21 bp, Homo sapiens
accaacccacuccaccguutt

SEQ ID NO: 1111 - DNA, 21 bp, Homo sapiens
aacggtggagtgggttggttc

SEQ ID NO: 1112 - DNA, 21 bp, Homo sapiens
cgguggaguggguugguuctt

SEQ ID NO: 1113 - DNA, 21 bp, Homo sapiens
gaaccaacccacuccaccgtt

SEQ ID NO: 1114 - DNA, 21 bp, Homo sapiens
aaggagaccctgaagtccaag

SEQ ID NO: 1115 - DNA, 21 bp, Homo sapiens
ggagacccugaaguccaagtt

SEQ ID NO: 1116 - DNA, 21 bp, Homo sapiens
cuuggacuucagggucucctt

SEQ ID NO: 1117 - DNA, 1122 bp, Homo sapiens; identical to SEQ ID NO: 936

SEQ ID NO: 1118 - DNA, 21 bp, Homo sapiens
aaggagaaggagctgaaggag

SEQ ID NO: 1119 - DNA, 21 bp, Homo sapiens
ggagaaggagcugaaggagtt

SEQ ID NO: 1120 - DNA, 21 bp, Homo sapiens
cuccuucagcuccuucucctt

SEQ ID NO: 1121 - DNA, 21 bp, Homo sapiens
aaggagctgaaggagatcagg

SEQ ID NO: 1122 - DNA, 21 bp, Homo sapiens
ggagcugaaggagaucaggtt

SEQ ID NO: 1123 - DNA, 21 bp, Homo sapiens
ccugaucuccuucagcucctt

SEQ ID NO: 1124 - DNA, 21 bp, Homo sapiens
aaggagatcagggtcttggac

SEQ ID NO: 1125 - DNA, 21 bp, Homo sapiens
ggagaucagggucuuggactt

SEQ ID NO: 1126 - DNA, 21 bp, Homo sapiens
guccaagacccugaucucctt

SEQ ID NO: 1127 - DNA, 18 bp, Homo sapiens
aaggccttcggaccagaa

SEQ ID NO: 1128 - DNA, 18 bp, Homo sapiens
ggccuucggaccagaatt

SEQ ID NO: 1129 - DNA, 18 bp, Homo sapiens
uucugguccgaaggcctt

SEQ ID NO: 1130 - DNA, 21 bp, Homo sapiens
aattgagagaggaattttcta

SEQ ID NO: 1131 - DNA, 21 bp, Homo sapiens
uugagagaggaauuuucuatt

SEQ ID NO: 1132 - DNA, 21 bp, Homo sapiens
uagaaaauuccucucucaatt

SEQ ID NO: 1133 - DNA, 21 bp, Homo sapiens
aattttctaaactccagaaca

SEQ ID NO: 1134 - DNA, 21 bp, Homo sapiens
uuuucuaaacuccagaacatt

SEQ ID NO: 1135 - DNA, 21 bp, Homo sapiens
uguucuggaguuuagaaaatt

SEQ ID NO: 1136 - DNA, 21 bp, Homo sapiens
aaactccagaacaagaccaag

SEQ ID NO: 1137 - DNA, 21 bp, Homo sapiens
acuccagaacaagaccaagtt

SEQ ID NO: 1138 - DNA, 21 bp, Homo sapiens
cuuggucuuguucuggagutt

SEQ ID NO: 1139 - DNA, 21 bp, Homo sapiens
aacaagaccaagctgacagtg

SEQ ID NO: 1140 - DNA, 21 bp, Homo sapiens
caagaccaagcugacagugtt

SEQ ID NO: 1141 - DNA, 21 bp, Homo sapiens
cacugucagcuuggucuugtt

SEQ ID NO: 1142 - DNA, 21 bp, Homo sapiens
aagaccaagctgacagtgctg

SEQ ID NO: 1143 - DNA, 21 bp, Homo sapiens
gaccaagcugacagugcugtt

SEQ ID NO: 1144 - DNA, 21 bp, Homo sapiens
cagcacugucagcuugguctt

SEQ ID NO: 1145 - DNA, 18 bp, Homo sapiens
aagctgacagtgctggaa

SEQ ID NO: 1146 - DNA, 18 bp, Homo sapiens
gcugacagugcuggaatt

SEQ ID NO: 1147 - DNA, 18 bp, Homo sapiens
uuccagcacugucagctt

SEQ ID NO: 1148 - DNA, 18 bp, Homo sapiens
aaggagacattctggatg

SEQ ID NO: 1149 - DNA, 18 bp, Homo sapiens
ggagacauucuggaugtt

SEQ ID NO: 1150 - DNA, 18 bp, Homo sapiens
cauccagaaugucucctt

SEQ ID NO: 1151 - DNA, 21 bp, Homo sapiens
aagagagcctgccaggacgtc

SEQ ID NO: 1152 - DNA, 21 bp, Homo sapiens
gagagccugccaggacguctt

SEQ ID NO: 1153 - DNA, 21 bp, Homo sapiens
gacguccuggcaggcucuctt

SEQ ID NO: 1154 - DNA, 18 bp, Homo sapiens
aatgtcaatgtgaaaggt

SEQ ID NO: 1155 - DNA, 18 bp, Homo sapiens
ugucaaugugaaaggutt

SEQ ID NO: 1156 - DNA, 18 bp, Homo sapiens
accuuucacauugacatt

SEQ ID NO: 1157 - DNA, 21 bp, Homo sapiens
aatgtgaaaggtacccagctc

SEQ ID NO: 1158 - DNA, 21 bp, Homo sapiens
ugugaaagguacccagcuctt

SEQ ID NO: 1159 - DNA, 21 bp, Homo sapiens
gagcuggguaccuuucacatt

SEQ ID NO: 1160 - DNA, 21 bp, Homo sapiens
aaaggtacccagctcctgtta

SEQ ID NO: 1161 - DNA, 21 bp, Homo sapiens
agguacccagcuccuguuatt

SEQ ID NO: 1162 - DNA, 21 bp, Homo sapiens
uaacaggagcuggguaccutt

SEQ ID NO: 1163 - DNA, 20 bp, Homo sapiens
aagctagtgtgccagtcttc

SEQ ID NO: 1164 - DNA, 20 bp, Homo sapiens
gcuagugugccagucuuctt

SEQ ID NO: 1165 - DNA, 20 bp, Homo sapiens
gaagacuggcacacuagctt

SEQ ID NO: 1166 - DNA, 21 bp, Homo sapiens
aactcctacaaggaaatcatc

SEQ ID NO: 1167 - DNA, 21 bp, Homo sapiens
cuccuacaaggaaaucauctt

SEQ ID NO: 1168 - DNA, 21 bp, Homo sapiens
gaugauuuccuuguaggagtt

SEQ ID NO: 1169 - DNA, 18 bp, Homo sapiens
aaggaaatcatccagaat

SEQ ID NO: 1170 - DNA, 18 bp, Homo sapiens
ggaaaucauccagaautt

SEQ ID NO: 1171 - DNA, 18 bp, Homo sapiens
auucuggaugauuucctt

SEQ ID NO: 1172 - DNA, 18 bp, Homo sapiens
aaatcatccagaatggcc

SEQ ID NO: 1173 - DNA, 18 bp, Homo sapiens
aucauccagaauggcctt

SEQ ID NO: 1174 - DNA, 18 bp, Homo sapiens
ggccauucuggaugautt

SEQ ID NO: 1175 - DNA, 18 bp, Homo sapiens
aatggccatgaagaagag

SEQ ID NO: 1176 - DNA, 18 bp, Homo sapiens
uggccaugaagaagagtt

SEQ ID NO: 1177 - DNA, 18 bp, Homo sapiens
cucuucuucauggccatt

SEQ ID NO: 1178 - DNA, 21 bp, Homo sapiens
aagaagagcctctggaaaaca

SEQ ID NO: 1179 - DNA, 21 bp, Homo sapiens
gaagagccucuggaaaacatt

SEQ ID NO: 1180 - DNA, 21 bp, Homo sapiens
uguuuuccagaggcucuuctt

SEQ ID NO: 1181 - DNA, 21 bp, Homo sapiens
aagagcctctggaaaacacat

SEQ ID NO: 1182 - DNA, 21 bp, Homo sapiens
gagccucuggaaaacacautt

SEQ ID NO: 1183 - DNA, 21 bp, Homo sapiens
auguguuuuccagaggcuctt

SEQ ID NO: 1184 - DNA, 21 bp, Homo sapiens
aaaacacatggcccgctccat

SEQ ID NO: 1185 - DNA, 21 bp, Homo sapiens
aacacauggcccgcuccautt

SEQ ID NO: 1186 - DNA, 21 bp, Homo sapiens
auggagcgggccauguguutt

SEQ ID NO: 1187 - DNA, 21 bp, Homo sapiens
aacacatggcccgctccatac

SEQ ID NO: 1188 - DNA, 21 bp, Homo sapiens
cacauggcccgcuccauactt

SEQ ID NO: 1189 - DNA, 21 bp, Homo sapiens
guauggagcgggccaugugtt

SEQ ID NO: 1190 - DNA, 18 bp, Homo sapiens
aaaaagcttgctgagaag

SEQ ID NO: 1191 - DNA, 18 bp, Homo sapiens
aaagcuugcugagaagtt

SEQ ID NO: 1192 - DNA, 18 bp, Homo sapiens
cuucucagcaagcuuutt

SEQ ID NO: 1193 - DNA, 18 bp, Homo sapiens
aaagcttgctgagaaggc

SEQ ID NO: 1194 - DNA, 18 bp, Homo sapiens
agcuugcugagaaggctt

SEQ ID NO: 1195 - DNA, 18 bp, Homo sapiens
gccuucucagcaagcutt

SEQ ID NO: 1196 - DNA, 21 bp, Homo sapiens
aaggctgtactggcggctaac

SEQ ID NO: 1197 - DNA, 21 bp, Homo sapiens
ggcuguacuggcggcuaactt

SEQ ID NO: 1198 - DNA, 21 bp, Homo sapiens
guuagccgccaguacagcctt

SEQ ID NO: 1199 - DNA, 21 bp, Homo sapiens
aacgggtggaatctgaaaaac

SEQ ID NO: 1200 - DNA, 21 bp, Homo sapiens
cggguggaaucugaaaaactt

SEQ ID NO: 1201 - DNA, 21 bp, Homo sapiens
guuuuucagauuccacccgtt

SEQ ID NO: 1202 - DNA, 21 bp, Homo sapiens
aatctgaaaaacggcggcacc

SEQ ID NO: 1203 - DNA, 21 bp, Homo sapiens
ucugaaaaacggcggcacctt

SEQ ID NO: 1204 - DNA, 21 bp, Homo sapiens
ggugccgccguuuuucagatt

SEQ ID NO: 1205 - DNA, 18 bp, Homo sapiens
aaaaacggcggcaccctg

SEQ ID NO: 1206 - DNA, 18 bp, Homo sapiens
aaacggcggcacccugtt

SEQ ID NO: 1207 - DNA, 18 bp, Homo sapiens
cagggugccgccguuutt

SEQ ID NO: 1208 - DNA, 18 bp, Homo sapiens
aaacggcggcaccctgta

SEQ ID NO: 1209 - DNA, 18 bp, Homo sapiens
acggcggcacccuguatt

SEQ ID NO: 1210 - DNA, 18 bp, Homo sapiens
uacagggugccgccgutt

SEQ ID NO: 1211 - DNA, 21 bp, Homo sapiens
aaggaagccgattcctttctg

SEQ ID NO: 1212 - DNA, 21 bp, Homo sapiens
ggaagccgauuccuuucugtt

SEQ ID NO: 1213 - DNA, 21 bp, Homo sapiens
cagaaaggaaucggcuucctt

SEQ ID NO: 1214 - DNA, 19 bp, Homo sapiens
aagccgattcctttctgct

SEQ ID NO: 1215 - DNA, 19 bp, Homo sapiens
gccgauuccuuucugcutt

SEQ ID NO: 1216 - DNA, 19 bp, Homo sapiens
agcagaaaggaaucggctt

SEQ ID NO: 1217 - DNA, 21 bp, Homo sapiens
aaacgaggccctgaacaacaa

SEQ ID NO: 1218 - DNA, 21 bp, Homo sapiens
acgaggcccugaacaacaatt

SEQ ID NO: 1219 - DNA, 21 bp, Homo sapiens
uuguuguucagggccucgutt

SEQ ID NO: 1220 - DNA, 21 bp, Homo sapiens
aacaacaatgggatcctgtca

SEQ ID NO: 1221 - DNA, 21 bp, Homo sapiens
caacaaugggauccugucatt

SEQ ID NO: 1222 - DNA, 21 bp, Homo sapiens
ugacaggaucccauuguugtt

SEQ ID NO: 1223 - DNA, 21 bp, Homo sapiens
aacaatgggatcctgtcaagt

SEQ ID NO: 1224 - DNA, 21 bp, Homo sapiens
caaugggauccugucaagutt

SEQ ID NO: 1225 - DNA, 21 bp, Homo sapiens
acuugacaggaucccauugtt

SEQ ID NO: 1226 - DNA, 21 bp, Homo sapiens
aatgggatcctgtcaagtgtt

SEQ ID NO: 1227 - DNA, 21 bp, Homo sapiens
ugggauccugucaaguguutt

SEQ ID NO: 1228 - DNA, 21 bp, Homo sapiens
aacacuugacaggaucccatt

SEQ ID NO: 1229 - DNA, 21 bp, Homo sapiens
aagtgttggaaagttctccac

SEQ ID NO: 1230 - DNA, 21 bp, Homo sapiens
guguuggaaaguucuccactt

SEQ ID NO: 1231 - DNA, 21 bp, Homo sapiens
guggagaacuuuccaacactt

SEQ ID NO: 1232 - DNA, 18 bp, Homo sapiens
aaagttctccactgttaa

SEQ ID NO: 1233 - DNA, 18 bp, Homo sapiens
aguucuccacuguuaatt

SEQ ID NO: 1234 - DNA, 18 bp, Homo sapiens
uuaacaguggagaacutt

SEQ ID NO: 1235 - DNA, 21 bp, Homo sapiens
aacccagtctatgttggcaat

SEQ ID NO: 1236 - DNA, 21 bp, Homo sapiens
cccagucuauguuggcaautt

SEQ ID NO: 1237 - DNA, 21 bp, Homo sapiens
auugccaacauagacugggtt

SEQ ID NO: 1238 - DNA, 21 bp, Homo sapiens
aatgtggcctgggcccacatt

SEQ ID NO: 1239 - DNA, 21 bp, Homo sapiens
uguggccugggcccacauutt

SEQ ID NO: 1240 - DNA, 21 bp, Homo sapiens
aaugugggcccaggccacatt

SEQ ID NO: 1241 - DNA, 21 bp, Homo sapiens
aagaaggccccaagcatccga

SEQ ID NO: 1242 - DNA, 21 bp, Homo sapiens
gaaggccccaagcauccgatt

SEQ ID NO: 1243 - DNA, 21 bp, Homo sapiens
ucggaugcuuggggccuuctt

SEQ ID NO: 1244 - DNA, 21 bp, Homo sapiens
aaggccccaagcatccgagga

SEQ ID NO: 1245 - DNA, 21 bp, Homo sapiens
ggccccaagcauccgaggatt

SEQ ID NO: 1246 - DNA, 21 bp, Homo sapiens
uccucggaugcuuggggcctt

SEQ ID NO: 1247 - DNA, 21 bp, Homo sapiens
aagcatccgaggacagttcta

SEQ ID NO: 1248 - DNA, 21 bp, Homo sapiens
gcauccgaggacaguucuatt

SEQ ID NO: 1249 - DNA, 21 bp, Homo sapiens
uagaacuguccucggaugctt

SEQ ID NO: 1250 - DNA, 21 bp, Homo sapiens
aaagctatgataaccttaatt

SEQ ID NO: 1251 - DNA, 21 bp, Homo sapiens
agcuaugauaaccuuaauutt

SEQ ID NO: 1252 - DNA, 21 bp, Homo sapiens
aauuaagguuaucauagcutt

SEQ ID NO: 1253 - DNA, 21 bp, Homo sapiens
aaccttaattacaccctgagc

SEQ ID NO: 1254 - DNA, 21 bp, Homo sapiens
ccuuaauuacacccugagctt

SEQ ID NO: 1255 - DNA, 21 bp, Homo sapiens
gcucaggguguaauuaaggtt

SEQ ID NO: 1256 - DNA, 21 bp, Homo sapiens
aattacaccctgagcaaagag

SEQ ID NO: 1257 - DNA, 21 bp, Homo sapiens
uuacacccugagcaaagagtt

SEQ ID NO: 1258 - DNA, 21 bp, Homo sapiens
cucuuugcucaggguguaatt

SEQ ID NO: 1259 - DNA, 21 bp, Homo sapiens
aaagagttcggcctccgcctt

SEQ ID NO: 1260 - DNA, 21 bp, Homo sapiens
agaguucggccuccgccuutt

SEQ ID NO: 1261 - DNA, 21 bp, Homo sapiens
aaggcggaggccgaacucutt

SEQ ID NO: 1262 - DNA, 18 bp, Homo sapiens
aaatagtgagcttcctac

SEQ ID NO: 1263 - DNA, 18 bp, Homo sapiens
auagugagcuuccuactt

SEQ ID NO: 1264 - DNA, 18 bp, Homo sapiens
guaggaagcucacuautt

SEQ ID NO: 1265 - DNA, 21 bp, Homo sapiens
aatttacacctatcgaccgcc

SEQ ID NO: 1266 - DNA, 21 bp, Homo sapiens
uuuacaccuaucgaccgcctt

SEQ ID NO: 1267 - DNA, 21 bp, Homo sapiens
ggcggucgauagguguaaatt

SEQ ID NO: 1268 - DNA, 18 bp, Homo sapiens
aaccgccacatagtcaca

SEQ ID NO: 1269 - DNA, 18 bp, Homo sapiens
ccgccacauagucacatt

SEQ ID NO: 1270 - DNA, 18 bp, Homo sapiens
ugugacuauguggcggtt

SEQ ID NO: 1271 - DNA, 21 bp, Homo sapiens
aaatagcgtattcaccttctc

SEQ ID NO: 1272 - DNA, 21 bp, Homo sapiens
auagcguauucaccuucuctt

SEQ ID NO: 1273 - DNA, 21 bp, Homo sapiens
gagaaggugaauacgcuautt

SEQ ID NO: 1274 - DNA, 21 bp, Homo sapiens
aagaaggctcagcgagatctg

SEQ ID NO: 1275 - DNA, 21 bp, Homo sapiens
gaaggcucagcgagaucugtt

SEQ ID NO: 1276 - DNA, 21 bp, Homo sapiens
cagaucucgcugagccuuctt

SEQ ID NO: 1277 - DNA, 21 bp, Homo sapiens
aaggctcagcgagatctggcg

SEQ ID NO: 1278 - DNA, 21 bp, Homo sapiens
ggcucagcgagaucuggcgtt

SEQ ID NO: 1279 - DNA, 21 bp, Homo sapiens
cgccagaucucgcugagcctt

SEQ ID NO: 1280 - DNA, 21 bp, Homo sapiens
aagccactctacagctgggag

SEQ ID NO: 1281 - DNA, 21 bp, Homo sapiens
gccacucuacagcugggagtt

SEQ ID NO: 1282 - DNA, 21 bp, Homo sapiens
cucccagcuguagaguggctt

SEQ ID NO: 1283 - DNA, 21 bp, Homo sapiens
aagccaagcagaaaacggtgg

SEQ ID NO: 1284 - DNA, 21 bp, Homo sapiens
gccaagcagaaaacgguggtt

SEQ ID NO: 1285 - DNA, 21 bp, Homo sapiens
ccaccguuuucugcuuggctt

SEQ ID NO: 1286 - DNA, 21 bp, Homo sapiens
aagcagaaaacggtggagtgg

SEQ ID NO: 1287 - DNA, 21 bp, Homo sapiens
gcagaaaacgguggaguggtt

SEQ ID NO: 1288 - DNA, 21 bp, Homo sapiens
ccacuccaccguuuucugctt

SEQ ID NO: 1289 - DNA, 21 bp, Homo sapiens
aaaacggtggagtgggttggt

SEQ ID NO: 1290 - DNA, 21 bp, Homo sapiens
aacgguggaguggguuggutt

SEQ ID NO: 1291 - DNA, 21 bp, Homo sapiens
accaacccacuccaccguutt

SEQ ID NO: 1292 - DNA, 21 bp, Homo sapiens
aacggtggagtgggttggttc

SEQ ID NO: 1293 - DNA, 21 bp, Homo sapiens
cgguggaguggguugguuctt

SEQ ID NO: 1294 - DNA, 21 bp, Homo sapiens
gaaccaacccacuccaccgtt

SEQ ID NO: 1295 - DNA, 21 bp, Homo sapiens
aaggagaccctgaagtccaag

SEQ ID NO: 1296 - DNA, 21 bp, Homo sapiens
ggagacccugaaguccaagtt

SEQ ID NO: 1297 - DNA, 21 bp, Homo sapiens
cuuggacuucagggucucctt

SEQ ID NO: 1298 - DNA, 987 bp, Homo sapiens
atggcccgcaccgtggtgctcatcaccggctgttcctcgggcatcggcctgcacttggccgtacgtctggcttcagatccatcccagagcttcaaagtgtatgccacgttgagggacctgaaaacacagggccggctgtgggaggcggcccgggccctggcatgccctccgggatccctggagacgttgcagctggacgtaagggactcaaaatccgtggccgctgcccgggaacgcgtgactgagggccgcgtggacgtgctggtgtgtaacgcaggcctgggcctgctggggccgctggaggcgctgggggaggacgccgtggcctctgtgctggacgtgaatgtagtagggactgtgcggatgctgcaggccttcctgccagacatgaagaggcgcggttcgggacgcgtgttggtgaccgggagcgtgggaggattgatggggctgcctttcaatgacgtttattgcgccagcaagttcgcgctcgaaggcttatgcgagagtctggcggttctgctgctgccctttggggtccacttgagcctgatcgagtgcggcccagtgcacaccgccttcatggagaaggtgttgggcagcccagaggaggtgctggaccgcacggacatccacaccttccaccgcttctaccaatacctcgcccacagcaagcaagtctttcgcgaggcggcgcagaaccctgaggaggtggcggaggtcttcctcaccgctttgcgcgccccgaagccgaccctgcgctacttcaccaccgagcgcttcctgcccctgctgcggatgcgcctggacgaccccagcggctccaactacgtcaccgccatgcaccgggaagtgttcggcgacgttccggcaaaggccgaggctggggccgaggctgggggcggggccgggcctggggcagaggacgaggccgggcgcagtgcggtgggggaccctgagctcggcgatcctccggccgccccgcagtaa

SEQ ID NO: 1299 - DNA, 21 bp, Homo sapiens
aaagtgtatgccacgttgagg

SEQ ID NO: 1300 - DNA, 21 bp, Homo sapiens
aguguaugccacguugaggtt

SEQ ID NO: 1301 - DNA, 21 bp, Homo sapiens
ccucaacguggcauacacutt

SEQ ID NO: 1302 - DNA, 21 bp, Homo sapiens
aaaacacagggccggctgtgg

SEQ ID NO: 1303 - DNA, 21 bp, Homo sapiens
aacacagggccggcuguggtt

SEQ ID NO: 1304 - DNA, 21 bp, Homo sapiens
ccacagccggcccuguguutt

SEQ ID NO: 1305 - DNA, 21 bp, Homo sapiens
aacacagggccggctgtggga

SEQ ID NO: 1306 - DNA, 21 bp, Homo sapiens
cacagggccggcugugggatt

SEQ ID NO: 1307 - DNA, 21 bp, Homo sapiens
ucccacagccggcccugugtt

SEQ ID NO: 1308 - DNA, 21 bp, Homo sapiens
aagggactcaaaatccgtggc

SEQ ID NO: 1309 - DNA, 21 bp, Homo sapiens
gggacucaaaauccguggctt

SEQ ID NO: 1310 - DNA, 21 bp, Homo sapiens
gccacggauuuugaguccctt

SEQ ID NO: 1311 - DNA, 21 bp, Homo sapiens
aaaatccgtggccgctgcccg

SEQ ID NO: 1312 - DNA, 21 bp, Homo sapiens
aauccguggccgcugcccgtt

SEQ ID NO: 1313 - DNA, 21 bp, Homo sapiens
cgggcagcggccacggauutt

SEQ ID NO: 1314 - DNA, 21 bp, Homo sapiens
aatccgtggccgctgcccggg

SEQ ID NO: 1315 - DNA, 21 bp, Homo sapiens
uccguggccgcugcccgggtt

SEQ ID NO: 1316 - DNA, 21 bp, Homo sapiens
cccgggcagcggccacggatt

SEQ ID NO: 1317 - DNA, 18 bp, Homo sapiens
aacgcgtgactgagggcc

SEQ ID NO: 1318 - DNA, 18 bp, Homo sapiens
cgcgugacugagggcctt

SEQ ID NO: 1319 - DNA, 18 bp, Homo sapiens
ggcccucagucacgcgtt

SEQ ID NO: 1320 - DNA, 21 bp, Homo sapiens
aacgcaggcctgggcctgctg

SEQ ID NO: 1321 - DNA, 21 bp, Homo sapiens
cgcaggccugggccugcugtt

SEQ ID NO: 1322 - DNA, 21 bp, Homo sapiens
cagcaggcccaggccugcgtt

SEQ ID NO: 1323 - DNA, 18 bp, Homo sapiens
aatgtagtagggactgtg

SEQ ID NO: 1324 - DNA, 18 bp, Homo sapiens
uguaguagggacugugtt

SEQ ID NO: 1325 - DNA, 18 bp, Homo sapiens
cacagucccuacuacatt

SEQ ID NO: 1326 - DNA, 21 bp, Homo sapiens
aagaggcgcggttcgggacgc

SEQ ID NO: 1327 - DNA, 21 bp, Homo sapiens
gaggcgcgguucgggacgctt

SEQ ID NO: 1328 - DNA, 21 bp, Homo sapiens
gcgucccgaaccgcgccuctt

SEQ ID NO: 1329 - DNA, 21 bp, Homo sapiens
aatgacgtttattgcgccagc

SEQ ID NO: 1330 - DNA, 21 bp, Homo sapiens
ugacguuuauugcgccagctt

SEQ ID NO: 1331 - DNA, 21 bp, Homo sapiens
gcuggcgcaauaaacgucatt

SEQ ID NO: 1332 - DNA, 18 bp, Homo sapiens
aagttcgcgctcgaaggc

SEQ ID NO: 1333 - DNA, 18 bp, Homo sapiens
guucgcgcucgaaggctt

SEQ ID NO: 1334 - DNA, 18 bp, Homo sapiens
gccuucgagcgcgaactt

SEQ ID NO: 1335 - DNA, 21 bp, Homo sapiens
aaggcttatgcgagagtctgg

SEQ ID NO: 1336 - DNA, 21 bp, Homo sapiens
ggcuuaugcgagagucuggtt

SEQ ID NO: 1337 - DNA, 21 bp, Homo sapiens
ccagacucucgcauaagcctt

SEQ ID NO: 1338 - DNA, 18 bp, Homo sapiens
aaggtgttgggcagccca

SEQ ID NO: 1339 - DNA, 18 bp, Homo sapiens
gguguugggcagcccatt

SEQ ID NO: 1340 - DNA, 18 bp, Homo sapiens
ugggcugcccaacacctt

SEQ ID NO: 1341 - DNA, 18 bp, Homo sapiens
aatacctcgcccacagca

SEQ ID NO: 1342 - DNA, 18 bp, Homo sapiens
uaccucgcccacagcatt

SEQ ID NO: 1343 - DNA, 18 bp, Homo sapiens
ugcugugggcgagguatt

SEQ ID NO: 1344 - DNA, 21 bp, Homo sapiens
aagcaagtctttcgcgaggcg

SEQ ID NO: 1345 - DNA, 21 bp, Homo sapiens
gcaagucuuucgcgaggcgtt

SEQ ID NO: 1346 - DNA, 21 bp, Homo sapiens
cgccucgcgaaagacuugctt

SEQ ID NO: 1347 - DNA, 21 bp, Homo sapiens
aagtctttcgcgaggcggcgc

SEQ ID NO: 1348 - DNA, 21 bp, Homo sapiens
gucuuucgcgaggcggcgctt

SEQ ID NO: 1349 - DNA, 21 bp, Homo sapiens
gcgccgccucgcgaaagactt

SEQ ID NO: 1350 - DNA, 21 bp, Homo sapiens
aaccctgaggaggtggcggag

SEQ ID NO: 1351 - DNA, 21 bp, Homo sapiens
cccugaggagguggcggagtt

SEQ ID NO: 1352 - DNA, 21 bp, Homo sapiens
cuccgccaccuccucagggtt

SEQ ID NO: 1353 - DNA, 21 bp, Homo sapiens
aagccgaccctgcgctacttc

SEQ ID NO: 1354 - DNA, 21 bp, Homo sapiens
gccgacccugcgcuacuuctt

SEQ ID NO: 1355 - DNA, 21 bp, Homo sapiens
gaaguagcgcagggucggctt

SEQ ID NO: 1356 - DNA, 18 bp, Homo sapiens
aactacgtcaccgccatg

SEQ ID NO: 1357 - DNA, 18 bp, Homo sapiens
cuacgucaccgccaugtt

SEQ ID NO: 1358 - DNA, 18 bp, Homo sapiens
cauggcggugacguagtt

SEQ ID NO: 1359 - DNA, 21 bp, Homo sapiens
aagtgttcggcgacgttccgg

SEQ ID NO: 1360 - DNA, 21 bp, Homo sapiens
guguucggcgacguuccggtt

SEQ ID NO: 1361 - DNA, 21 bp, Homo sapiens
ccggaacgucgccgaacactt

SEQ ID NO: 1362 - DNA, 21 bp, Homo sapiens
aaaggccgaggctggggccga

SEQ ID NO: 1363 - DNA, 21 bp, Homo sapiens
aggccgaggcuggggccgatt

SEQ ID NO: 1364 - DNA, 21 bp, Homo sapiens
ucggccccagccucggccutt

SEQ ID NO: 1365 - DNA, 424 bp, Homo sapiens
ggaaatggcaccttgggatgagctgtcacagcaagactgacttctgtcaccaccctttacatcacggcttcaattatttctatgggatctctttgaccaatctgagagactgcaagcccggagagggcagtgtcttcaccacgggcttcaagaggctggtcttcctccccctgcagatcgtcggggtcaccctccttacccttgctgcactcaattgtctggggctactccacgtgcctctaggcgtttttttcagccttctcttcctagcagccctaatcctgacccttttcttgggcttccttcattacttccggcccctgaactgcttcatgatgaggaactacgagatcattcagcagcccatgtcctatgacaatctcacccagaggctaacggtggaggcggcccagttcatacagcg

SEQ ID NO: 1366 - DNA, 21 bp, Homo sapiens
aaatggcaccttgggatgagc

SEQ ID NO: 1367 - DNA, 21 bp, Homo sapiens
auggcaccuugggaugagctt

SEQ ID NO: 1368 - DNA, 21 bp, Homo sapiens
gcucaucccaaggugccautt

SEQ ID NO: 1369 - DNA, 21 bp, Homo sapiens
aagactgacttctgtcaccac

SEQ ID NO: 1370 - DNA, 21 bp, Homo sapiens
gacugacuucugucaccactt

SEQ ID NO: 1371 - DNA, 21 bp, Homo sapiens
guggugacagaagucaguctt

SEQ ID NO: 1372 - DNA, 21 bp, Homo sapiens
aattatttctatgggatctct

SEQ ID NO: 1373 - DNA, 21 bp, Homo sapiens
uuauuucuaugggaucucutt

SEQ ID NO: 1374 - DNA, 21 bp, Homo sapiens
agagaucccauagaaauaatt

SEQ ID NO: 1375 - DNA, 21 bp, Homo sapiens
aatctgagagactgcaagccc

SEQ ID NO: 1376 - DNA, 21 bp, Homo sapiens
ucugagagacugcaagccctt

SEQ ID NO: 1377 - DNA, 21 bp, Homo sapiens
gggcuugcagucucucagatt

SEQ ID NO: 1378 - DNA, 18 bp, Homo sapiens
aagcccggagagggcagt

SEQ ID NO: 1379 - DNA, 18 bp, Homo sapiens
gcccggagagggcagutt

SEQ ID NO: 1380 - DNA, 18 bp, Homo sapiens
acugcccucuccgggctt

SEQ ID NO: 1381 - DNA, 21 bp, Homo sapiens
aagaggctggtcttcctcccc

SEQ ID NO: 1382 - DNA, 21 bp, Homo sapiens
gaggcuggucuuccucccctt

SEQ ID NO: 1383 - DNA, 21 bp, Homo sapiens
ggggaggaagaccagccuctt

SEQ ID NO: 1384 - DNA, 21 bp, Homo sapiens
aattgtctggggctactccac

SEQ ID NO: 1385 - DNA, 21 bp, Homo sapiens
uugucuggggcuacuccactt

SEQ ID NO: 1386 - DNA, 21 bp, Homo sapiens
guggaguagccccagacaatt

SEQ ID NO: 1387 - DNA, 21 bp, Homo sapiens
aatcctgacccttttcttggg

SEQ ID NO: 1388 - DNA, 21 bp, Homo sapiens
uccugacccuuuucuugggtt

SEQ ID NO: 1389 - DNA, 21 bp, Homo sapiens
cccaagaaaagggucaggatt

SEQ ID NO: 1390 - DNA, 21 bp, Homo sapiens
aactgcttcatgatgaggaac

SEQ ID NO: 1391 - DNA, 21 bp, Homo sapiens
cugcuucaugaugaggaactt

SEQ ID NO: 1392 - DNA, 21 bp, Homo sapiens
guuccucaucaugaagcagtt

SEQ ID NO: 1393 - DNA, 19 bp, Homo sapiens
aactacgagatcattcagc

SEQ ID NO: 1394 - DNA, 19 bp, Homo sapiens
cuacgagaucauucagctt

SEQ ID NO: 1395 - DNA, 19 bp, Homo sapiens
gcugaaugaucucguagtt

SEQ ID NO: 1396 - DNA, 21 bp, Homo sapiens
aatctcacccagaggctaacg

SEQ ID NO: 1397 - DNA, 21 bp, Homo sapiens
ucucacccagaggcuaacgtt

SEQ ID NO: 1398 - DNA, 21 bp, Homo sapiens
cguuagccucugggugagatt

SEQ ID NO: 1399 - DNA, 21 bp, Homo sapiens
aacggtggaggcggcccagtt

SEQ ID NO: 1400 - DNA, 21 bp, Homo sapiens
cgguggaggcggcccaguutt

SEQ ID NO: 1401 - DNA, 21 bp, Homo sapiens
aacugggccgccuccaccgtt

SEQ ID NO: 1402 - DNA, 20 bp, Artificial Sequence
  Synthetic
atgcaggttcagtgccagca

SEQ ID NO: 1403 - DNA, 22 bp, Artificial Sequence
  Synthetic
ttaaaaagatgaatggaataag